(19) United States
(12) Patent Application Publication
(10) Pub. No.: US 2022/0165962 A1
(43) Pub. Date: May 26, 2022

(54) HETEROCYCLIC COMPOUND AND ORGANIC LIGHT-EMITTING DEVICE INCLUDING SAME

(71) Applicant: LT MATERIALS CO., LTD., Yongin-si, Gyeonggi-do (KR)

(72) Inventors: Hye-Su JI, Yongin-si (KR); Gi-Back LEE, Yongin-si (KR); Won-Jang JEONG, Yongin-si (KR); Dong-Jun KIM, Yongin-si (KR)

(73) Assignee: LT MATERIALS CO., LTD., Yongin-si, Gyeonggi-do (KR)

(21) Appl. No.: 17/600,349

(22) PCT Filed: Jul. 28, 2020
(86) PCT No.: PCT/KR2020/009911

§ 371 Date: Sep. 30, 2021

(30) Foreign Application Priority Data
Aug. 6, 2019 (KR) 10-2019-0095682

(51) Int. Cl. H01L 51/00; C07D 401/14; C09K 11/06; C07D 471/04

(52) U.S. Cl. CPC H01L 51/0072; C07D 401/14; C09K 11/06; H01L 51/5278; H01L 51/0052; C07D 471/04; H01L 51/0058; H01L 51/0067

(57) ABSTRACT

The present specification relates to a heterocyclic compound represented by Chemical Formula 1, and an organic light emitting device including the same.

### TECHNICAL FIELD

The present specification relates to a heterocyclic compound, and an organic light emitting device including the same.

The present specification claims priority to and the benefits of Korean Patent Application No. 10-2019-0095682, filed with the Korean Intellectual Property Office on Aug. 6, 2019, the entire contents of which are incorporated herein by reference.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### DISCLOSURE

### Technical Problem

The present specification is directed to providing a heterocyclic compound, and an organic light emitting device including the same.

### Technical Solution

One embodiment of the present specification provides a heterocyclic compound represented by the following Chemical Formula 1.

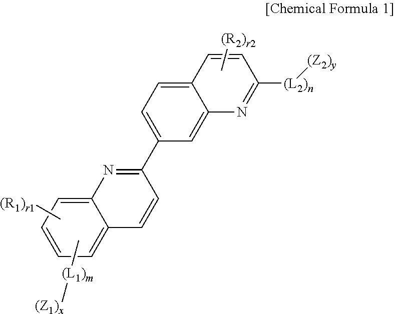

In Chemical Formula 1,

L₁ and L₂ are the same as or different from each other, and each independently a direct bond; a substituted or unsubstituted C6 to C60 arylene group; or a substituted or unsubstituted C2 to C60 heteroarylene group,

Z₁ and Z₂ are the same as or different from each other, and each independently hydrogen; deuterium; a substituted or unsubstituted C6 to C60 aryl group; or a substituted or unsubstituted C2 to C60 heteroaryl group,

R₁ and R₂ are the same as or different from each other, and each independently hydrogen; deuterium; a halogen group; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,

r1 is an integer of 1 to 3,

r2 is 1 or 2,

m, n, x and y are each an integer of 1 to 5,

when r2 is 2, R₂s are the same as or different from each other, and

when r1, m, n, x and y are each 2 or greater, substituents in the parentheses are the same as or different from each other.

Another embodiment of the present application provides an organic light emitting device including a first electrode; a second electrode provided opposite to the first electrode; and an organic material layer provided between the first electrode and the second electrode, wherein the organic material layer includes the heterocyclic compound represented by Chemical Formula 1.

Another embodiment of the present application provides an organic light emitting device including a first electrode; a first stack provided on the first electrode and including a first light emitting layer; a charge generation layer provided on the first stack; a second stack provided on the charge generation layer and including a second light emitting layer; and a second electrode provided on the second stack, wherein the charge generation layer includes the heterocyclic compound represented by Chemical Formula 1.

### Advantageous Effects

A compound described in the present specification can be used as a material of an organic material layer of an organic light emitting device. In the organic light emitting device, the compound is capable of performing a role of a hole injection material, a hole transfer material, a light emitting material, an electron transfer material, an electron injection material or the like. Particularly, the compound can be used as an electron transfer layer material or a charge generation layer material of an organic light emitting device.

Particularly, by Chemical Formula 1 having 2,7′-biquinoline as a central skeleton, a lower driving voltage is obtained than in a device including biquinoline bonding in different forms, light efficiency is enhanced, and device lifetime properties are enhanced by thermal stability.

### DESCRIPTION OF DRAWINGS

FIG. 1 to FIG. 5 each illustrate a lamination structure of an organic light emitting device according to one embodiment of the present specification.

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
307: Charge Generation Layer
400: Cathode

### MODE FOR DISCLOSURE

Hereinafter, the present specification will be described in more detail.

In the present specification, a certain part “including” certain constituents means capable of further including other constituents, and does not exclude other constituents unless particularly stated on the contrary.

In the present specification, the term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; and an amine group, or being unsubstituted, or being substituted with a substituent linking two or more substituents selected from among the substituents illustrated above, or being unsubstituted, and R, R′ and R″ are the same as or different from each other, and each independently hydrogen; deuterium; a cyano group; a C1 to C60 alkyl group; a C3 to C60 cycloalkyl group; a C6 to C60 aryl group; or a C2 to C60 heteroaryl group.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring thereof, and the like, but are not limited thereto.

In the present specification, the fluorenyl group may be substituted, and adjacent substituents may bond to each other to form a ring.

When the fluorenyl group is substituted,

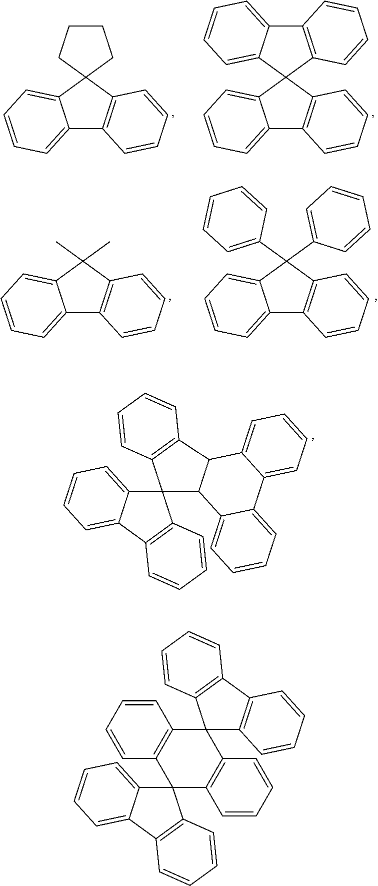

and the like may be included, however, the structure is not limited thereto.

In the present specification, the heteroaryl group includes O, S, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrazinyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the examples of the aryl group and the heteroaryl group described above may be applied to the arylene group and the heteroarylene group except that they are a divalent group.

One embodiment of the present specification provides a heterocyclic compound represented by Chemical Formula 1.

In one embodiment of the present specification, Chemical Formula 1 may be represented by any one of the following Chemical Formulae 2 to 5.

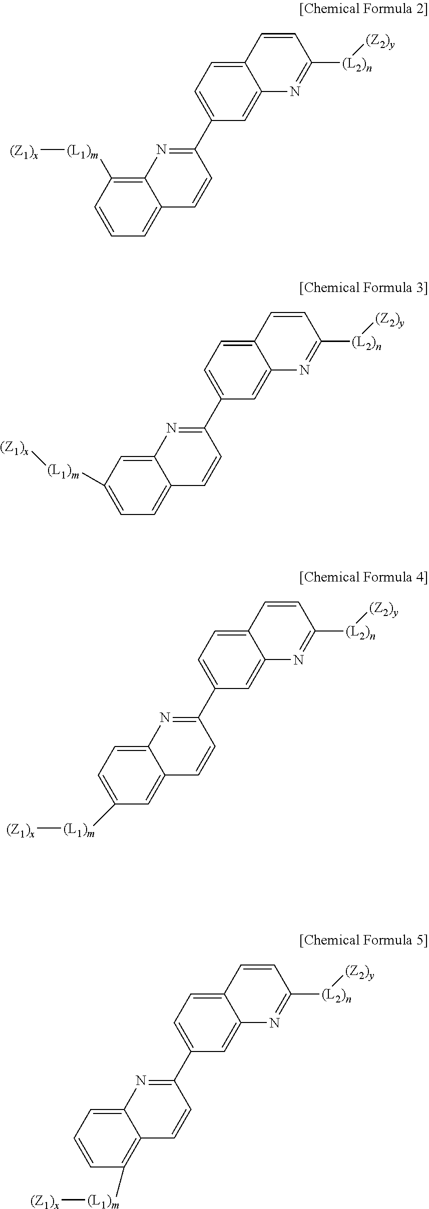

In Chemical Formulae 2 to 5,

each substituent has the same definition as in Chemical Formula 1.

In one embodiment of the present specification, L₁ and L₂ are the same as or different from each other, and each independently a direct bond; a substituted or unsubstituted C6 to C60 arylene group; or a substituted or unsubstituted C2 to C60 heteroarylene group.

In one embodiment of the present specification, L₁ and L₂ are the same as or different from each other, and each independently a direct bond; a substituted or unsubstituted C6 to C30 arylene group; or a substituted or unsubstituted C2 to C30 heteroarylene group.

In one embodiment of the present specification, L₁ is a direct bond; a substituted or unsubstituted phenylene group; a substituted or unsubstituted biphenylene group; a substituted or unsubstituted naphthylene group; a substituted or unsubstituted phenanthrenylene group; a substituted or unsubstituted pyrenylene group; a substituted or unsubstituted triphenylenylene group; a substituted or unsubstituted divalent pyridine group; a substituted or unsubstituted divalent pyrimidine group; or a substituted or unsubstituted divalent triazine group.

In one embodiment of the present specification, L₁ is a direct bond; a phenylene group unsubstituted or substituted with an aryl group or a heteroaryl group; a biphenylene group; a naphthylene group; a phenanthrenylene group; a pyrenylene group; a triphenylenylene group; a divalent pyridine group unsubstituted or substituted with an aryl group; a divalent pyrimidine group unsubstituted or substituted with an aryl group; or a divalent triazine group unsubstituted or substituted with an aryl group.

In one embodiment of the present specification, L₁ is a direct bond; a phenylene group unsubstituted or substituted with one or more substituents selected from the group consisting of a phenyl group, a pyridine group, a quinolinyl group and a phenanthrolinyl group; a biphenylene group; a naphthylene group; a phenanthrenylene group; a pyrenylene group; a triphenylenylene group; a divalent pyridine group unsubstituted or substituted with a phenyl group; a divalent pyrimidine group unsubstituted or substituted with a phenyl group; or a divalent triazine group unsubstituted or substituted with a phenyl group.

In one embodiment of the present specification, L₂ is a direct bond; or a substituted or unsubstituted C6 to C30 arylene group.

In one embodiment of the present specification, L₂ is a direct bond; a substituted or unsubstituted phenylene group; a substituted or unsubstituted naphthylene group; or a substituted or unsubstituted anthracenylene group.

In one embodiment of the present specification, L₂ is a direct bond; a phenylene group; a naphthylene group; or an anthracenylene group.

In one embodiment of the present specification, Z₁ is hydrogen; deuterium; a substituted or unsubstituted C6 to C60 aryl group; or a substituted or unsubstituted C2 to C60 heteroaryl group.

In one embodiment of the present specification, Z₁ is hydrogen; deuterium; a substituted or unsubstituted C6 to C30 aryl group; or a substituted or unsubstituted C2 to C30 heteroaryl group.

In one embodiment of the present specification, Z₁ is hydrogen; deuterium; a substituted or unsubstituted phenyl group; a substituted or unsubstituted biphenyl group; a substituted or unsubstituted naphthyl group; a substituted or unsubstituted phenanthrenyl group; a substituted or unsubstituted pyrenyl group; a substituted or unsubstituted triphenylenyl group; a substituted or unsubstituted pyridine group; a substituted or unsubstituted pyrimidine group; a substituted or unsubstituted triazine group; a substituted or unsubstituted benzimidazole group; a substituted or unsubstituted carbazole group; a substituted or unsubstituted quinoline group; or a substituted or unsubstituted phenanthroline group.

In one embodiment of the present specification, Z₁ is hydrogen; deuterium; a phenyl group unsubstituted or substituted with an aryl group or a heteroaryl group; a biphenyl group; a naphthyl group; a phenanthrenyl group; a pyrenyl group; a triphenylenyl group; a pyridine group unsubstituted or substituted with an aryl group; a pyrimidine group unsubstituted or substituted with an aryl group; a triazine group unsubstituted or substituted with an aryl group; a benzimidazole group unsubstituted or substituted with an aryl group; a carbazole group unsubstituted or substituted with an aryl group; a quinoline group; or a phenanthroline group unsubstituted or substituted with an aryl group.

In one embodiment of the present specification, Z₁ is hydrogen; deuterium; a phenyl group unsubstituted or substituted with one or more substituents selected from the group consisting of a phenyl group, a pyridine group, a quinolinyl group and a phenanthrolinyl group; a biphenyl group; a naphthyl group; a phenanthrenyl group; a pyrenyl group; a triphenylenyl group; a pyridine group unsubstituted or substituted with a phenyl group; a pyrimidine group unsubstituted or substituted with a phenyl group; a triazine group unsubstituted or substituted with a phenyl group; a benzimidazole group unsubstituted or substituted with a phenyl group; a carbazole group unsubstituted or substituted with a phenyl group; a quinoline group; or a phenanthroline group unsubstituted or substituted with a phenyl group or a naphthyl group.

In one embodiment of the present specification, Z₂ is a substituted or unsubstituted C6 to C60 aryl group; or a substituted or unsubstituted C2 to C60 heteroaryl group.

In one embodiment of the present specification, Z₂ is a substituted or unsubstituted C2 to C60 heteroaryl group.

In one embodiment of the present specification, Z₂ is a substituted or unsubstituted C2 to C30 heteroaryl group.

In one embodiment of the present specification, Z₂ is a substituted or unsubstituted C2 to C30 heteroaryl group including at least one N.

In one embodiment of the present specification, Z₂ is a substituted or unsubstituted pyridine group; a substituted or unsubstituted pyrimidine group; a substituted or unsubstituted pyrazine group; a substituted or unsubstituted triazine group; a substituted or unsubstituted quinoline group; a substituted or unsubstituted quinazoline group; a substituted or unsubstituted benzoquinoline group; or a substituted or unsubstituted phenanthroline group.

In one embodiment of the present specification, Z₂ is a pyridine group; a pyrimidine group unsubstituted or substituted with an aryl group; a pyrazine group; a triazine group unsubstituted or substituted with an aryl group; a quinoline group; a quinazoline group; a benzoquinoline group; or a phenanthroline group unsubstituted or substituted with an aryl group.

In one embodiment of the present specification, Z₂ is a pyridine group; a pyrimidine group unsubstituted or substituted with a phenyl group or a pyridine group; a pyrazine group; a triazine group unsubstituted or substituted with a phenyl group; a quinoline group; a quinazoline group; a benzoquinoline group; or a phenanthroline group unsubstituted or substituted with a phenyl group or a naphthyl group.

In one embodiment of the present specification, L₁ is a direct bond, and when Z₁ is hydrogen, L₂ is a direct bond; or a substituted or unsubstituted C6 to C30 arylene group, and Z₂ is a C2 to C30 heteroaryl group unsubstituted or substituted with an aryl group.

In one embodiment of the present specification, R₁ and R₂ are the same as or different from each other, and each independently hydrogen; deuterium; a halogen group; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group.

In one embodiment of the present specification, R₁ and R₂ are the same as or different from each other, and each independently hydrogen; deuterium; a substituted or unsubstituted C1 to C30 alkyl group; a substituted or unsubstituted C6 to C30 aryl group; or a substituted or unsubstituted C2 to C30 heteroaryl group.

In one embodiment of the present specification, R₁ and R₂ are the same as or different from each other, and each independently hydrogen; or deuterium.

In one embodiment of the present specification, R₁ and R₂ are hydrogen.

In one embodiment of the present specification, Chemical Formula 1 may be represented by any one of the following compounds, but is not limited thereto.

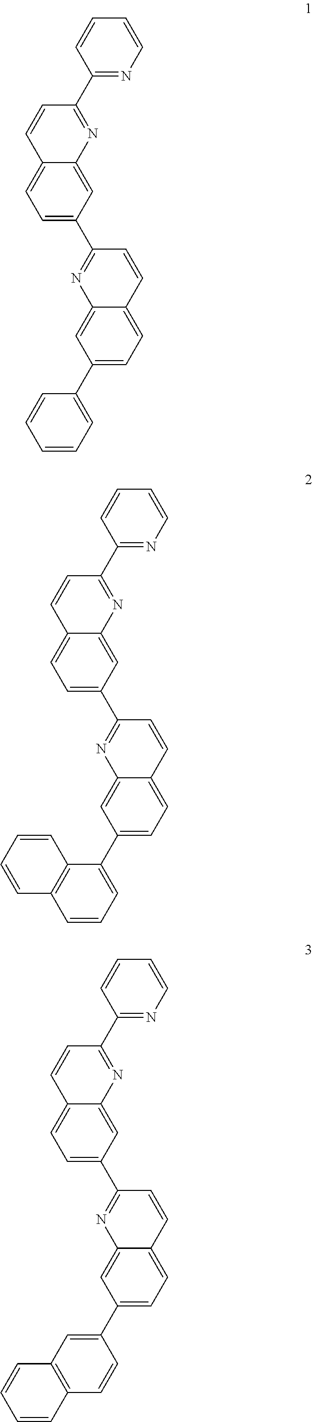

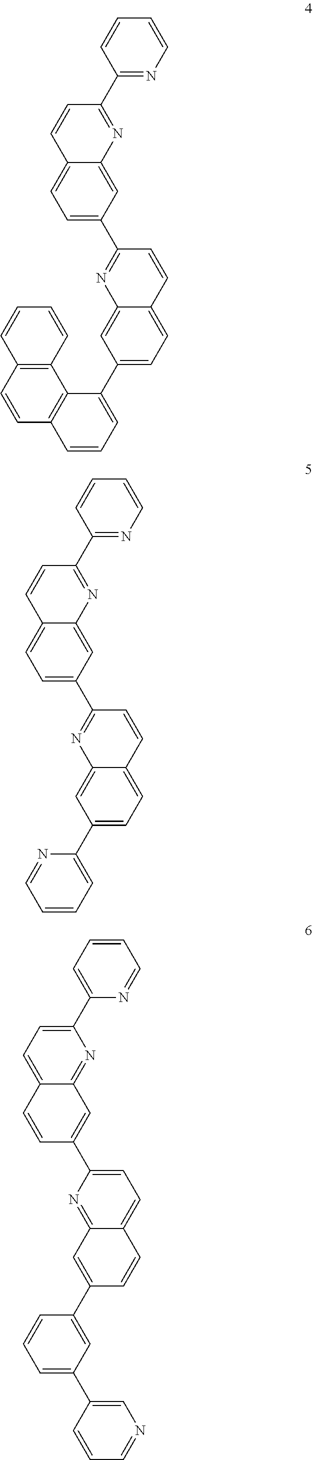

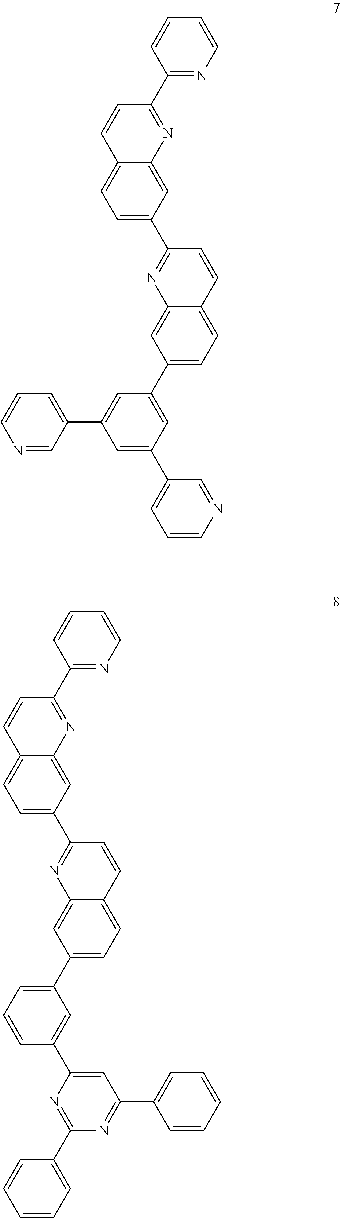 [SMILES: c1ccc(-c2ccc3ccc(-c4ccc5ccc(-c6cc(-c7cccnc7)cc(-c7cccnc7)c6)cc5n4)cc3n2)nc1 ; c1ccc(-c2cc(-c3cccc(-c4ccc5ccc(-c6ccc7ccc(-c8ccccn8)nc7c6)nc5c4)c3)nc(-c3ccccc3)n2)cc1]

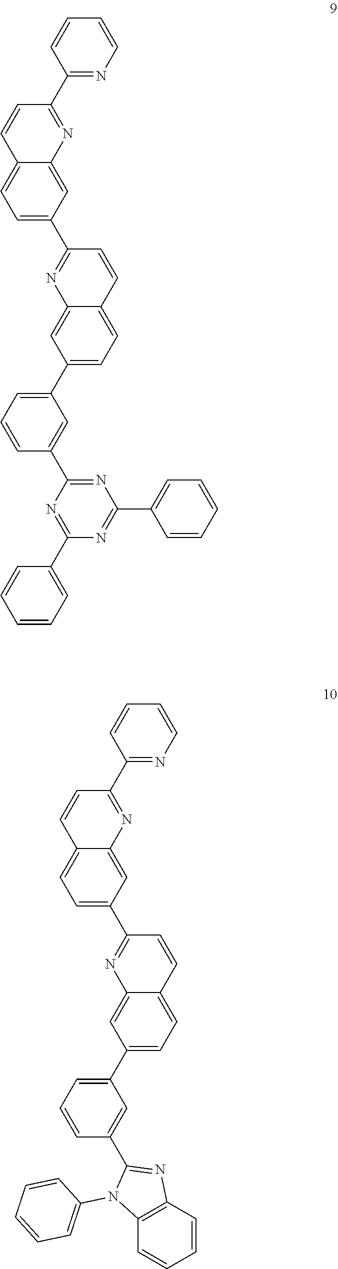 [SMILES: c1ccc(-c2nc(-c3ccccc3)nc(-c3cccc(-c4ccc5ccc(-c6ccc7ccc(-c8ccccn8)nc7c6)nc5c4)c3)n2)cc1 ; c1ccc(-n2c(-c3cccc(-c4ccc5ccc(-c6ccc7ccc(-c8ccccn8)nc7c6)nc5c4)c3)nc3ccccc32)cc1]

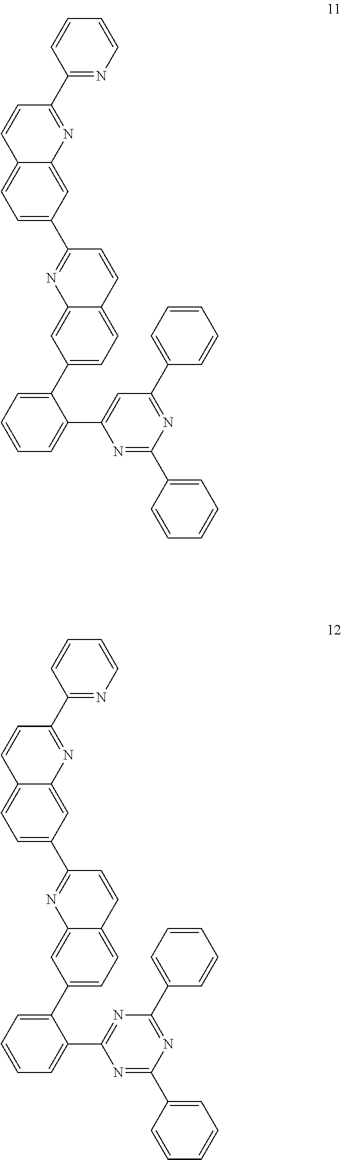 [SMILES: c1ccc(-c2cc(-c3ccccc3-c3ccc4ccc(-c5ccc6ccc(-c7ccccn7)nc6c5)nc4c3)nc(-c3ccccc3)n2)cc1 ; c1ccc(-c2nc(-c3ccccc3)nc(-c3ccccc3-c3ccc4ccc(-c5ccc6ccc(-c7ccccn7)nc6c5)nc4c3)n2)cc1]

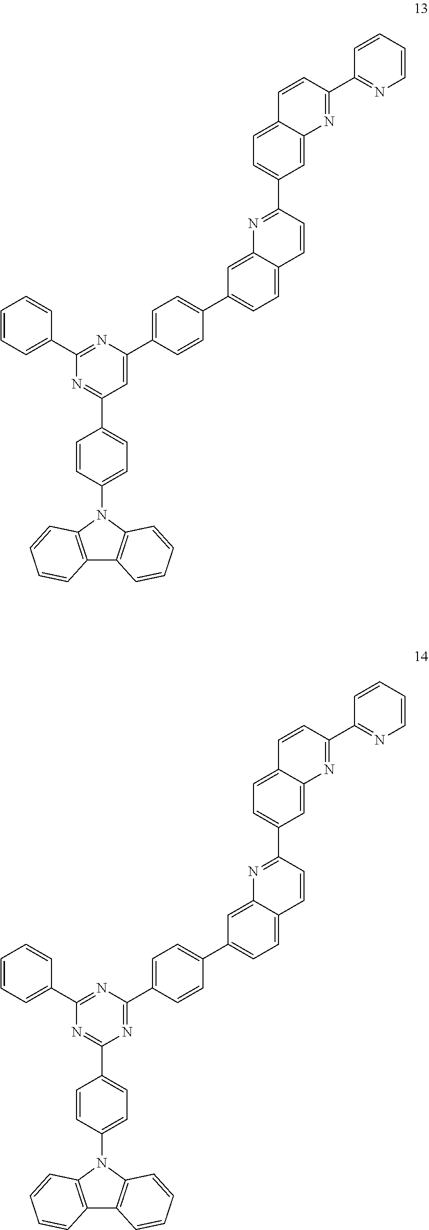 [SMILES: c1ccc(-c2nc(-c3ccc(-c4ccc5ccc(-c6ccc7ccc(-c8ccccn8)nc7c6)nc5c4)cc3)cc(-c3ccc(-n4c5ccccc5c5ccccc54)cc3)n2)cc1 ; c1ccc(-c2nc(-c3ccc(-c4ccc5ccc(-c6ccc7ccc(-c8ccccn8)nc7c6)nc5c4)cc3)nc(-c3ccc(-n4c5ccccc5c5ccccc54)cc3)n2)cc1]

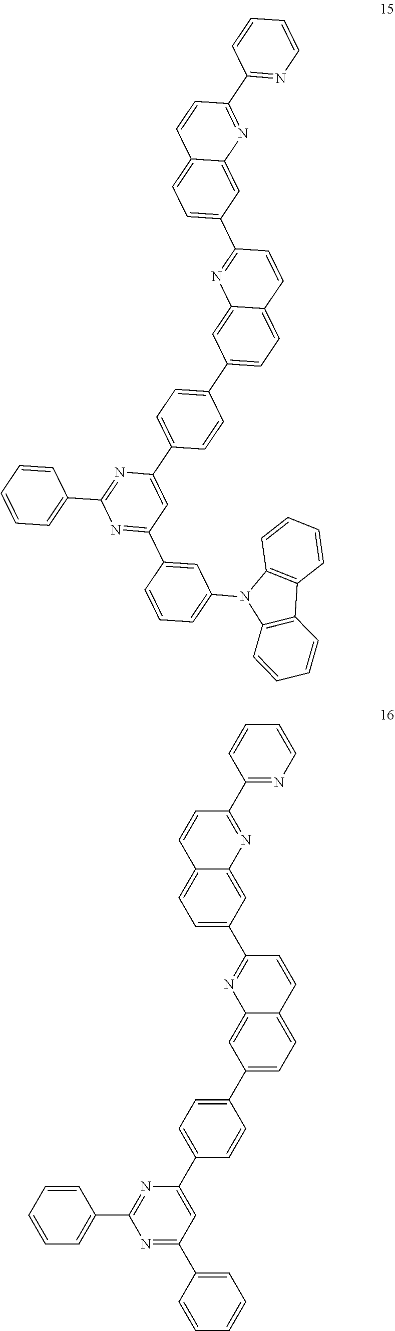

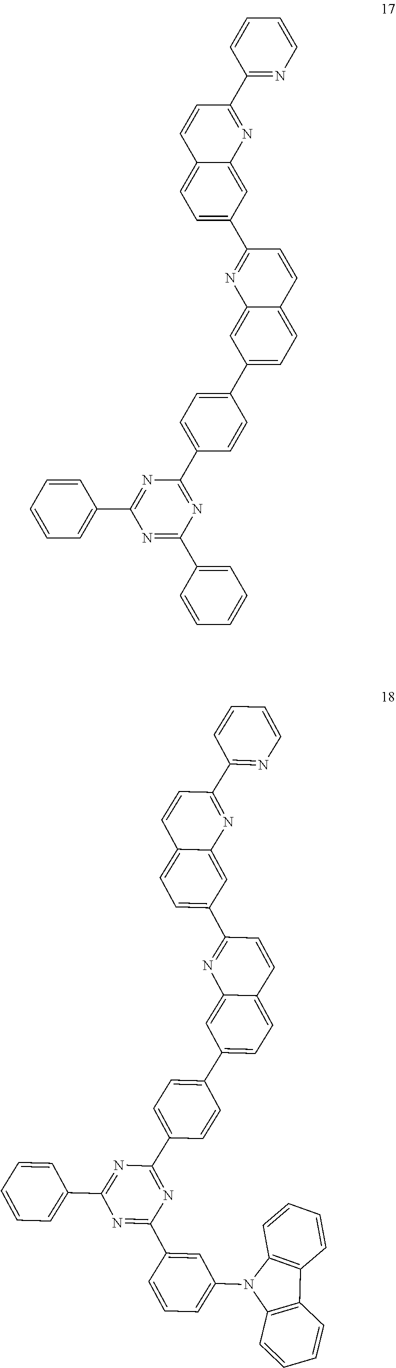 [SMILES: c1ccc(-c2nc(-c3ccccc3)nc(-c3ccc(-c4ccc5ccc(-c6ccc7ccc(-c8ccccn8)nc7c6)nc5c4)cc3)n2)cc1 ; c1ccc(-c2nc(-c3ccc(-c4ccc5ccc(-c6ccc7ccc(-c8ccccn8)nc7c6)nc5c4)cc3)nc(-c3cccc(-n4c5ccccc5c5ccccc54)c3)n2)cc1]

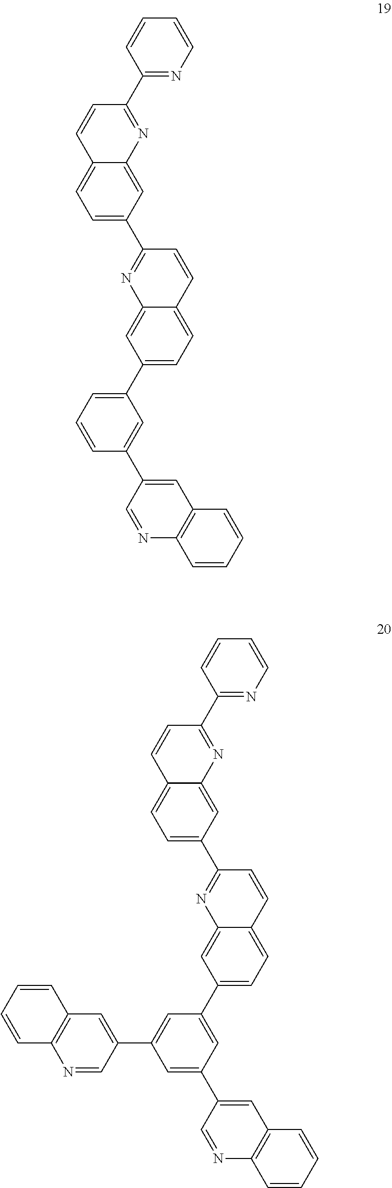 [SMILES: c1ccc(-c2ccc3ccc(-c4ccc5ccc(-c6cccc(-c7cnc8ccccc8c7)c6)cc5n4)cc3n2)nc1 ; c1ccc(-c2ccc3ccc(-c4ccc5ccc(-c6cc(-c7cnc8ccccc8c7)cc(-c7cnc8ccccc8c7)c6)cc5n4)cc3n2)nc1]

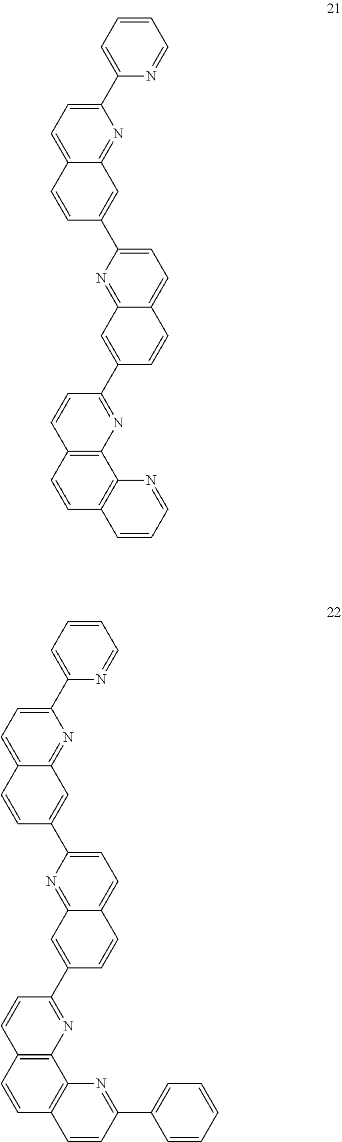 [SMILES: c1ccc(-c2ccc3ccc(-c4ccc5ccc(-c6ccc7ccc8cccnc8c7n6)cc5n4)cc3n2)nc1 ; c1ccc(-c2ccc3ccc4ccc(-c5ccc6ccc(-c7ccc8ccc(-c9ccccn9)nc8c7)nc6c5)nc4c3n2)cc1]

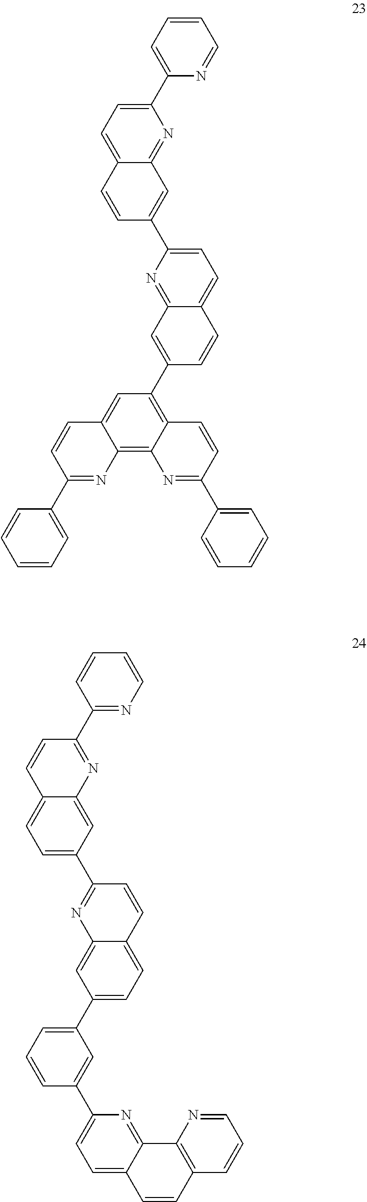 [SMILES: c1ccc(-c2ccc3cc(-c4ccc5ccc(-c6ccc7ccc(-c8ccccn8)nc7c6)nc5c4)c4ccc(-c5ccccc5)nc4c3n2)cc1 ; c1ccc(-c2ccc3ccc(-c4ccc5ccc(-c6cccc(-c7ccc8ccc9cccnc9c8n7)c6)cc5n4)cc3n2)nc1]

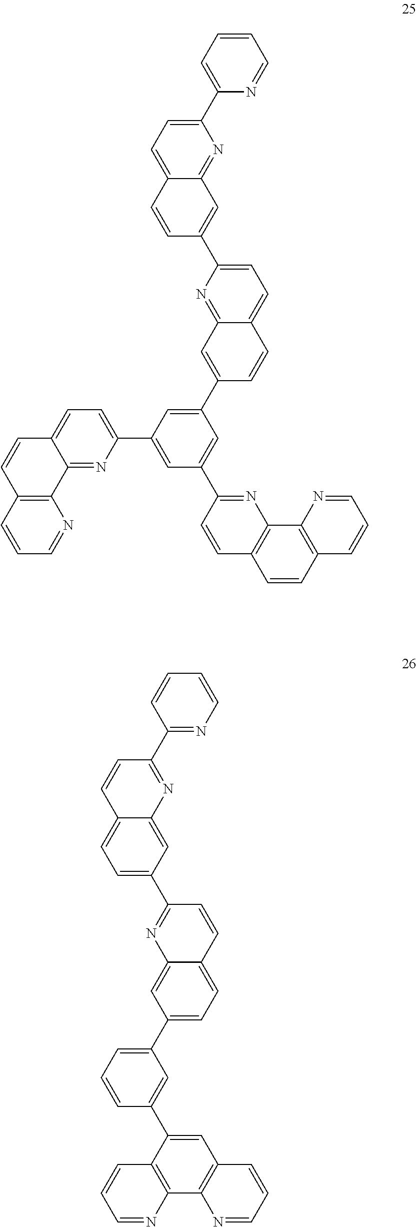 [SMILES: c1ccc(-c2ccc3ccc(-c4ccc5ccc(-c6cc(-c7ccc8ccc9cccnc9c8n7)cc(-c7ccc8ccc9cccnc9c8n7)c6)cc5n4)cc3n2)nc1 ; c1ccc(-c2ccc3ccc(-c4ccc5ccc(-c6cccc(-c7cc8cccnc8c8ncccc78)c6)cc5n4)cc3n2)nc1]

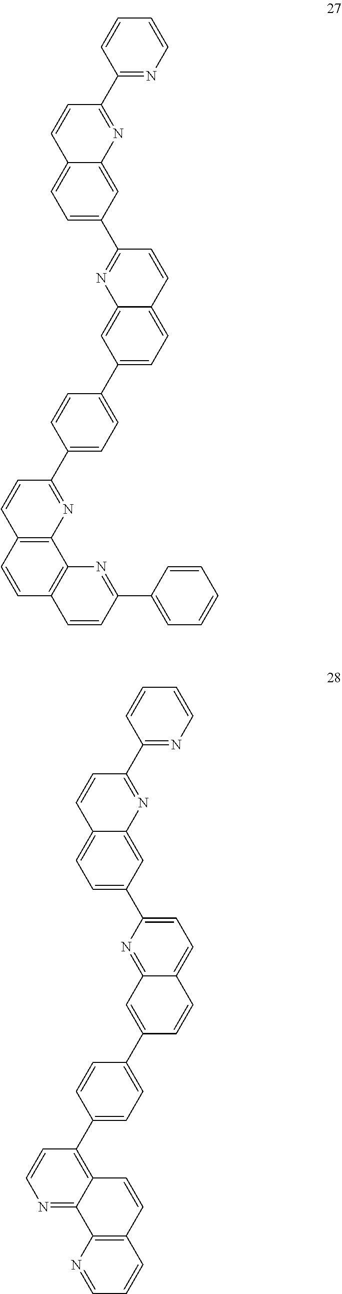 [SMILES: c1ccc(-c2ccc3ccc4ccc(-c5ccc(-c6ccc7ccc(-c8ccc9ccc(-c%10ccccn%10)nc9c8)nc7c6)cc5)nc4c3n2)cc1 ; c1ccc(-c2ccc3ccc(-c4ccc5ccc(-c6ccc(-c7ccnc8c7ccc7cccnc78)cc6)cc5n4)cc3n2)nc1]

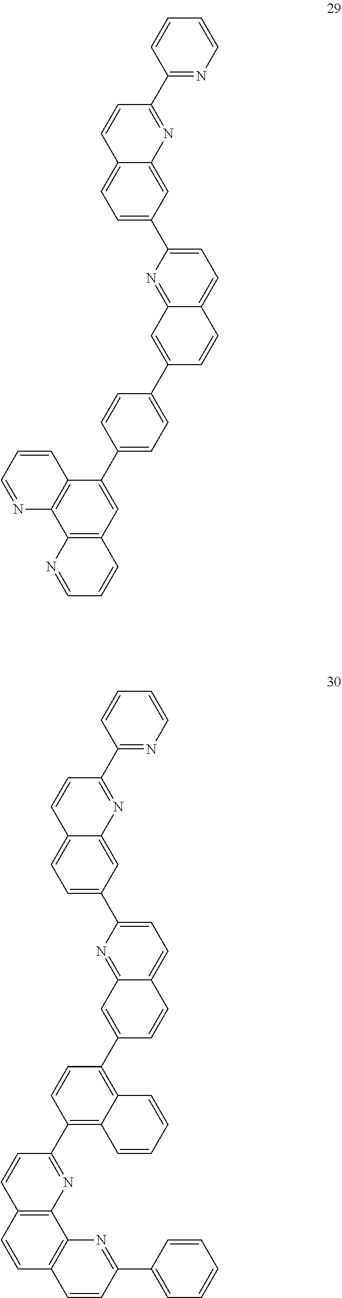 [SMILES: c1ccc(-c2ccc3ccc(-c4ccc5ccc(-c6ccc(-c7cc8cccnc8c8ncccc78)cc6)cc5n4)cc3n2)nc1 ; c1ccc(-c2ccc3ccc4ccc(-c5ccc(-c6ccc7ccc(-c8ccc9ccc(-c%10ccccn%10)nc9c8)nc7c6)c6ccccc56)nc4c3n2)cc1]

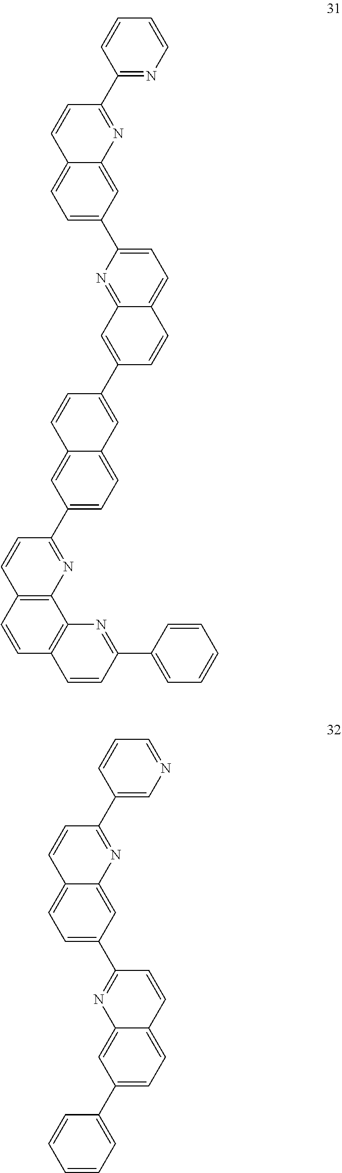 [SMILES: c1ccc(-c2ccc3ccc4ccc(-c5ccc6cc(-c7ccc8ccc(-c9ccc%10ccc(-c%11ccccn%11)nc%10c9)nc8c7)ccc6c5)nc4c3n2)cc1 ; c1ccc(-c2ccc3ccc(-c4ccc5ccc(-c6cccnc6)nc5c4)nc3c2)cc1]

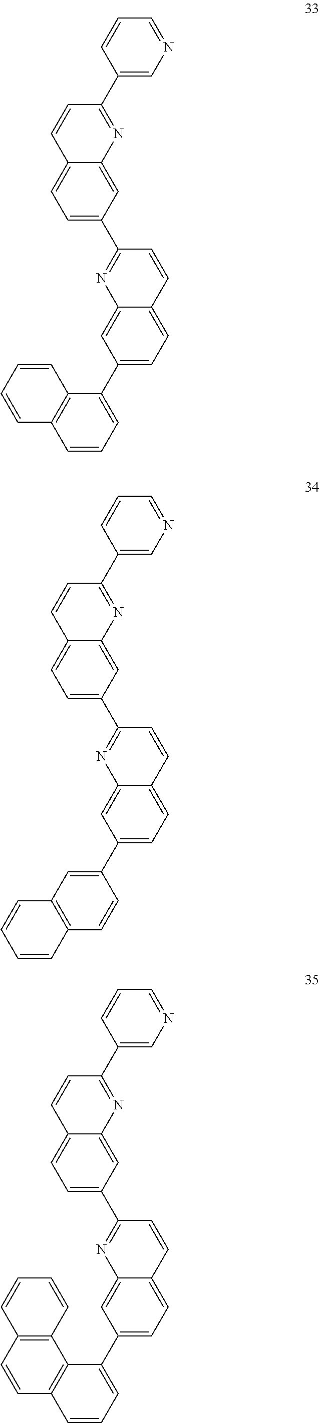 [SMILES: c1cncc(-c2ccc3ccc(-c4ccc5ccc(-c6cccc7ccccc67)cc5n4)cc3n2)c1 ; c1cncc(-c2ccc3ccc(-c4ccc5ccc(-c6ccc7ccccc7c6)cc5n4)cc3n2)c1 ; c1cncc(-c2ccc3ccc(-c4ccc5ccc(-c6cccc7ccc8ccccc8c67)cc5n4)cc3n2)c1]

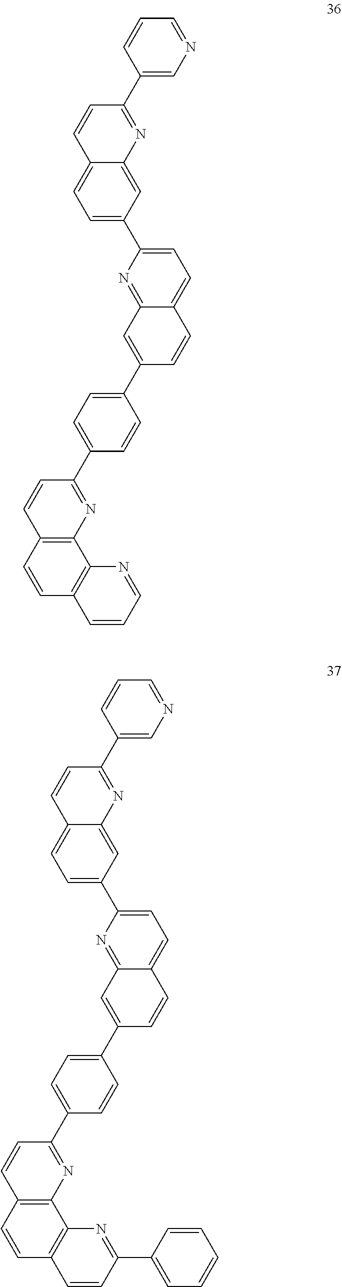 [SMILES: c1cncc(-c2ccc3ccc(-c4ccc5ccc(-c6ccc(-c7ccc8ccc9cccnc9c8n7)cc6)cc5n4)cc3n2)c1 ; c1ccc(-c2ccc3ccc4ccc(-c5ccc(-c6ccc7ccc(-c8ccc9ccc(-c%10cccnc%10)nc9c8)nc7c6)cc5)nc4c3n2)cc1]

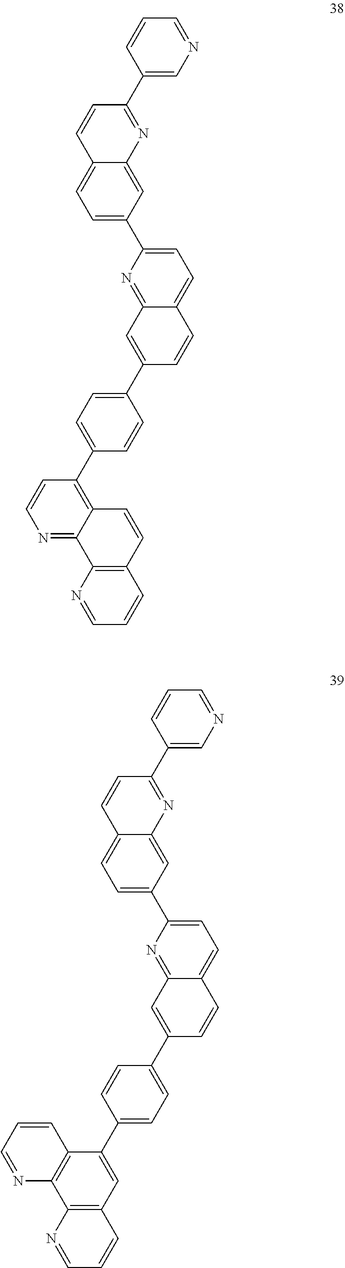 [SMILES: c1cncc(-c2ccc3ccc(-c4ccc5ccc(-c6ccc(-c7ccnc8c7ccc7cccnc78)cc6)cc5n4)cc3n2)c1 ; c1cncc(-c2ccc3ccc(-c4ccc5ccc(-c6ccc(-c7cc8cccnc8c8ncccc78)cc6)cc5n4)cc3n2)c1]

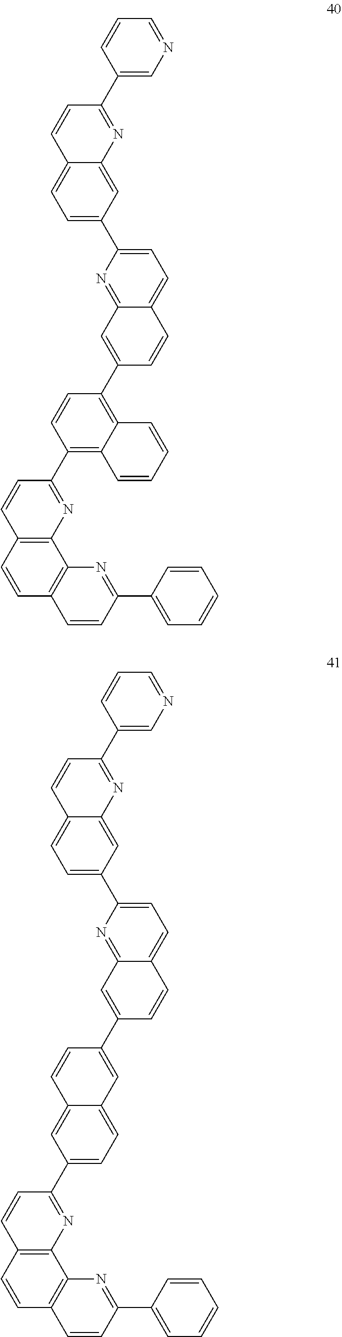 [SMILES: c1ccc(-c2ccc3ccc4ccc(-c5ccc(-c6ccc7ccc(-c8ccc9ccc(-c%10cccnc%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11cccnc%11)nc%10c9)nc8c7)ccc6c5)nc4c3n2)cc1]

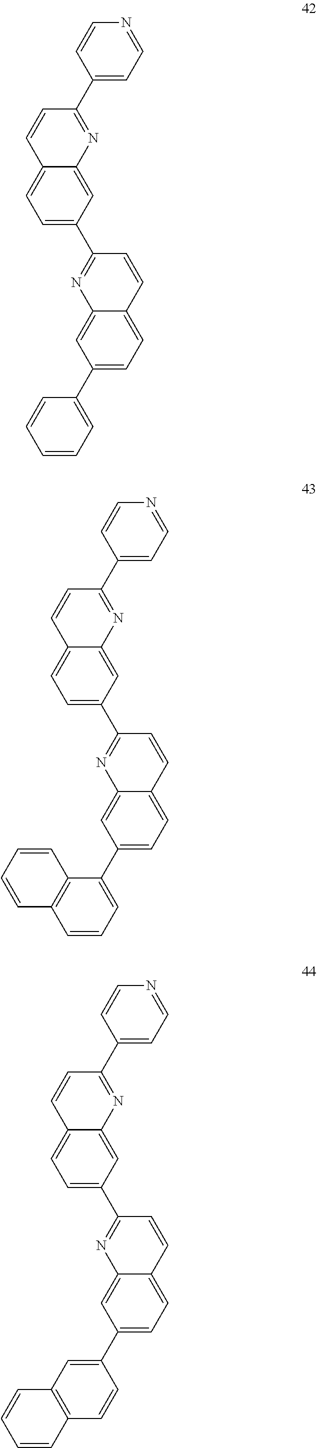 [SMILES: c1ccc(-c2ccc3ccc(-c4ccc5ccc(-c6ccncc6)nc5c4)nc3c2)cc1 ; c1ccc2c(-c3ccc4ccc(-c5ccc6ccc(-c7ccncc7)nc6c5)nc4c3)cccc2c1 ; c1ccc2cc(-c3ccc4ccc(-c5ccc6ccc(-c7ccncc7)nc6c5)nc4c3)ccc2c1]

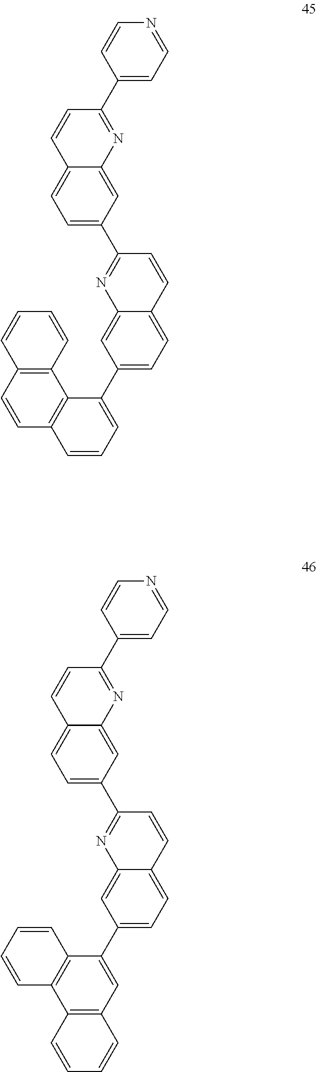 [SMILES: c1ccc2c(c1)ccc1cccc(-c3ccc4ccc(-c5ccc6ccc(-c7ccncc7)nc6c5)nc4c3)c12 ; c1ccc2c(c1)cc(-c1ccc3ccc(-c4ccc5ccc(-c6ccncc6)nc5c4)nc3c1)c1ccccc12]

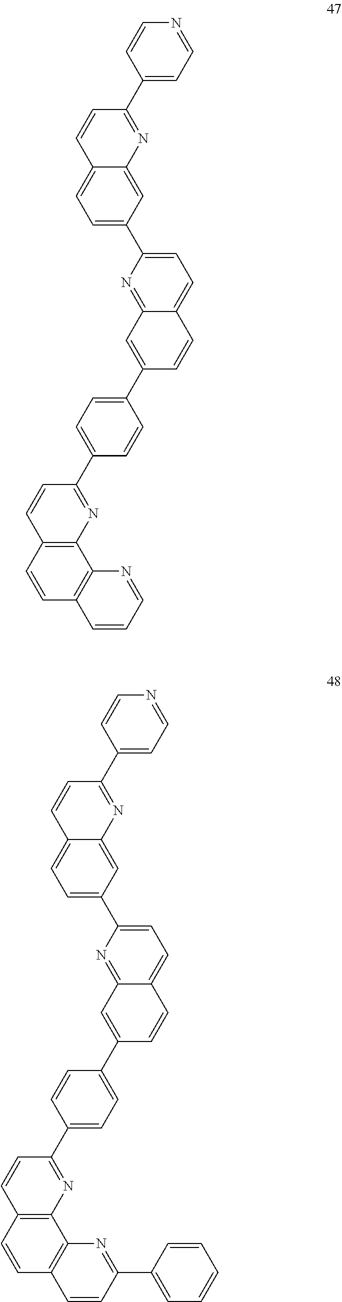 [SMILES: c1cnc2c(c1)ccc1ccc(-c3ccc(-c4ccc5ccc(-c6ccc7ccc(-c8ccncc8)nc7c6)nc5c4)cc3)nc12 ; c1ccc(-c2ccc3ccc4ccc(-c5ccc(-c6ccc7ccc(-c8ccc9ccc(-c%10ccncc%10)nc9c8)nc7c6)cc5)nc4c3n2)cc1]

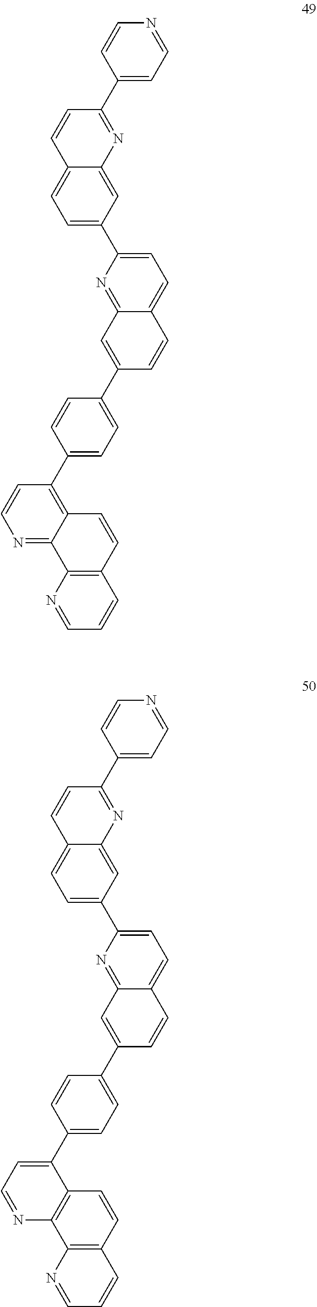 [SMILES: c1cnc2c(c1)ccc1c(-c3ccc(-c4ccc5ccc(-c6ccc7ccc(-c8ccncc8)nc7c6)nc5c4)cc3)ccnc12 ; c1cnc2c(c1)ccc1c(-c3ccc(-c4ccc5ccc(-c6ccc7ccc(-c8ccncc8)nc7c6)nc5c4)cc3)ccnc12]

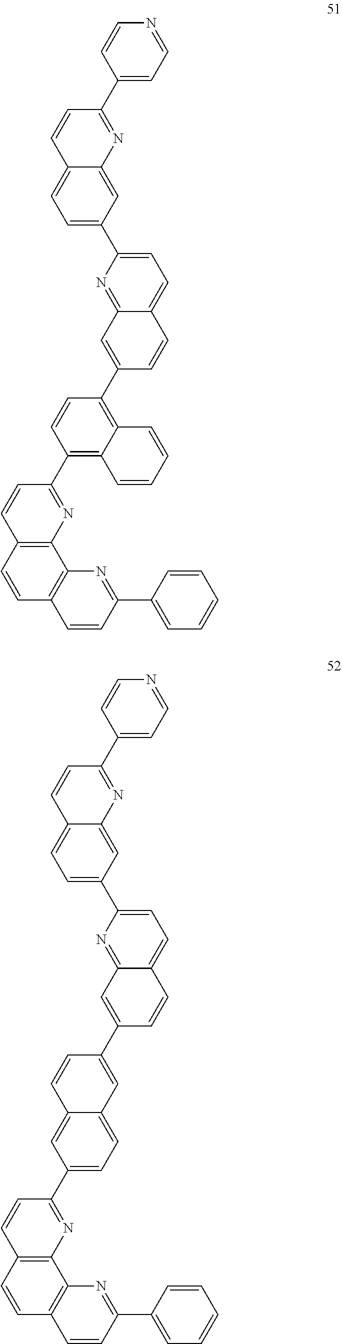 [SMILES: c1ccc(-c2ccc3ccc4ccc(-c5ccc(-c6ccc7ccc(-c8ccc9ccc(-c%10ccncc%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11ccncc%11)nc%10c9)nc8c7)ccc6c5)nc4c3n2)cc1]

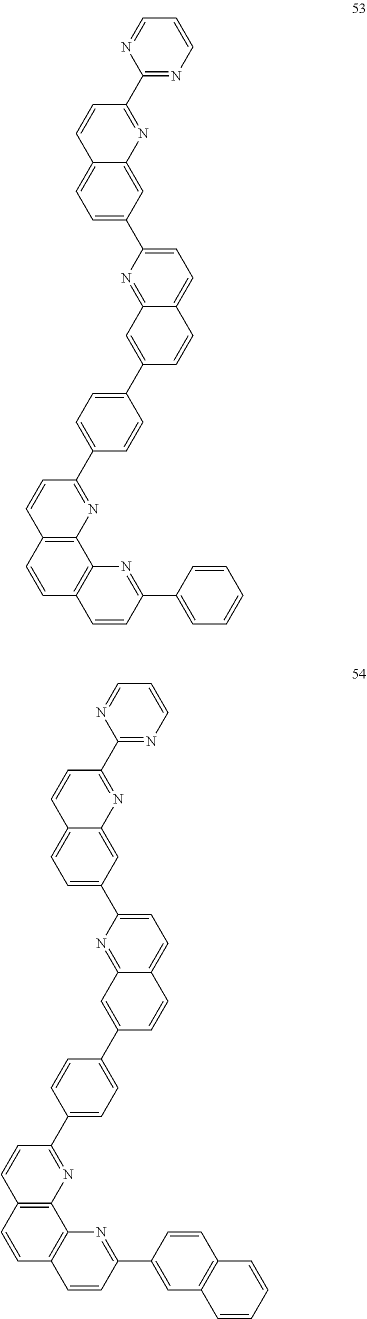 [SMILES: c1ccc(-c2ccc3ccc4ccc(-c5ccc(-c6ccc7ccc(-c8ccc9ccc(-c%10ncccn%10)nc9c8)nc7c6)cc5)nc4c3n2)cc1 ; c1cnc(-c2ccc3ccc(-c4ccc5ccc(-c6ccc(-c7ccc8ccc9ccc(-c%10ccc%11ccccc%11c%10)nc9c8n7)cc6)cc5n4)cc3n2)nc1]

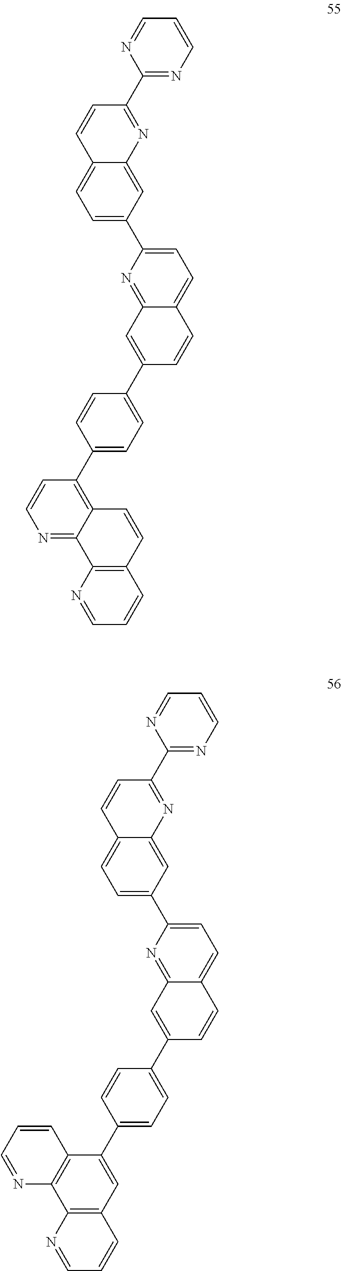 [SMILES: c1cnc(-c2ccc3ccc(-c4ccc5ccc(-c6ccc(-c7ccnc8c7ccc7cccnc78)cc6)cc5n4)cc3n2)nc1 ; c1cnc(-c2ccc3ccc(-c4ccc5ccc(-c6ccc(-c7cc8cccnc8c8ncccc78)cc6)cc5n4)cc3n2)nc1]

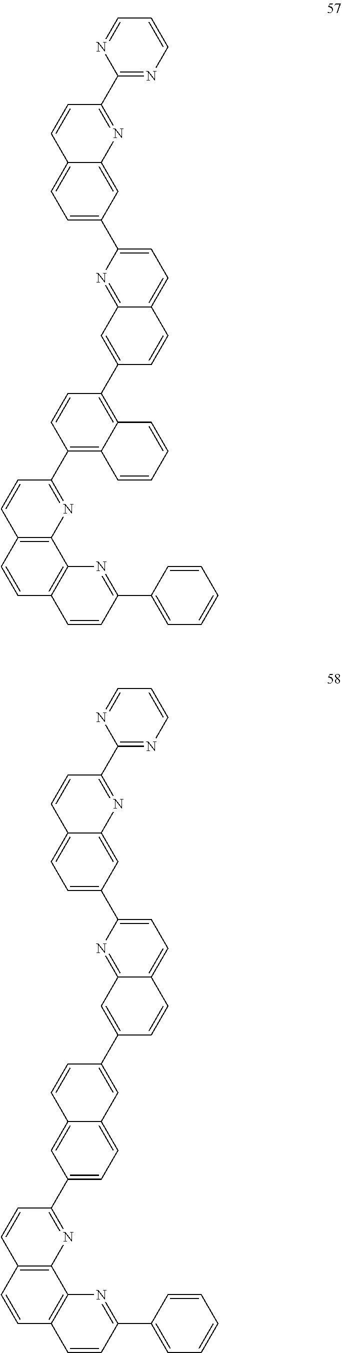 [SMILES: c1ccc(-c2ccc3ccc4ccc(-c5ccc(-c6ccc7ccc(-c8ccc9ccc(-c%10ncccn%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11ncccn%11)nc%10c9)nc8c7)ccc6c5)nc4c3n2)cc1]

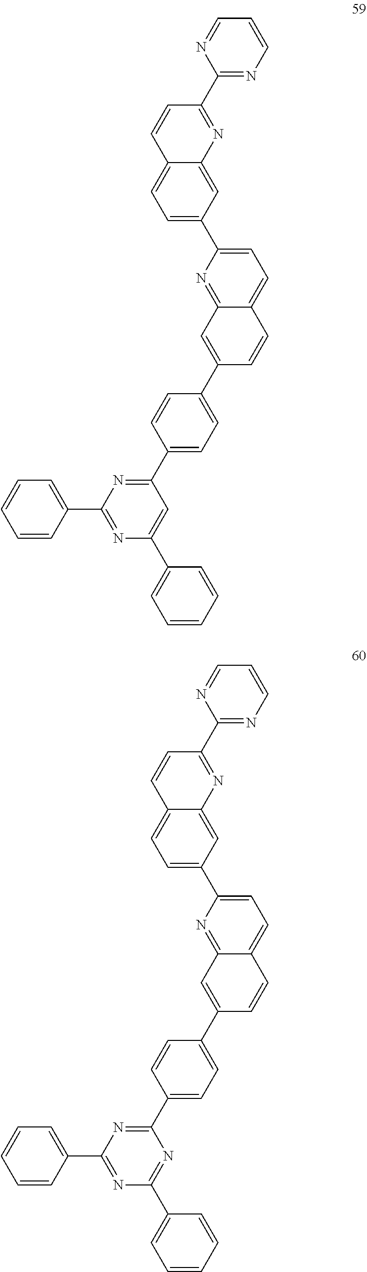

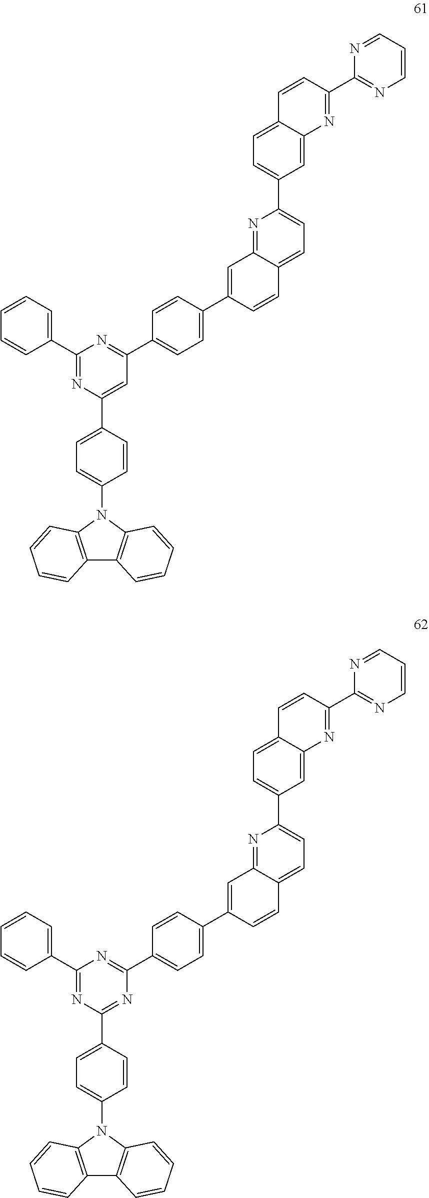 [SMILES: c1ccc(-c2nc(-c3ccc(-c4ccc5ccc(-c6ccc7ccc(-c8ncccn8)nc7c6)nc5c4)cc3)cc(-c3ccc(-n4c5ccccc5c5ccccc54)cc3)n2)cc1 ; c1ccc(-c2nc(-c3ccc(-c4ccc5ccc(-c6ccc7ccc(-c8ncccn8)nc7c6)nc5c4)cc3)nc(-c3ccc(-n4c5ccccc5c5ccccc54)cc3)n2)cc1]

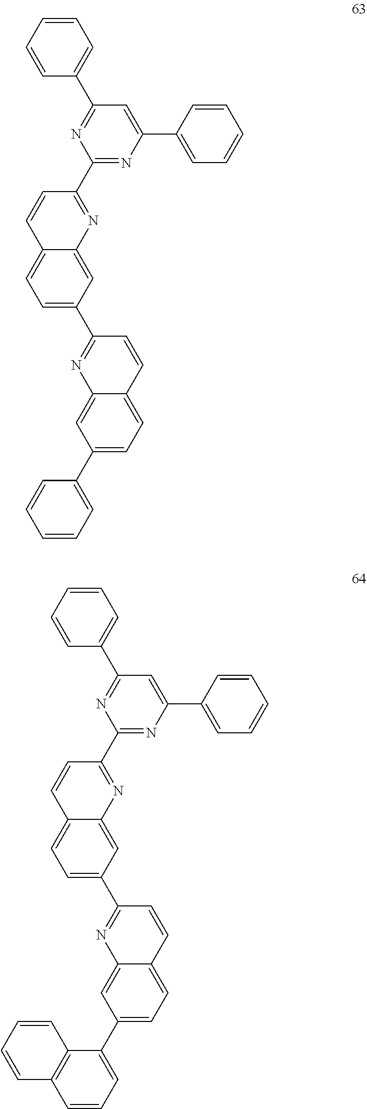 [SMILES: c1ccc(-c2ccc3ccc(-c4ccc5ccc(-c6nc(-c7ccccc7)cc(-c7ccccc7)n6)nc5c4)nc3c2)cc1 ; c1ccc(-c2cc(-c3ccccc3)nc(-c3ccc4ccc(-c5ccc6ccc(-c7cccc8ccccc78)cc6n5)cc4n3)n2)cc1]

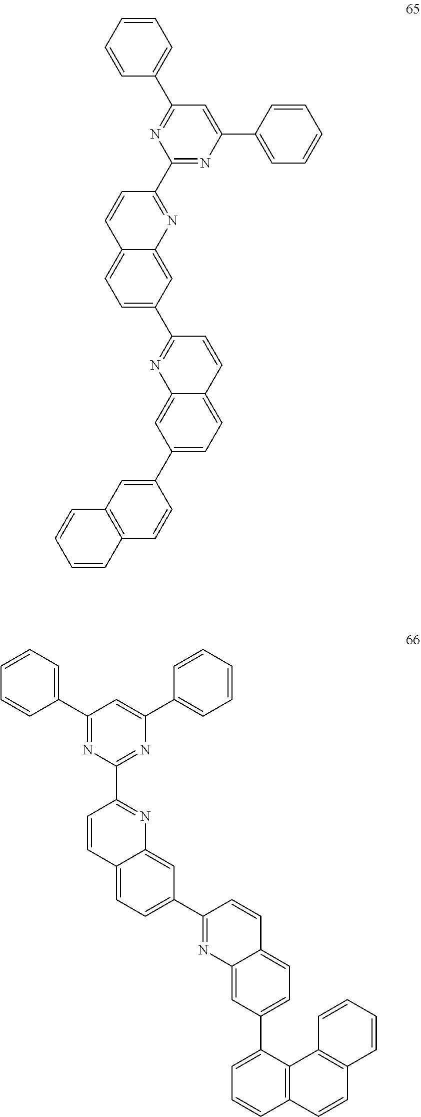 [SMILES: c1ccc(-c2cc(-c3ccccc3)nc(-c3ccc4ccc(-c5ccc6ccc(-c7ccc8ccccc8c7)cc6n5)cc4n3)n2)cc1 ; c1ccc(-c2cc(-c3ccccc3)nc(-c3ccc4ccc(-c5ccc6ccc(-c7cccc8ccc9ccccc9c78)cc6n5)cc4n3)n2)cc1]

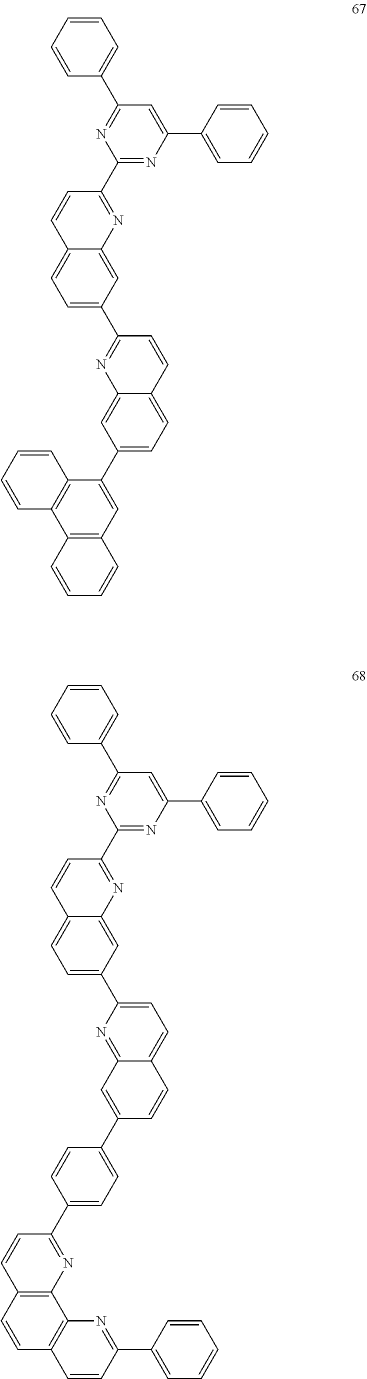 [SMILES: c1ccc(-c2cc(-c3ccccc3)nc(-c3ccc4ccc(-c5ccc6ccc(-c7cc8ccccc8c8ccccc78)cc6n5)cc4n3)n2)cc1 ; c1ccc(-c2cc(-c3ccccc3)nc(-c3ccc4ccc(-c5ccc6ccc(-c7ccc(-c8ccc9ccc%10ccc(-c%11ccccc%11)nc%10c9n8)cc7)cc6n5)cc4n3)n2)cc1]

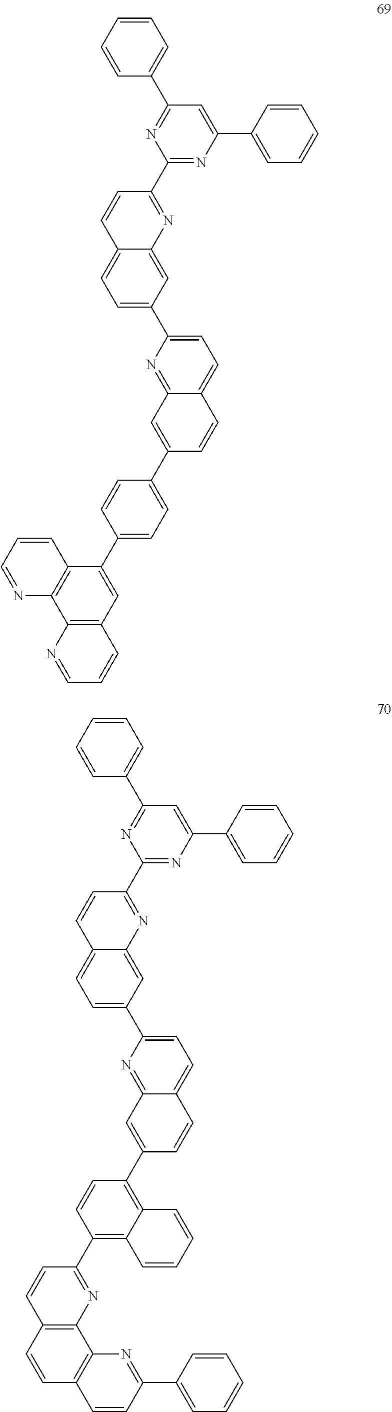

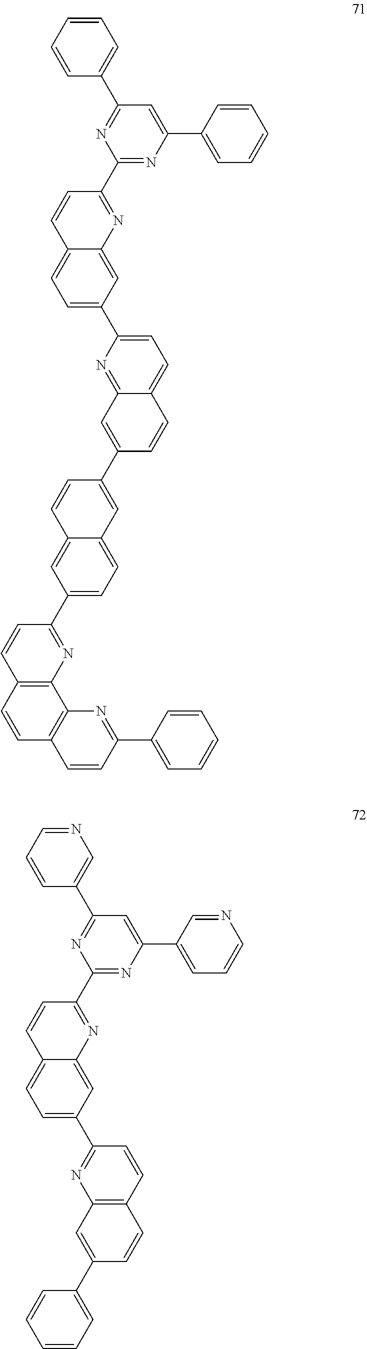 [SMILES: c1ccc(-c2cc(-c3ccccc3)nc(-c3ccc4ccc(-c5ccc6ccc(-c7ccc8cc(-c9ccc%10ccc%11ccc(-c%12ccccc%12)nc%11c%10n9)ccc8c7)cc6n5)cc4n3)n2)cc1 ; c1ccc(-c2ccc3ccc(-c4ccc5ccc(-c6nc(-c7cccnc7)cc(-c7cccnc7)n6)nc5c4)nc3c2)cc1]

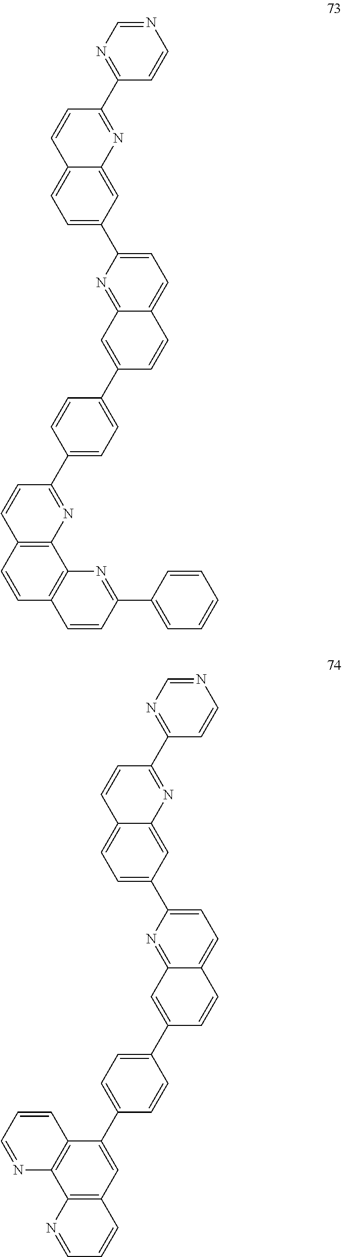 [SMILES: c1ccc(-c2ccc3ccc4ccc(-c5ccc(-c6ccc7ccc(-c8ccc9ccc(-c%10ccncn%10)nc9c8)nc7c6)cc5)nc4c3n2)cc1 ; c1cnc2c(c1)cc(-c1ccc(-c3ccc4ccc(-c5ccc6ccc(-c7ccncn7)nc6c5)nc4c3)cc1)c1cccnc12]

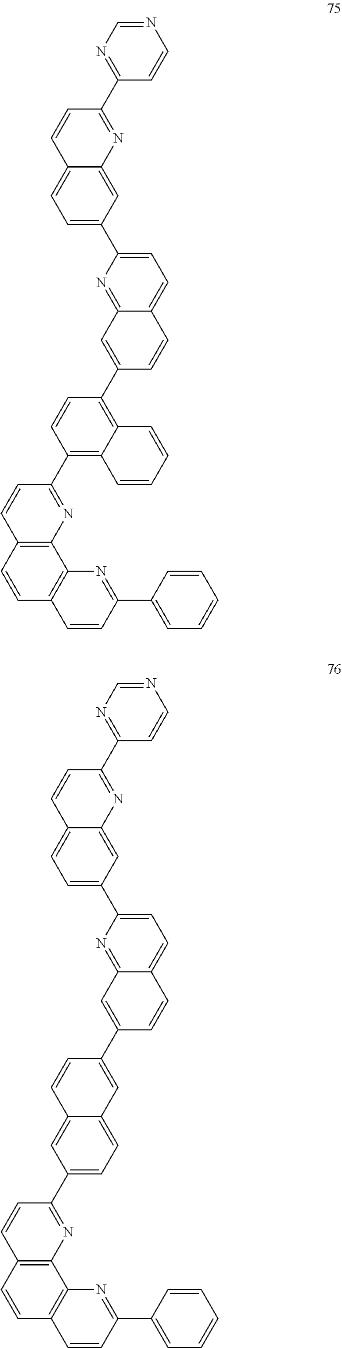 [SMILES: c1ccc(-c2ccc3ccc4ccc(-c5ccc(-c6ccc7ccc(-c8ccc9ccc(-c%10ccncn%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11ccncn%11)nc%10c9)nc8c7)ccc6c5)nc4c3n2)cc1]

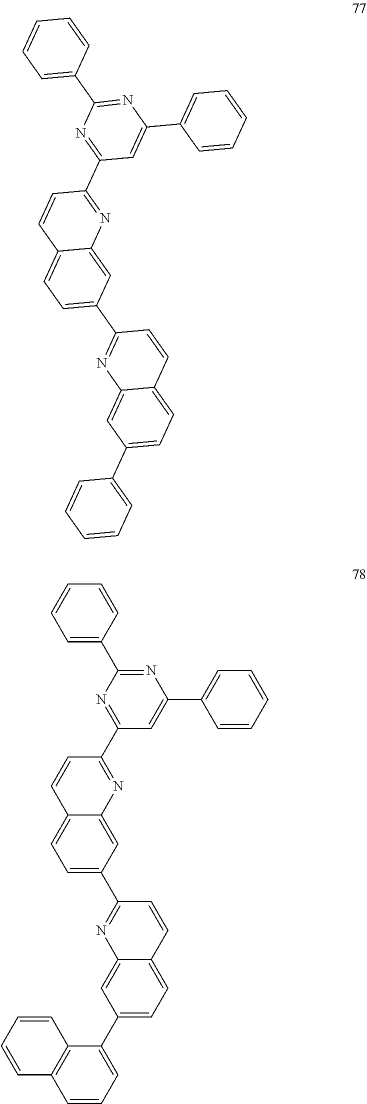 [SMILES: c1ccc(-c2ccc3ccc(-c4ccc5ccc(-c6cc(-c7ccccc7)nc(-c7ccccc7)n6)nc5c4)nc3c2)cc1 ; c1ccc(-c2cc(-c3ccc4ccc(-c5ccc6ccc(-c7cccc8ccccc78)cc6n5)cc4n3)nc(-c3ccccc3)n2)cc1]

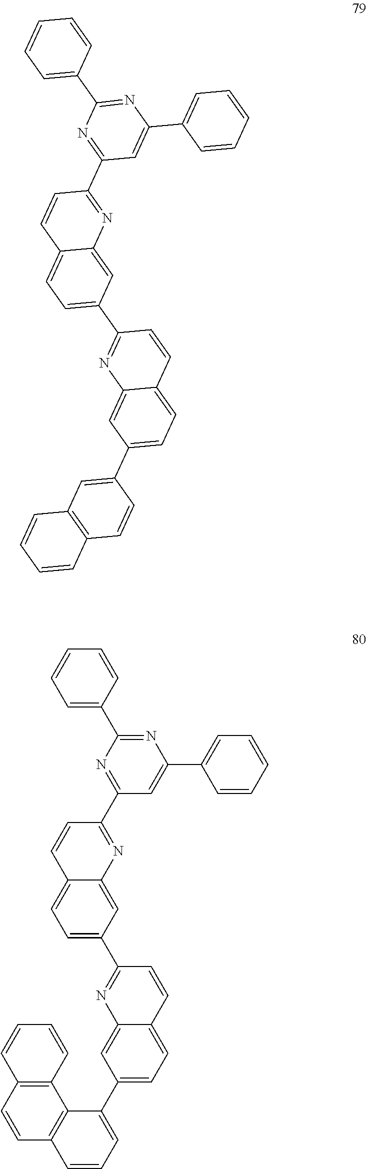 [SMILES: c1ccc(-c2cc(-c3ccc4ccc(-c5ccc6ccc(-c7ccc8ccccc8c7)cc6n5)cc4n3)nc(-c3ccccc3)n2)cc1 ; c1ccc(-c2cc(-c3ccc4ccc(-c5ccc6ccc(-c7cccc8ccc9ccccc9c78)cc6n5)cc4n3)nc(-c3ccccc3)n2)cc1]

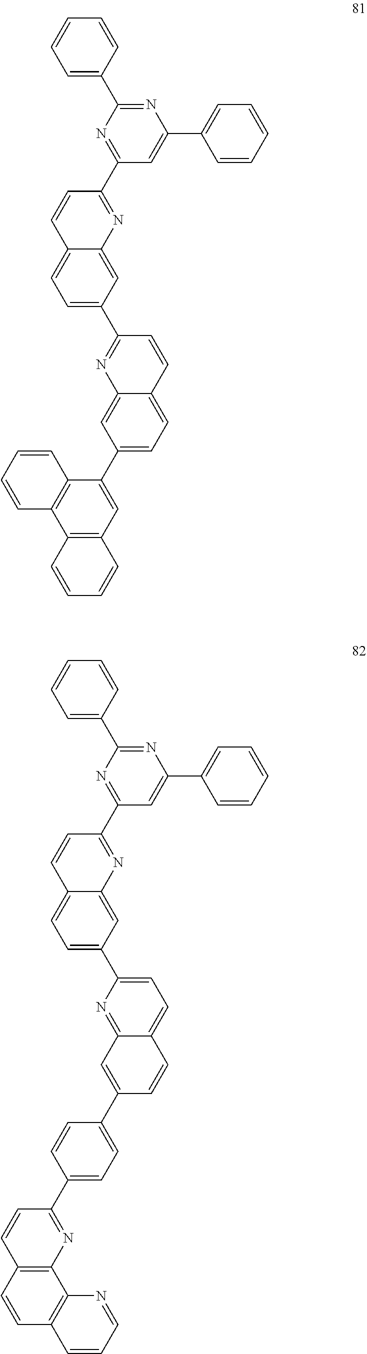 [SMILES: c1ccc(-c2cc(-c3ccc4ccc(-c5ccc6ccc(-c7cc8ccccc8c8ccccc78)cc6n5)cc4n3)nc(-c3ccccc3)n2)cc1 ; c1ccc(-c2cc(-c3ccc4ccc(-c5ccc6ccc(-c7ccc(-c8ccc9ccc%10cccnc%10c9n8)cc7)cc6n5)cc4n3)nc(-c3ccccc3)n2)cc1]

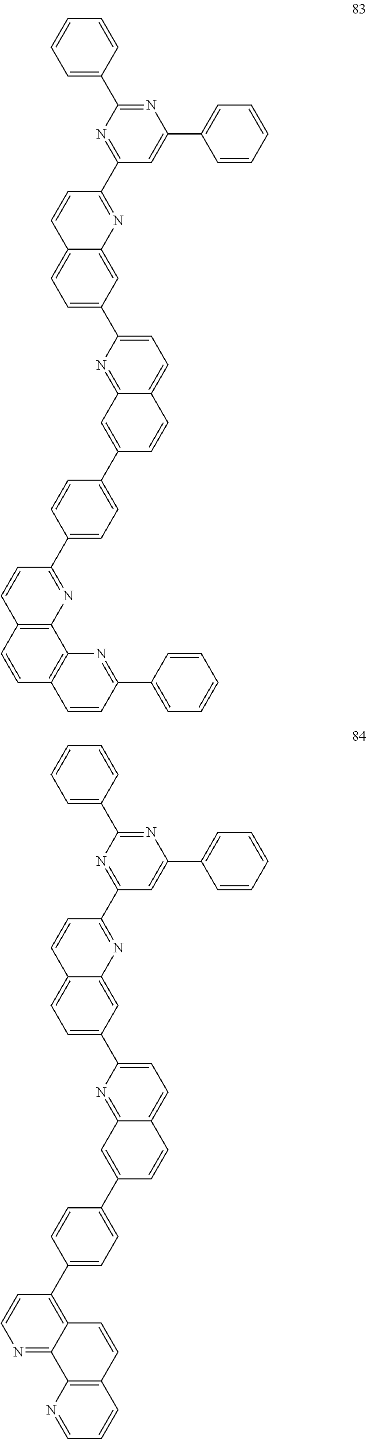

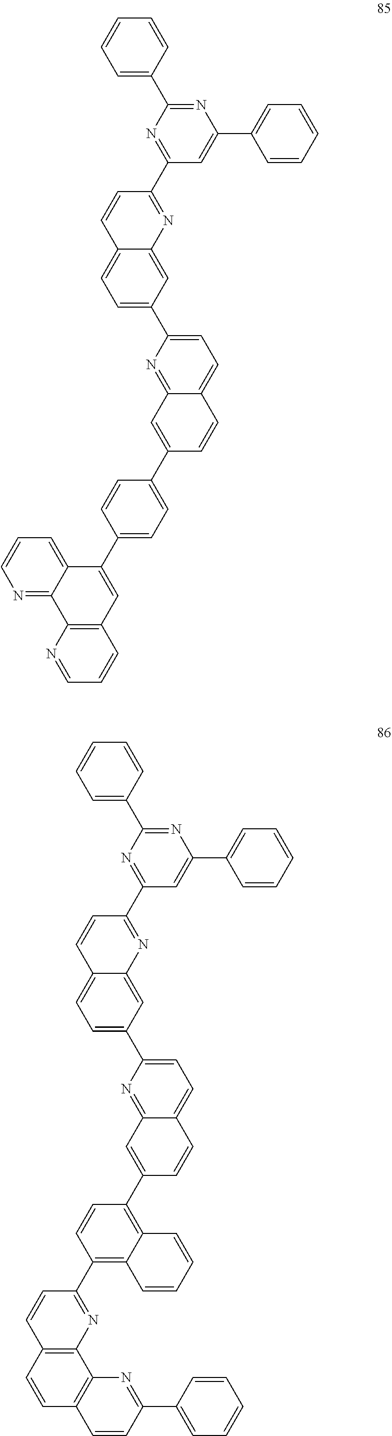 [SMILES: c1ccc(-c2cc(-c3ccc4ccc(-c5ccc6ccc(-c7ccc(-c8cc9cccnc9c9ncccc89)cc7)cc6n5)cc4n3)nc(-c3ccccc3)n2)cc1 ; c1ccc(-c2cc(-c3ccc4ccc(-c5ccc6ccc(-c7ccc(-c8ccc9ccc%10ccc(-c%11ccccc%11)nc%10c9n8)c8ccccc78)cc6n5)cc4n3)nc(-c3ccccc3)n2)cc1]

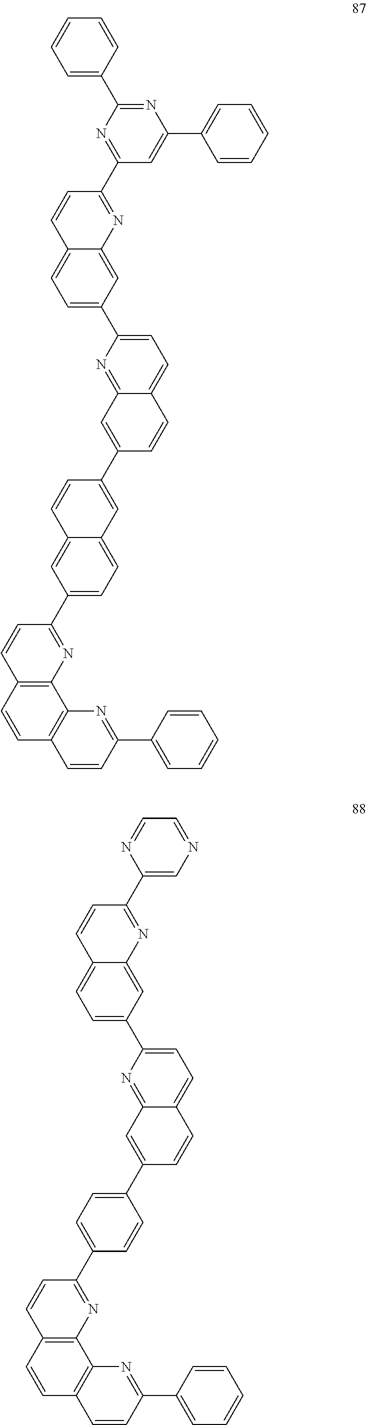 [SMILES: c1ccc(-c2cc(-c3ccc4ccc(-c5ccc6ccc(-c7ccc8cc(-c9ccc%10ccc%11ccc(-c%12ccccc%12)nc%11c%10n9)ccc8c7)cc6n5)cc4n3)nc(-c3ccccc3)n2)cc1 ; c1ccc(-c2ccc3ccc4ccc(-c5ccc(-c6ccc7ccc(-c8ccc9ccc(-c%10cnccn%10)nc9c8)nc7c6)cc5)nc4c3n2)cc1]

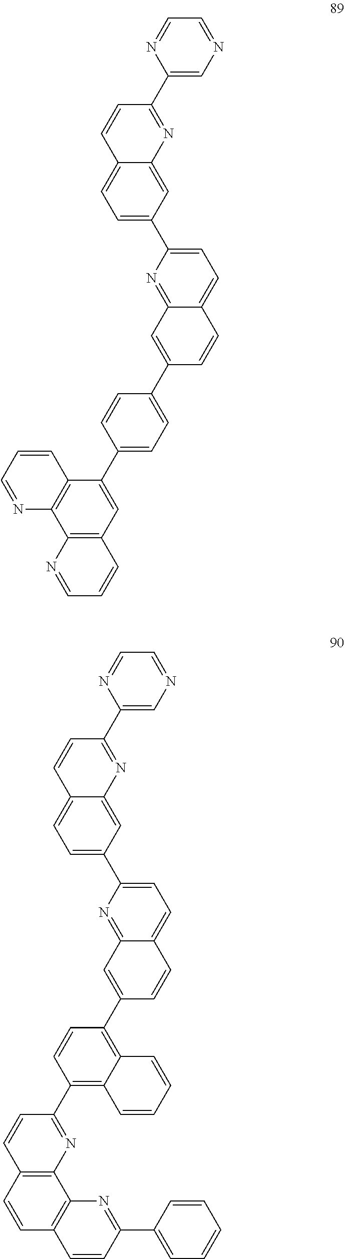 [SMILES: c1cnc2c(c1)cc(-c1ccc(-c3ccc4ccc(-c5ccc6ccc(-c7cnccn7)nc6c5)nc4c3)cc1)c1cccnc12 ; c1ccc(-c2ccc3ccc4ccc(-c5ccc(-c6ccc7ccc(-c8ccc9ccc(-c%10cnccn%10)nc9c8)nc7c6)c6ccccc56)nc4c3n2)cc1]

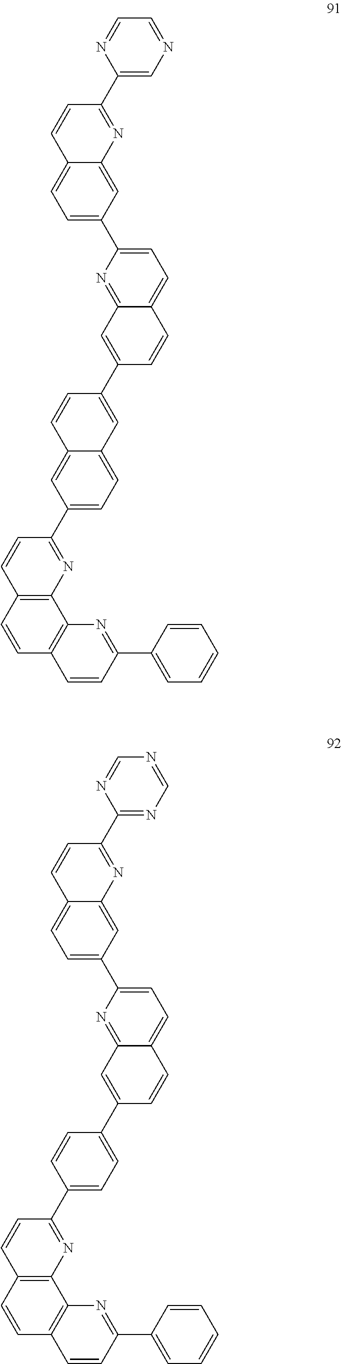 [SMILES: c1ccc(-c2ccc3ccc4ccc(-c5ccc6cc(-c7ccc8ccc(-c9ccc%10ccc(-c%11cnccn%11)nc%10c9)nc8c7)ccc6c5)nc4c3n2)cc1 ; c1ccc(-c2ccc3ccc4ccc(-c5ccc(-c6ccc7ccc(-c8ccc9ccc(-c%10ncncn%10)nc9c8)nc7c6)cc5)nc4c3n2)cc1]

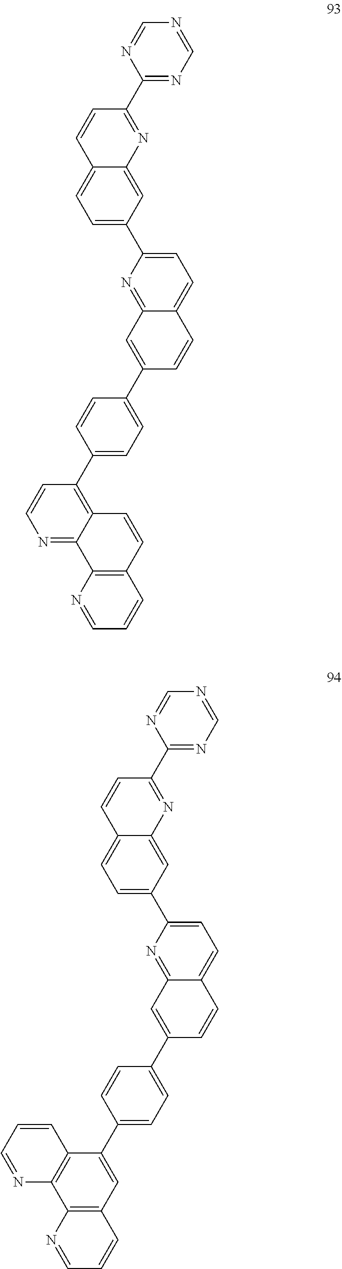 [SMILES: c1cnc2c(c1)ccc1c(-c3ccc(-c4ccc5ccc(-c6ccc7ccc(-c8ncncn8)nc7c6)nc5c4)cc3)ccnc12 ; c1cnc2c(c1)cc(-c1ccc(-c3ccc4ccc(-c5ccc6ccc(-c7ncncn7)nc6c5)nc4c3)cc1)c1cccnc12]

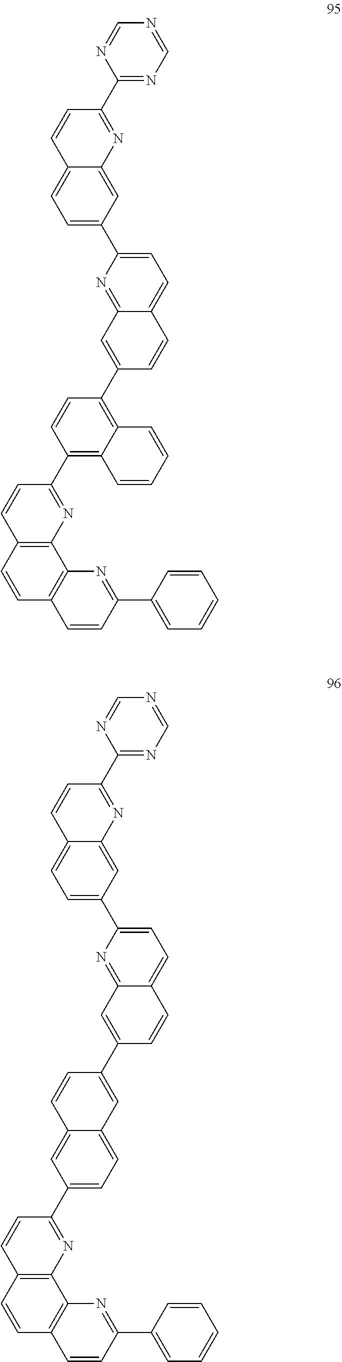 [SMILES: c1ccc(-c2ccc3ccc4ccc(-c5ccc(-c6ccc7ccc(-c8ccc9ccc(-c%10ncncn%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11ncncn%11)nc%10c9)nc8c7)ccc6c5)nc4c3n2)cc1]

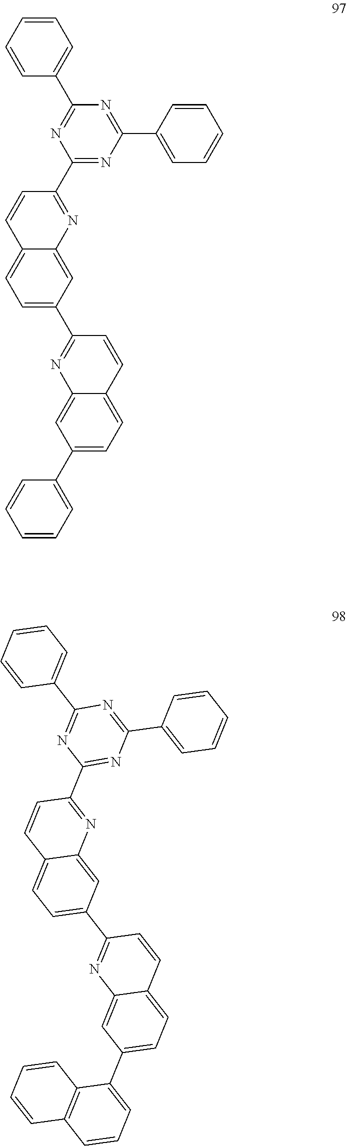 [SMILES: c1ccc(-c2ccc3ccc(-c4ccc5ccc(-c6nc(-c7ccccc7)nc(-c7ccccc7)n6)nc5c4)nc3c2)cc1 ; c1ccc(-c2nc(-c3ccccc3)nc(-c3ccc4ccc(-c5ccc6ccc(-c7cccc8ccccc78)cc6n5)cc4n3)n2)cc1]

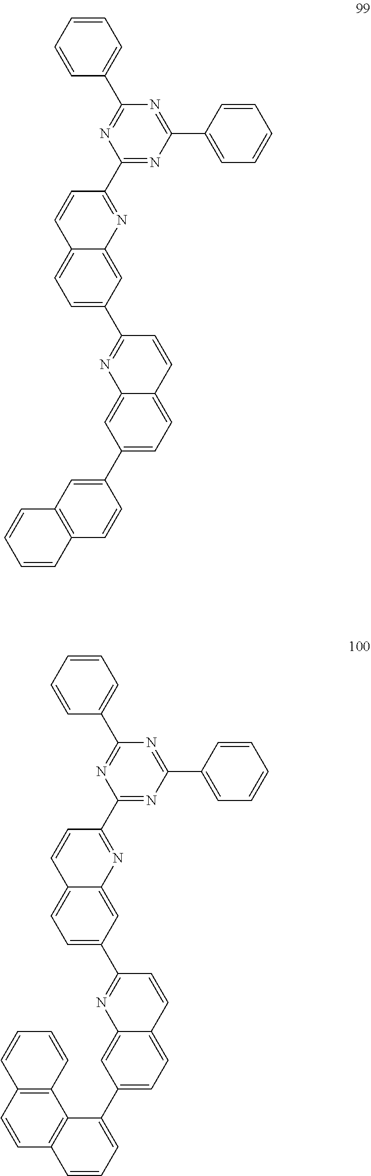 [SMILES: c1ccc(-c2nc(-c3ccccc3)nc(-c3ccc4ccc(-c5ccc6ccc(-c7ccc8ccccc8c7)cc6n5)cc4n3)n2)cc1 ; c1ccc(-c2nc(-c3ccccc3)nc(-c3ccc4ccc(-c5ccc6ccc(-c7cccc8ccc9ccccc9c78)cc6n5)cc4n3)n2)cc1]

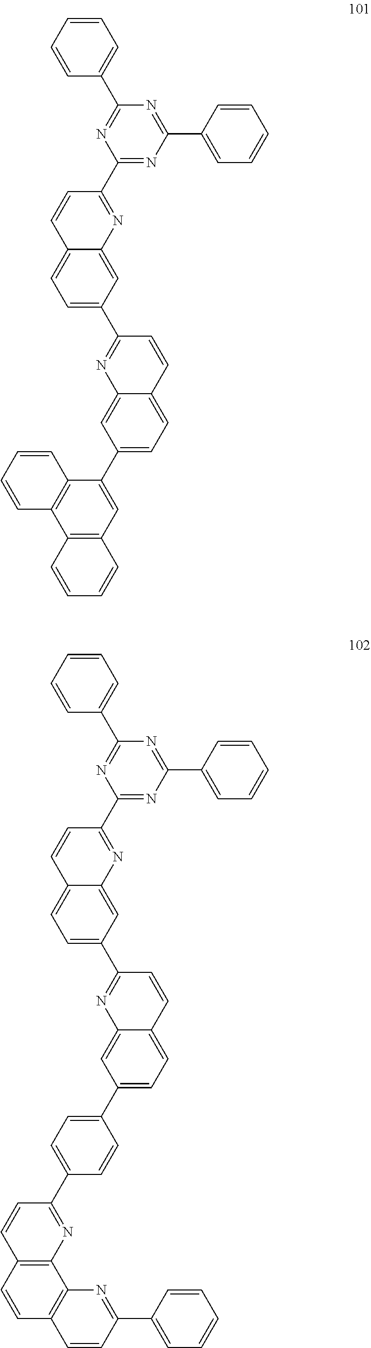 [SMILES: c1ccc(-c2nc(-c3ccccc3)nc(-c3ccc4ccc(-c5ccc6ccc(-c7cc8ccccc8c8ccccc78)cc6n5)cc4n3)n2)cc1 ; c1ccc(-c2ccc3ccc4ccc(-c5ccc(-c6ccc7ccc(-c8ccc9ccc(-c%10nc(-c%11ccccc%11)nc(-c%11ccccc%11)n%10)nc9c8)nc7c6)cc5)nc4c3n2)cc1]

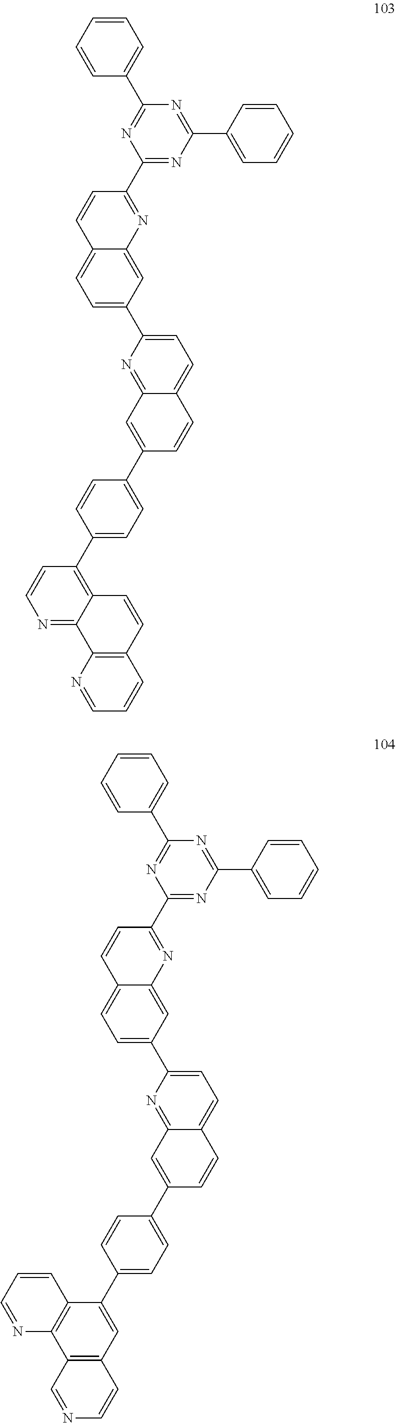

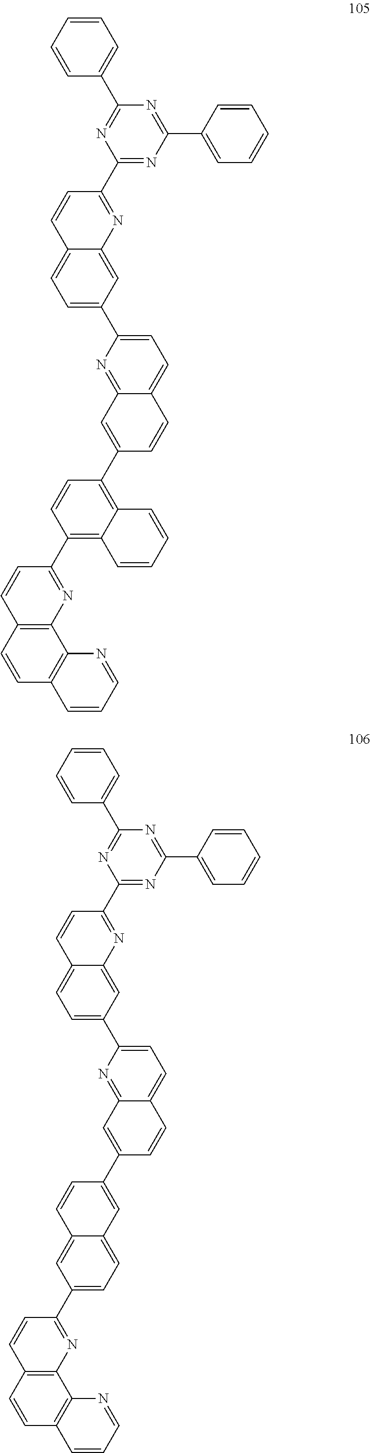

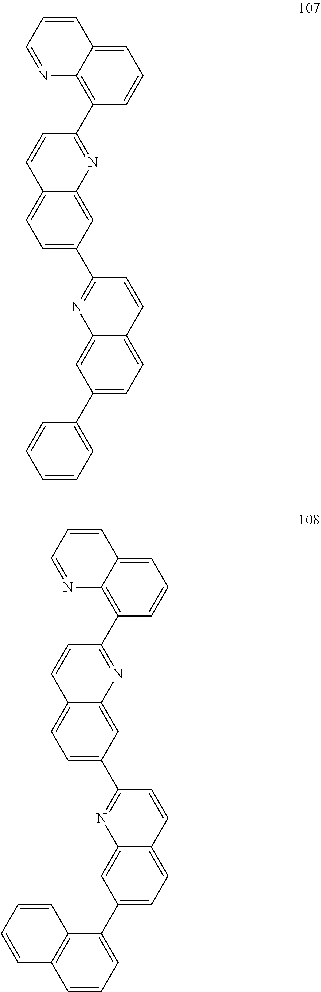 [SMILES: c1ccc(-c2ccc3ccc(-c4ccc5ccc(-c6cccc7cccnc67)nc5c4)nc3c2)cc1 ; c1ccc2c(-c3ccc4ccc(-c5ccc6ccc(-c7cccc8cccnc78)nc6c5)nc4c3)cccc2c1]

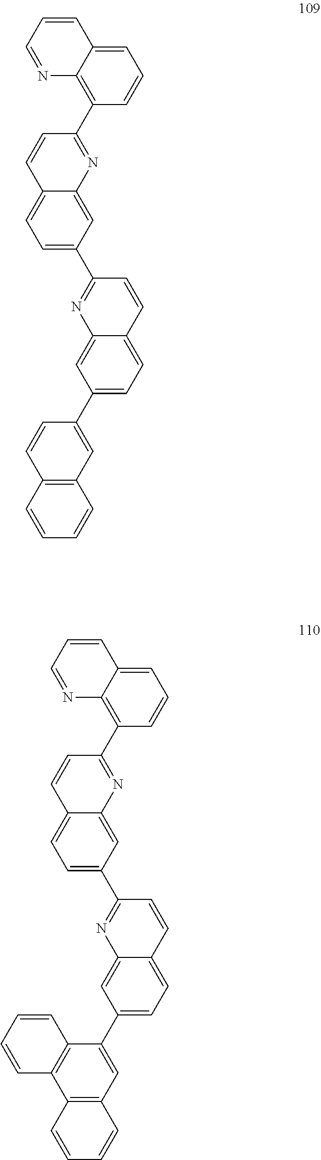 [SMILES: c1ccc2cc(-c3ccc4ccc(-c5ccc6ccc(-c7cccc8cccnc78)nc6c5)nc4c3)ccc2c1 ; c1cnc2c(-c3ccc4ccc(-c5ccc6ccc(-c7cc8ccccc8c8ccccc78)cc6n5)cc4n3)cccc2c1]

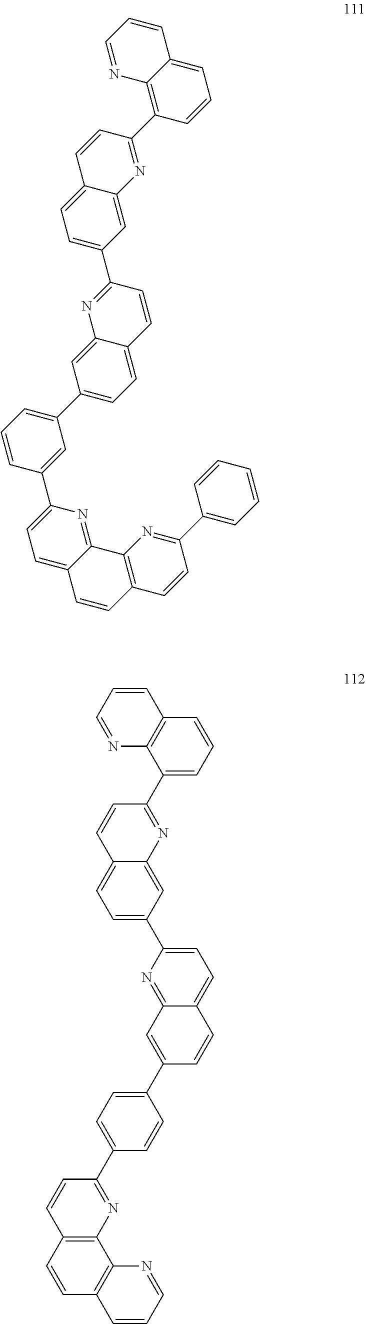 [SMILES: c1ccc(-c2ccc3ccc4ccc(-c5cccc(-c6ccc7ccc(-c8ccc9ccc(-c%10cccc%11cccnc%10%11)nc9c8)nc7c6)c5)nc4c3n2)cc1 ; c1cnc2c(-c3ccc4ccc(-c5ccc6ccc(-c7ccc(-c8ccc9ccc%10cccnc%10c9n8)cc7)cc6n5)cc4n3)cccc2c1]

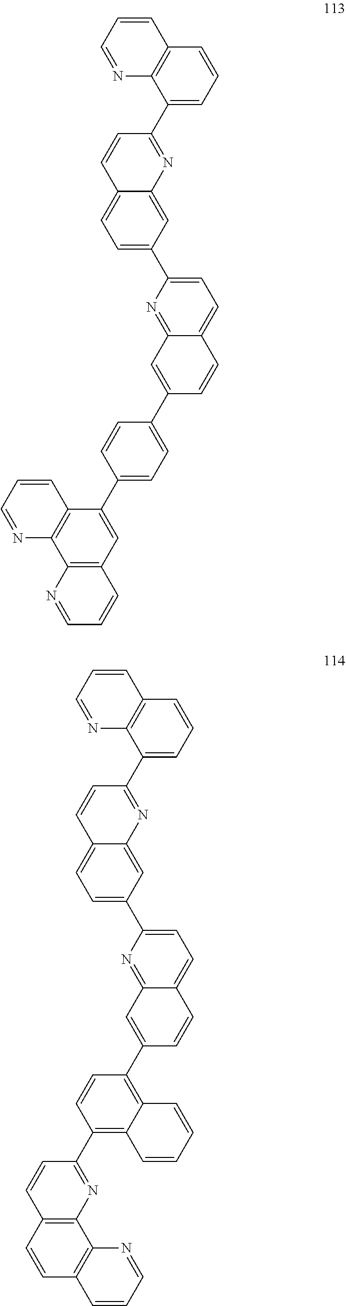

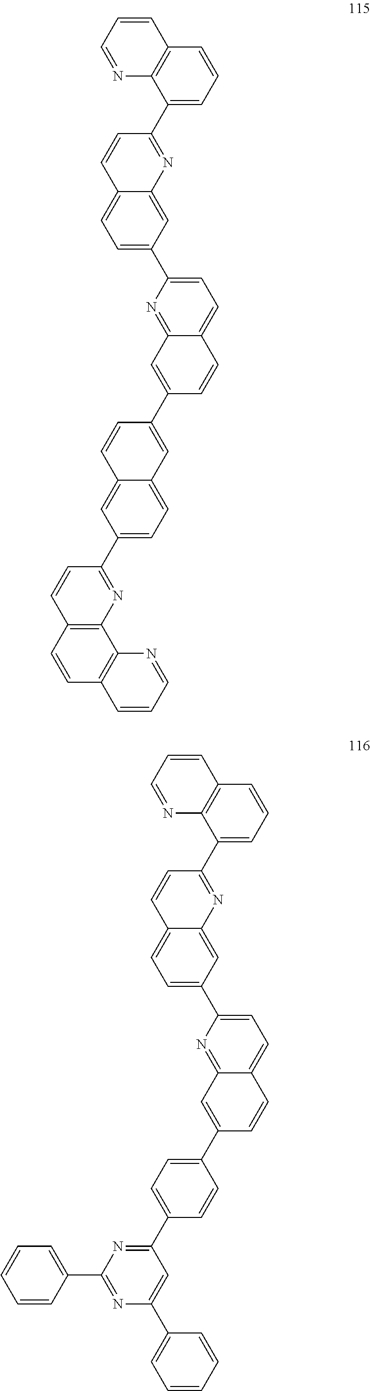 [SMILES: c1cnc2c(-c3ccc4ccc(-c5ccc6ccc(-c7ccc8cc(-c9ccc%10ccc%11cccnc%11c%10n9)ccc8c7)cc6n5)cc4n3)cccc2c1 ; c1ccc(-c2cc(-c3ccc(-c4ccc5ccc(-c6ccc7ccc(-c8cccc9cccnc89)nc7c6)nc5c4)cc3)nc(-c3ccccc3)n2)cc1]

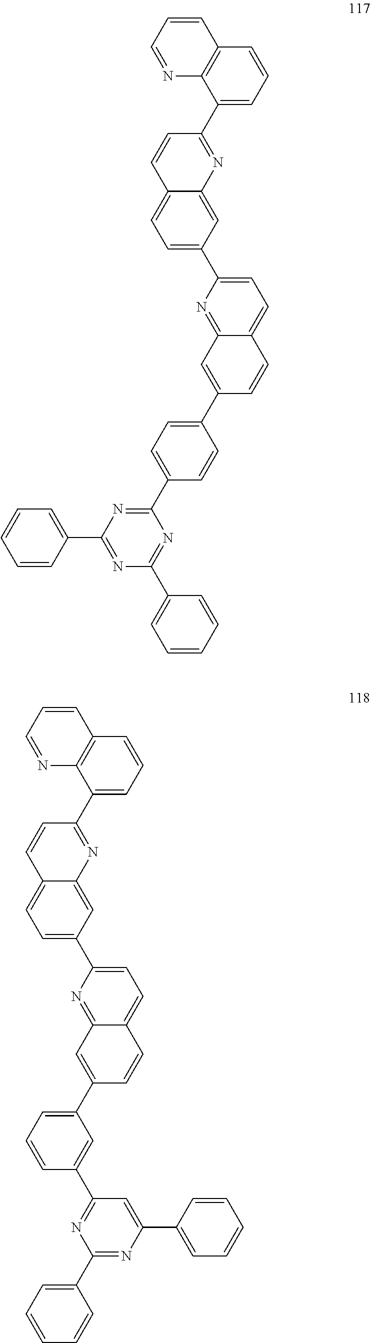 [SMILES: c1ccc(-c2nc(-c3ccccc3)nc(-c3ccc(-c4ccc5ccc(-c6ccc7ccc(-c8cccc9cccnc89)nc7c6)nc5c4)cc3)n2)cc1 ; c1ccc(-c2cc(-c3cccc(-c4ccc5ccc(-c6ccc7ccc(-c8cccc9cccnc89)nc7c6)nc5c4)c3)nc(-c3ccccc3)n2)cc1]

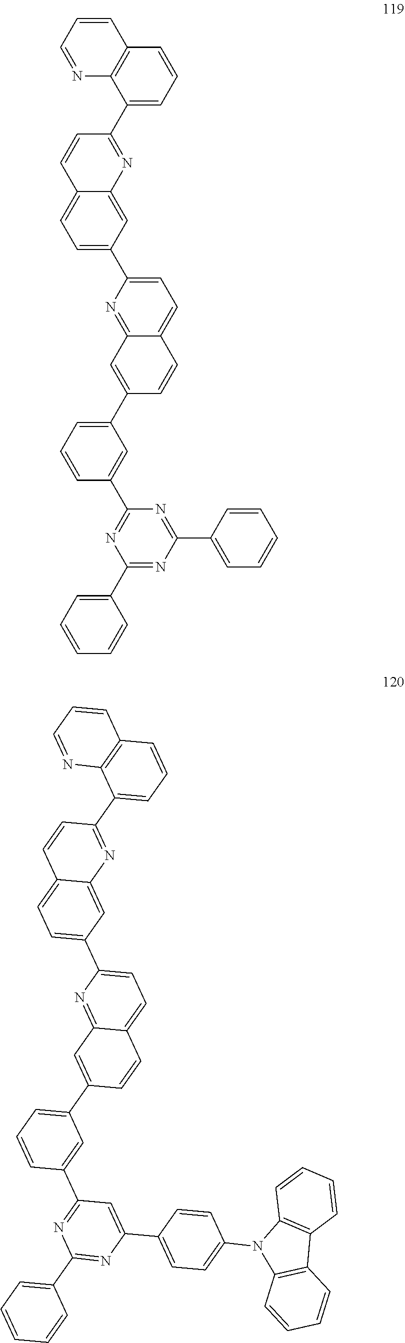 [SMILES: c1ccc(-c2nc(-c3ccccc3)nc(-c3cccc(-c4ccc5ccc(-c6ccc7ccc(-c8cccc9cccnc89)nc7c6)nc5c4)c3)n2)cc1 ; c1ccc(-c2nc(-c3ccc(-n4c5ccccc5c5ccccc54)cc3)cc(-c3cccc(-c4ccc5ccc(-c6ccc7ccc(-c8cccc9cccnc89)nc7c6)nc5c4)c3)n2)cc1]

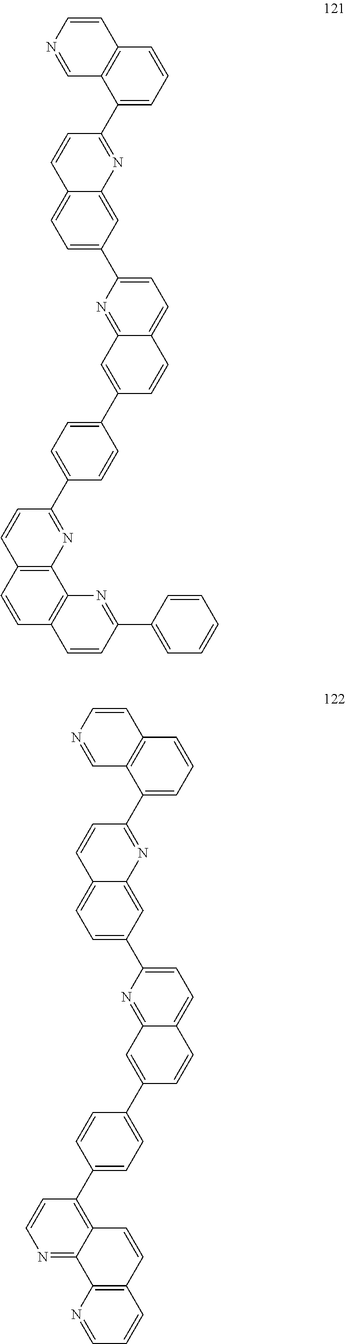 [SMILES: c1ccc(-c2ccc3ccc4ccc(-c5ccc(-c6ccc7ccc(-c8ccc9ccc(-c%10cccc%11ccncc%10%11)nc9c8)nc7c6)cc5)nc4c3n2)cc1 ; c1cc(-c2ccc3ccc(-c4ccc5ccc(-c6ccc(-c7ccnc8c7ccc7cccnc78)cc6)cc5n4)cc3n2)c2cnccc2c1]

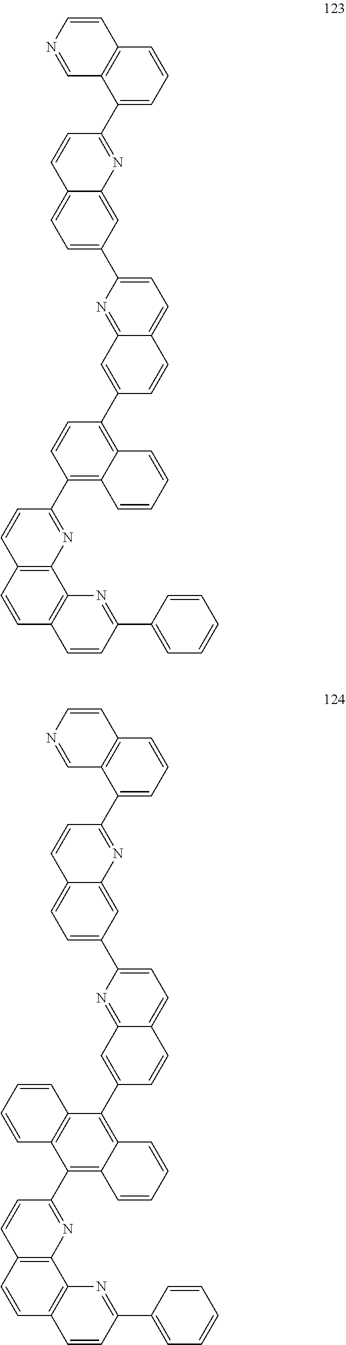 [SMILES: c1ccc(-c2ccc3ccc4ccc(-c5ccc(-c6ccc7ccc(-c8ccc9ccc(-c%10cccc%11ccncc%10%11)nc9c8)nc7c6)c6ccccc56)nc4c3n2)cc1 ; c1ccc(-c2ccc3ccc4ccc(-c5c6ccccc6c(-c6ccc7ccc(-c8ccc9ccc(-c%10cccc%11ccncc%10%11)nc9c8)nc7c6)c6ccccc56)nc4c3n2)cc1]

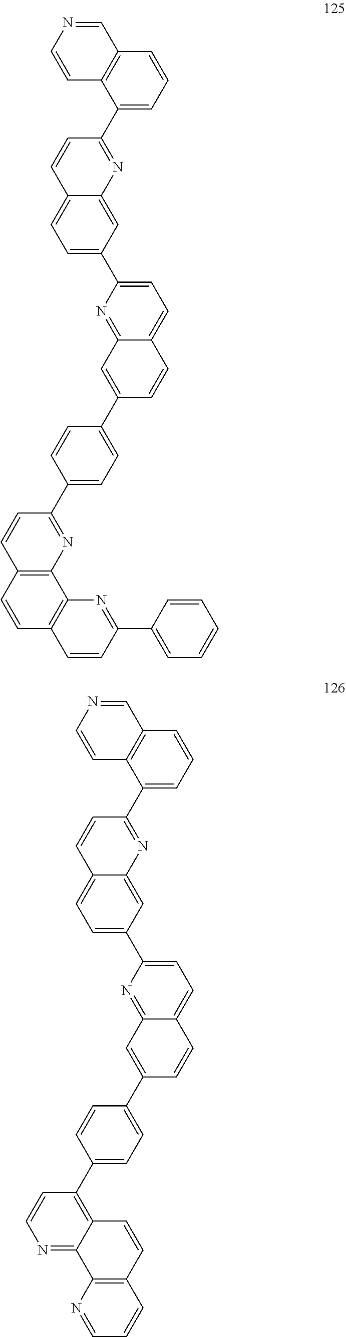

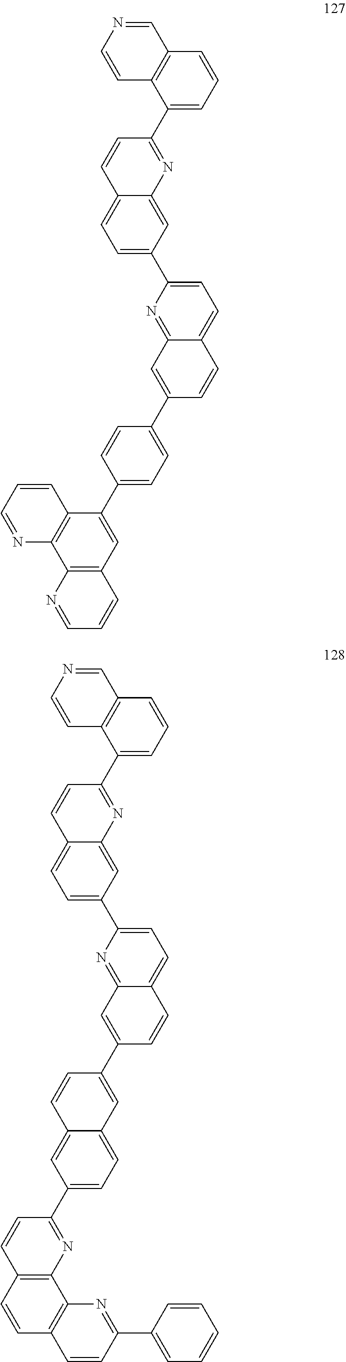

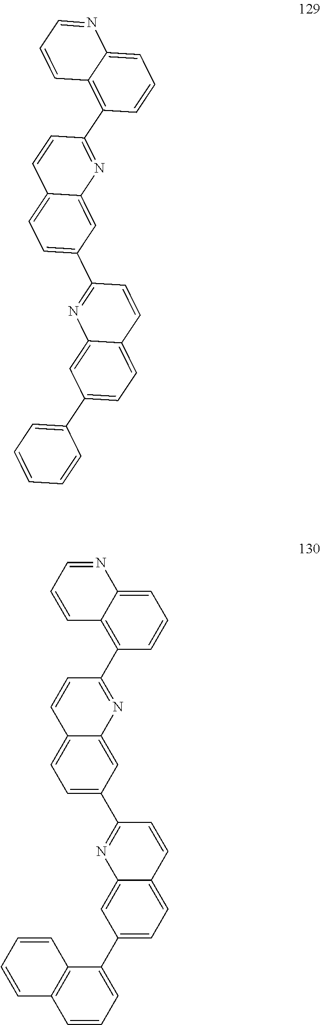 [SMILES: c1ccc(-c2ccc3ccc(-c4ccc5ccc(-c6cccc7ncccc67)nc5c4)nc3c2)cc1 ; c1ccc2c(-c3ccc4ccc(-c5ccc6ccc(-c7cccc8ncccc78)nc6c5)nc4c3)cccc2c1]

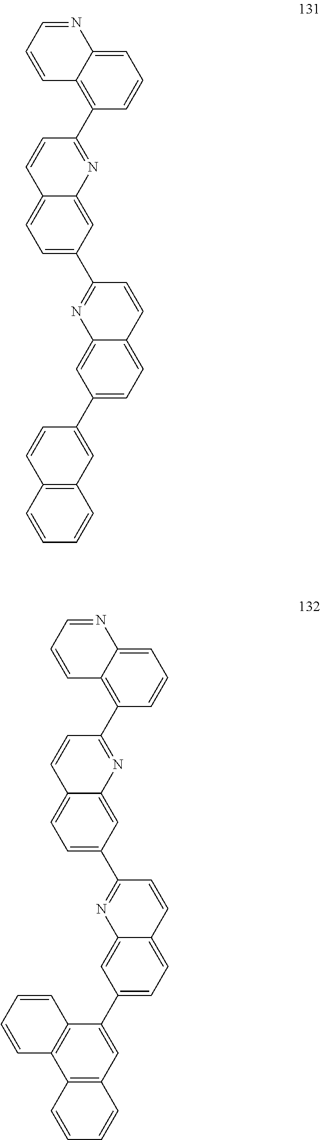 [SMILES: c1ccc2cc(-c3ccc4ccc(-c5ccc6ccc(-c7cccc8ncccc78)nc6c5)nc4c3)ccc2c1 ; c1ccc2c(c1)cc(-c1ccc3ccc(-c4ccc5ccc(-c6cccc7ncccc67)nc5c4)nc3c1)c1ccccc12]

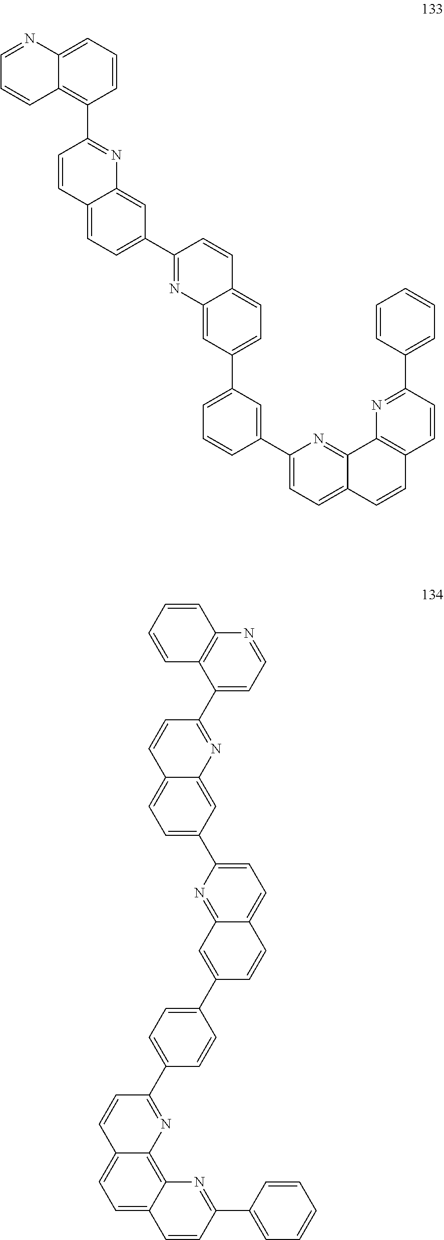 [SMILES: c1ccc(-c2ccc3ccc4ccc(-c5cccc(-c6ccc7ccc(-c8ccc9ccc(-c%10cccc%11ncccc%10%11)nc9c8)nc7c6)c5)nc4c3n2)cc1 ; c1ccc(-c2ccc3ccc4ccc(-c5ccc(-c6ccc7ccc(-c8ccc9ccc(-c%10ccnc%11ccccc%10%11)nc9c8)nc7c6)cc5)nc4c3n2)cc1]

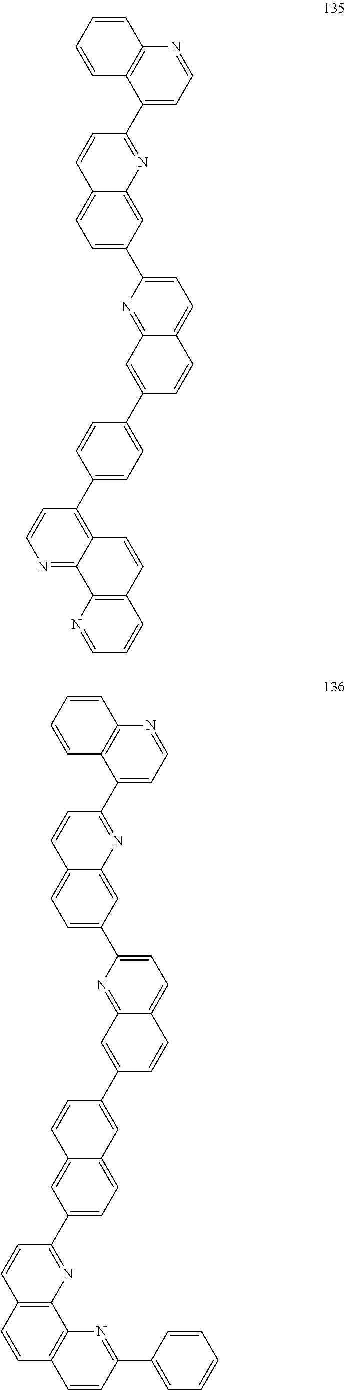

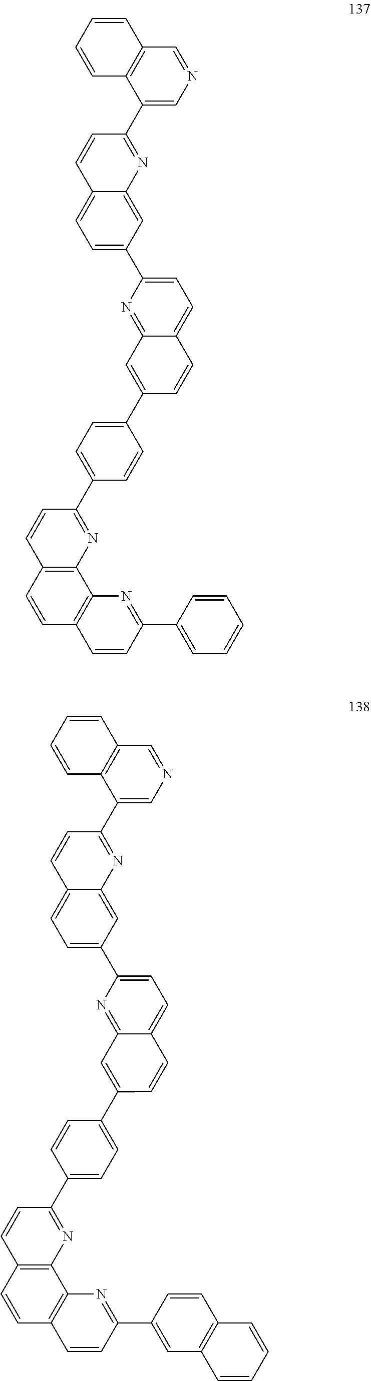 [SMILES: c1ccc(-c2ccc3ccc4ccc(-c5ccc(-c6ccc7ccc(-c8ccc9ccc(-c%10cncc%11ccccc%10%11)nc9c8)nc7c6)cc5)nc4c3n2)cc1 ; c1ccc2cc(-c3ccc4ccc5ccc(-c6ccc(-c7ccc8ccc(-c9ccc%10ccc(-c%11cncc%12ccccc%11%12)nc%10c9)nc8c7)cc6)nc5c4n3)ccc2c1]

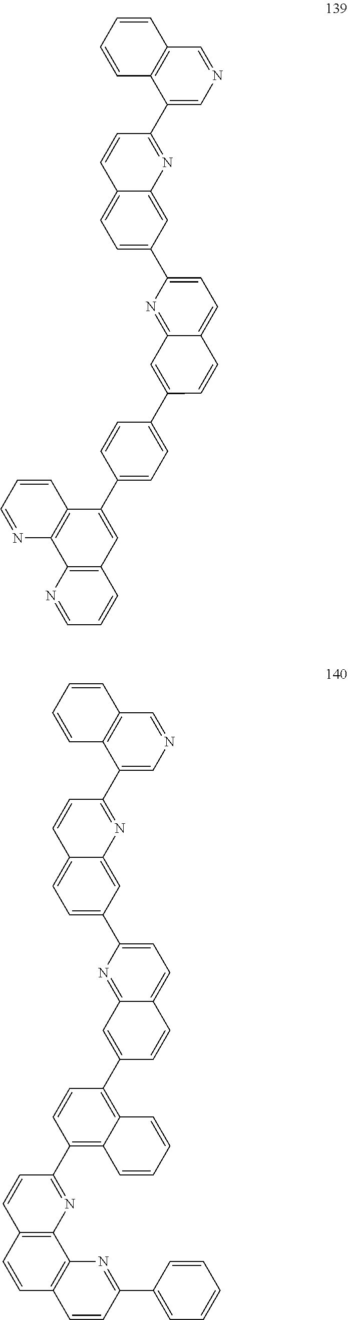 [SMILES: c1ccc2c(-c3ccc4ccc(-c5ccc6ccc(-c7ccc(-c8cc9cccnc9c9ncccc89)cc7)cc6n5)cc4n3)cncc2c1 ; c1ccc(-c2ccc3ccc4ccc(-c5ccc(-c6ccc7ccc(-c8ccc9ccc(-c%10cncc%11ccccc%10%11)nc9c8)nc7c6)c6ccccc56)nc4c3n2)cc1]

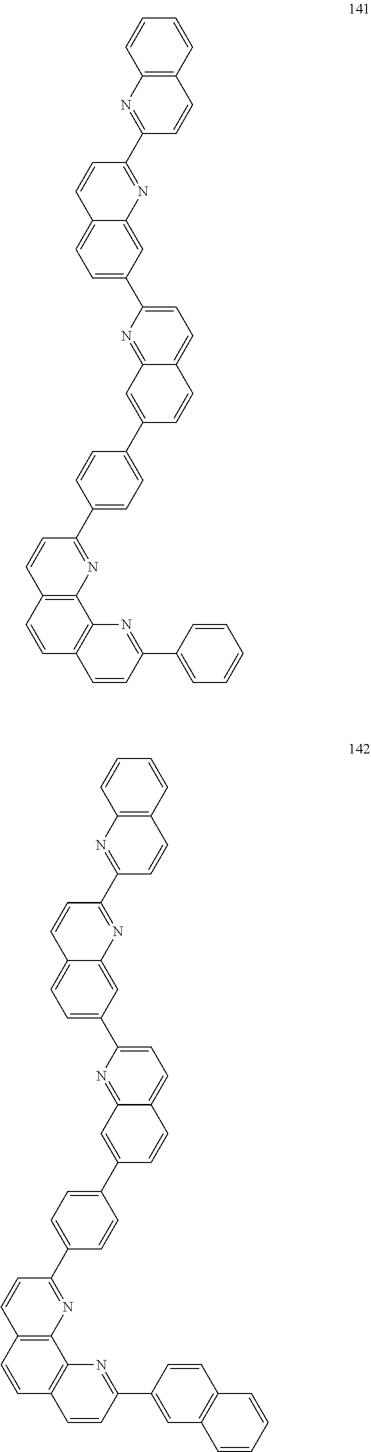 [SMILES: c1ccc(-c2ccc3ccc4ccc(-c5ccc(-c6ccc7ccc(-c8ccc9ccc(-c%10ccc%11ccccc%11n%10)nc9c8)nc7c6)cc5)nc4c3n2)cc1 ; c1ccc2cc(-c3ccc4ccc5ccc(-c6ccc(-c7ccc8ccc(-c9ccc%10ccc(-c%11ccc%12ccccc%12n%11)nc%10c9)nc8c7)cc6)nc5c4n3)ccc2c1]

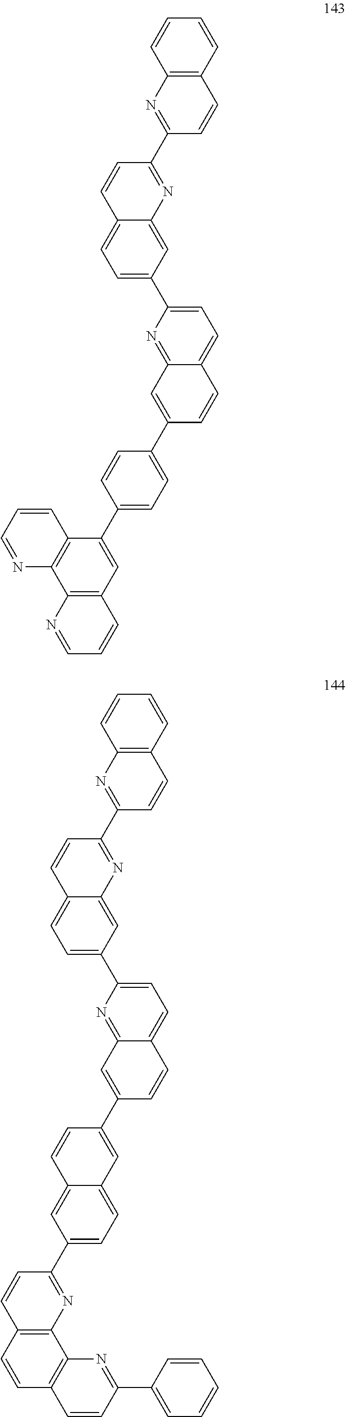

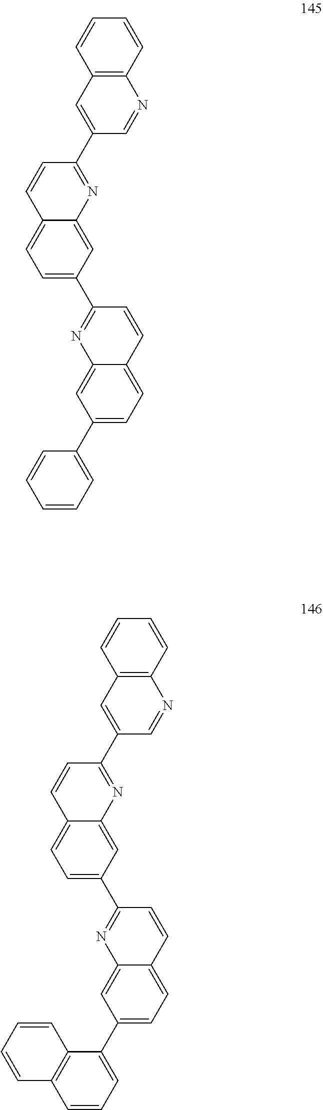 [SMILES: c1ccc(-c2ccc3ccc(-c4ccc5ccc(-c6cnc7ccccc7c6)nc5c4)nc3c2)cc1 ; c1ccc2ncc(-c3ccc4ccc(-c5ccc6ccc(-c7cccc8ccccc78)cc6n5)cc4n3)cc2c1]

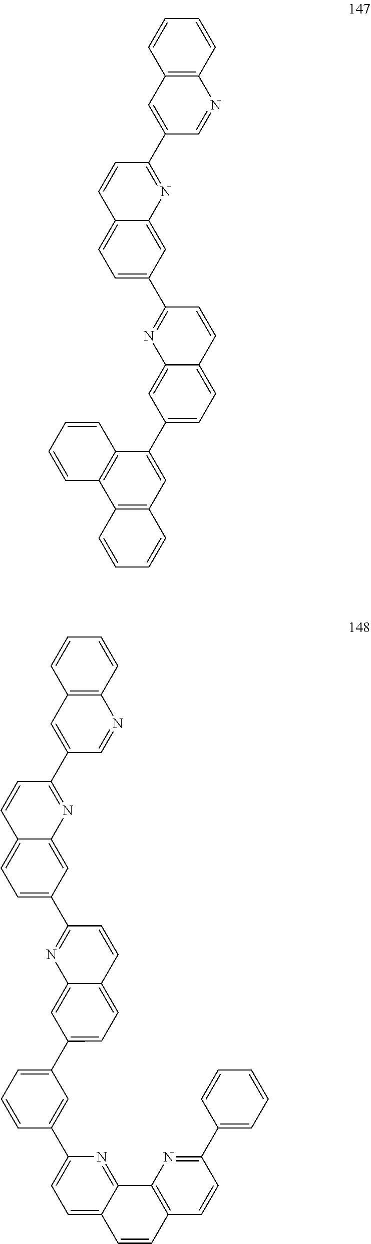 [SMILES: c1ccc2ncc(-c3ccc4ccc(-c5ccc6ccc(-c7cc8ccccc8c8ccccc78)cc6n5)cc4n3)cc2c1 ; c1ccc(-c2ccc3ccc4ccc(-c5cccc(-c6ccc7ccc(-c8ccc9ccc(-c%10cnc%11ccccc%11c%10)nc9c8)nc7c6)c5)nc4c3n2)cc1]

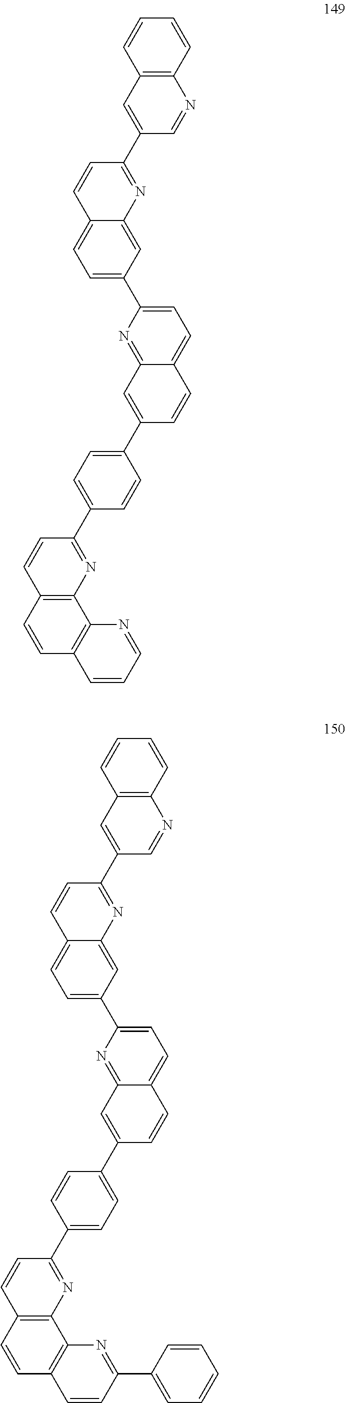 [SMILES: c1ccc2ncc(-c3ccc4ccc(-c5ccc6ccc(-c7ccc(-c8ccc9ccc%10cccnc%10c9n8)cc7)cc6n5)cc4n3)cc2c1 ; c1ccc(-c2ccc3ccc4ccc(-c5ccc(-c6ccc7ccc(-c8ccc9ccc(-c%10cnc%11ccccc%11c%10)nc9c8)nc7c6)cc5)nc4c3n2)cc1]

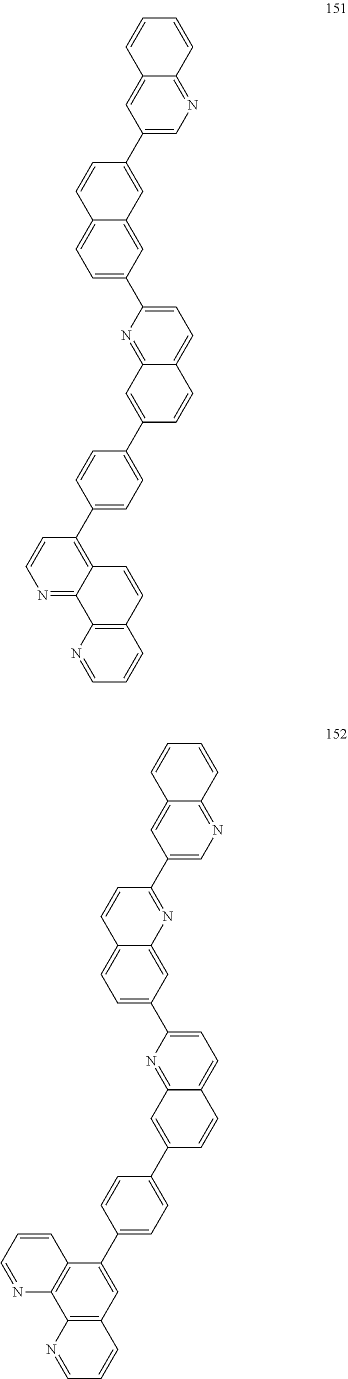 [SMILES: c1ccc2ncc(-c3ccc4ccc(-c5ccc6ccc(-c7ccc(-c8ccnc9c8ccc8cccnc89)cc7)cc6n5)cc4c3)cc2c1 ; c1ccc2ncc(-c3ccc4ccc(-c5ccc6ccc(-c7ccc(-c8cc9cccnc9c9ncccc89)cc7)cc6n5)cc4n3)cc2c1]

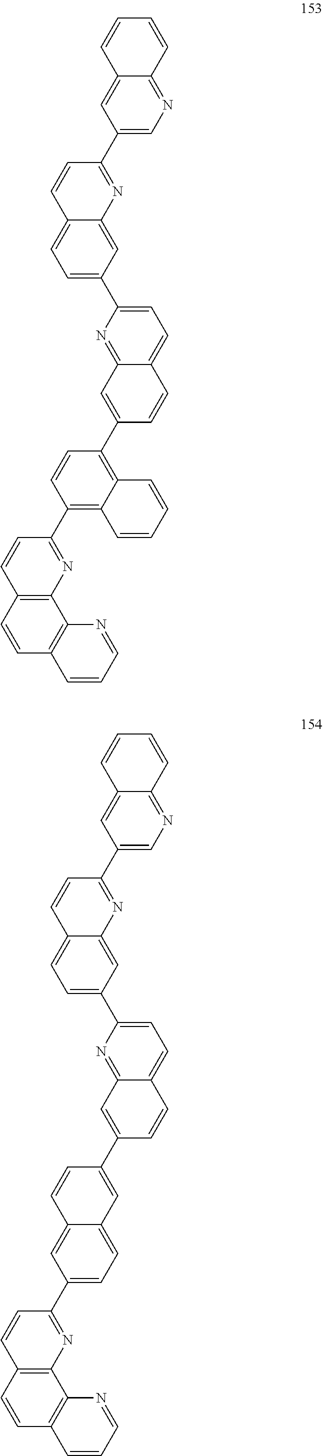 [SMILES: c1ccc2ncc(-c3ccc4ccc(-c5ccc6ccc(-c7ccc(-c8ccc9ccc%10cccnc%10c9n8)c8ccccc78)cc6n5)cc4n3)cc2c1 ; c1ccc2ncc(-c3ccc4ccc(-c5ccc6ccc(-c7ccc8cc(-c9ccc%10ccc%11cccnc%11c%10n9)ccc8c7)cc6n5)cc4n3)cc2c1]

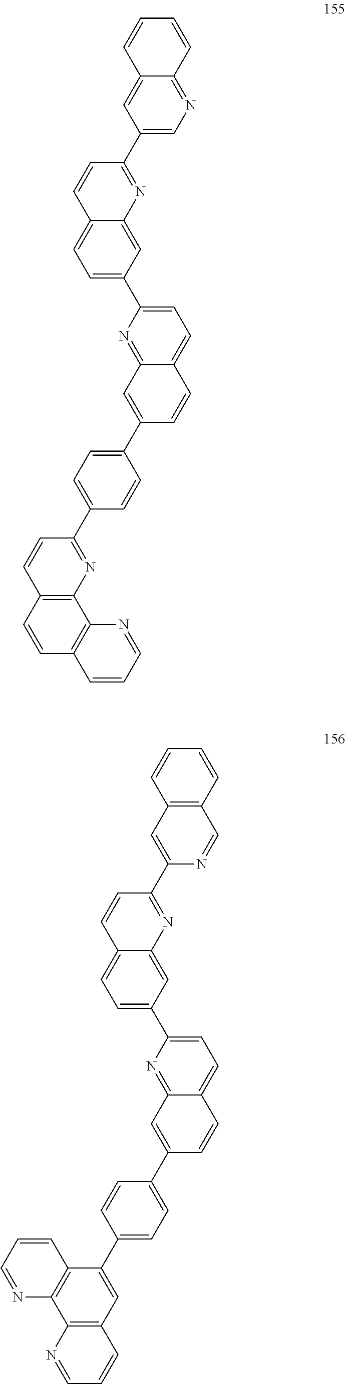 [SMILES: c1ccc2ncc(-c3ccc4ccc(-c5ccc6ccc(-c7ccc(-c8ccc9ccc%10cccnc%10c9n8)cc7)cc6n5)cc4n3)cc2c1 ; c1ccc2cc(-c3ccc4ccc(-c5ccc6ccc(-c7ccc(-c8cc9cccnc9c9ncccc89)cc7)cc6n5)cc4n3)ncc2c1]

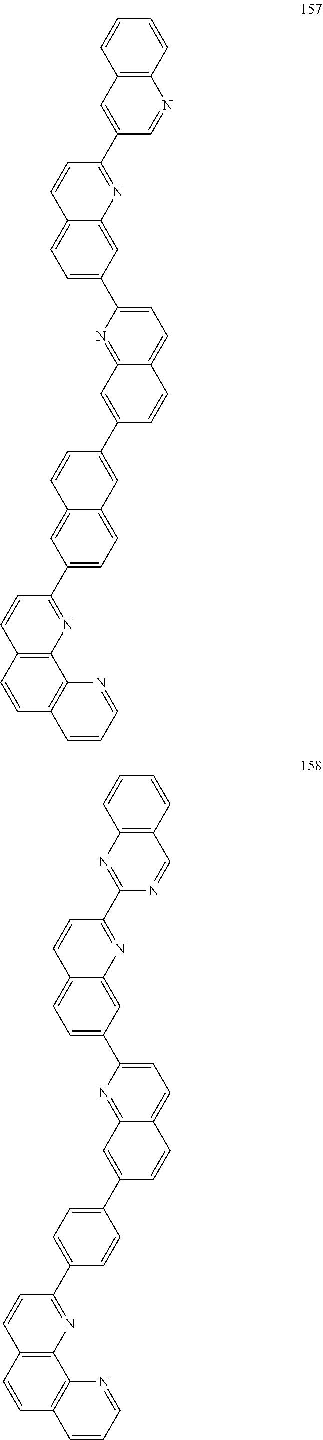

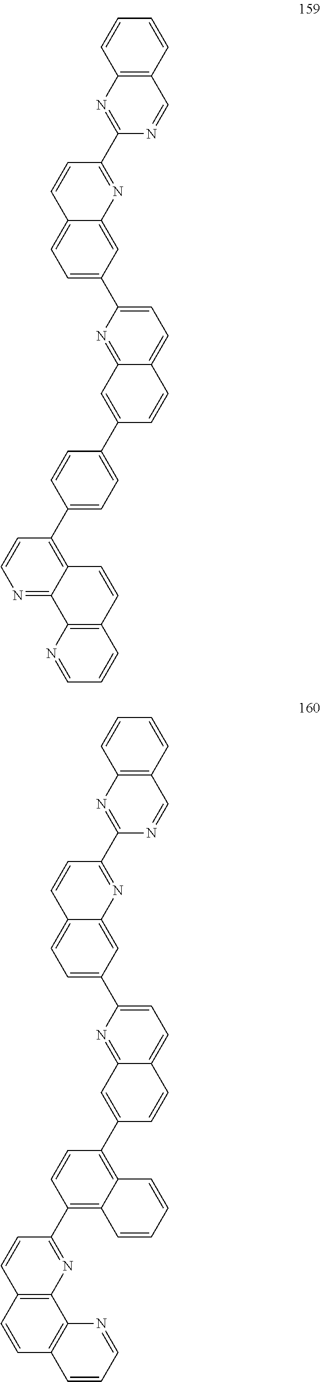

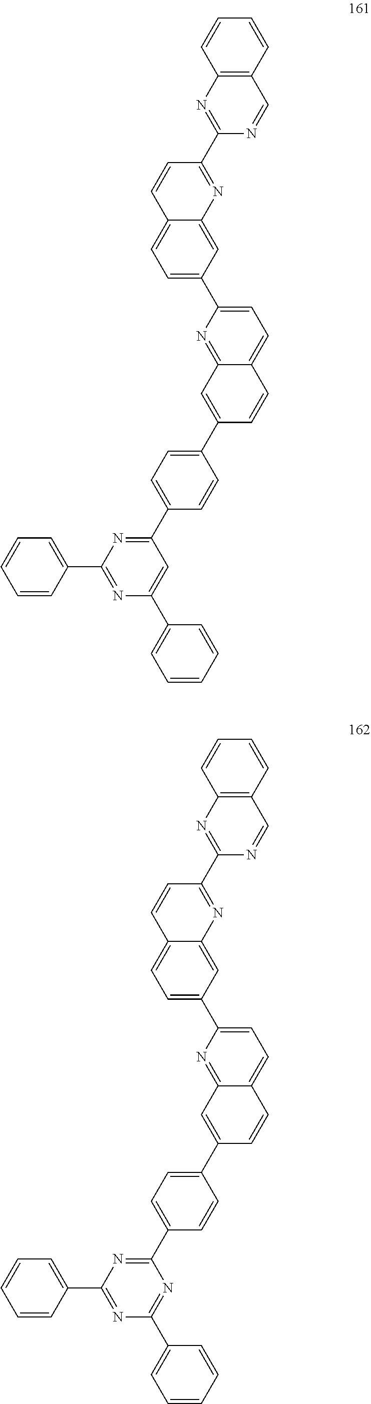 [SMILES: c1ccc(-c2cc(-c3ccc(-c4ccc5ccc(-c6ccc7ccc(-c8ncc9ccccc9n8)nc7c6)nc5c4)cc3)nc(-c3ccccc3)n2)cc1 ; c1ccc(-c2nc(-c3ccccc3)nc(-c3ccc(-c4ccc5ccc(-c6ccc7ccc(-c8ncc9ccccc9n8)nc7c6)nc5c4)cc3)n2)cc1]

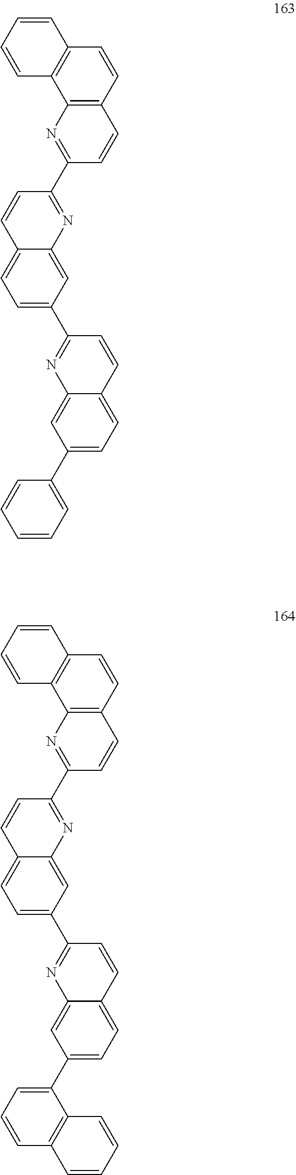 [SMILES: c1ccc(-c2ccc3ccc(-c4ccc5ccc(-c6ccc7ccc8ccccc8c7n6)nc5c4)nc3c2)cc1 ; c1ccc2c(-c3ccc4ccc(-c5ccc6ccc(-c7ccc8ccc9ccccc9c8n7)nc6c5)nc4c3)cccc2c1]

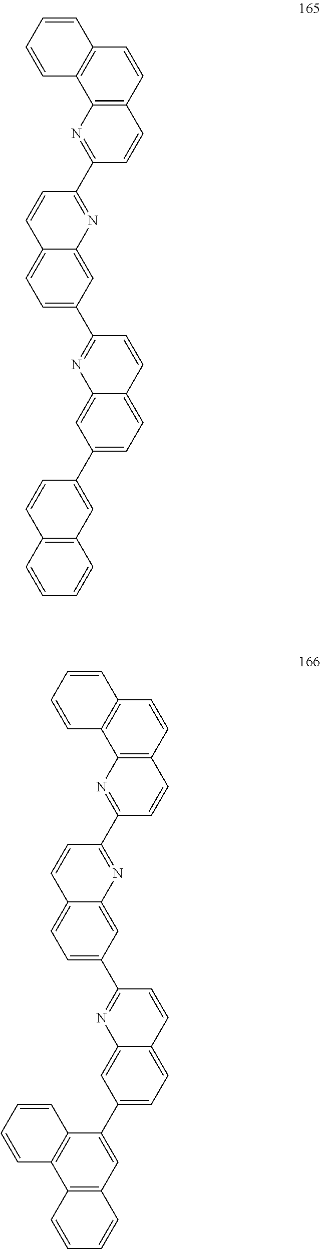 [SMILES: c1ccc2cc(-c3ccc4ccc(-c5ccc6ccc(-c7ccc8ccc9ccccc9c8n7)nc6c5)nc4c3)ccc2c1 ; c1ccc2c(c1)cc(-c1ccc3ccc(-c4ccc5ccc(-c6ccc7ccc8ccccc8c7n6)nc5c4)nc3c1)c1ccccc12]

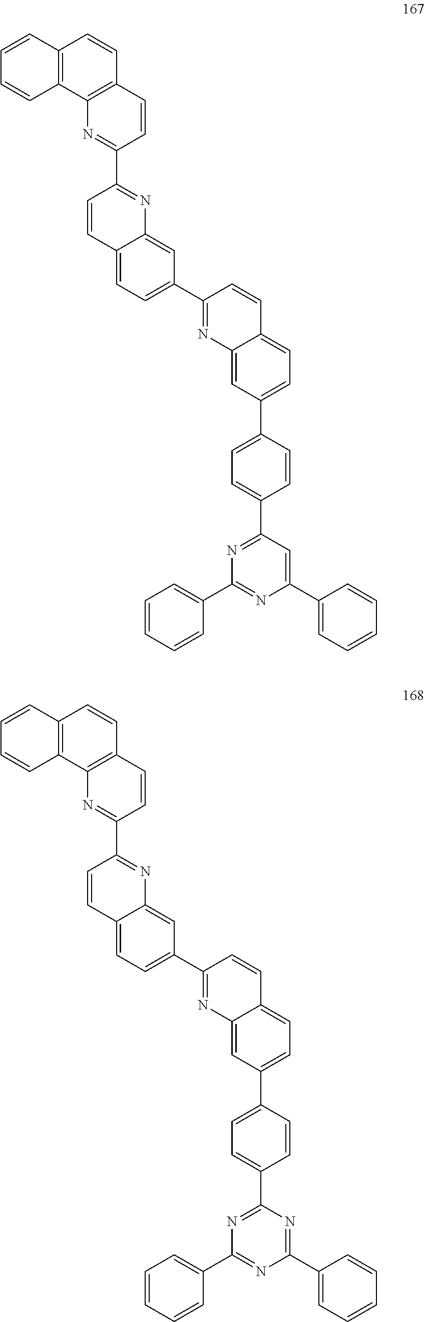 [SMILES: c1ccc(-c2cc(-c3ccc(-c4ccc5ccc(-c6ccc7ccc(-c8ccc9ccc%10ccccc%10c9n8)nc7c6)nc5c4)cc3)nc(-c3ccccc3)n2)cc1 ; c1ccc(-c2nc(-c3ccccc3)nc(-c3ccc(-c4ccc5ccc(-c6ccc7ccc(-c8ccc9ccc%10ccccc%10c9n8)nc7c6)nc5c4)cc3)n2)cc1]

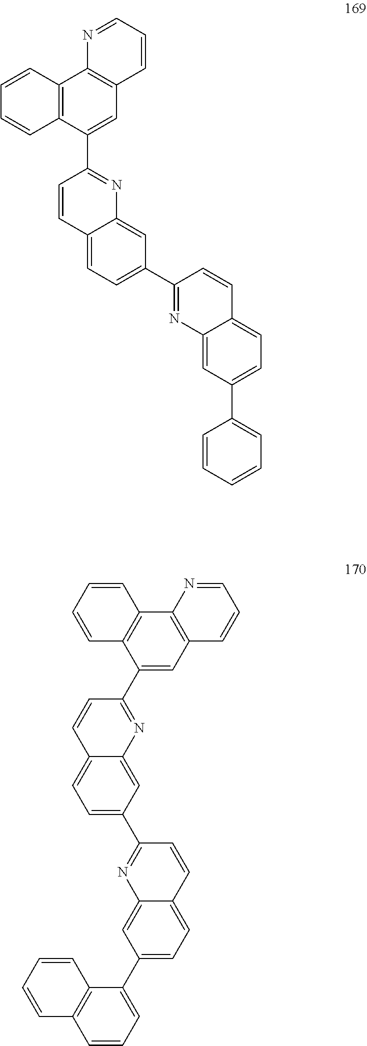 [SMILES: c1ccc(-c2ccc3ccc(-c4ccc5ccc(-c6cc7cccnc7c7ccccc67)nc5c4)nc3c2)cc1 ; c1ccc2c(-c3ccc4ccc(-c5ccc6ccc(-c7cc8cccnc8c8ccccc78)nc6c5)nc4c3)cccc2c1]

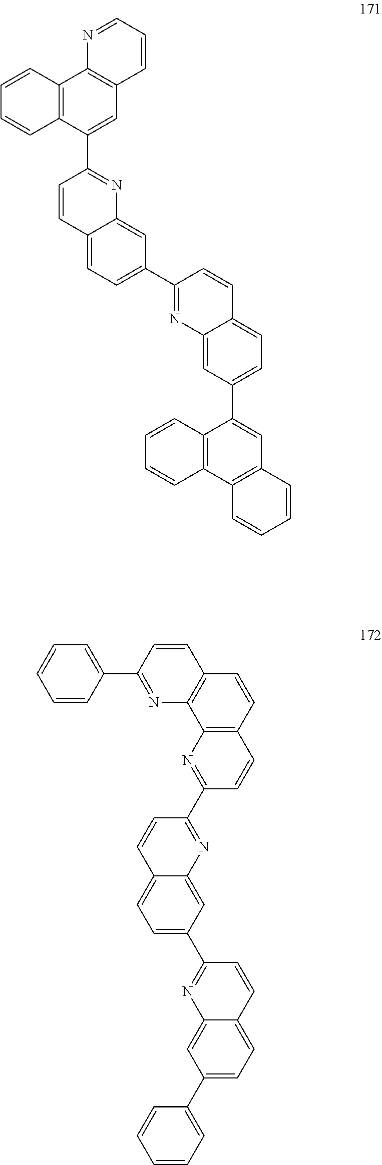 [SMILES: c1ccc2c(c1)cc(-c1ccc3ccc(-c4ccc5ccc(-c6cc7cccnc7c7ccccc67)nc5c4)nc3c1)c1ccccc12 ; c1ccc(-c2ccc3ccc(-c4ccc5ccc(-c6ccc7ccc8ccc(-c9ccccc9)nc8c7n6)nc5c4)nc3c2)cc1]

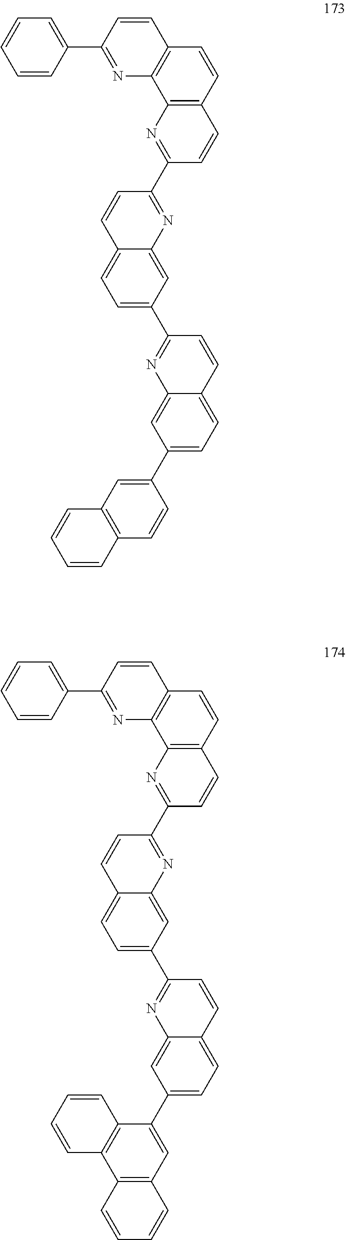 [SMILES: c1ccc(-c2ccc3ccc4ccc(-c5ccc6ccc(-c7ccc8ccc(-c9ccc%10ccccc%10c9)cc8n7)cc6n5)nc4c3n2)cc1 ; c1ccc(-c2ccc3ccc4ccc(-c5ccc6ccc(-c7ccc8ccc(-c9cc%10ccccc%10c%10ccccc9%10)cc8n7)cc6n5)nc4c3n2)cc1]

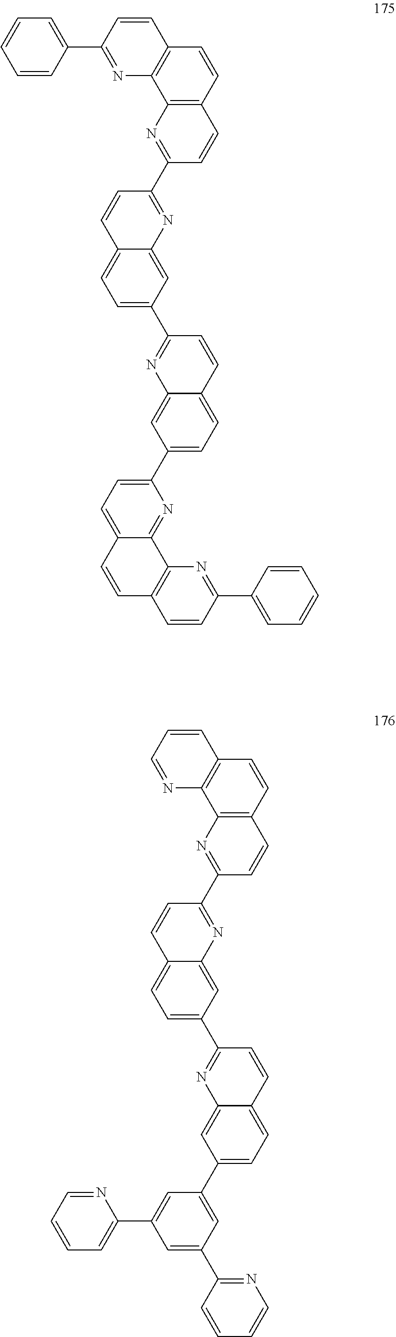 [SMILES: c1ccc(-c2ccc3ccc4ccc(-c5ccc6ccc(-c7ccc8ccc(-c9ccc%10ccc%11ccc(-c%12ccccc%12)nc%11c%10n9)nc8c7)nc6c5)nc4c3n2)cc1 ; c1ccc(-c2cc(-c3ccc4ccc(-c5ccc6ccc(-c7ccc8ccc9cccnc9c8n7)nc6c5)nc4c3)cc(-c3ccccn3)c2)nc1]

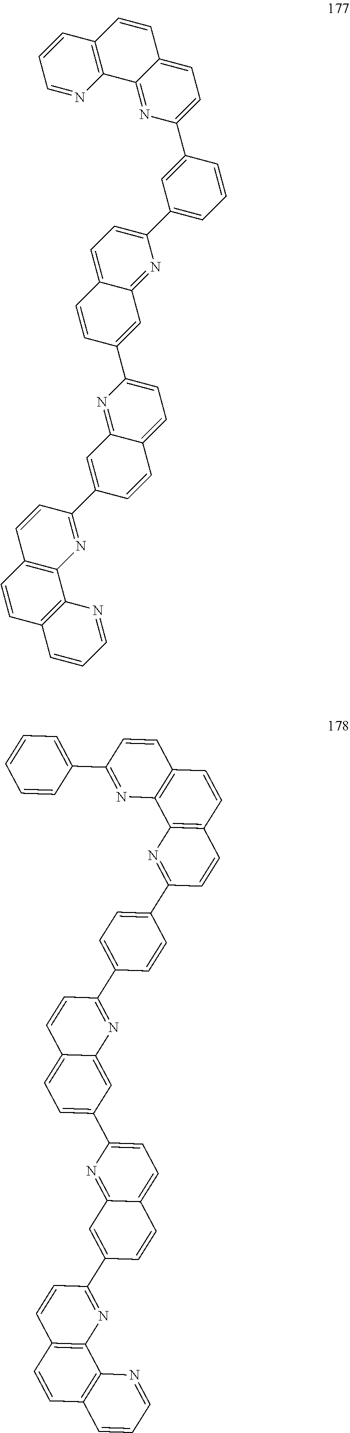 [SMILES: c1cc(-c2ccc3ccc(-c4ccc5ccc(-c6ccc7ccc8cccnc8c7n6)cc5n4)cc3n2)cc(-c2ccc3ccc4cccnc4c3n2)c1 ; c1ccc(-c2ccc3ccc4ccc(-c5ccc(-c6ccc7ccc(-c8ccc9ccc(-c%10ccc%11ccc%12cccnc%12c%11n%10)cc9n8)cc7n6)cc5)nc4c3n2)cc1]

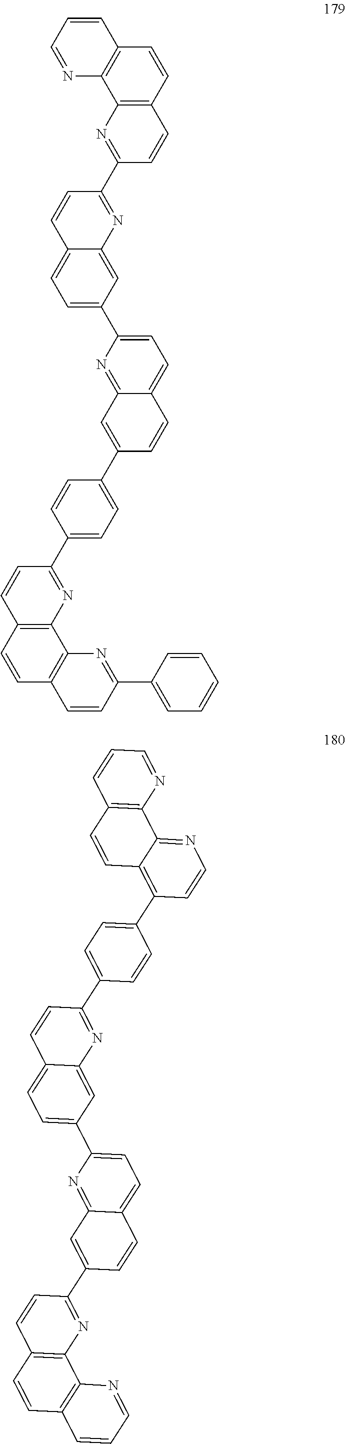

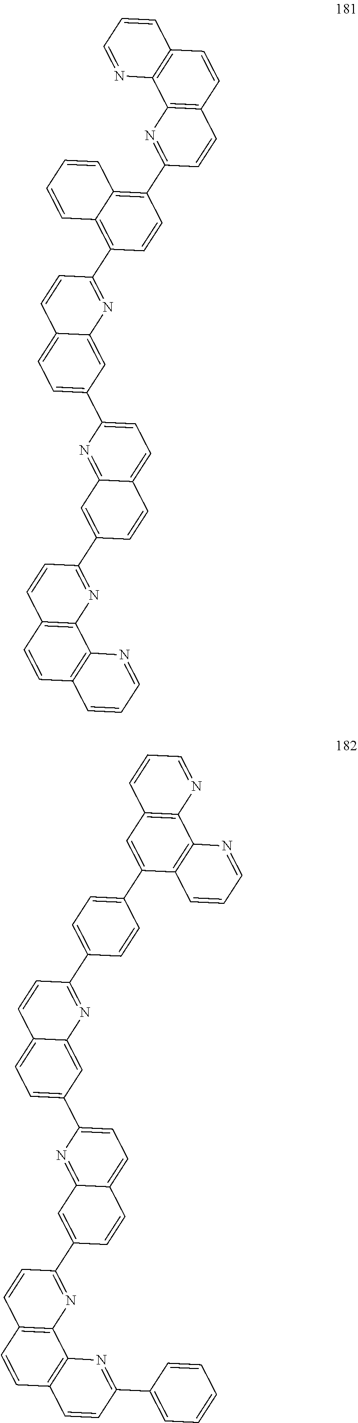 [SMILES: c1cnc2c(c1)ccc1ccc(-c3ccc4ccc(-c5ccc6ccc(-c7ccc(-c8ccc9ccc%10cccnc%10c9n8)c8ccccc78)nc6c5)nc4c3)nc12 ; c1ccc(-c2ccc3ccc4ccc(-c5ccc6ccc(-c7ccc8ccc(-c9ccc(-c%10cc%11cccnc%11c%11ncccc%10%11)cc9)nc8c7)nc6c5)nc4c3n2)cc1]

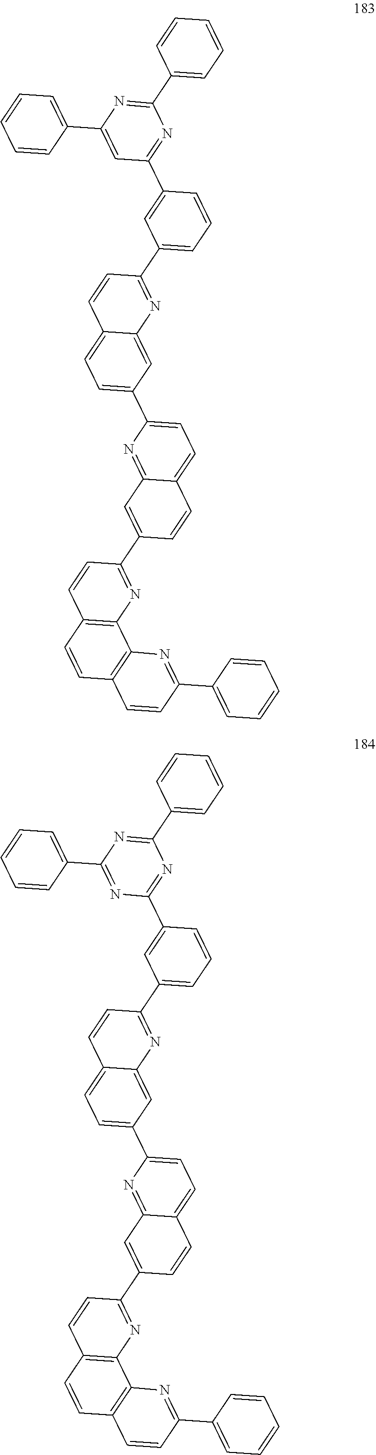

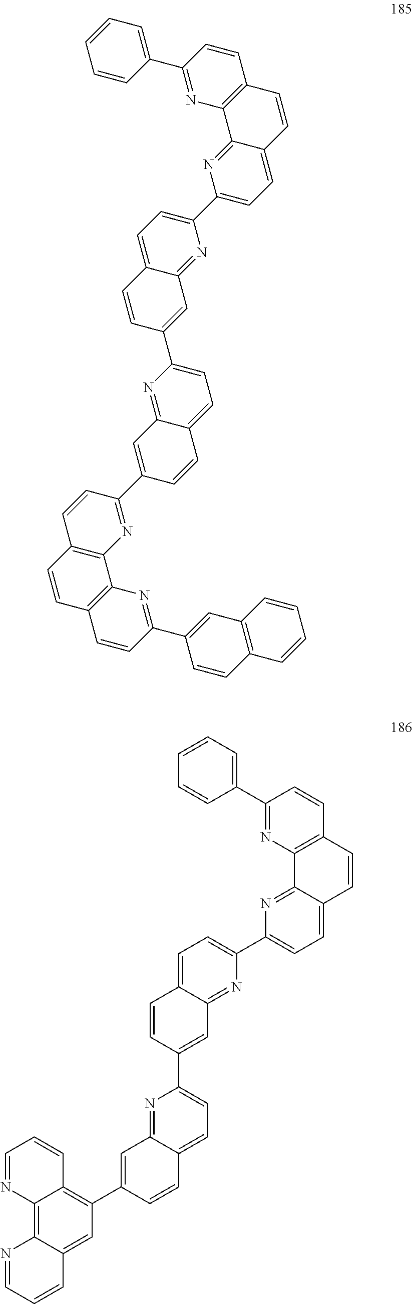 [SMILES: c1ccc(-c2ccc3ccc4ccc(-c5ccc6ccc(-c7ccc8ccc(-c9ccc%10ccc%11ccc(-c%12ccc%13ccccc%13c%12)nc%11c%10n9)cc8n7)cc6n5)nc4c3n2)cc1 ; c1ccc(-c2ccc3ccc4ccc(-c5ccc6ccc(-c7ccc8ccc(-c9cc%10cccnc%10c%10ncccc9%10)cc8n7)cc6n5)nc4c3n2)cc1]

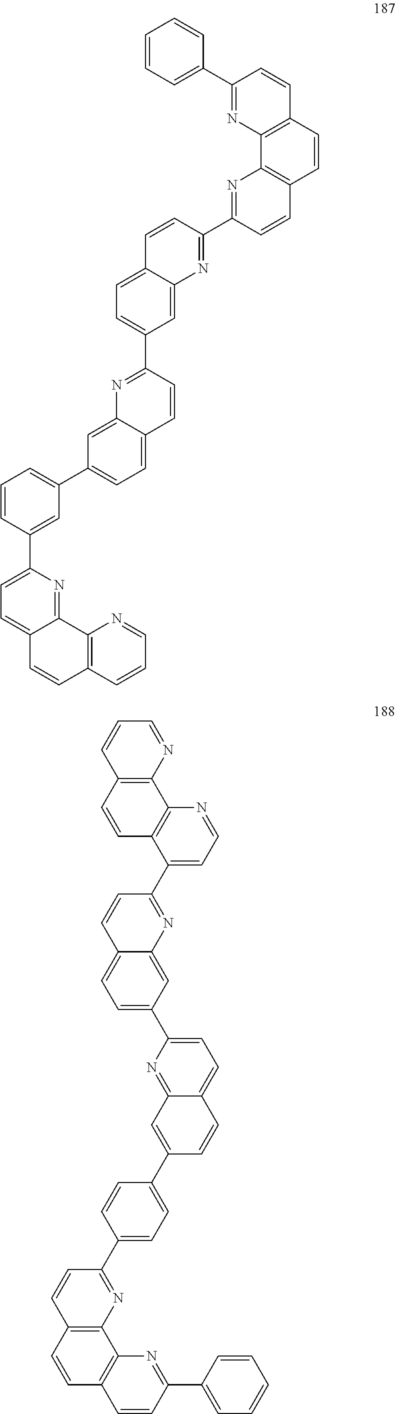

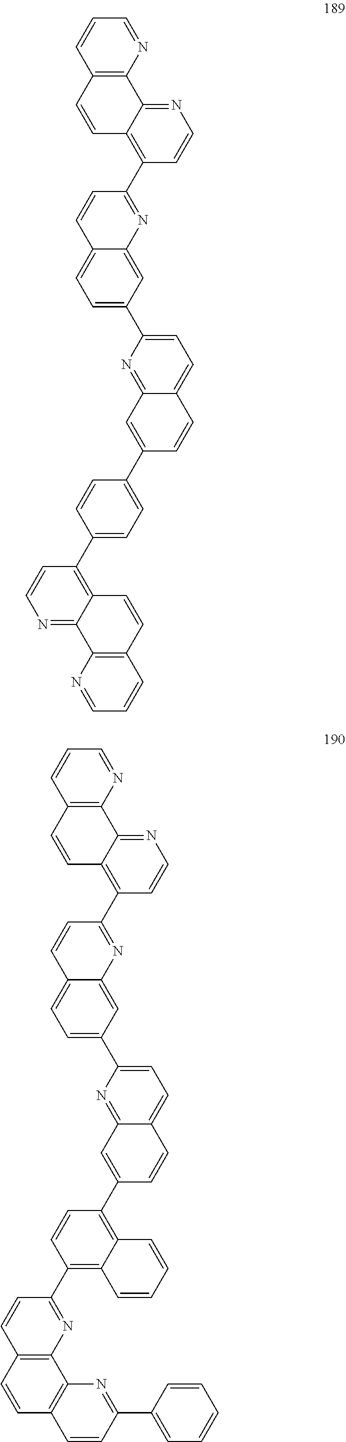

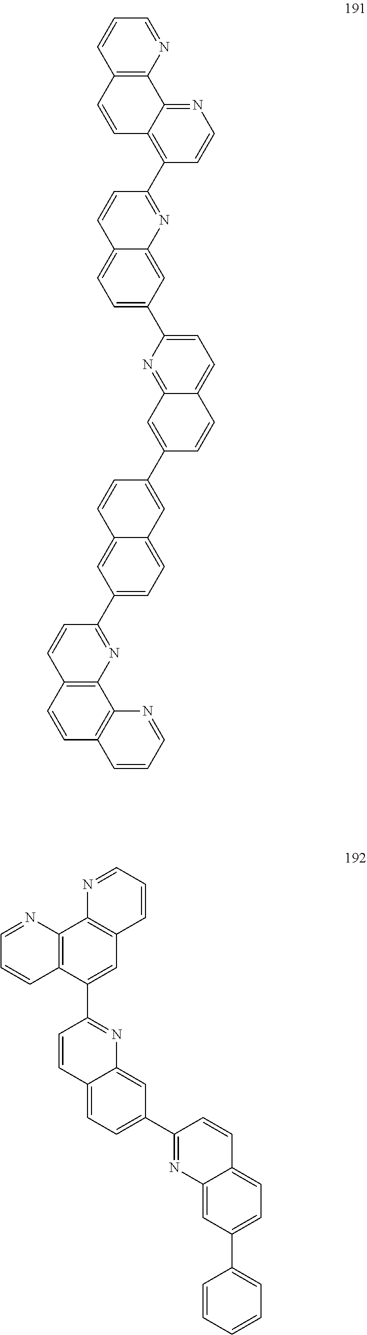 [SMILES: c1cnc2c(c1)ccc1ccc(-c3ccc4cc(-c5ccc6ccc(-c7ccc8ccc(-c9ccnc%10c9ccc9cccnc9%10)nc8c7)nc6c5)ccc4c3)nc12 ; c1ccc(-c2ccc3ccc(-c4ccc5ccc(-c6cc7cccnc7c7ncccc67)nc5c4)nc3c2)cc1]

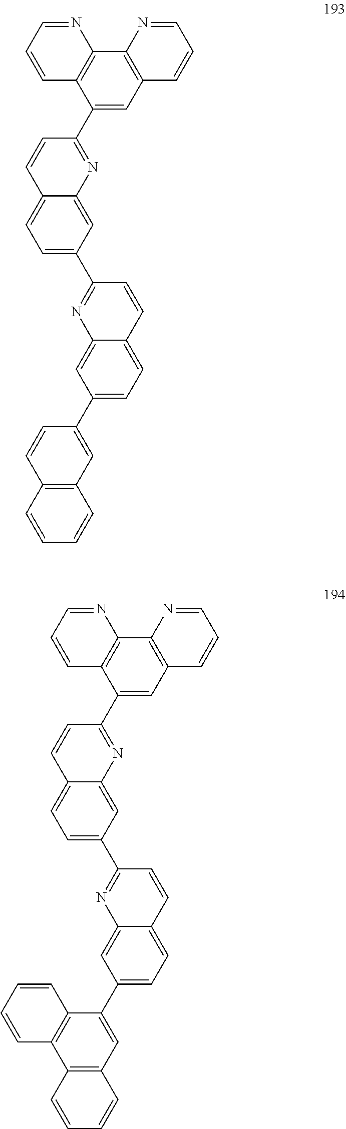 [SMILES: c1ccc2cc(-c3ccc4ccc(-c5ccc6ccc(-c7cc8cccnc8c8ncccc78)nc6c5)nc4c3)ccc2c1 ; c1ccc2c(c1)cc(-c1ccc3ccc(-c4ccc5ccc(-c6cc7cccnc7c7ncccc67)nc5c4)nc3c1)c1ccccc12]

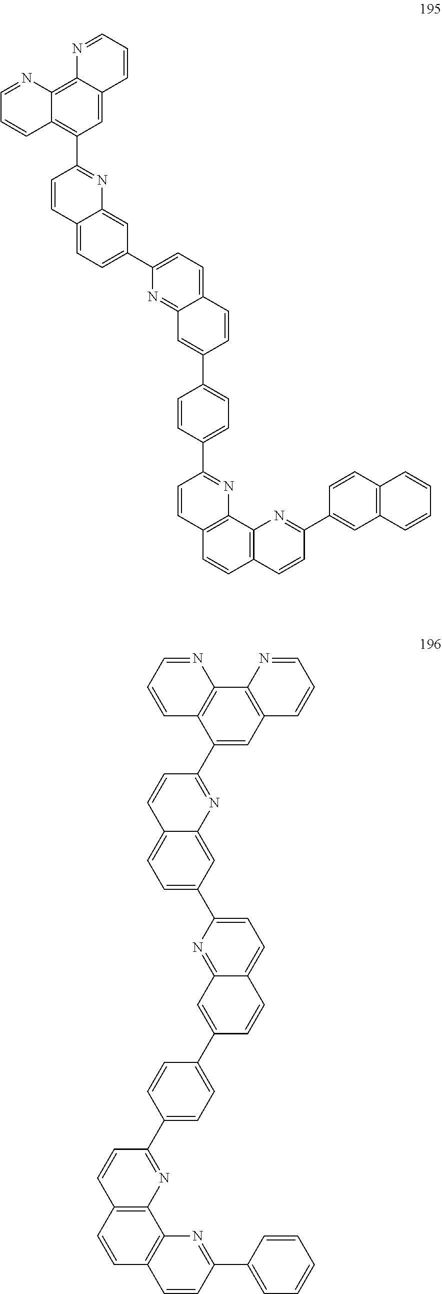 [SMILES: c1ccc2cc(-c3ccc4ccc5ccc(-c6ccc(-c7ccc8ccc(-c9ccc%10ccc(-c%11cc%12cccnc%12c%12ncccc%11%12)nc%10c9)nc8c7)cc6)nc5c4n3)ccc2c1 ; c1ccc(-c2ccc3ccc4ccc(-c5ccc(-c6ccc7ccc(-c8ccc9ccc(-c%10cc%11cccnc%11c%11ncccc%10%11)nc9c8)nc7c6)cc5)nc4c3n2)cc1]

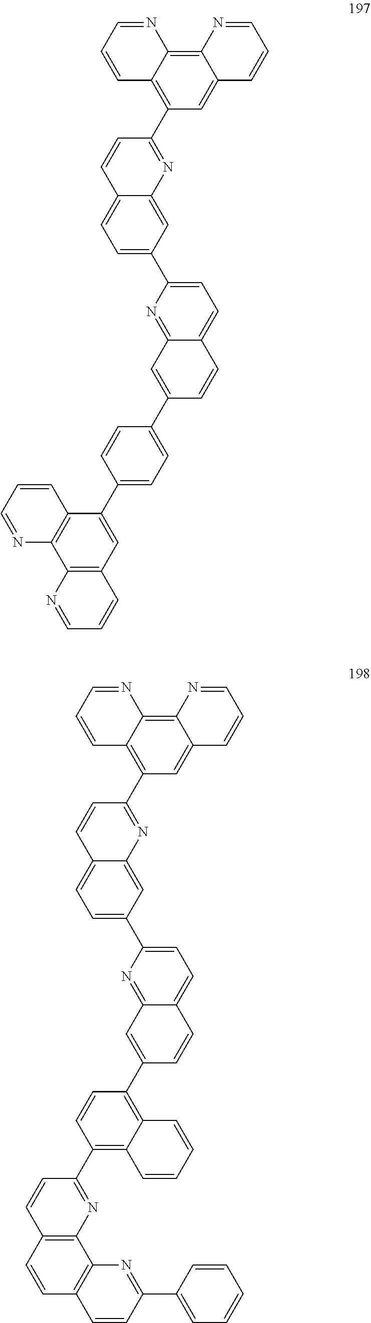 [SMILES: c1cnc2c(c1)cc(-c1ccc(-c3ccc4ccc(-c5ccc6ccc(-c7cc8cccnc8c8ncccc78)nc6c5)nc4c3)cc1)c1cccnc12 ; c1ccc(-c2ccc3ccc4ccc(-c5ccc(-c6ccc7ccc(-c8ccc9ccc(-c%10cc%11cccnc%11c%11ncccc%10%11)nc9c8)nc7c6)c6ccccc56)nc4c3n2)cc1]

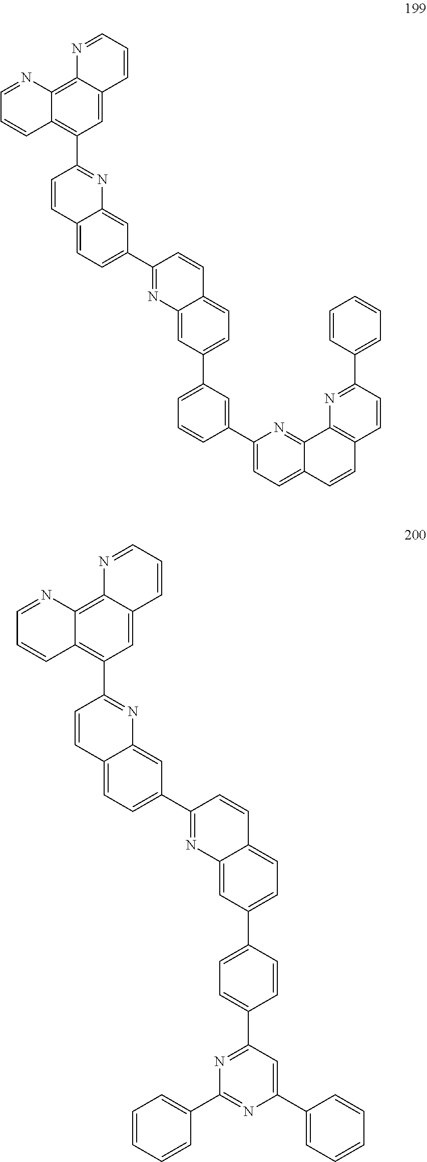 [SMILES: c1ccc(-c2ccc3ccc4ccc(-c5cccc(-c6ccc7ccc(-c8ccc9ccc(-c%10cc%11cccnc%11c%11ncccc%10%11)nc9c8)nc7c6)c5)nc4c3n2)cc1 ; c1ccc(-c2cc(-c3ccc(-c4ccc5ccc(-c6ccc7ccc(-c8cc9cccnc9c9ncccc89)nc7c6)nc5c4)cc3)nc(-c3ccccc3)n2)cc1]

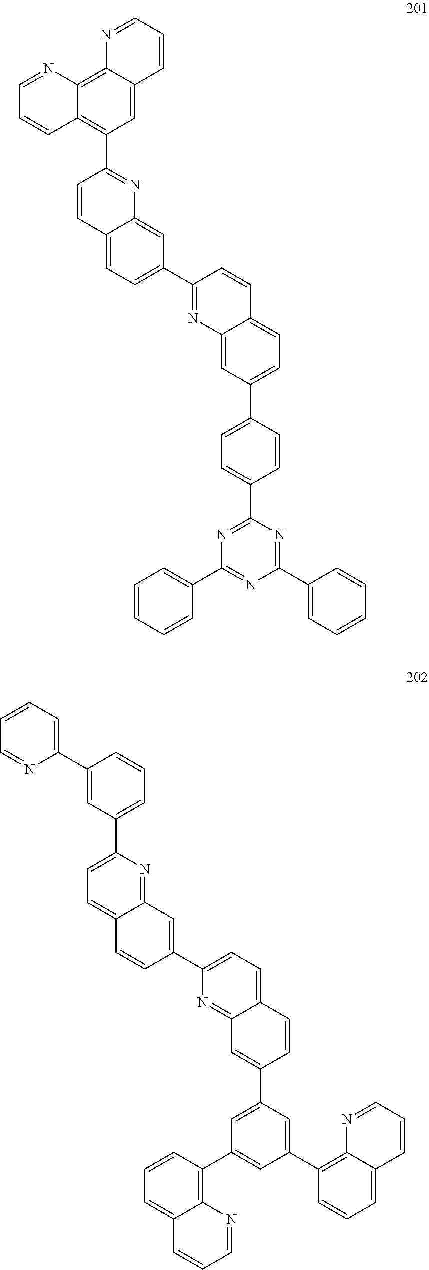 [SMILES: c1ccc(-c2nc(-c3ccccc3)nc(-c3ccc(-c4ccc5ccc(-c6ccc7ccc(-c8cc9cccnc9c9ncccc89)nc7c6)nc5c4)cc3)n2)cc1 ; c1ccc(-c2cccc(-c3ccc4ccc(-c5ccc6ccc(-c7cc(-c8cccc9cccnc89)cc(-c8cccc9cccnc89)c7)cc6n5)cc4n3)c2)nc1]

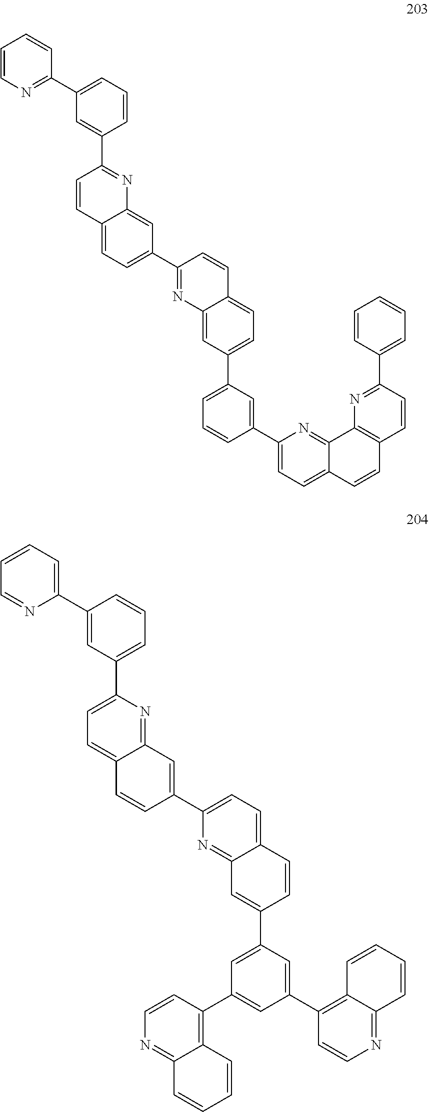 [SMILES: c1ccc(-c2ccc3ccc4ccc(-c5cccc(-c6ccc7ccc(-c8ccc9ccc(-c%10cccc(-c%11ccccn%11)c%10)nc9c8)nc7c6)c5)nc4c3n2)cc1 ; c1ccc(-c2cccc(-c3ccc4ccc(-c5ccc6ccc(-c7cc(-c8ccnc9ccccc89)cc(-c8ccnc9ccccc89)c7)cc6n5)cc4n3)c2)nc1]

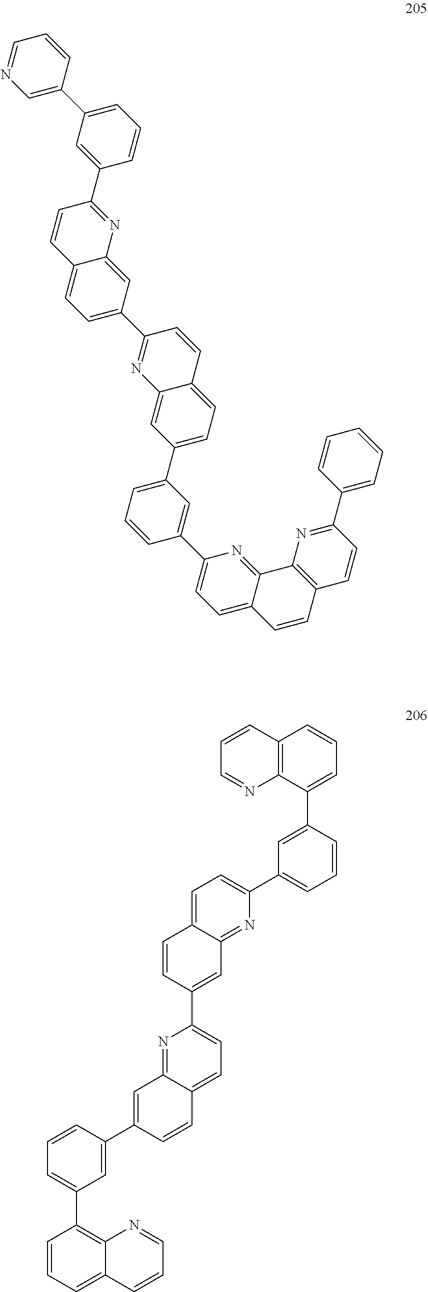 [SMILES: c1ccc(-c2ccc3ccc4ccc(-c5cccc(-c6ccc7ccc(-c8ccc9ccc(-c%10cccc(-c%11cccnc%11)c%10)nc9c8)nc7c6)c5)nc4c3n2)cc1 ; c1cc(-c2ccc3ccc(-c4ccc5ccc(-c6cccc(-c7cccc8cccnc78)c6)nc5c4)nc3c2)cc(-c2cccc3cccnc23)c1]

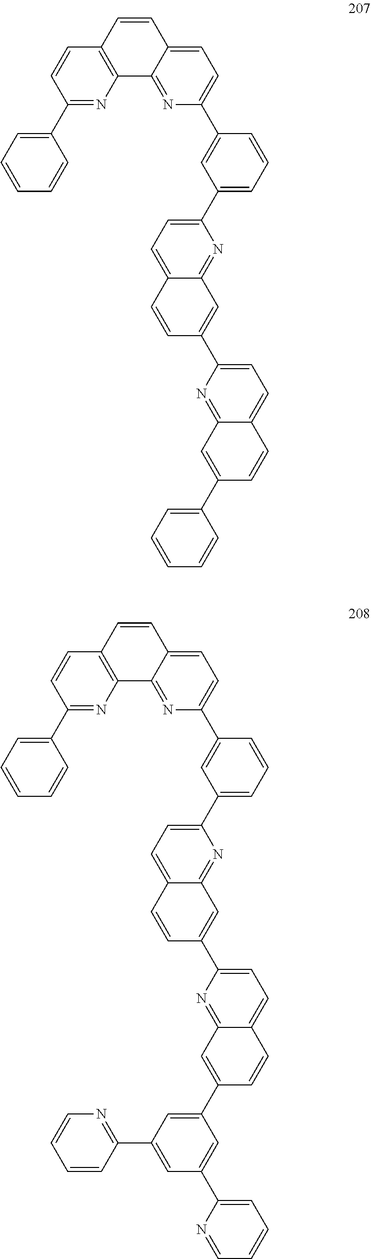 [SMILES: c1ccc(-c2ccc3ccc(-c4ccc5ccc(-c6cccc(-c7ccc8ccc9ccc(-c%10ccccc%10)nc9c8n7)c6)nc5c4)nc3c2)cc1 ; c1ccc(-c2ccc3ccc4ccc(-c5cccc(-c6ccc7ccc(-c8ccc9ccc(-c%10cc(-c%11ccccn%11)cc(-c%11ccccn%11)c%10)cc9n8)cc7n6)c5)nc4c3n2)cc1]

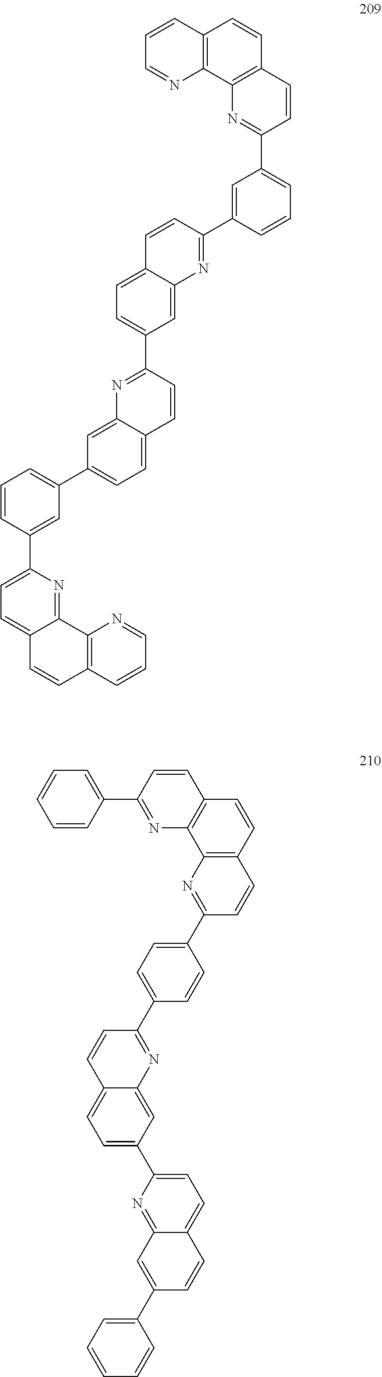 [SMILES: c1cc(-c2ccc3ccc(-c4ccc5ccc(-c6cccc(-c7ccc8ccc9cccnc9c8n7)c6)nc5c4)nc3c2)cc(-c2ccc3ccc4cccnc4c3n2)c1 ; c1ccc(-c2ccc3ccc(-c4ccc5ccc(-c6ccc(-c7ccc8ccc9ccc(-c%10ccccc%10)nc9c8n7)cc6)nc5c4)nc3c2)cc1]

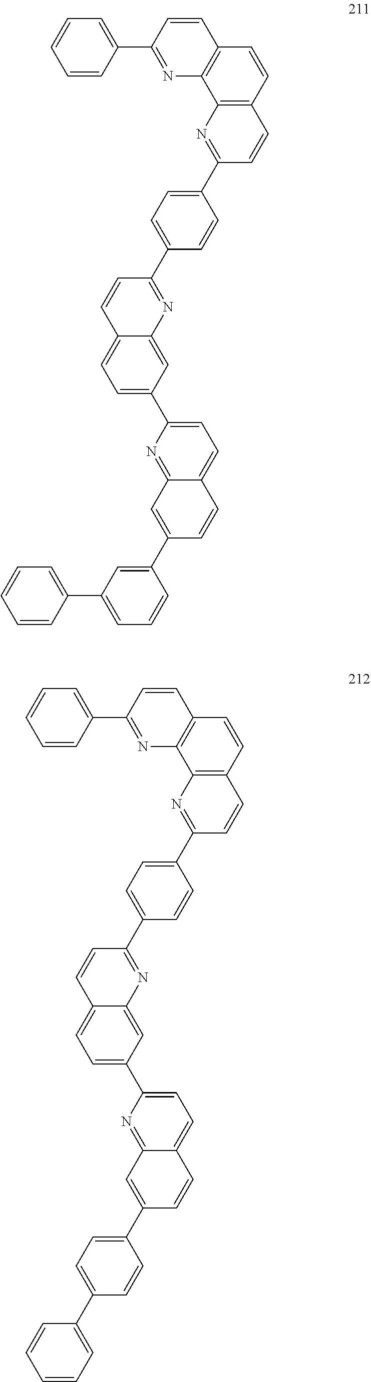 [SMILES: c1ccc(-c2cccc(-c3ccc4ccc(-c5ccc6ccc(-c7ccc(-c8ccc9ccc%10ccc(-c%11ccccc%11)nc%10c9n8)cc7)nc6c5)nc4c3)c2)cc1 ; c1ccc(-c2ccc(-c3ccc4ccc(-c5ccc6ccc(-c7ccc(-c8ccc9ccc%10ccc(-c%11ccccc%11)nc%10c9n8)cc7)nc6c5)nc4c3)cc2)cc1]

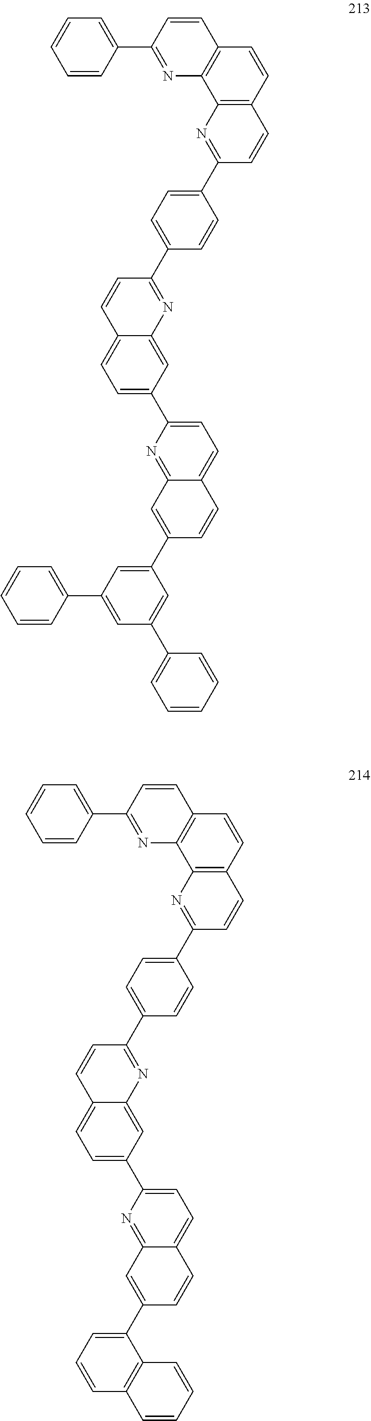 [SMILES: c1ccc(-c2cc(-c3ccccc3)cc(-c3ccc4ccc(-c5ccc6ccc(-c7ccc(-c8ccc9ccc%10ccc(-c%11ccccc%11)nc%10c9n8)cc7)nc6c5)nc4c3)c2)cc1 ; c1ccc(-c2ccc3ccc4ccc(-c5ccc(-c6ccc7ccc(-c8ccc9ccc(-c%10cccc%11ccccc%10%11)cc9n8)cc7n6)cc5)nc4c3n2)cc1]

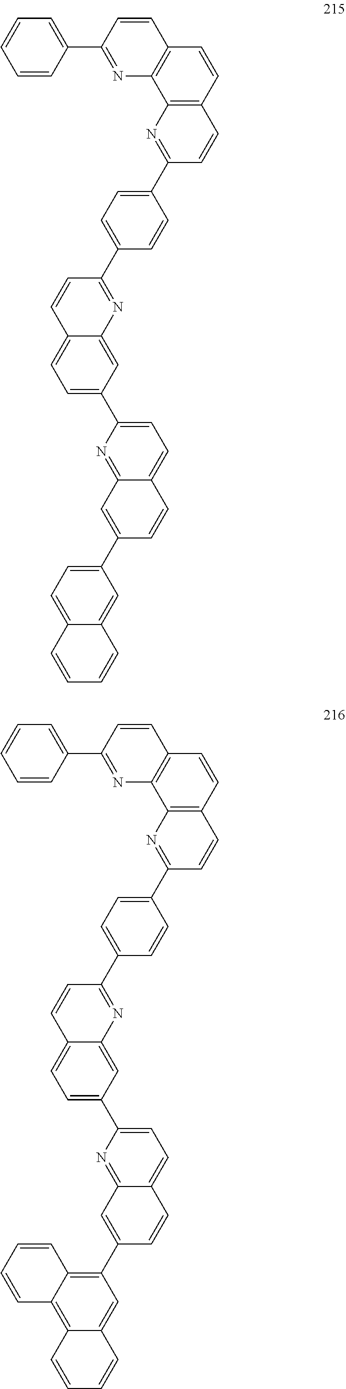

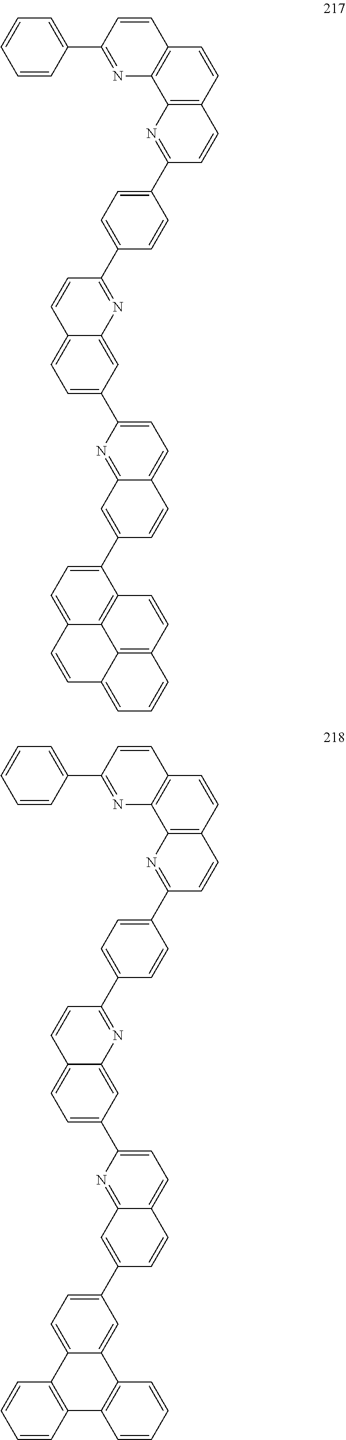

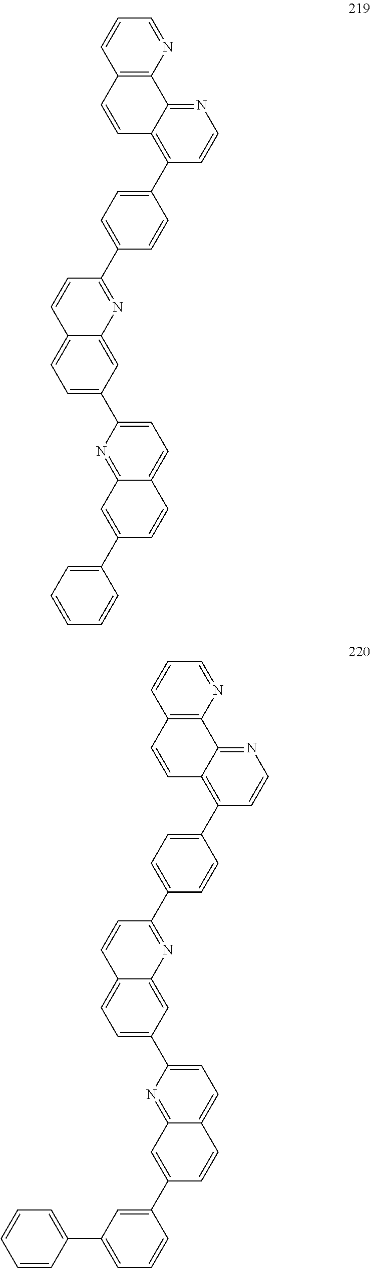 [SMILES: c1ccc(-c2ccc3ccc(-c4ccc5ccc(-c6ccc(-c7ccnc8c7ccc7cccnc78)cc6)nc5c4)nc3c2)cc1 ; c1ccc(-c2cccc(-c3ccc4ccc(-c5ccc6ccc(-c7ccc(-c8ccnc9c8ccc8cccnc89)cc7)nc6c5)nc4c3)c2)cc1]

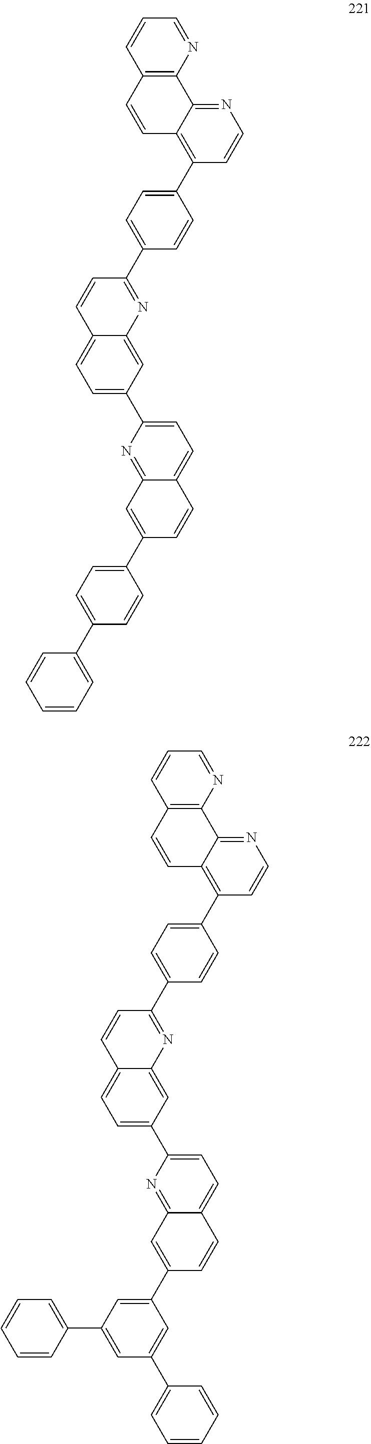 [SMILES: c1ccc(-c2ccc(-c3ccc4ccc(-c5ccc6ccc(-c7ccc(-c8ccnc9c8ccc8cccnc89)cc7)nc6c5)nc4c3)cc2)cc1 ; c1ccc(-c2cc(-c3ccccc3)cc(-c3ccc4ccc(-c5ccc6ccc(-c7ccc(-c8ccnc9c8ccc8cccnc89)cc7)nc6c5)nc4c3)c2)cc1]

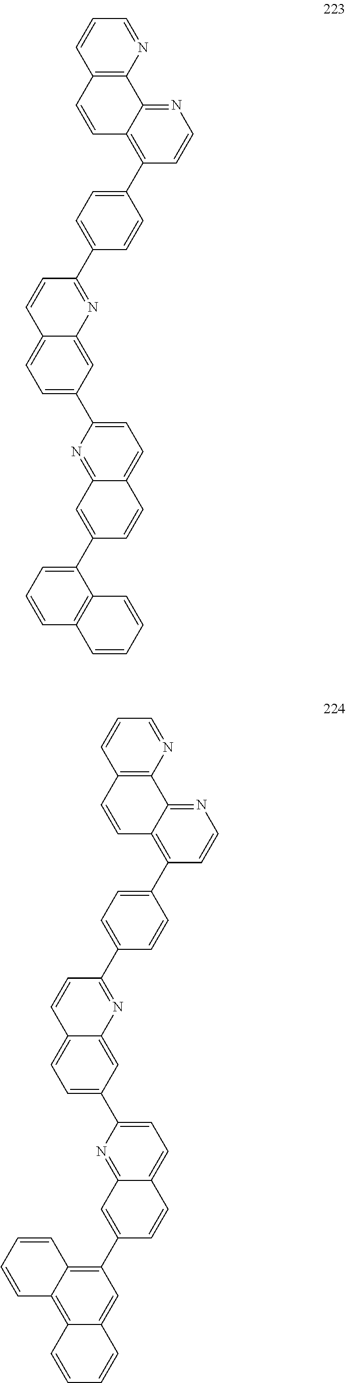 [SMILES: c1ccc2c(-c3ccc4ccc(-c5ccc6ccc(-c7ccc(-c8ccnc9c8ccc8cccnc89)cc7)nc6c5)nc4c3)cccc2c1 ; c1ccc2c(c1)cc(-c1ccc3ccc(-c4ccc5ccc(-c6ccc(-c7ccnc8c7ccc7cccnc78)cc6)nc5c4)nc3c1)c1ccccc12]

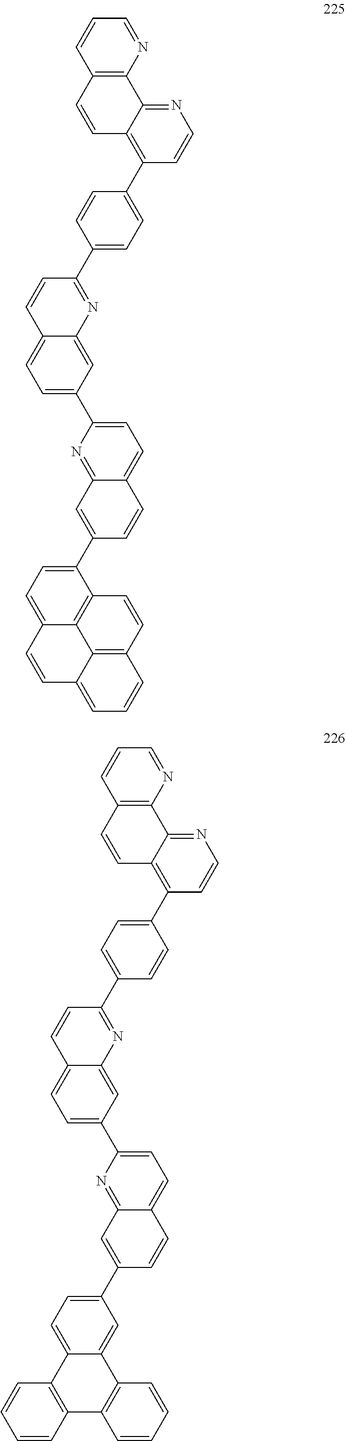

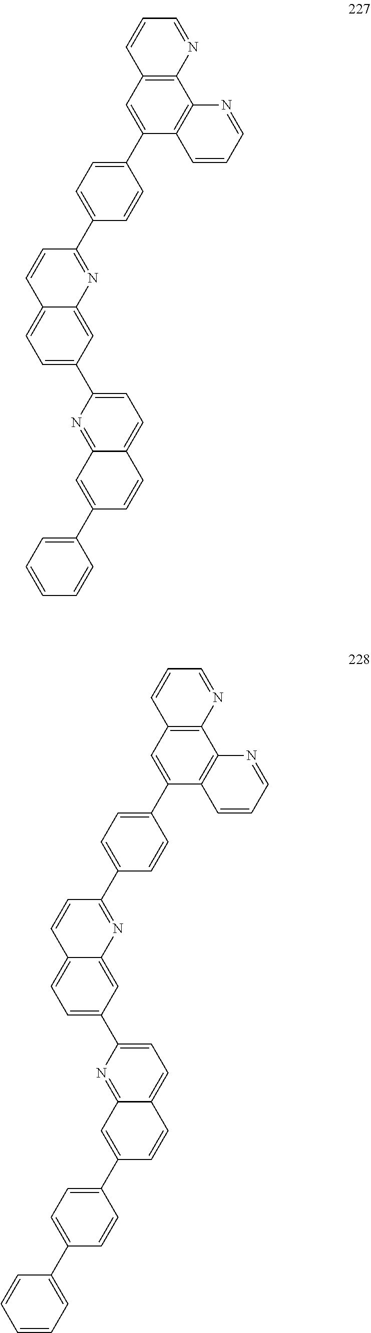 [SMILES: c1ccc(-c2ccc3ccc(-c4ccc5ccc(-c6ccc(-c7cc8cccnc8c8ncccc78)cc6)nc5c4)nc3c2)cc1 ; c1ccc(-c2ccc(-c3ccc4ccc(-c5ccc6ccc(-c7ccc(-c8cc9cccnc9c9ncccc89)cc7)nc6c5)nc4c3)cc2)cc1]

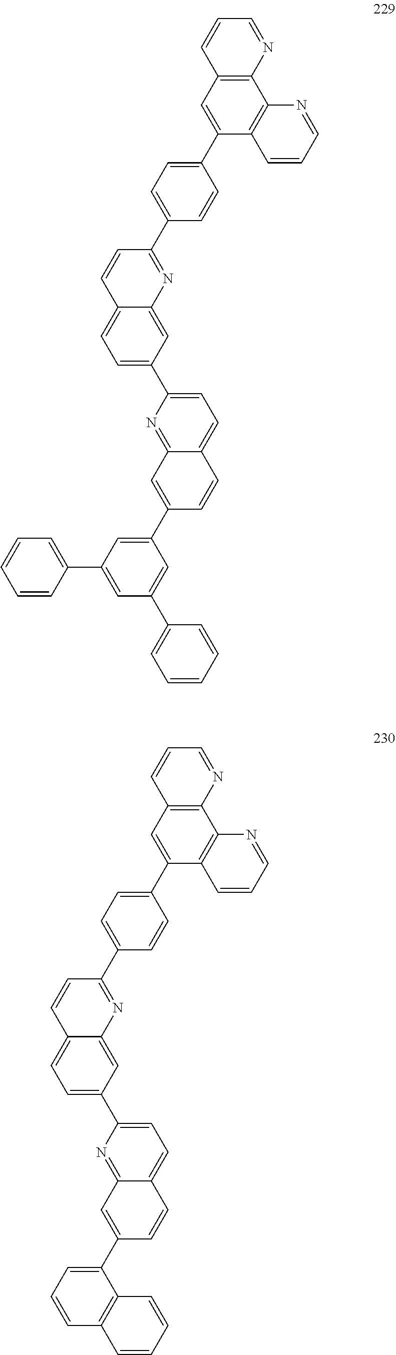 [SMILES: c1ccc(-c2cc(-c3ccccc3)cc(-c3ccc4ccc(-c5ccc6ccc(-c7ccc(-c8cc9cccnc9c9ncccc89)cc7)nc6c5)nc4c3)c2)cc1 ; c1ccc2c(-c3ccc4ccc(-c5ccc6ccc(-c7ccc(-c8cc9cccnc9c9ncccc89)cc7)nc6c5)nc4c3)cccc2c1]

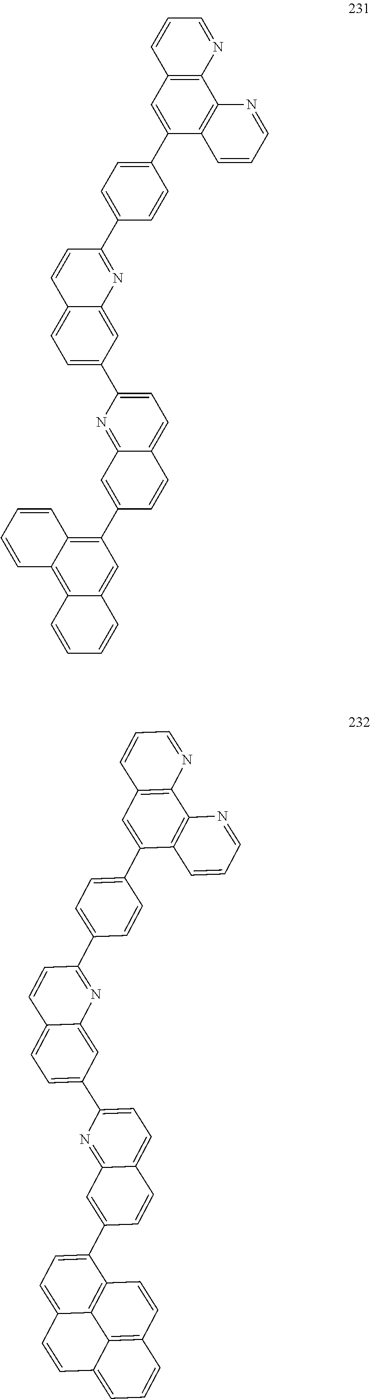 [SMILES: c1ccc2c(c1)cc(-c1ccc3ccc(-c4ccc5ccc(-c6ccc(-c7cc8cccnc8c8ncccc78)cc6)nc5c4)nc3c1)c1ccccc12 ; c1cnc2c(c1)cc(-c1ccc(-c3ccc4ccc(-c5ccc6ccc(-c7ccc8ccc9cccc%10ccc7c8c9%10)cc6n5)cc4n3)cc1)c1cccnc12]

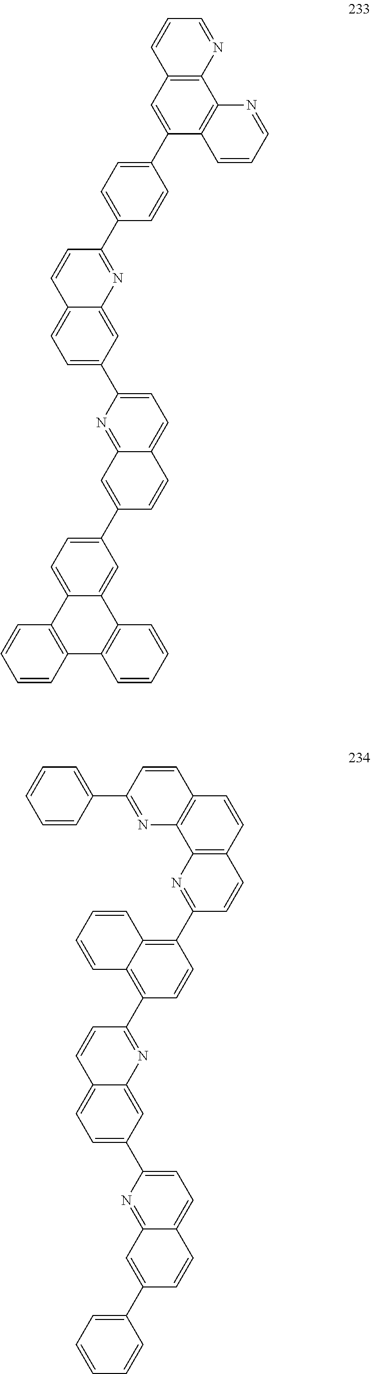 [SMILES: c1cnc2c(c1)cc(-c1ccc(-c3ccc4ccc(-c5ccc6ccc(-c7ccc8c9ccccc9c9ccccc9c8c7)cc6n5)cc4n3)cc1)c1cccnc12 ; c1ccc(-c2ccc3ccc(-c4ccc5ccc(-c6ccc(-c7ccc8ccc9ccc(-c%10ccccc%10)nc9c8n7)c7ccccc67)nc5c4)nc3c2)cc1]

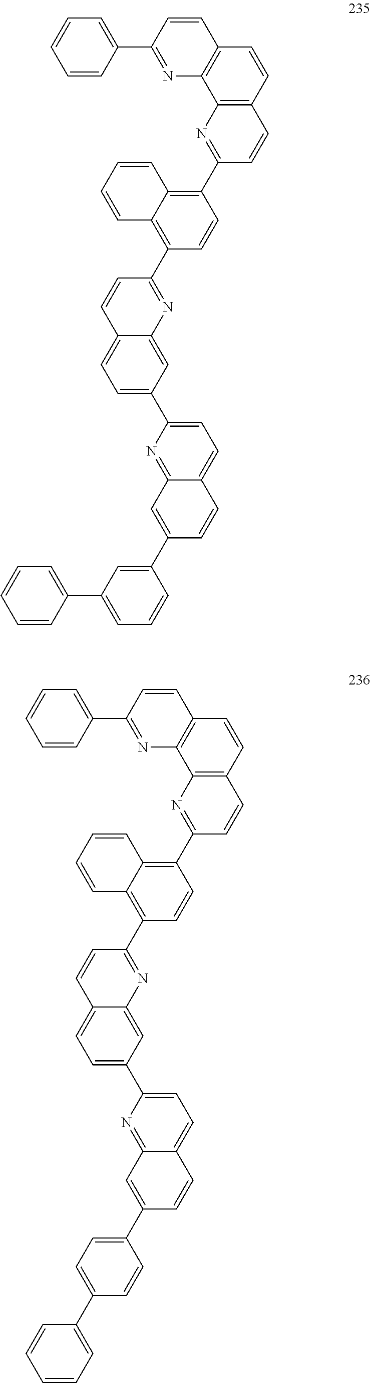 [SMILES: c1ccc(-c2cccc(-c3ccc4ccc(-c5ccc6ccc(-c7ccc(-c8ccc9ccc%10ccc(-c%11ccccc%11)nc%10c9n8)c8ccccc78)nc6c5)nc4c3)c2)cc1 ; c1ccc(-c2ccc(-c3ccc4ccc(-c5ccc6ccc(-c7ccc(-c8ccc9ccc%10ccc(-c%11ccccc%11)nc%10c9n8)c8ccccc78)nc6c5)nc4c3)cc2)cc1]

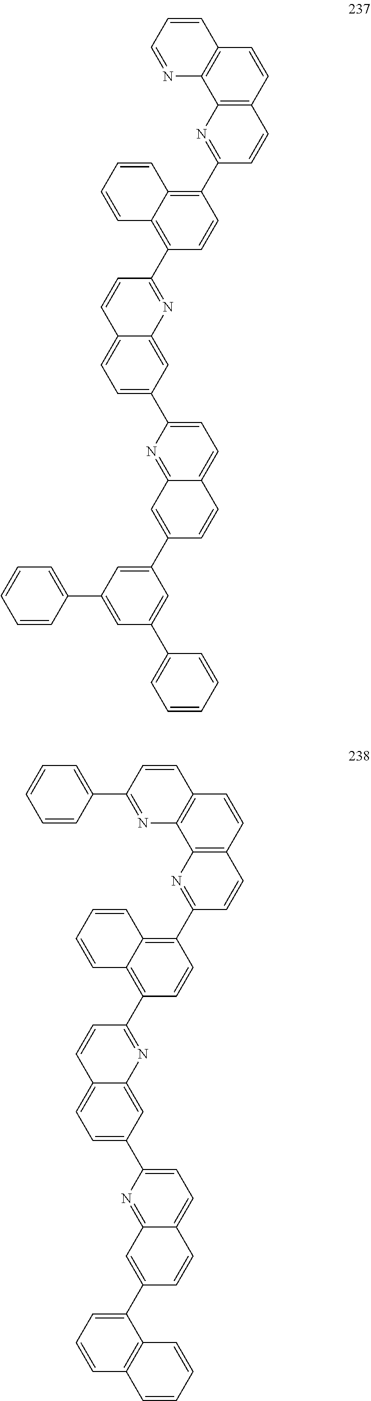 [SMILES: c1ccc(-c2cc(-c3ccccc3)cc(-c3ccc4ccc(-c5ccc6ccc(-c7ccc(-c8ccc9ccc%10cccnc%10c9n8)c8ccccc78)nc6c5)nc4c3)c2)cc1 ; c1ccc(-c2ccc3ccc4ccc(-c5ccc(-c6ccc7ccc(-c8ccc9ccc(-c%10cccc%11ccccc%10%11)cc9n8)cc7n6)c6ccccc56)nc4c3n2)cc1]

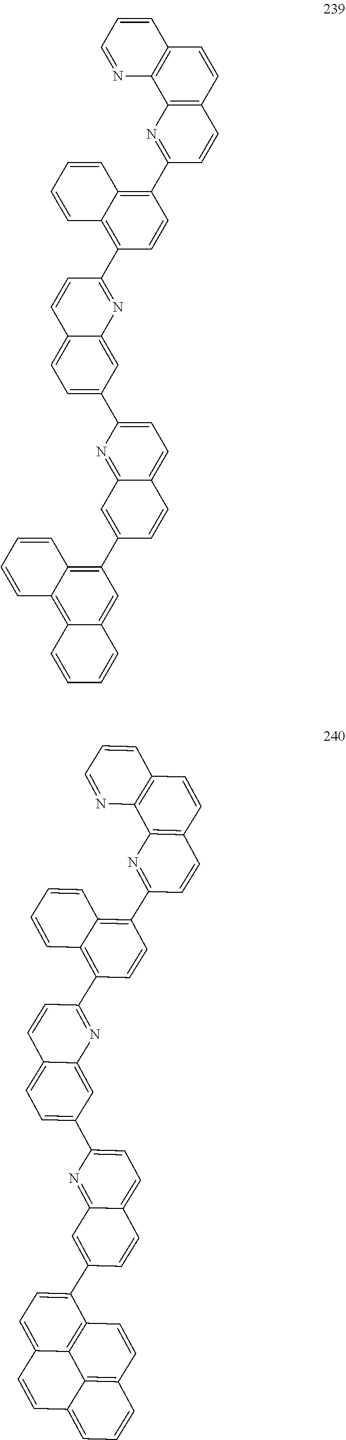 [SMILES: c1ccc2c(c1)cc(-c1ccc3ccc(-c4ccc5ccc(-c6ccc(-c7ccc8ccc9cccnc9c8n7)c7ccccc67)nc5c4)nc3c1)c1ccccc12 ; c1cnc2c(c1)ccc1ccc(-c3ccc(-c4ccc5ccc(-c6ccc7ccc(-c8ccc9ccc%10cccc%11ccc8c9c%10%11)cc7n6)cc5n4)c4ccccc34)nc12]

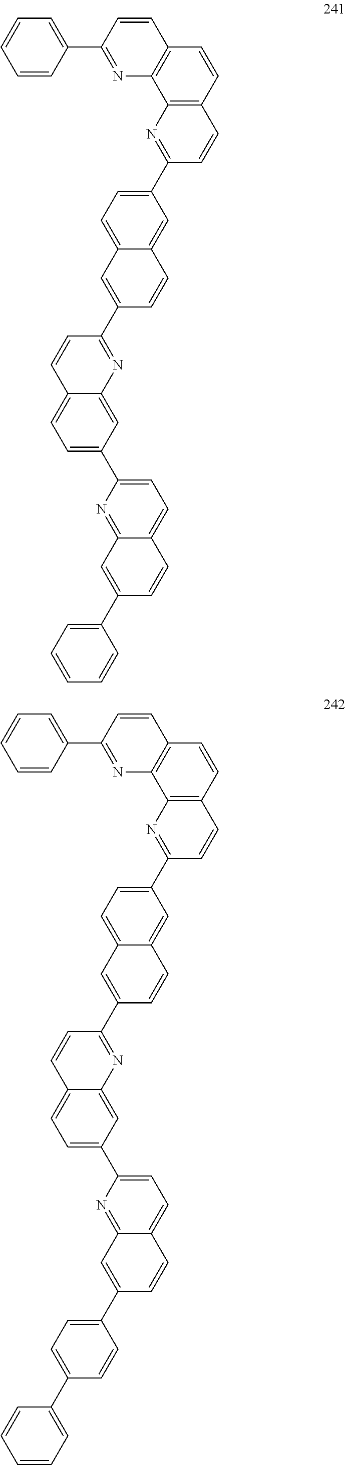

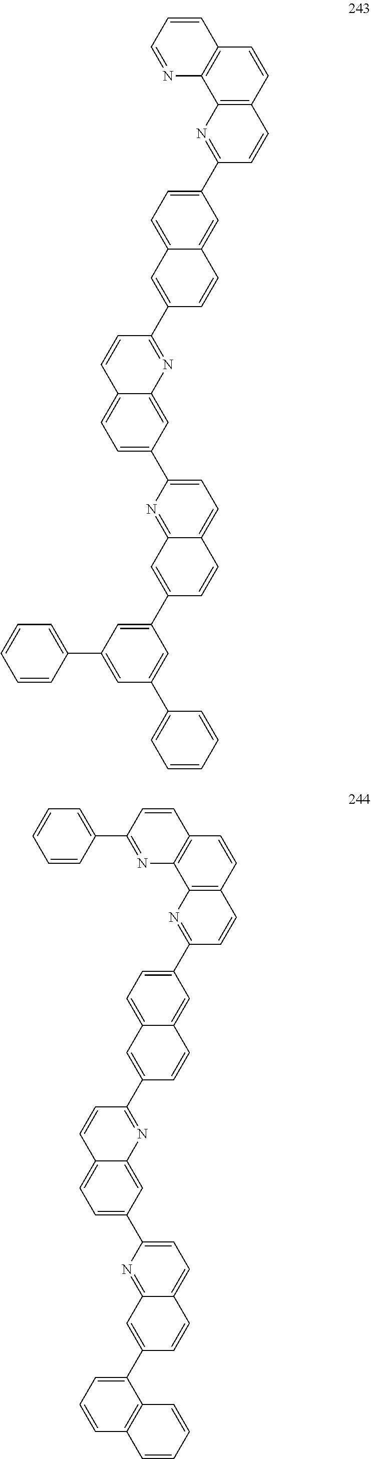

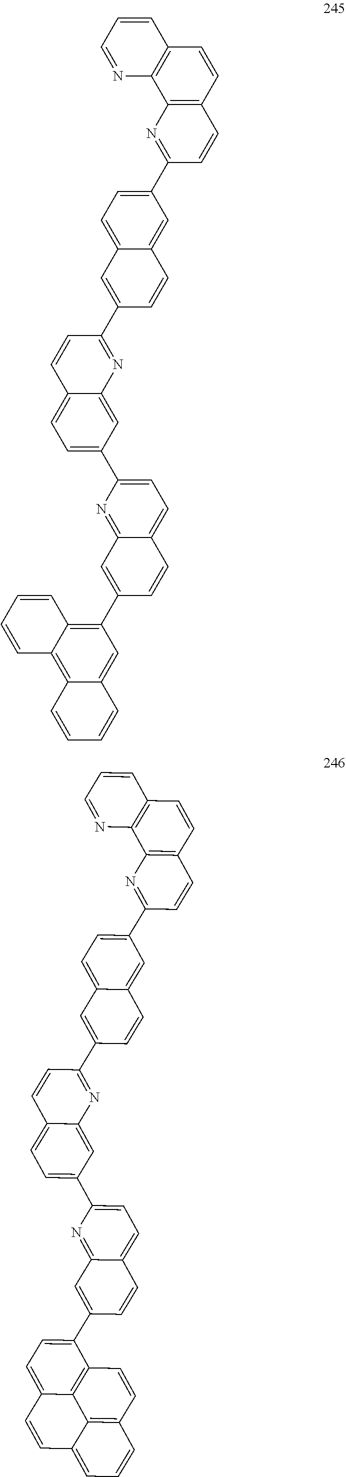

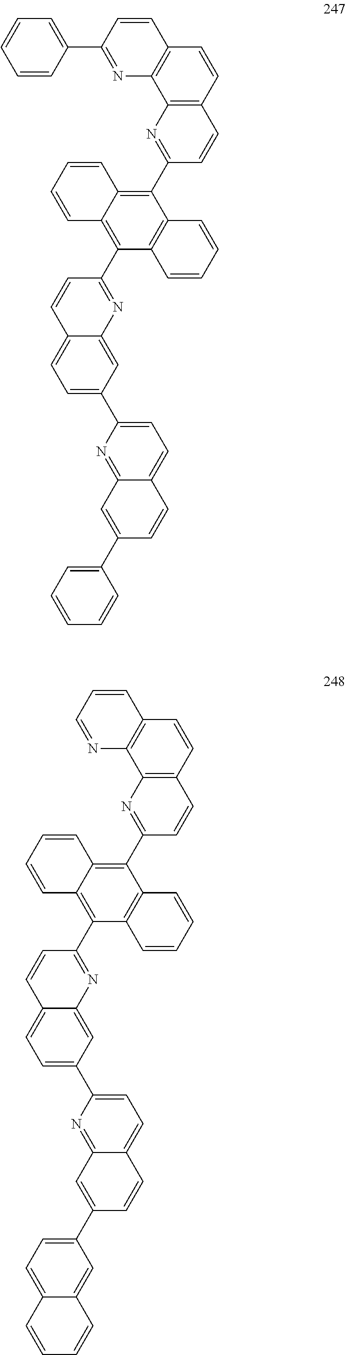 [SMILES: c1ccc(-c2ccc3ccc(-c4ccc5ccc(-c6c7ccccc7c(-c7ccc8ccc9ccc(-c%10ccccc%10)nc9c8n7)c7ccccc67)nc5c4)nc3c2)cc1 ; c1ccc2cc(-c3ccc4ccc(-c5ccc6ccc(-c7c8ccccc8c(-c8ccc9ccc%10cccnc%10c9n8)c8ccccc78)nc6c5)nc4c3)ccc2c1]

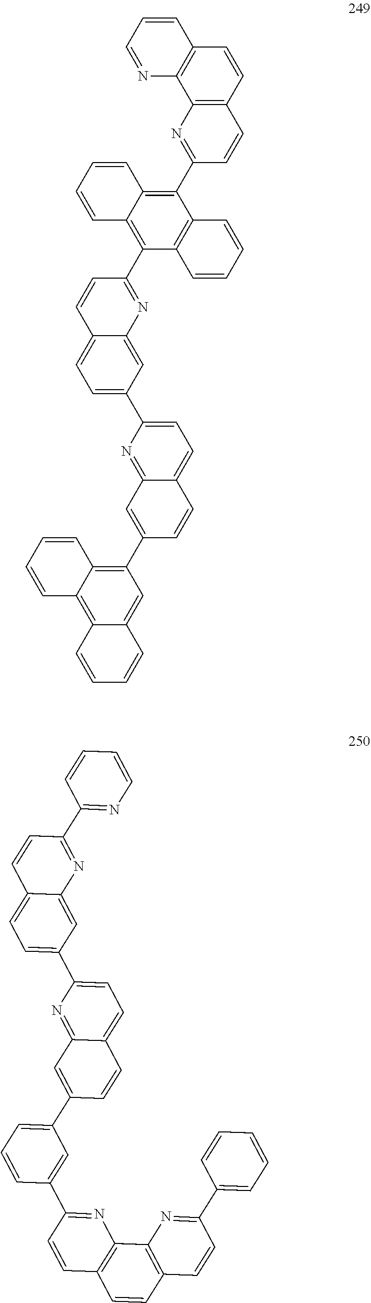 [SMILES: c1ccc2c(c1)cc(-c1ccc3ccc(-c4ccc5ccc(-c6c7ccccc7c(-c7ccc8ccc9cccnc9c8n7)c7ccccc67)nc5c4)nc3c1)c1ccccc12 ; c1ccc(-c2ccc3ccc4ccc(-c5cccc(-c6ccc7ccc(-c8ccc9ccc(-c%10ccccn%10)nc9c8)nc7c6)c5)nc4c3n2)cc1]

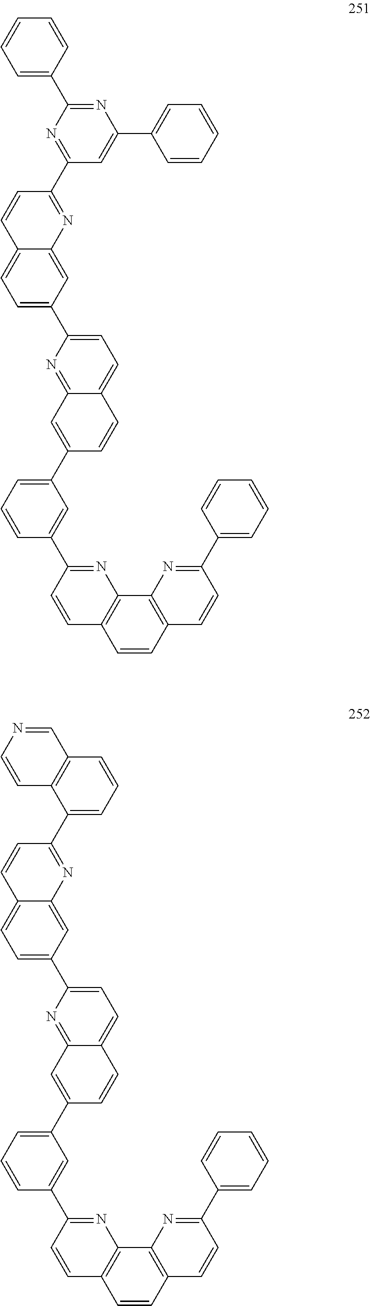 [SMILES: c1ccc(-c2cc(-c3ccc4ccc(-c5ccc6ccc(-c7cccc(-c8ccc9ccc%10ccc(-c%11ccccc%11)nc%10c9n8)c7)cc6n5)cc4n3)nc(-c3ccccc3)n2)cc1 ; c1ccc(-c2ccc3ccc4ccc(-c5cccc(-c6ccc7ccc(-c8ccc9ccc(-c%10cccc%11cnccc%10%11)nc9c8)nc7c6)c5)nc4c3n2)cc1]

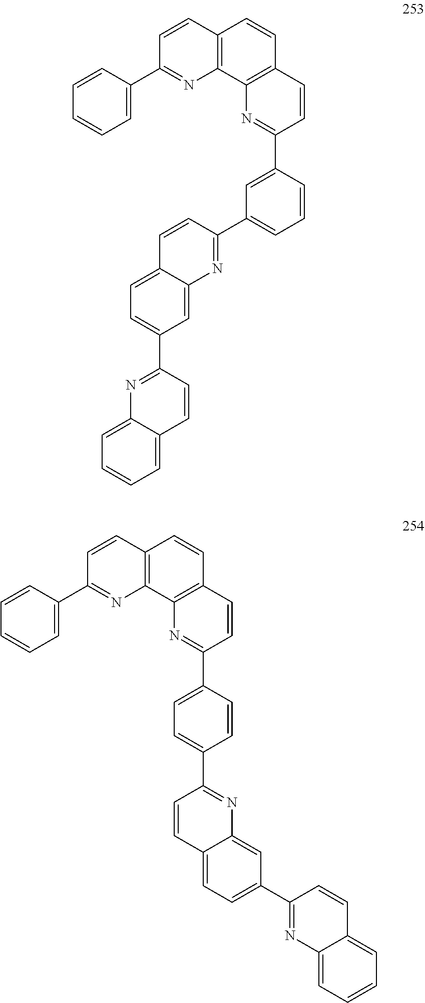 [SMILES: c1ccc(-c2ccc3ccc4ccc(-c5cccc(-c6ccc7ccc(-c8ccc9ccccc9n8)cc7n6)c5)nc4c3n2)cc1 ; c1ccc(-c2ccc3ccc4ccc(-c5ccc(-c6ccc7ccc(-c8ccc9ccccc9n8)cc7n6)cc5)nc4c3n2)cc1]

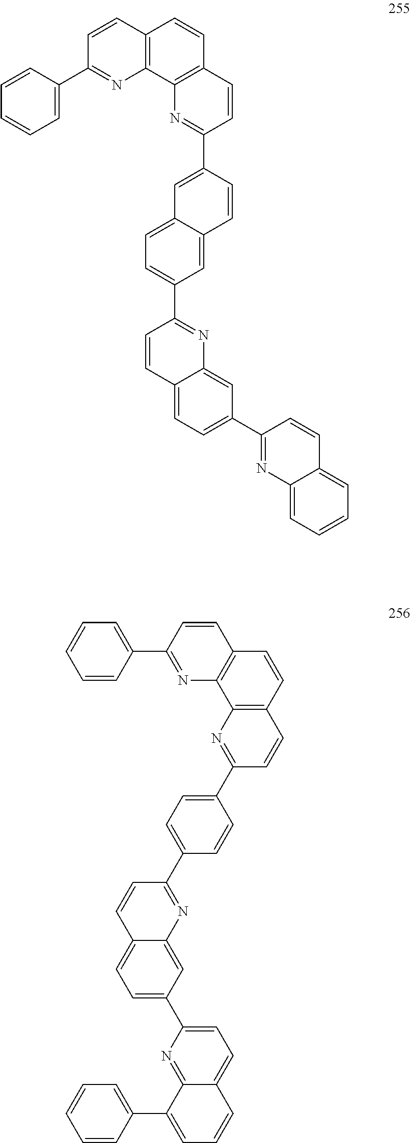 [SMILES: c1ccc(-c2ccc3ccc4ccc(-c5ccc6cc(-c7ccc8ccc(-c9ccc%10ccccc%10n9)cc8n7)ccc6c5)nc4c3n2)cc1 ; c1ccc(-c2ccc3ccc4ccc(-c5ccc(-c6ccc7ccc(-c8ccc9cccc(-c%10ccccc%10)c9n8)cc7n6)cc5)nc4c3n2)cc1]

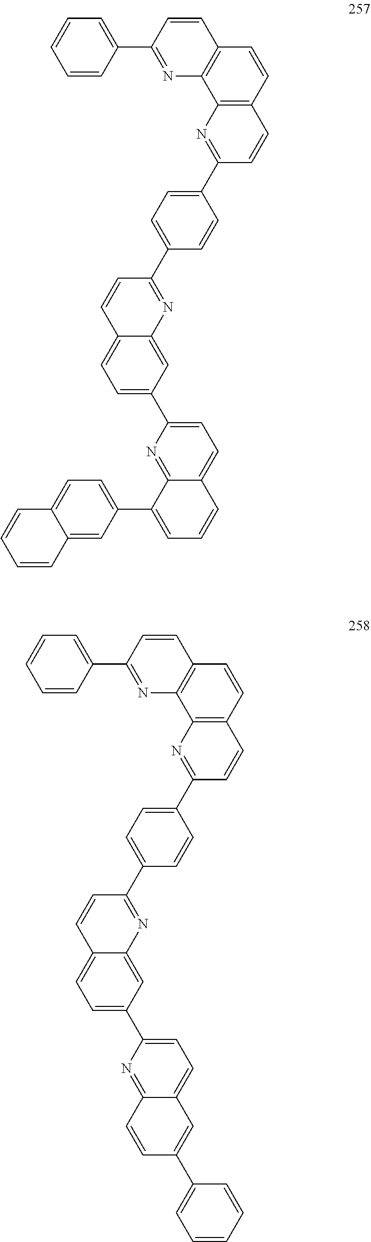 [SMILES: c1ccc(-c2ccc3ccc4ccc(-c5ccc(-c6ccc7ccc(-c8ccc9cccc(-c%10ccc%11ccccc%11c%10)c9n8)cc7n6)cc5)nc4c3n2)cc1 ; c1ccc(-c2ccc3nc(-c4ccc5ccc(-c6ccc(-c7ccc8ccc9ccc(-c%10ccccc%10)nc9c8n7)cc6)nc5c4)ccc3c2)cc1]

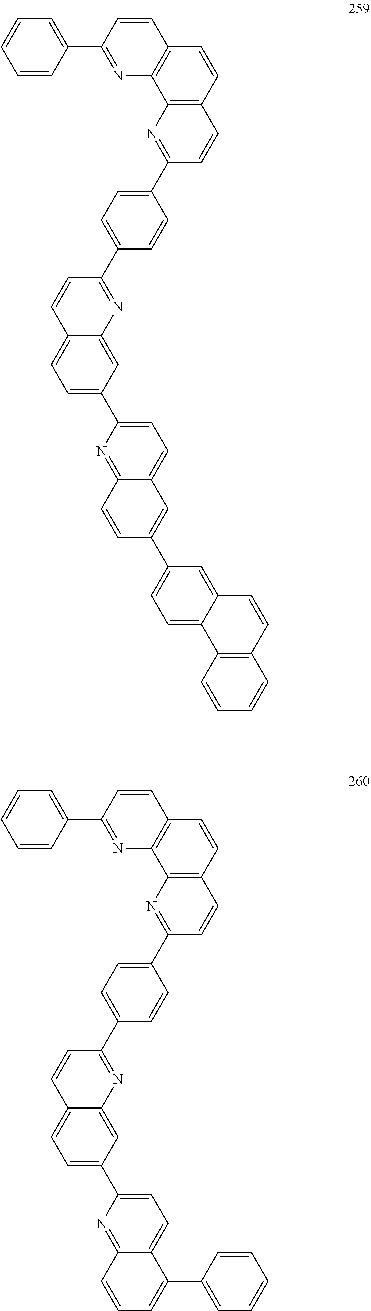 [SMILES: c1ccc(-c2ccc3ccc4ccc(-c5ccc(-c6ccc7ccc(-c8ccc9cc(-c%10ccc%11c(ccc%12ccccc%12%11)c%10)ccc9n8)cc7n6)cc5)nc4c3n2)cc1 ; c1ccc(-c2ccc3ccc4ccc(-c5ccc(-c6ccc7ccc(-c8ccc9c(-c%10ccccc%10)cccc9n8)cc7n6)cc5)nc4c3n2)cc1]

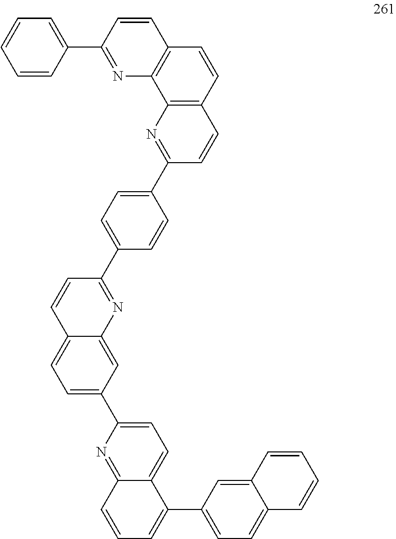 [SMILES: c1ccc(-c2ccc3ccc4ccc(-c5ccc(-c6ccc7ccc(-c8ccc9c(-c%10ccc%11ccccc%11c%10)cccc9n8)cc7n6)cc5)nc4c3n2)cc1]

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

One embodiment of the present specification provides an organic light emitting device including a first electrode; a second electrode; and an organic material layer provided between the first electrode and the second electrode, wherein the organic material layer includes the heterocyclic compound represented by Chemical Formula 1.

In one embodiment of the present specification, the first electrode may be an anode, and the second electrode may be a cathode.

In another embodiment of the present specification, the first electrode may be a cathode, and the second electrode may be an anode.

In one embodiment of the present specification, the organic light emitting device may be a blue organic light emitting device, and the heterocyclic compound according to Chemical Formula 1 may be used as a material of the blue organic light emitting device. For example, the heterocyclic compound according to Chemical Formula 1 may be included in an electron transfer layer, a charge generation layer or a hole blocking layer of the blue organic light emitting device.

In another embodiment of the present specification, the organic light emitting device may be a green organic light emitting device, and the heterocyclic compound according to Chemical Formula 1 may be used as a material of the green organic light emitting device. For example, the heterocyclic compound according to Chemical Formula 1 may be included in an electron transfer layer, a charge generation layer or a hole blocking layer of the green organic light emitting device.

In another embodiment of the present specification, the organic light emitting device may be a red organic light emitting device, and the heterocyclic compound according to Chemical Formula 1 may be used as a material of the red organic light emitting device. For example, the heterocyclic compound according to Chemical Formula 1 may be included in an electron transfer layer, a charge generation layer or a hole blocking layer of the red organic light emitting device.

Specific descriptions on the heterocyclic compound represented by Chemical Formula 1 are the same as the descriptions provided above.

The organic light emitting device of the present specification may be manufactured using common organic light emitting device manufacturing methods and materials except that one or more of the organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present specification may be formed in a single layer structure, but may be formed in a multilayer structure in which two or more organic material layers are laminated. For example, the organic light emitting device of the present disclosure may have a structure including a hole injection layer, a hole transfer layer, a light emitting layer, an electron transfer layer, an electron injection layer and the like as the organic material layer. However, the structure of the organic light emitting device is not limited thereto, and may include a smaller number of organic material layers.

In the organic light emitting device of the present specification, the organic material layer includes an electron transfer layer, and the electron transfer layer may include the heterocyclic compound of Chemical Formula 1. When using the heterocyclic compound as an electron transfer material, HOMO and LUMO may be adjusted by introducing various substituents, and excellent electron transfer efficiency is obtained.

In the organic light emitting device of the present specification, the organic material layer includes a hole blocking layer, and the hole blocking layer may include the heterocyclic compound of Chemical Formula 1.

When using the heterocyclic compound of Chemical Formula 1 as a hole blocking layer material, holes are trapped in a light emitting layer so that the holes moving from an anode may effectively emit light in the light emitting layer, and excitons are effectively formed thereby. Accordingly, driving and efficiency of the device may be enhanced.

In the organic light emitting device of the present specification, the organic material layer includes a charge generation layer, and the charge generation layer may include the heterocyclic compound of Chemical Formula 1.

The organic light emitting device of the present disclosure may further include one, two or more layers selected from the group consisting of a light emitting layer, a hole injection layer, a hole transfer layer, an electron injection layer, an electron transfer layer, an electron blocking layer and a hole blocking layer.

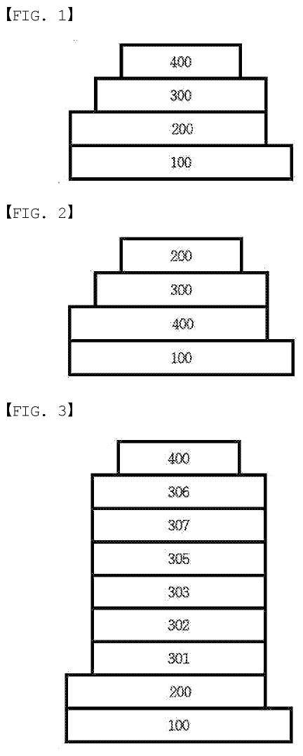

FIG. 1 to FIG. 5 illustrate a lamination order of electrodes and organic material layers of an organic light emitting device according to one embodiment of the present specification. However, the scope of the present application is not limited to these diagrams, and structures of organic light emitting devices known in the art may also be used in the present application.

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

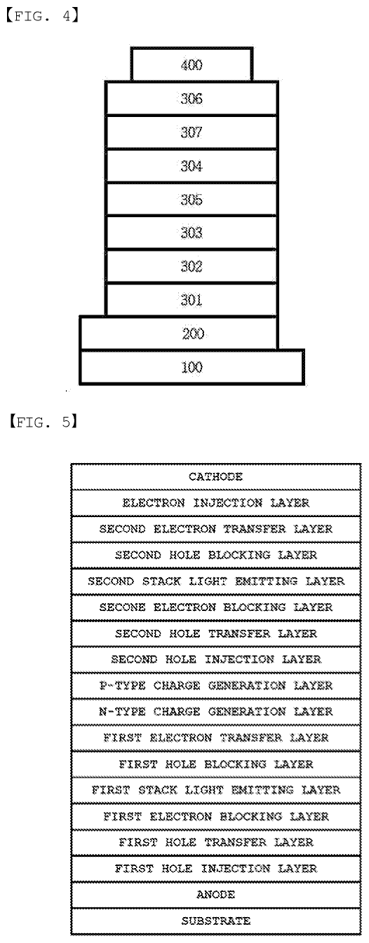

FIG. 3 and FIG. 4 illustrate organic light emitting devices of Examples 2 and 3 of the present specification as cases of the organic material layer being a multilayer.

However, the scope of the present application is not limited to such a lamination structure, and as necessary, layers other than the light emitting layer may not be included, and other necessary functional layers may be further added.

The organic material layer including the heterocyclic compound represented by Chemical Formula 1 may further include other materials as necessary.

In addition, the organic light emitting device according to one embodiment of the present specification includes an anode, a cathode, and two or more stacks provided between the anode and the cathode, the two or more stacks each independently include a light emitting layer, a charge generation layer is included between the two or more stacks, and the charge generation layer includes the heterocyclic compound represented by Chemical Formula 1.

The organic light emitting device according to one embodiment of the present specification includes a first electrode; a first stack provided on the first electrode and including a first light emitting layer; a charge generation layer provided on the first stack; a second stack provided on the charge generation layer and including a second light emitting layer; and a second electrode provided on the second stack, wherein the charge generation layer may include the heterocyclic compound represented by Chemical Formula 1.

The organic light emitting device according to one embodiment of the present specification includes a first electrode; a second electrode; and an organic material layer provided between the first electrode and the second electrode, wherein the organic material layer includes two or more stacks, and the two or more stacks each independently include a light emitting layer, a charge generation layer is included between the two or more stacks, and the charge generation layer may include the heterocyclic compound represented by Chemical Formula 1.

The organic light emitting device according to one embodiment of the present specification includes a first electrode; a second electrode; and an organic material layer provided between the first electrode and the second electrode, wherein the organic material layer includes a first stack including a first light emitting layer; a charge generation layer provided on the first stack; and a second stack including a second light emitting layer provided on the charge generation layer, and the charge generation layer may include the heterocyclic compound represented by Chemical Formula 1.

In addition, the organic light emitting device according to one embodiment of the present specification includes an anode, a first stack provided on the anode and including a first light emitting layer, a charge generation layer provided on the first stack, a second stack provided on the charge generation layer and including a second light emitting layer, and a cathode provided on the second stack. Herein, the charge generation layer may include the heterocyclic compound represented by Chemical Formula 1. When the heterocyclic compound is included in the charge generation layer, an organic light emitting device having superior driving voltage and efficiency is provided by a hole migration-friendly biquinoline skeleton and an electron-friendly substituent structure.

The organic light emitting device according to one embodiment of the present specification includes a first electrode; a first stack provided on the first electrode and including a first light emitting layer; a charge generation layer provided on the first stack; a second stack provided on the charge generation layer and including a second light emitting layer; and a second electrode provided on the second stack, wherein the charge generation layer is an N-type charge generation layer, and the N-type charge generation layer may include the heterocyclic compound represented by Chemical Formula 1.

In addition, the first stack and the second stack may each independently further include one or more types of the hole injection layer, the hole transfer layer, the hole blocking layer, the electron transfer layer, the electron injection layer and the like described above.

The charge generation layer may be an N-type charge generation layer or a P-type charge generation layer, and the N-type charge generation layer may further include a dopant known in the art in addition to the heterocyclic compound represented by Chemical Formula 1.

As the organic light emitting device according to one embodiment of the present specification, an organic light emitting device having a 2-stack tandem structure is illustrated in FIG. 5.

Herein, the first electron blocking layer, the first hole blocking layer, the second hole blocking layer and the like described in FIG. 5 may not be included in some cases.

In the organic light emitting device according to one embodiment of the present specification, materials other than the heterocyclic compound represented by Chemical Formula 1 are illustrated below, however, these are for illustrative purposes only and not for limiting the scope of the present application, and the materials may be replaced by materials known in the art.

As the anode material, materials having relatively large work function may be used, and transparent conductive oxides, metals, conductive polymers or the like may be used. Specific examples of the anode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene](PEDOT), polypyrrole and polyaniline, and the like, but are not limited thereto.

As the cathode material, materials having relatively small work function may be used, and metals, metal oxides, conductive polymers or the like may be used. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrenesulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used in addition to the heterocyclic compound, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected and used as a host material of a light emitting layer.

The organic light emitting device according to one embodiment of the present specification may be a top-emission type, a bottom-emission type or a dual-emission type depending on the materials used.

The heterocyclic compound according to one embodiment of the present specification may also be used in an organic electronic device including an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### [Preparation Example 1] Preparation of Compound 1

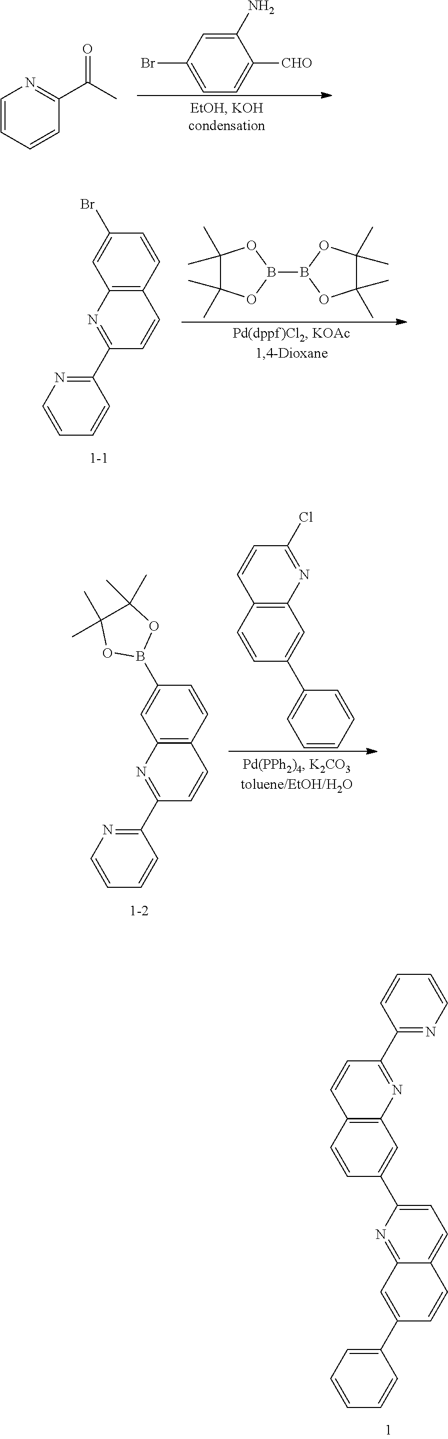

### 1) Preparation of Compound 1-1

After dissolving 1-(pyridin-2-yl)ethanone (10 g, 82.5 mmol) and 2-amino-4-bromobenzaldehyde (16.5 g, 82.5 mmol) in ethanol (EtOH) (100 mL), KOH (82.5 mmol) was introduced to the reaction container, and the result was heated to 80° C. After the reaction was completed, the result was cooled to room temperature, and then extracted with distilled water and ethyl acetate. The extracted organic layer was dried with anhydrous Na₂SO₄, and then filtered. The solvent of the filtered organic layer was removed using a rotary evaporator, and the result was purified with column chromatography using dichloromethane and hexane as a developing solvent to obtain target Compound 1-1 (19 g, 80%).

### 2) Preparation of Compound 1-2

After dissolving Compound 1-1 (21.1 g, 74.3 mmol) and 4,4,4′,4′,5,5,5′,5′-octamethyl-2,2′-bi(1,3,2-dioxaborolane) (37.7 g, 148.6 mmol) in 1,4-dioxane (200 mL), Pd(dppf)Cl₂ ([1,1′-bis(diphenylphosphino) ferrocene]dichloropalladium (II)) (2.3 g, 37.1 mmol) and potassium acetate (KOAc) (8.3 g, 222.9 mmol) were introduced thereto, and the result was stirred for 2 hours. After the reaction was completed, the result was cooled to room temperature, and then extracted with distilled water and dichloromethane. The extracted organic layer was dried with anhydrous Na₂SO₄, and then filtered. The solvent of the filtered organic layer was removed using a rotary evaporator, and the result was purified with column chromatography using dichloromethane and hexane as a developing solvent to obtain target Compound 1-2 (20.2 g, 82%).

### 3) Preparation of Compound 1

After dissolving Compound 1-2 (20.2 g, 60.9 mmol) and 2-chloro-7-phenylquinoline (14.6 g, 60.9 mmol) in 1,4-toluene/ethanol/H₂O (200 mL), Pd(PPh₃) 4 (tetrakis(triphenylphosphine)palladium(0)) (3.5 g, 3.0 mmol) and KOAc (8.3 g, 182.7 mmol) were introduced thereto, and the result was stirred for 2 hours. After the reaction was completed, the result was cooled to room temperature, and then extracted with distilled water and dichloromethane. The extracted organic layer was dried with anhydrous Na₂SO₄, and then filtered. The solvent of the filtered organic layer was removed using a rotary evaporator, and the result was purified with column chromatography using dichloromethane and hexane as a developing solvent to obtain target Compound 1 (18.2 g, 73%)

Target compounds were synthesized in the same manner as in Preparation Example 1 except that Intermediate A of the following Table 1 was used instead of 2-chloro-7-phenylquinoline.

TABLE 1

| Compound | | | |
|---|---|---|---|
| No. | Intermediate A | Target Compound | Yield |
| 3 | 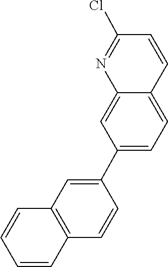 [SMILES: Clc1ccc2ccc(-c3ccc4ccccc4c3)cc2n1] | 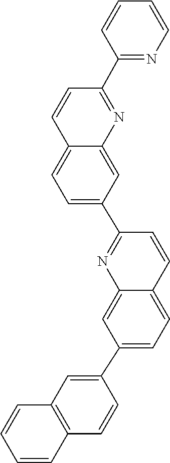 [SMILES: c1ccc(-c2ccc3ccc(-c4ccc5ccc(-c6ccc7ccccc7c6)cc5n4)cc3n2)nc1] | 72% |
| 8 | 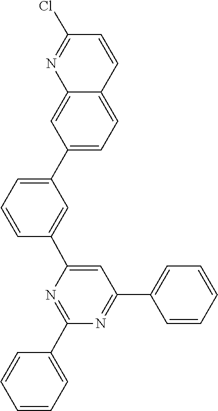 [SMILES: Clc1ccc2ccc(-c3cccc(-c4cc(-c5ccccc5)nc(-c5ccccc5)n4)c3)cc2n1] | 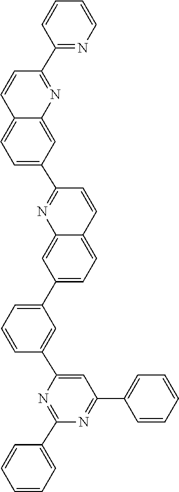 [SMILES: c1ccc(-c2cc(-c3cccc(-c4ccc5ccc(-c6ccc7ccc(-c8ccccn8)nc7c6)nc5c4)c3)nc(-c3ccccc3)n2)cc1] | 67% |
| 12 | 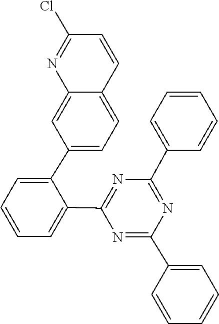 [SMILES: Clc1ccc2ccc(-c3ccccc3-c3nc(-c4ccccc4)nc(-c4ccccc4)n3)cc2n1] | 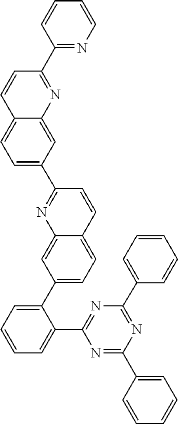 [SMILES: c1ccc(-c2nc(-c3ccccc3)nc(-c3ccccc3-c3ccc4ccc(-c5ccc6ccc(-c7ccccn7)nc6c5)nc4c3)n2)cc1] | 66% |
| 15 | 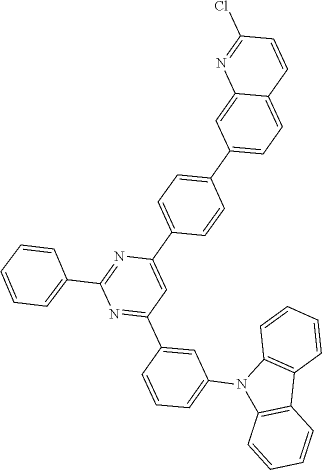 [SMILES: Clc1ccc2ccc(-c3ccc(-c4cc(-c5cccc(-n6c7ccccc7c7ccccc76)c5)nc(-c5ccccc5)n4)cc3)cc2n1] | 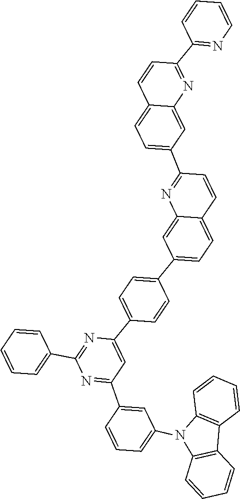 [SMILES: c1ccc(-c2nc(-c3ccc(-c4ccc5ccc(-c6ccc7ccc(-c8ccccn8)nc7c6)nc5c4)cc3)cc(-c3cccc(-n4c5ccccc5c5ccccc54)c3)n2)cc1] | 70% |
| 17 | 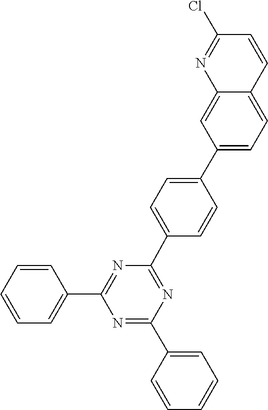 [SMILES: Clc1ccc2ccc(-c3ccc(-c4nc(-c5ccccc5)nc(-c5ccccc5)n4)cc3)cc2n1] | 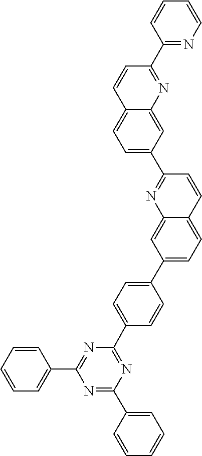 [SMILES: c1ccc(-c2nc(-c3ccccc3)nc(-c3ccc(-c4ccc5ccc(-c6ccc7ccc(-c8ccccn8)nc7c6)nc5c4)cc3)n2)cc1] | 71% |
| 19 | 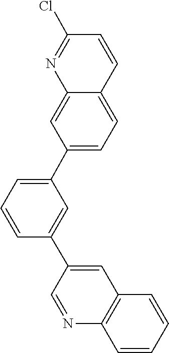 [SMILES: Clc1ccc2ccc(-c3cccc(-c4cnc5ccccc5c4)c3)cc2n1] | 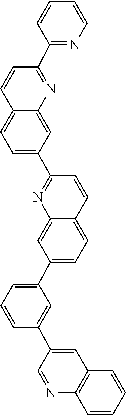 [SMILES: c1ccc(-c2ccc3ccc(-c4ccc5ccc(-c6cccc(-c7cnc8ccccc8c7)c6)cc5n4)cc3n2)nc1] | 65% |
| 22 | 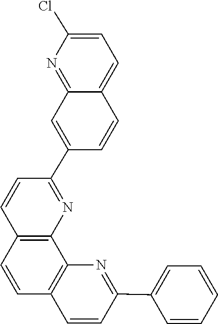 [SMILES: Clc1ccc2ccc(-c3ccc4ccc5ccc(-c6ccccc6)nc5c4n3)cc2n1] | 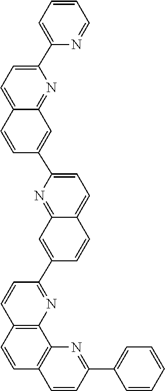 [SMILES: c1ccc(-c2ccc3ccc4ccc(-c5ccc6ccc(-c7ccc8ccc(-c9ccccn9)nc8c7)nc6c5)nc4c3n2)cc1] | 68% |
| 27 | 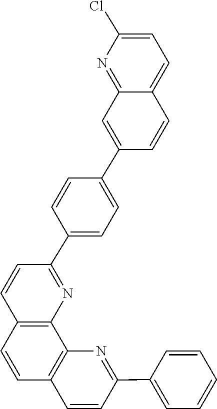 [SMILES: Clc1ccc2ccc(-c3ccc(-c4ccc5ccc6ccc(-c7ccccc7)nc6c5n4)cc3)cc2n1] | 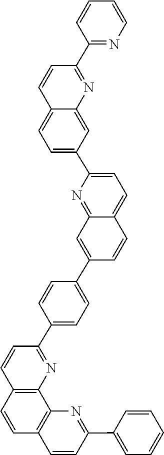 [SMILES: c1ccc(-c2ccc3ccc4ccc(-c5ccc(-c6ccc7ccc(-c8ccc9ccc(-c%10ccccn%10)nc9c8)nc7c6)cc5)nc4c3n2)cc1] | 72% |
| 30 | 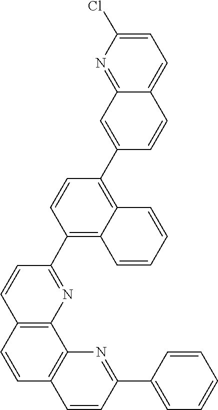 [SMILES: Clc1ccc2ccc(-c3ccc(-c4ccc5ccc6ccc(-c7ccccc7)nc6c5n4)c4ccccc34)cc2n1] | 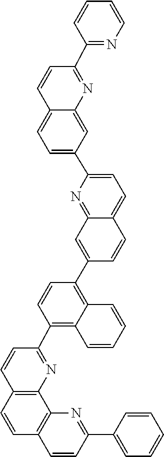 [SMILES: c1ccc(-c2ccc3ccc4ccc(-c5ccc(-c6ccc7ccc(-c8ccc9ccc(-c%10ccccn%10)nc9c8)nc7c6)c6ccccc56)nc4c3n2)cc1] | 73% |
| 250 | 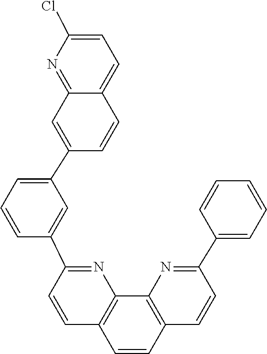 [SMILES: Clc1ccc2ccc(-c3cccc(-c4ccc5ccc6ccc(-c7ccccc7)nc6c5n4)c3)cc2n1] | 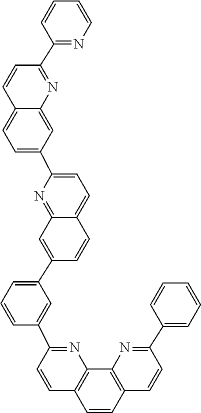 [SMILES: c1ccc(-c2ccc3ccc4ccc(-c5cccc(-c6ccc7ccc(-c8ccc9ccc(-c%10ccccn%10)nc9c8)nc7c6)c5)nc4c3n2)cc1] | 70% |

Target compounds were synthesized in the same manner as in Preparation Example 1 except that 1-(pyridin-3-yl)ethanone was used instead of 1-(pyridin-2-yl)ethanone, and Intermediate B of the following Table 2 was used instead of 2-chloro-7-phenylquinoline.

TABLE 2

| Compound | | | |
|---|---|---|---|
| No. | Intermediate B | Target Compound | Yield |
| 33 | 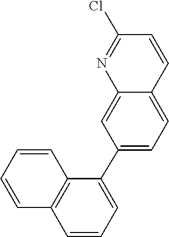 [SMILES: Clc1ccc2ccc(-c3cccc4ccccc34)cc2n1] | 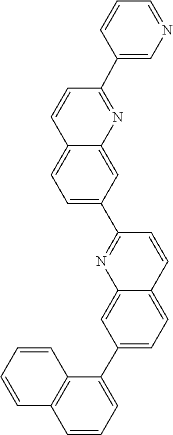 [SMILES: c1cncc(-c2ccc3ccc(-c4ccc5ccc(-c6cccc7ccccc67)cc5n4)cc3n2)c1] | 69% |
| 37 | 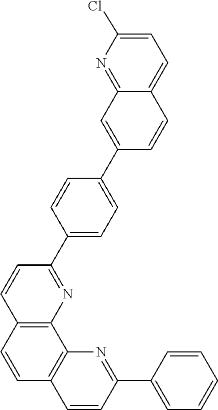 [SMILES: Clc1ccc2ccc(-c3ccc(-c4ccc5ccc6ccc(-c7ccccc7)nc6c5n4)cc3)cc2n1] | 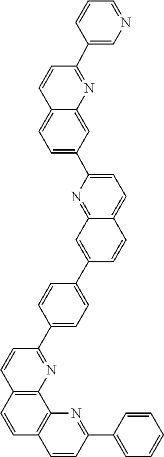 [SMILES: c1ccc(-c2ccc3ccc4ccc(-c5ccc(-c6ccc7ccc(-c8ccc9ccc(-c%10cccnc%10)nc9c8)nc7c6)cc5)nc4c3n2)cc1] | 67% |
| 40 | 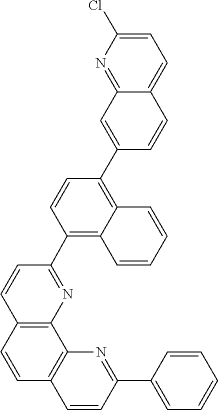 [SMILES: Clc1ccc2ccc(-c3ccc(-c4ccc5ccc6ccc(-c7ccccc7)nc6c5n4)c4ccccc34)cc2n1] | 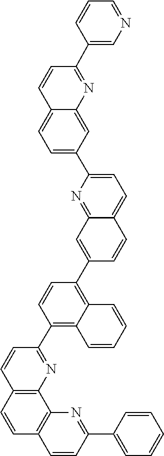 [SMILES: c1ccc(-c2ccc3ccc4ccc(-c5ccc(-c6ccc7ccc(-c8ccc9ccc(-c%10cccnc%10)nc9c8)nc7c6)c6ccccc56)nc4c3n2)cc1] | 65% |

Target compounds were synthesized in the same manner as in Preparation Example 1 except that 1-(pyridin-4-yl)ethanone was used instead of 1-(pyridin-2-yl)ethanone, and Intermediate C of the following Table 3 was used instead of 2-chloro-7-phenylquinoline.

TABLE 3

| Compound | | | |
|---|---|---|---|
| No. | Intermediate C | Target Compound | Yield |
| 42 | 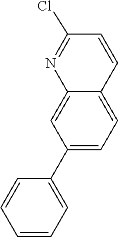 [SMILES: Clc1ccc2ccc(-c3ccccc3)cc2n1] | 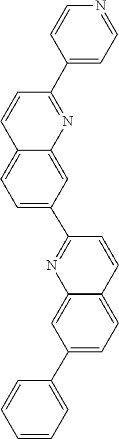 [SMILES: c1ccc(-c2ccc3ccc(-c4ccc5ccc(-c6ccncc6)nc5c4)nc3c2)cc1] | 66% |
| 45 | 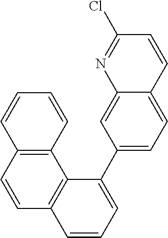 [SMILES: Clc1ccc2ccc(-c3cccc4ccc5ccccc5c34)cc2n1] | 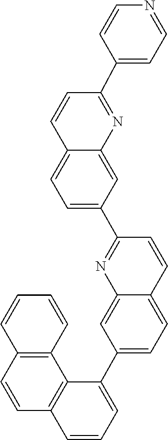 [SMILES: c1ccc2c(c1)ccc1cccc(-c3ccc4ccc(-c5ccc6ccc(-c7ccncc7)nc6c5)nc4c3)c12] | 71% |
| 48 | 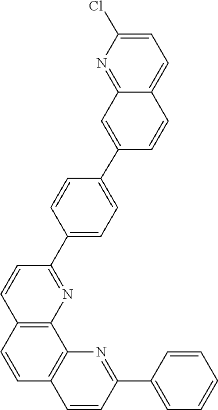 [SMILES: Clc1ccc2ccc(-c3ccc(-c4ccc5ccc6ccc(-c7ccccc7)nc6c5n4)cc3)cc2n1] | 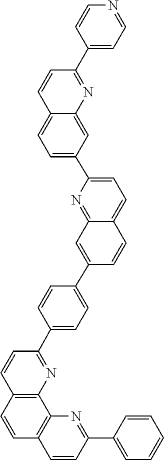 [SMILES: c1ccc(-c2ccc3ccc4ccc(-c5ccc(-c6ccc7ccc(-c8ccc9ccc(-c%10ccncc%10)nc9c8)nc7c6)cc5)nc4c3n2)cc1] | 73% |
| 51 | 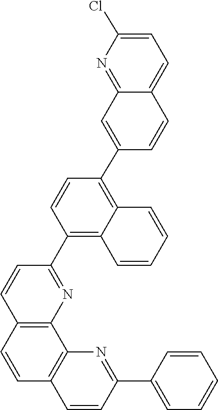 [SMILES: Clc1ccc2ccc(-c3ccc(-c4ccc5ccc6ccc(-c7ccccc7)nc6c5n4)c4ccccc34)cc2n1] | 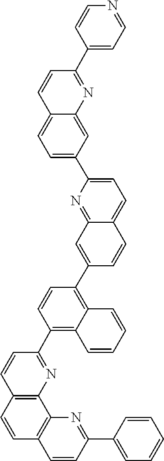 [SMILES: c1ccc(-c2ccc3ccc4ccc(-c5ccc(-c6ccc7ccc(-c8ccc9ccc(-c%10ccncc%10)nc9c8)nc7c6)c6ccccc56)nc4c3n2)cc1] | 71% |

Target compounds were synthesized in the same manner as in Preparation Example 1 except that 1-(pyrimidin-2-yl)ethanone was used instead of 1-(pyridin-2-yl)ethanone, and Intermediate D of the following Table 4 was used instead of 2-chloro-7-phenylquinoline.

TABLE 4

| Compound | | | |
|---|---|---|---|
| No. | Intermediate D | Target Compound | Yield |
| 54 | 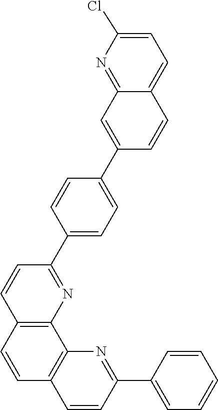 [SMILES: Clc1ccc2ccc(-c3ccc(-c4ccc5ccc6ccc(-c7ccccc7)nc6c5n4)cc3)cc2n1] | 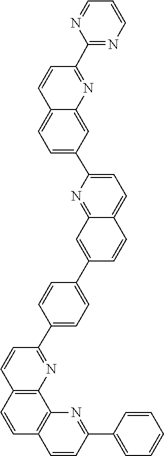 [SMILES: c1ccc(-c2ccc3ccc4ccc(-c5ccc(-c6ccc7ccc(-c8ccc9ccc(-c%10ncccn%10)nc9c8)nc7c6)cc5)nc4c3n2)cc1] | 71% |
| 59 | 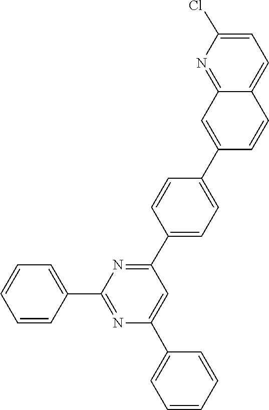 [SMILES: Clc1ccc2ccc(-c3ccc(-c4cc(-c5ccccc5)nc(-c5ccccc5)n4)cc3)cc2n1] | 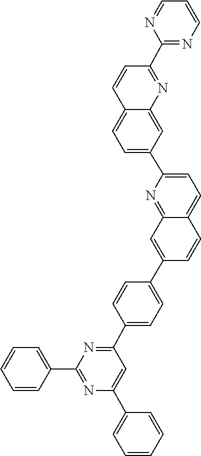 [SMILES: c1ccc(-c2cc(-c3ccc(-c4ccc5ccc(-c6ccc7ccc(-c8ncccn8)nc7c6)nc5c4)cc3)nc(-c3ccccc3)n2)cc1] | 70% |

Target compounds were synthesized in the same manner as in Preparation Example 1 except that 1-(4,6-diphenylpyrimidin-2-yl)ethanone was used instead of 1-(pyridin-2-yl)ethanone, and Intermediate E of the following Table 5 was used instead of 2-chloro-7-phenylquinoline.

TABLE 5

| Compound | | | |
|---|---|---|---|
| No. | Intermediate E | Target Compound | Yield |
| 64 | 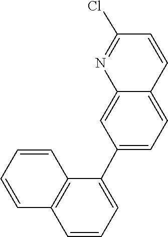 [SMILES: Clc1ccc2ccc(-c3cccc4ccccc34)cc2n1] | 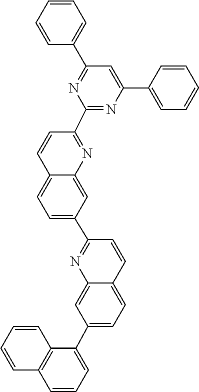 [SMILES: c1ccc(-c2cc(-c3ccccc3)nc(-c3ccc4ccc(-c5ccc6ccc(-c7cccc8ccccc78)cc6n5)cc4n3)n2)cc1] | 67% |
| 68 | 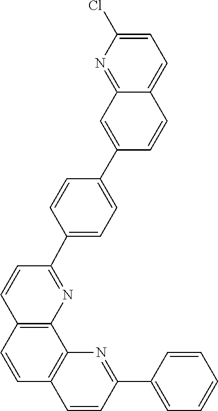 [SMILES: Clc1ccc2ccc(-c3ccc(-c4ccc5ccc6ccc(-c7ccccc7)nc6c5n4)cc3)cc2n1] | 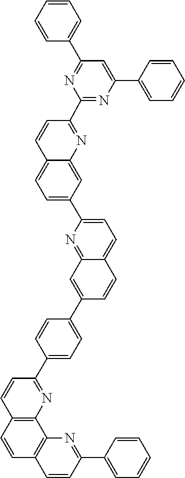 [SMILES: c1ccc(-c2cc(-c3ccccc3)nc(-c3ccc4ccc(-c5ccc6ccc(-c7ccc(-c8ccc9ccc%10ccc(-c%11ccccc%11)nc%10c9n8)cc7)cc6n5)cc4n3)n2)cc1] | 72% |

A target compound was synthesized in the same manner as in Preparation Example 1 except that 1-(4,6-di(pyridin-3-yl)pyrimidin-2-yl)ethanone was used instead of 1-(pyridin-2-yl)ethanone, and Intermediate F of the following Table 6 was used instead of 2-chloro-7-phenylquinoline.

TABLE 6

| Compound | | | |
|---|---|---|---|
| No. | Intermediate F | Target Compound | Yield |
| 72 | 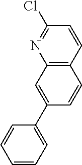 [SMILES: Clc1ccc2ccc(-c3ccccc3)cc2n1] | 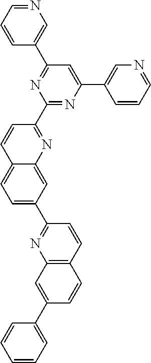 [SMILES: c1ccc(-c2ccc3ccc(-c4ccc5ccc(-c6nc(-c7cccnc7)cc(-c7cccnc7)n6)nc5c4)nc3c2)cc1] | 69% |

A target compound was synthesized in the same manner as in Preparation Example 1 except that 1-(pyrimidin-4-yl)ethanone was used instead of 1-(pyridin-2-yl)ethanone, and Intermediate G of the following Table 7 was used instead of 2-chloro-7-phenylquinoline.

TABLE 7

| Compound | | | |
|---|---|---|---|
| No. | Intermediate G | Target Compound | Yield |
| 73 | 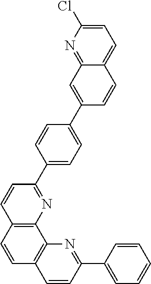 [SMILES: Clc1ccc2ccc(-c3ccc(-c4ccc5ccc6ccc(-c7ccccc7)nc6c5n4)cc3)cc2n1] | 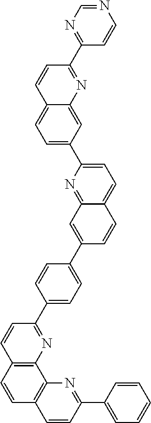 [SMILES: c1ccc(-c2ccc3ccc4ccc(-c5ccc(-c6ccc7ccc(-c8ccc9ccc(-c%10ccncn%10)nc9c8)nc7c6)cc5)nc4c3n2)cc1] | 76% |

Target compounds were synthesized in the same manner as in Preparation Example 1 except that 1-(2,6-diphenylpyrimidin-4-yl)ethanone was used instead of 1-(pyridin-2-yl)ethanone, and Intermediate H of the following Table 8 was used instead of 2-chloro-7-phenylquinoline.

TABLE 8

| Compound No. | Intermediate H | Target Compound | Yield |
|---|---|---|---|
| 79 | 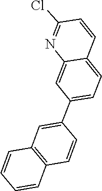 [SMILES: Clc1ccc2ccc(-c3ccc4ccccc4c3)cc2n1] | 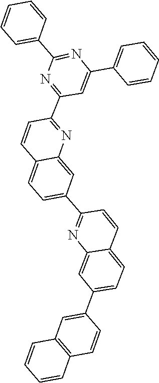 [SMILES: c1ccc(-c2cc(-c3ccc4ccc(-c5ccc6ccc(-c7ccc8ccccc8c7)cc6n5)cc4n3)nc(-c3ccccc3)n2)cc1] | 71% |
| 80 | 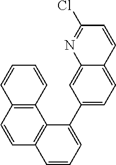 [SMILES: Clc1ccc2ccc(-c3cccc4ccc5ccccc5c34)cc2n1] | 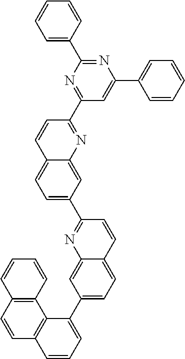 [SMILES: c1ccc(-c2cc(-c3ccc4ccc(-c5ccc6ccc(-c7cccc8ccc9ccccc9c78)cc6n5)cc4n3)nc(-c3ccccc3)n2)cc1] | 69% |
| 83 | 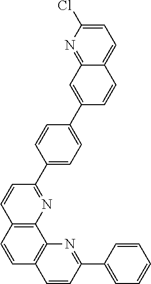 [SMILES: Clc1ccc2ccc(-c3ccc(-c4ccc5ccc6ccc(-c7ccccc7)nc6c5n4)cc3)cc2n1] | 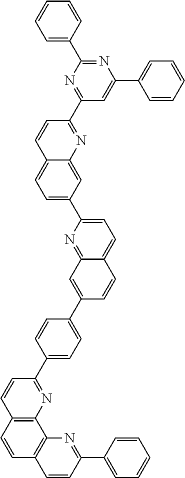 [SMILES: c1ccc(-c2cc(-c3ccc4ccc(-c5ccc6ccc(-c7ccc(-c8ccc9ccc%10ccc(-c%11ccccc%11)nc%10c9n8)cc7)cc6n5)cc4n3)nc(-c3ccccc3)n2)cc1] | 73% |
| 87 | 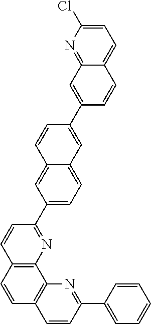 [SMILES: Clc1ccc2ccc(-c3ccc4cc(-c5ccc6ccc7ccc(-c8ccccc8)nc7c6n5)ccc4c3)cc2n1] | 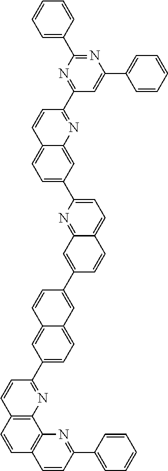 [SMILES: c1ccc(-c2cc(-c3ccc4ccc(-c5ccc6ccc(-c7ccc8cc(-c9ccc%10ccc%11ccc(-c%12ccccc%12)nc%11c%10n9)ccc8c7)cc6n5)cc4n3)nc(-c3ccccc3)n2)cc1] | 73% |
| 251 | 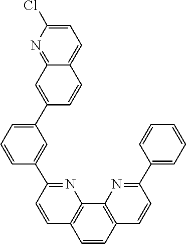 [SMILES: Clc1ccc2ccc(-c3cccc(-c4ccc5ccc6ccc(-c7ccccc7)nc6c5n4)c3)cc2n1] | 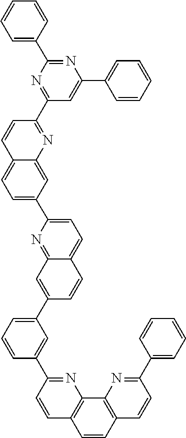 [SMILES: c1ccc(-c2cc(-c3ccc4ccc(-c5ccc6ccc(-c7cccc(-c8ccc9ccc%10ccc(-c%11ccccc%11)nc%10c9n8)c7)cc6n5)cc4n3)nc(-c3ccccc3)n2)cc1] | 71% |

A target compound was synthesized in the same manner as in Preparation Example 1 except that 1-(pyrazin-2-yl)ethanone was used instead of 1-(pyridin-2-yl)ethanone, and Intermediate I of the following Table 9 was used instead of 2-chloro-7-phenylquinoline.

TABLE 9

| Compound | | | |
|---|---|---|---|
| No. | Intermediate I | Target Compound | Yield |
| 90 | 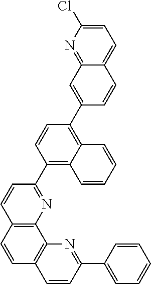 [SMILES: Clc1ccc2ccc(-c3ccc(-c4ccc5ccc6ccc(-c7ccccc7)nc6c5n4)c4ccccc34)cc2n1] | 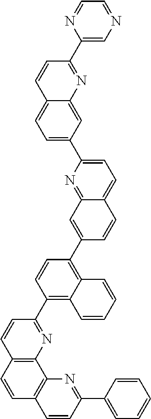 [SMILES: c1ccc(-c2ccc3ccc4ccc(-c5ccc(-c6ccc7ccc(-c8ccc9ccc(-c%10cnccn%10)nc9c8)nc7c6)c6ccccc56)nc4c3n2)cc1] | 65% |

A target compound was synthesized in the same manner as in Preparation Example 1 except that 1-(1,3,5-triazin-2-yl)ethanone was used instead of 1-(pyridin-2-yl)ethanone, and Intermediate J of the following Table 10 was used instead of 2-chloro-7-phenylquinoline.

TABLE 10

| Compound | | | |
|---|---|---|---|
| No. | Intermediate J | Target Compound | Yield |
| 92 | 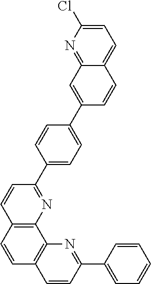 [SMILES: Clc1ccc2ccc(-c3ccc(-c4ccc5ccc6ccc(-c7ccccc7)nc6c5n4)cc3)cc2n1] | 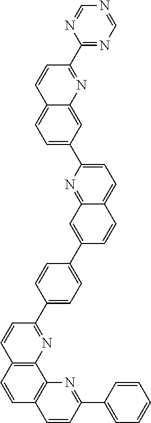 [SMILES: c1ccc(-c2ccc3ccc4ccc(-c5ccc(-c6ccc7ccc(-c8ccc9ccc(-c%10ncncn%10)nc9c8)nc7c6)cc5)nc4c3n2)cc1] | 72% |

Target compounds were synthesized in the same manner as in Preparation Example 1 except that 1-(4,6-diphenyl-1,3,5-triazin-2-yl)ethanone was used instead of 1-(pyridin-2-yl)ethanone, and Intermediate K of the following Table 11 was used instead of 2-chloro-7-phenylquinoline.

TABLE 11

| Compound No. | Intermediate K | Target Compound | Yield |
|---|---|---|---|
| 97 | 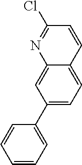 [SMILES: Clc1ccc2ccc(-c3ccccc3)cc2n1] | 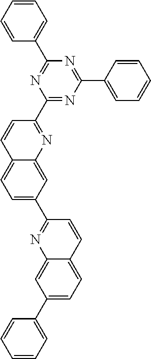 [SMILES: c1ccc(-c2ccc3ccc(-c4ccc5ccc(-c6nc(-c7ccccc7)nc(-c7ccccc7)n6)nc5c4)nc3c2)cc1] | 70% |
| 100 | 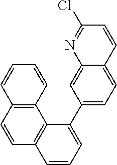 [SMILES: Clc1ccc2ccc(-c3cccc4ccc5ccccc5c34)cc2n1] | 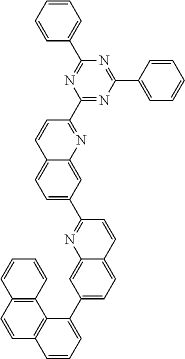 [SMILES: c1ccc(-c2nc(-c3ccccc3)nc(-c3ccc4ccc(-c5ccc6ccc(-c7cccc8ccc9ccccc9c78)cc6n5)cc4n3)n2)cc1] | 80% |
| 102 | 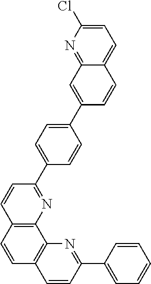 [SMILES: Clc1ccc2ccc(-c3ccc(-c4ccc5ccc6ccc(-c7ccccc7)nc6c5n4)cc3)cc2n1] | 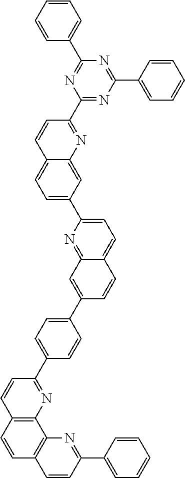 [SMILES: c1ccc(-c2ccc3ccc4ccc(-c5ccc(-c6ccc7ccc(-c8ccc9ccc(-c%10nc(-c%11ccccc%11)nc(-c%11ccccc%11)n%10)nc9c8)nc7c6)cc5)nc4c3n2)cc1] | 70% |
| 105 | 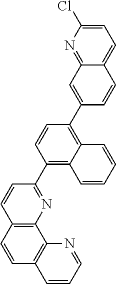 [SMILES: Clc1ccc2ccc(-c3ccc(-c4ccc5ccc6cccnc6c5n4)c4ccccc34)cc2n1] | 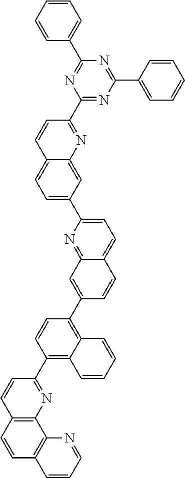 [SMILES: c1ccc(-c2nc(-c3ccccc3)nc(-c3ccc4ccc(-c5ccc6ccc(-c7ccc(-c8ccc9ccc%10cccnc%10c9n8)c8ccccc78)cc6n5)cc4n3)n2)cc1] | 81% |

Target compounds were synthesized in the same manner as in Preparation Example 1 except that 1-(quinolin-8-yl)ethanone was used instead of 1-(pyridin-2-yl)ethanone, and Intermediate L of the following Table 12 was used instead of 2-chloro-7-phenylquinoline.

TABLE 12

| Compound | | | |
|---|---|---|---|
| No. | Intermediate L | Target Compound | Yield |
| 108 | 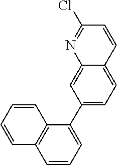 [SMILES: Clc1ccc2ccc(-c3cccc4ccccc34)cc2n1] | 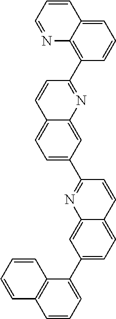 [SMILES: c1ccc2c(-c3ccc4ccc(-c5ccc6ccc(-c7cccc8cccnc78)nc6c5)nc4c3)cccc2c1] | 75% |
| 111 | 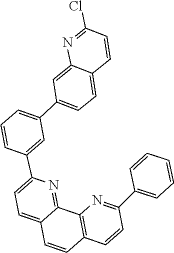 [SMILES: Clc1ccc2ccc(-c3cccc(-c4ccc5ccc6ccc(-c7ccccc7)nc6c5n4)c3)cc2n1] | 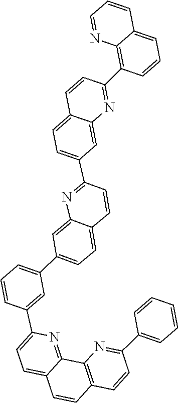 [SMILES: c1ccc(-c2ccc3ccc4ccc(-c5cccc(-c6ccc7ccc(-c8ccc9ccc(-c%10cccc%11cccnc%10%11)nc9c8)nc7c6)c5)nc4c3n2)cc1] | 71% |
| 114 | 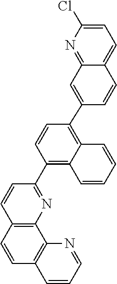 [SMILES: Clc1ccc2ccc(-c3ccc(-c4ccc5ccc6cccnc6c5n4)c4ccccc34)cc2n1] | 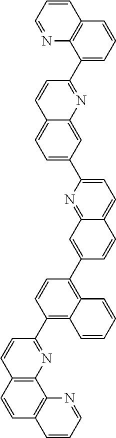 [SMILES: c1cnc2c(-c3ccc4ccc(-c5ccc6ccc(-c7ccc(-c8ccc9ccc%10cccnc%10c9n8)c8ccccc78)cc6n5)cc4n3)cccc2c1] | 72% |
| 116 | 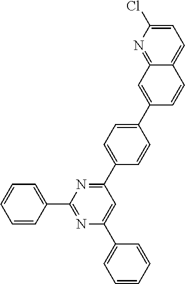 [SMILES: Clc1ccc2ccc(-c3ccc(-c4cc(-c5ccccc5)nc(-c5ccccc5)n4)cc3)cc2n1] | 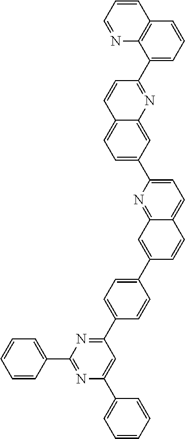 [SMILES: c1ccc(-c2cc(-c3ccc(-c4ccc5ccc(-c6ccc7ccc(-c8cccc9cccnc89)nc7c6)nc5c4)cc3)nc(-c3ccccc3)n2)cc1] | 66% |
| 120 | 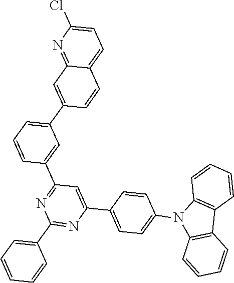 [SMILES: Clc1ccc2ccc(-c3cccc(-c4cc(-c5ccc(-n6c7ccccc7c7ccccc76)cc5)nc(-c5ccccc5)n4)c3)cc2n1] | 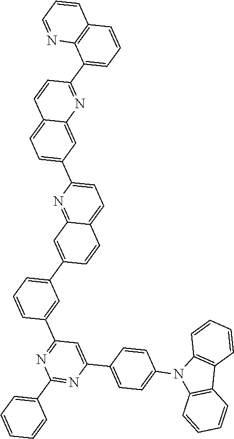 [SMILES: c1ccc(-c2nc(-c3ccc(-n4c5ccccc5c5ccccc54)cc3)cc(-c3cccc(-c4ccc5ccc(-c6ccc7ccc(-c8cccc9cccnc89)nc7c6)nc5c4)c3)n2)cc1] | 70% |

Target compounds were synthesized in the same manner as in Preparation Example 1 except that 1-(isoquinolin-8-yl)ethanone was used instead of 1-(pyridin-2-yl)ethanone, and Intermediate M of the following Table 13 was used instead of 2-chloro-7-phenylquinoline.

TABLE 13

| Compound | | | |
|---|---|---|---|
| No. | Intermediate M | Target Compound | Yield |
| 121 | 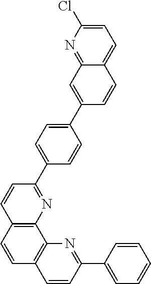 [SMILES: Clc1ccc2ccc(-c3ccc(-c4ccc5ccc6ccc(-c7ccccc7)nc6c5n4)cc3)cc2n1] | 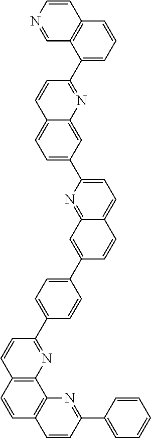 [SMILES: c1ccc(-c2ccc3ccc4ccc(-c5ccc(-c6ccc7ccc(-c8ccc9ccc(-c%10cccc%11ccncc%10%11)nc9c8)nc7c6)cc5)nc4c3n2)cc1] | 70% |
| 124 | 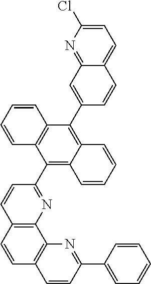 [SMILES: Clc1ccc2ccc(-c3c4ccccc4c(-c4ccc5ccc6ccc(-c7ccccc7)nc6c5n4)c4ccccc34)cc2n1] | 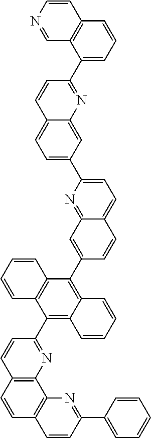 [SMILES: c1ccc(-c2ccc3ccc4ccc(-c5c6ccccc6c(-c6ccc7ccc(-c8ccc9ccc(-c%10cccc%11ccncc%10%11)nc9c8)nc7c6)c6ccccc56)nc4c3n2)cc1] | 68% |

A target compound was synthesized in the same manner as in Preparation Example 1 except that 1-(isoquinolin-5-yl)ethanone was used instead of 1-(pyridin-2-yl)ethanone, and Intermediate N of the following Table 14 was used instead of 2-chloro-7-phenylquinoline.

TABLE 14

| Compound | | | |
|---|---|---|---|
| No. | Intermediate N | Target Compound | Yield |
| 128 | 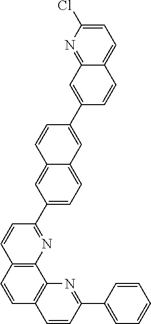 [SMILES: Clc1ccc2ccc(-c3ccc4cc(-c5ccc6ccc7ccc(-c8ccccc8)nc7c6n5)ccc4c3)cc2n1] | 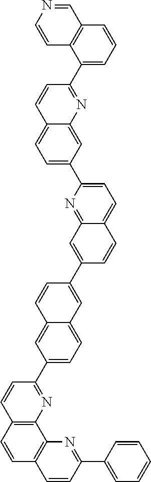 [SMILES: c1ccc(-c2ccc3ccc4ccc(-c5ccc6cc(-c7ccc8ccc(-c9ccc%10ccc(-c%11cccc%12cnccc%11%12)nc%10c9)nc8c7)ccc6c5)nc4c3n2)cc1] | 75% |

A target compound was synthesized in the same manner as in Preparation Example 1 except that 1-(quinolin-5-yl)ethanone was used instead of 1-(pyridin-2-yl)ethanone, and Intermediate O of the following Table 15 was used instead of 2-chloro-7-phenylquinoline.

TABLE 15

| Compound | | | |
|---|---|---|---|
| No. | Intermediate O | Target Compound | Yield |
| 133 | 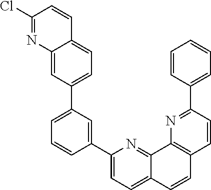 [SMILES: Clc1ccc2ccc(-c3cccc(-c4ccc5ccc6ccc(-c7ccccc7)nc6c5n4)c3)cc2n1] | 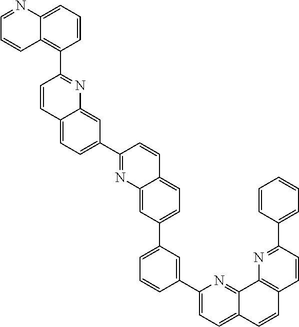 [SMILES: c1ccc(-c2ccc3ccc4ccc(-c5cccc(-c6ccc7ccc(-c8ccc9ccc(-c%10cccc%11ncccc%10%11)nc9c8)nc7c6)c5)nc4c3n2)cc1] | 69% |

Target compounds were synthesized in the same manner as in Preparation Example 1 except that 1-(isoquinolin-4-yl)ethanone was used instead of 1-(pyridin-2-yl)ethanone, and Intermediate P of the following Table 16 was used instead of 2-chloro-7-phenylquinoline.

TABLE 16

| Compound | | | |
|---|---|---|---|
| No. | Intermediate P | Target Compound | Yield |
| 137 | 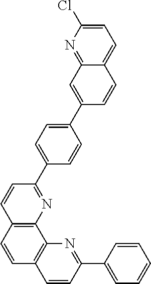 [SMILES: Clc1ccc2ccc(-c3ccc(-c4ccc5ccc6ccc(-c7ccccc7)nc6c5n4)cc3)cc2n1] | 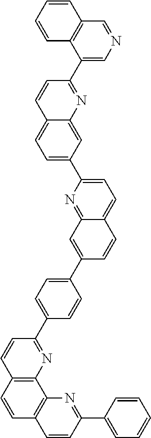 [SMILES: c1ccc(-c2ccc3ccc4ccc(-c5ccc(-c6ccc7ccc(-c8ccc9ccc(-c%10cncc%11ccccc%10%11)nc9c8)nc7c6)cc5)nc4c3n2)cc1] | 75% |
| 140 | 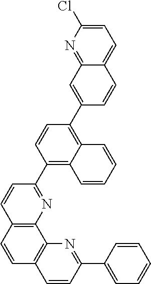 [SMILES: Clc1ccc2ccc(-c3ccc(-c4ccc5ccc6ccc(-c7ccccc7)nc6c5n4)c4ccccc34)cc2n1] | 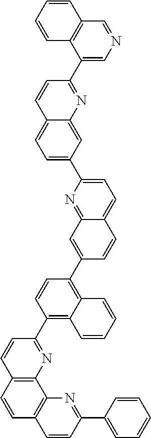 [SMILES: c1ccc(-c2ccc3ccc4ccc(-c5ccc(-c6ccc7ccc(-c8ccc9ccc(-c%10cncc%11ccccc%10%11)nc9c8)nc7c6)c6ccccc56)nc4c3n2)cc1] | 77% |

Target compounds were synthesized in the same manner as in Preparation Example 1 except that 1-(quinolin-3-yl) ethanone was used instead of 1-(pyridin-2-yl) ethanone, and Intermediate Q of the following Table 17 was used instead of 2-chloro-7-phenylquinoline.

TABLE 17

| Compound | | | |
|---|---|---|---|
| No. | Intermediate Q | Target Compound | Yield |
| 145 | 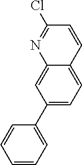 [SMILES: Clc1ccc2ccc(-c3ccccc3)cc2n1] | 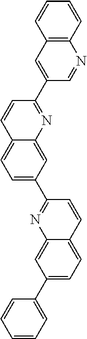 [SMILES: c1ccc(-c2ccc3ccc(-c4ccc5ccc(-c6cnc7ccccc7c6)nc5c4)nc3c2)cc1] | 70% |
| 147 | 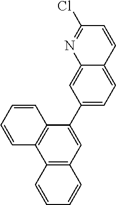 [SMILES: Clc1ccc2ccc(-c3cc4ccccc4c4ccccc34)cc2n1] | 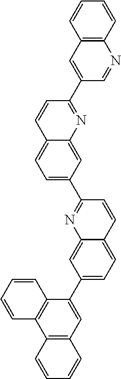 [SMILES: c1ccc2ncc(-c3ccc4ccc(-c5ccc6ccc(-c7cc8ccccc8c8ccccc78)cc6n5)cc4n3)cc2c1] | 77% |
| 150 | 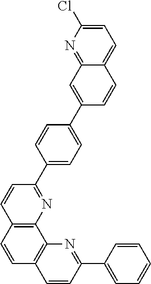 [SMILES: Clc1ccc2ccc(-c3ccc(-c4ccc5ccc6ccc(-c7ccccc7)nc6c5n4)cc3)cc2n1] | 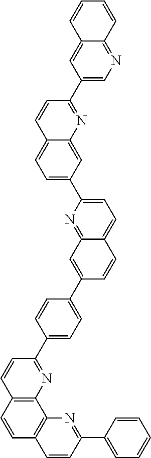 [SMILES: c1ccc(-c2ccc3ccc4ccc(-c5ccc(-c6ccc7ccc(-c8ccc9ccc(-c%10cnc%11ccccc%11c%10)nc9c8)nc7c6)cc5)nc4c3n2)cc1] | 61% |

A target compound was synthesized in the same manner as in Preparation Example 1 except that 1-(benzo[h]quinolin-2-yl)ethanone was used instead of 1-(pyridin-2-yl)ethanone, and Intermediate R of the following Table 18 was used instead of 2-chloro-7-phenylquinoline.

TABLE 18

| Compound | | | |
|---|---|---|---|
| No. | Intermediate R | Target Compound | Yield |
| 168 | 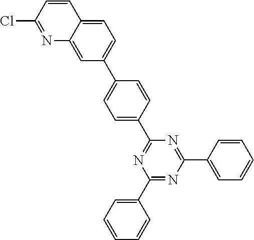 [SMILES: Clc1ccc2ccc(-c3ccc(-c4nc(-c5ccccc5)nc(-c5ccccc5)n4)cc3)cc2n1] | 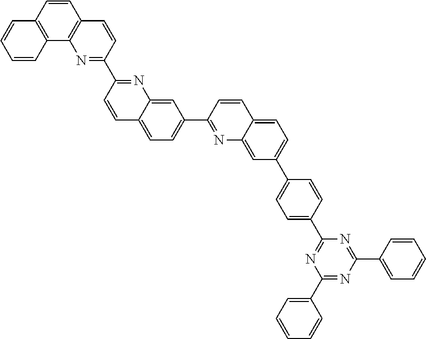 [SMILES: c1ccc(-c2nc(-c3ccccc3)nc(-c3ccc(-c4ccc5ccc(-c6ccc7ccc(-c8ccc9ccc%10ccccc%10c9n8)nc7c6)nc5c4)cc3)n2)cc1] | 66% |

A target compound was synthesized in the same manner as in Preparation Example 1 except that 1-(benzo[h]quinolin-6-yl)ethanone was used instead of 1-(pyridin-2-yl)ethanone, and Intermediate S of the following Table 19 was used instead of 2-chloro-7-phenylquinoline.

TABLE 19

| Compound | | | |
|---|---|---|---|
| No. | Intermediate S | Target Compound | Yield |
| 170 | 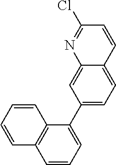 [SMILES: Clc1ccc2ccc(-c3cccc4ccccc34)cc2n1] | 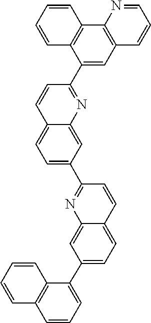 [SMILES: c1ccc2c(-c3ccc4ccc(-c5ccc6ccc(-c7cc8cccnc8c8ccccc78)nc6c5)nc4c3)cccc2c1] | 72% |

Target compounds were synthesized in the same manner as in Preparation Example 1 except that 1-(9-phenyl-1,10-phenanthrolin-2-yl)ethanone was used instead of 1-(pyridin-2-yl)ethanone, and Intermediate T of the following Table 20 was used instead of 2-chloro-7-phenylquinoline.

TABLE 20

| Compound No. | Intermediate T | Target Compound | Yield |
|---|---|---|---|
| 172 | 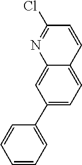 [SMILES: Clc1ccc2ccc(-c3ccccc3)cc2n1] | 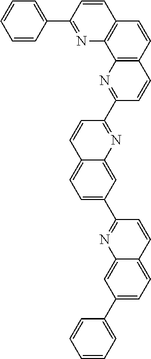 [SMILES: c1ccc(-c2ccc3ccc(-c4ccc5ccc(-c6ccc7ccc8ccc(-c9ccccc9)nc8c7n6)nc5c4)nc3c2)cc1] | 72% |
| 175 | 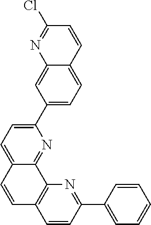 [SMILES: Clc1ccc2ccc(-c3ccc4ccc5ccc(-c6ccccc6)nc5c4n3)cc2n1] | 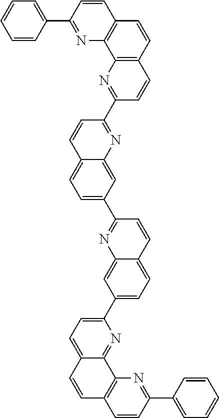 [SMILES: c1ccc(-c2ccc3ccc4ccc(-c5ccc6ccc(-c7ccc8ccc(-c9ccc%10ccc%11ccc(-c%12ccccc%12)nc%11c%10n9)nc8c7)nc6c5)nc4c3n2)cc1] | 69% |

A target compound was synthesized in the same manner as in Preparation Example 1 except that 1-(1,10-phenanthrolin-2-yl)ethanone was used instead of 1-(pyridin-2-yl)ethanone, and Intermediate U of the following Table 21 was used instead of 2-chloro-7-phenylquinoline.

TABLE 21

| Compound | | | |
|---|---|---|---|
| No. | Intermediate U | Target Compound | Yield |
| 179 | 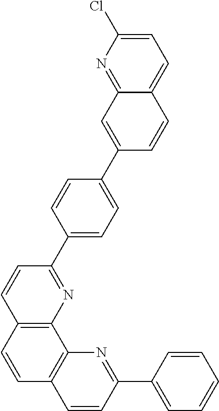 [SMILES: Clc1ccc2ccc(-c3ccc(-c4ccc5ccc6ccc(-c7ccccc7)nc6c5n4)cc3)cc2n1] | 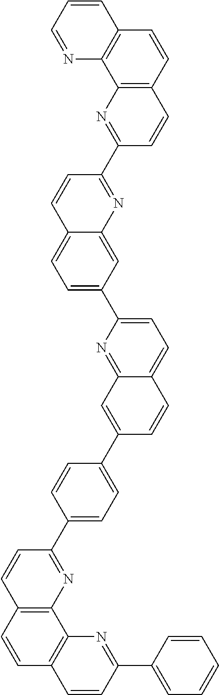 [SMILES: c1ccc(-c2ccc3ccc4ccc(-c5ccc(-c6ccc7ccc(-c8ccc9ccc(-c%10ccc%11ccc%12cccnc%12c%11n%10)nc9c8)nc7c6)cc5)nc4c3n2)cc1] | 75% |

A target compound was synthesized in the same manner as in Preparation Example 1 except that 1-(1,10-phenanthrolin-5-yl)ethanone was used instead of 1-(pyridin-2-yl)ethanone, and Intermediate V of the following Table 22 was used instead of 2-chloro-7-phenylquinoline.

TABLE 22

| Compound | | | |
|---|---|---|---|
| No. | Intermediate V | Target Compound | Yield |
| 182 | 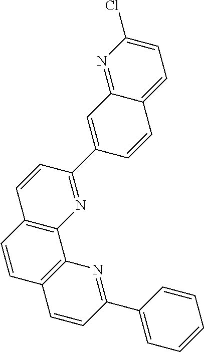 [SMILES: Clc1ccc2ccc(-c3ccc4ccc5ccc(-c6ccccc6)nc5c4n3)cc2n1] | 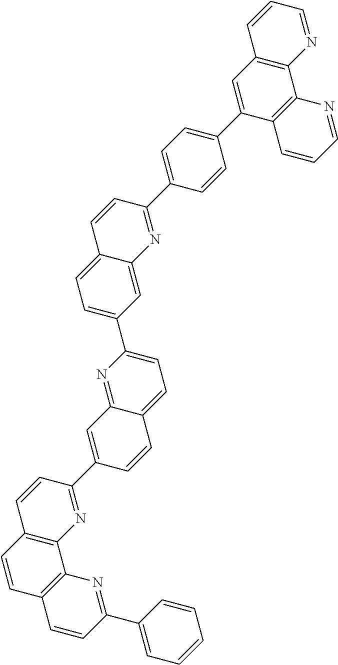 [SMILES: c1ccc(-c2ccc3ccc4ccc(-c5ccc6ccc(-c7ccc8ccc(-c9ccc(-c%10cc%11cccnc%11c%11ncccc%10%11)cc9)nc8c7)nc6c5)nc4c3n2)cc1] | 69% |

A target compound was synthesized in the same manner as in Preparation Example 1 except that 1-(3-(4,6-diphenyl-1,3,5-triazin-2-yl)phenyl)ethanone was used instead of 1-(pyridin-2-yl)ethanone, and Intermediate W of the following Table 23 was used instead of 2-chloro-7-phenylquinoline.

TABLE 23

| Compound | | | |
|---|---|---|---|
| No. | Intermediate W | Target Compound | Yield |
| 184 | 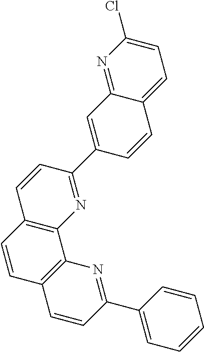 [SMILES: Clc1ccc2ccc(-c3ccc4ccc5ccc(-c6ccccc6)nc5c4n3)cc2n1] | 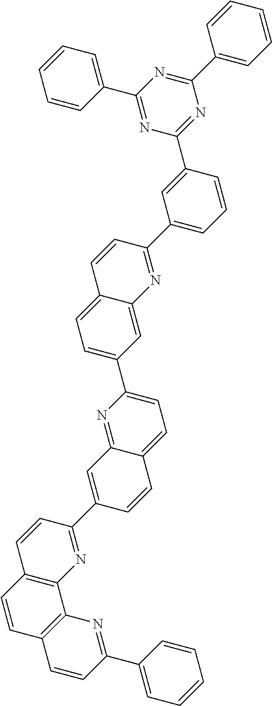 [SMILES: c1ccc(-c2ccc3ccc4ccc(-c5ccc6ccc(-c7ccc8ccc(-c9cccc(-c%10nc(-c%11ccccc%11)nc(-c%11ccccc%11)n%10)c9)nc8c7)nc6c5)nc4c3n2)cc1] | 69% |

A target compound was synthesized in the same manner as in Preparation Example 1 except that 1-(1,10-phenanthrolin-4-yl)ethanone was used instead of 1-(pyridin-2-yl)ethanone, and Intermediate X of the following Table 24 was used instead of 2-chloro-7-phenylquinoline.

TABLE 24

| Compound | | | |
|---|---|---|---|
| No. | Intermediate X | Target Compound | Yield |
| 188 | 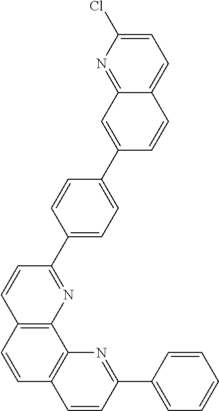 [SMILES: Clc1ccc2ccc(-c3ccc(-c4ccc5ccc6ccc(-c7ccccc7)nc6c5n4)cc3)cc2n1] | 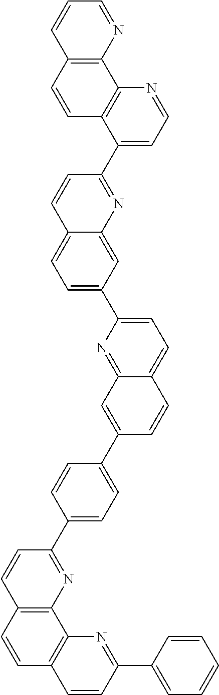 [SMILES: c1ccc(-c2ccc3ccc4ccc(-c5ccc(-c6ccc7ccc(-c8ccc9ccc(-c%10ccnc%11c%10ccc%10cccnc%10%11)nc9c8)nc7c6)cc5)nc4c3n2)cc1] | 66% |

Target compounds were synthesized in the same manner as in Preparation Example 1 except that 1-(1,10-phenanthrolin-5-yl)ethanone was used instead of 1-(pyridin-2-yl)ethanone, and Intermediate Y of the following Table 25 was used instead of 2-chloro-7-phenylquinoline.

TABLE 25

| Compound | | | |
|---|---|---|---|
| No. | Intermediate Y | Target Compound | Yield |
| 192 | 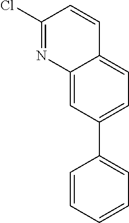 [SMILES: Clc1ccc2ccc(-c3ccccc3)cc2n1] | 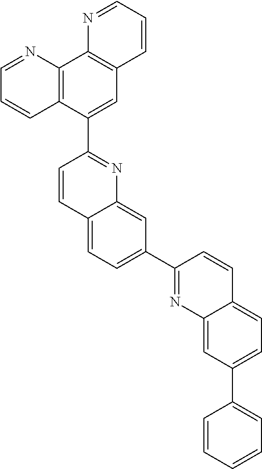 [SMILES: c1ccc(-c2ccc3ccc(-c4ccc5ccc(-c6cc7cccnc7c7ncccc67)nc5c4)nc3c2)cc1] | 71% |
| 194 | 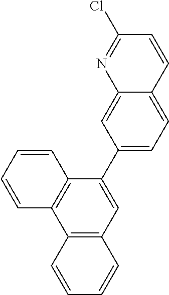 [SMILES: Clc1ccc2ccc(-c3cc4ccccc4c4ccccc34)cc2n1] | 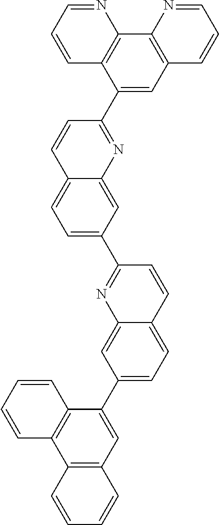 [SMILES: c1ccc2c(c1)cc(-c1ccc3ccc(-c4ccc5ccc(-c6cc7cccnc7c7ncccc67)nc5c4)nc3c1)c1ccccc12] | 69% |
| 196 | 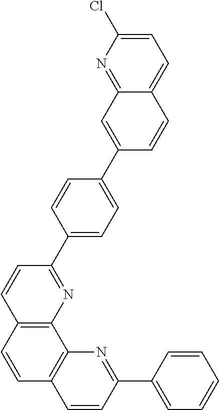 [SMILES: Clc1ccc2ccc(-c3ccc(-c4ccc5ccc6ccc(-c7ccccc7)nc6c5n4)cc3)cc2n1] | 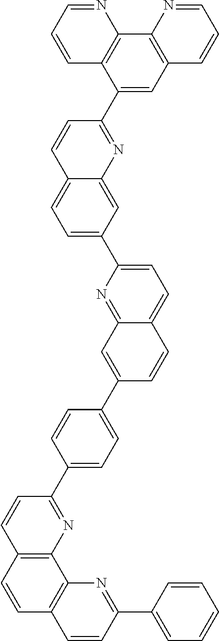 [SMILES: c1ccc(-c2ccc3ccc4ccc(-c5ccc(-c6ccc7ccc(-c8ccc9ccc(-c%10cc%11cccnc%11c%11ncccc%10%11)nc9c8)nc7c6)cc5)nc4c3n2)cc1] | 72% |
| 199 | 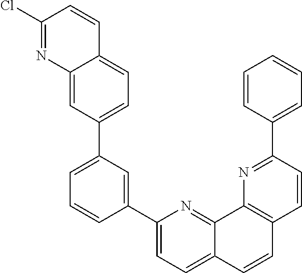 [SMILES: Clc1ccc2ccc(-c3cccc(-c4ccc5ccc6ccc(-c7ccccc7)nc6c5n4)c3)cc2n1] | 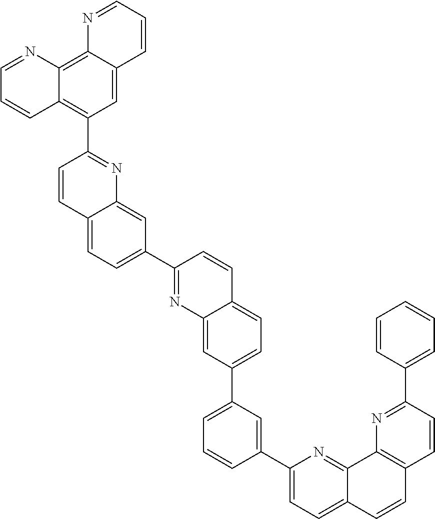 [SMILES: c1ccc(-c2ccc3ccc4ccc(-c5cccc(-c6ccc7ccc(-c8ccc9ccc(-c%10cc%11cccnc%11c%11ncccc%10%11)nc9c8)nc7c6)c5)nc4c3n2)cc1] | 73% |

Target compounds were synthesized in the same manner as in Preparation Example 1 except that 1-(3-(pyridin-2-yl)phenyl)ethanone was used instead of 1-(pyridin-2-yl)ethanone, and Intermediate Z of the following Table 26 was used instead of 2-chloro-7-phenylquinoline.

TABLE 26

| Compound | | | |
|---|---|---|---|
| No. | Intermediate Z | Target Compound | Yield |
| 203 | 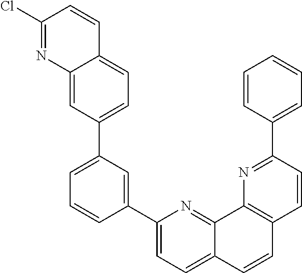 [SMILES: Clc1ccc2ccc(-c3cccc(-c4ccc5ccc6ccc(-c7ccccc7)nc6c5n4)c3)cc2n1] | 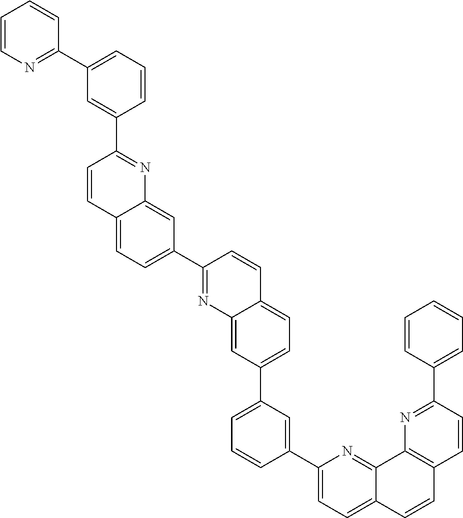 [SMILES: c1ccc(-c2ccc3ccc4ccc(-c5cccc(-c6ccc7ccc(-c8ccc9ccc(-c%10cccc(-c%11ccccn%11)c%10)nc9c8)nc7c6)c5)nc4c3n2)cc1] | 78% |
| 205 | 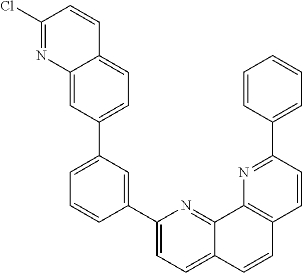 [SMILES: Clc1ccc2ccc(-c3cccc(-c4ccc5ccc6ccc(-c7ccccc7)nc6c5n4)c3)cc2n1] | 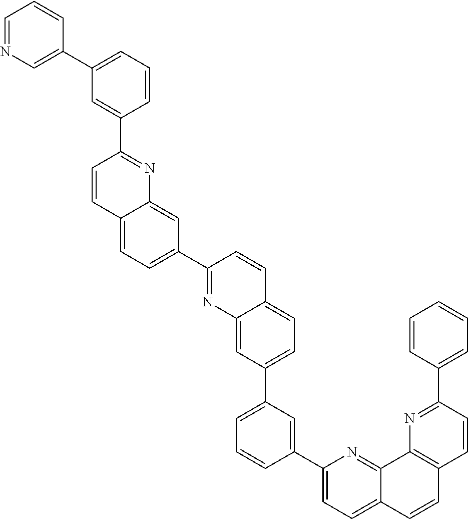 [SMILES: c1ccc(-c2ccc3ccc4ccc(-c5cccc(-c6ccc7ccc(-c8ccc9ccc(-c%10cccc(-c%11cccnc%11)c%10)nc9c8)nc7c6)c5)nc4c3n2)cc1] | 71% |

A target compound was synthesized in the same manner as in Preparation Example 1 except that 1-(3-(9-phenyl-1,10-phenanthrolin-2-yl)phenyl)ethanone was used instead of 1-(pyridin-2-yl)ethanone, and Intermediate A-1 of the following Table 27 was used instead of 2-chloro-7-phenylquinoline.

TABLE 27

| Compound | | | |
|---|---|---|---|
| No. | Intermediate A-1 | Target Compound | Yield |
| 207 | 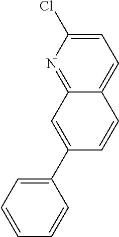 [SMILES: Clc1ccc2ccc(-c3ccccc3)cc2n1] | 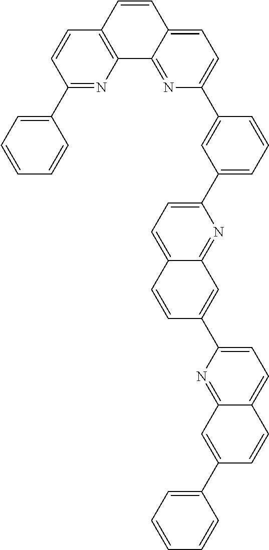 [SMILES: c1ccc(-c2ccc3ccc(-c4ccc5ccc(-c6cccc(-c7ccc8ccc9ccc(-c%10ccccc%10)nc9c8n7)c6)nc5c4)nc3c2)cc1] | 78% |

Target compounds were synthesized in the same manner as in Preparation Example 1 except that 1-(4-(9-phenyl-1,10-phenanthrolin-2-yl)phenyl)ethanone was used instead of 1-(pyridin-2-yl)ethanone, and Intermediate B-1 of the following Table 28 was used instead of 2-chloro-7-phenylquinoline.

TABLE 28

| Compound | | | |
|---|---|---|---|
| No. | Intermediate B-1 | Target Compound | Yield |
| 210 | 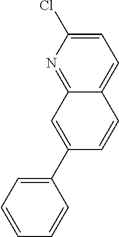 [SMILES: Clc1ccc2ccc(-c3ccccc3)cc2n1] | 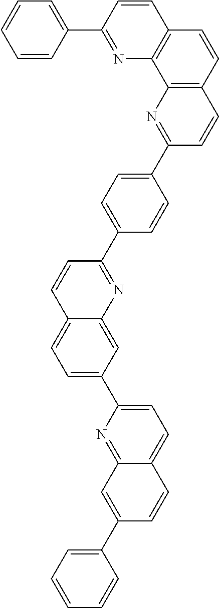 [SMILES: c1ccc(-c2ccc3ccc(-c4ccc5ccc(-c6ccc(-c7ccc8ccc9ccc(-c%10ccccc%10)nc9c8n7)cc6)nc5c4)nc3c2)cc1] | 78% |
| 211 | 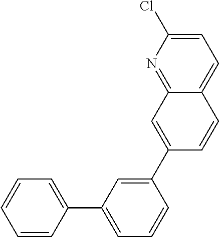 [SMILES: Clc1ccc2ccc(-c3cccc(-c4ccccc4)c3)cc2n1] | 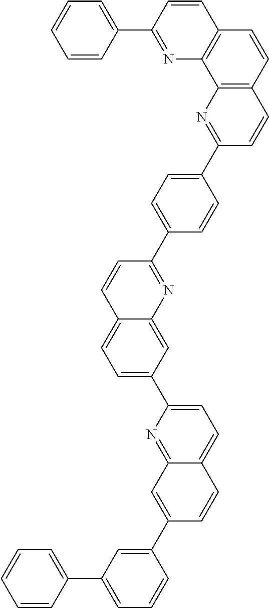 [SMILES: c1ccc(-c2cccc(-c3ccc4ccc(-c5ccc6ccc(-c7ccc(-c8ccc9ccc%10ccc(-c%11ccccc%11)nc%10c9n8)cc7)nc6c5)nc4c3)c2)cc1] | 77% |
| 214 | 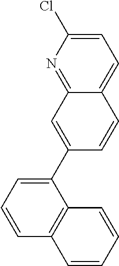 [SMILES: Clc1ccc2ccc(-c3cccc4ccccc34)cc2n1] | 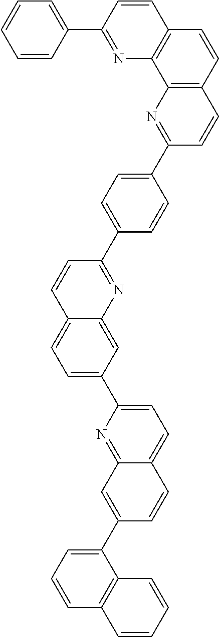 [SMILES: c1ccc(-c2ccc3ccc4ccc(-c5ccc(-c6ccc7ccc(-c8ccc9ccc(-c%10cccc%11ccccc%10%11)cc9n8)cc7n6)cc5)nc4c3n2)cc1] | 75% |

A target compound was synthesized in the same manner as in Preparation Example 1 except that 1-(4-(1,10-phenanthrolin-4-yl)phenyl)ethanone was used instead of 1-(pyridin-2-yl)ethanone, and Intermediate C-1 of the following Table 29 was used instead of 2-chloro-7-phenylquinoline.

TABLE 29

| Compound | | | |
|---|---|---|---|
| No. | Intermediate C-1 | Target Compound | Yield |
| 219 | 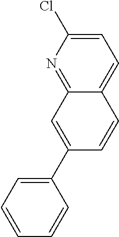 [SMILES: Clc1ccc2ccc(-c3ccccc3)cc2n1] | 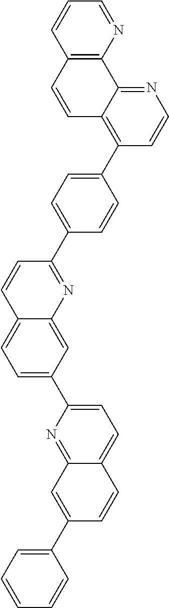 [SMILES: c1ccc(-c2ccc3ccc(-c4ccc5ccc(-c6ccc(-c7ccnc8c7ccc7cccnc78)cc6)nc5c4)nc3c2)cc1] | 69% |

A target compound was synthesized in the same manner as in Preparation Example 1 except that 1-(4-(1,10-phenanthrolin-5-yl)phenyl)ethanone was used instead of 1-(pyridin-2-yl)ethanone, and Intermediate D-1 of the following Table 30 was used instead of 2-chloro-7-phenylquinoline.

TABLE 30

| Com- | | | |
|---|---|---|---|
| pound | | | |
| No. | Intermediate D-1 | Target Compound | Yield |
| 230 | 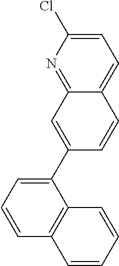 [SMILES: Clc1ccc2ccc(-c3cccc4ccccc34)cc2n1] | 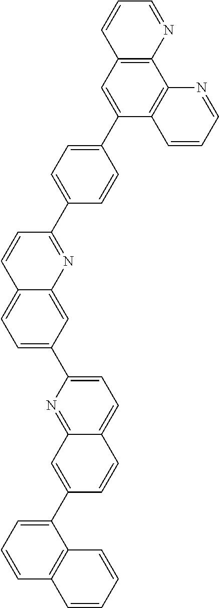 [SMILES: c1ccc2c(-c3ccc4ccc(-c5ccc6ccc(-c7ccc(-c8cc9cccnc9c9ncccc89)cc7)nc6c5)nc4c3)cccc2c1] | 66% |

Target compounds were synthesized in the same manner as in Preparation Example 1 except that 1-(4-(9-phenyl-1,10-phenanthrolin-2-yl) naphthalen-1-yl) ethanone was used instead of 1-(pyridin-2-yl)ethanone, and Intermediate E-1 of the following Table 31 was used instead of 2-chloro-7-phenylquinoline.

TABLE 31

| Com- | | | |
|---|---|---|---|
| pound | | | |
| No. | Intermediate E-1 | Target Compound | Yield |
| 234 | 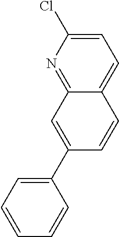 [SMILES: Clc1ccc2ccc(-c3ccccc3)cc2n1] | 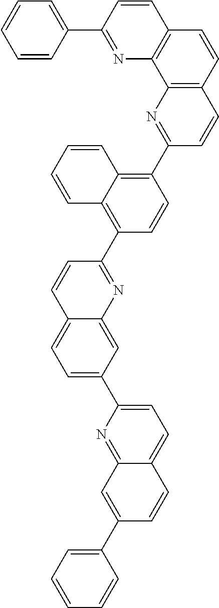 [SMILES: c1ccc(-c2ccc3ccc(-c4ccc5ccc(-c6ccc(-c7ccc8ccc9ccc(-c%10ccccc%10)nc9c8n7)c7ccccc67)nc5c4)nc3c2)cc1] | 66% |
| 238 | 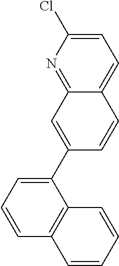 [SMILES: Clc1ccc2ccc(-c3cccc4ccccc34)cc2n1] | 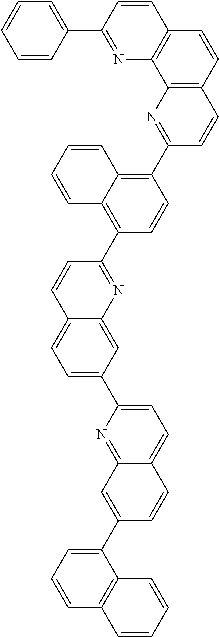 [SMILES: c1ccc(-c2ccc3ccc4ccc(-c5ccc(-c6ccc7ccc(-c8ccc9ccc(-c%10cccc%11ccccc%10%11)cc9n8)cc7n6)c6ccccc56)nc4c3n2)cc1] | 73% |

A target compound was synthesized in the same manner as in Preparation Example 1 except that 1-(6-(9-phenyl-1,10-phenanthrolin-2-yl) naphthalen-2-yl) ethanone was used instead of 1-(pyridin-2-yl)ethanone, and Intermediate F-1 of the following Table 32 was used instead of 2-chloro-7-phenylquinoline.

TABLE 32

| Com- | | | |
|---|---|---|---|
| pound | | | |
| No. | Intermediate F-1 | Target Compound | Yield |
| 241 | 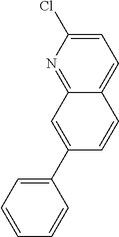 [SMILES: Clc1ccc2ccc(-c3ccccc3)cc2n1] | 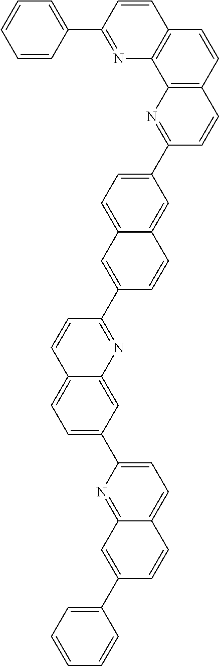 [SMILES: c1ccc(-c2ccc3ccc(-c4ccc5ccc(-c6ccc7cc(-c8ccc9ccc%10ccc(-c%11ccccc%11)nc%10c9n8)ccc7c6)nc5c4)nc3c2)cc1] | 69% |

A target compound was synthesized in the same manner as in Preparation Example 1 except that 1-(10-(9-phenyl-1,10-phenanthrolin-2-yl) anthracen-9-yl) ethanone was used instead of 1-(pyridin-2-yl)ethanone, and Intermediate G-1 of the following Table 33 was used instead of 2-chloro-7-phenylquinoline.

TABLE 33

| Com- | | | |
|---|---|---|---|
| pound | | | |
| No. | Intermediate G-1 | Target Compound | Yield |
| 247 | 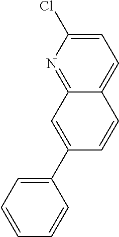 [SMILES: Clc1ccc2ccc(-c3ccccc3)cc2n1] | 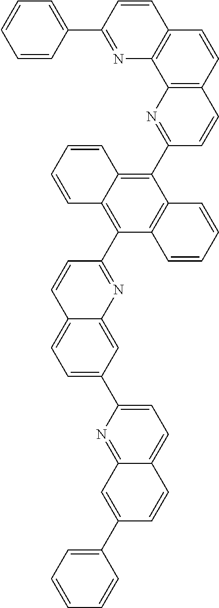 [SMILES: c1ccc(-c2ccc3ccc(-c4ccc5ccc(-c6c7ccccc7c(-c7ccc8ccc9ccc(-c%10ccccc%10)nc9c8n7)c7ccccc67)nc5c4)nc3c2)cc1] | 73% |

Target compounds were synthesized in the same manner as in Preparation Example 1 except that 2-chloroquinoline was used instead of 2-chloro-7-phenylquinoline, and Intermediate H-1 of the following Table 34 was used instead of 1-(pyridin-2-yl) ethanone.

TABLE 34

| Compound | | | |
|---|---|---|---|
| No. | Intermediate H-1 | Target Compound | Yield |
| 253 | 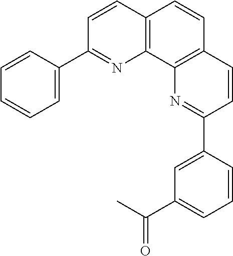 [SMILES: CC(=O)c1cccc(-c2ccc3ccc4ccc(-c5ccccc5)nc4c3n2)c1] | 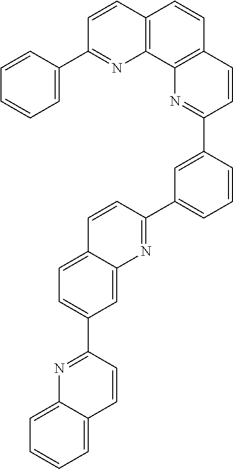 [SMILES: c1ccc(-c2ccc3ccc4ccc(-c5cccc(-c6ccc7ccc(-c8ccc9ccccc9n8)cc7n6)c5)nc4c3n2)cc1] | 77% |
| 254 | 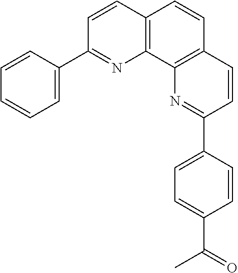 [SMILES: CC(=O)c1ccc(-c2ccc3ccc4ccc(-c5ccccc5)nc4c3n2)cc1] | 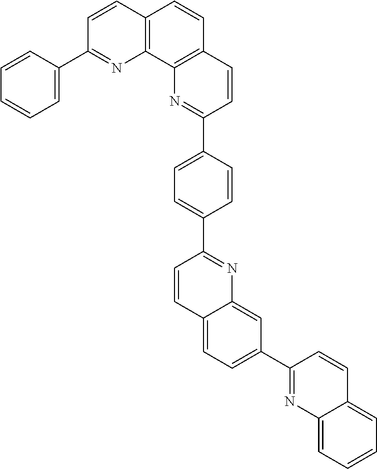 [SMILES: c1ccc(-c2ccc3ccc4ccc(-c5ccc(-c6ccc7ccc(-c8ccc9ccccc9n8)cc7n6)cc5)nc4c3n2)cc1] | 71% |
| 255 | 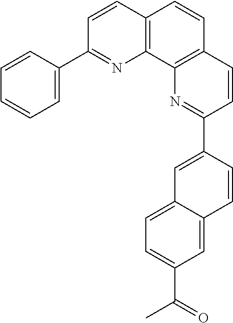 [SMILES: CC(=O)c1ccc2cc(-c3ccc4ccc5ccc(-c6ccccc6)nc5c4n3)ccc2c1] | 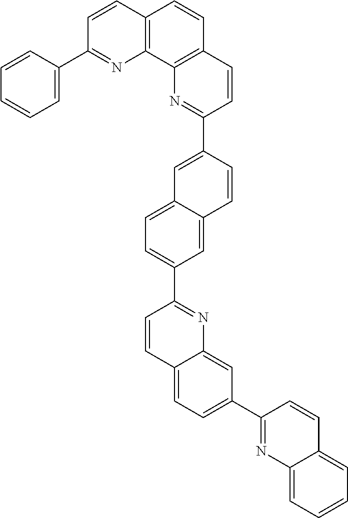 [SMILES: c1ccc(-c2ccc3ccc4ccc(-c5ccc6cc(-c7ccc8ccc(-c9ccc%10ccccc%10n9)cc8n7)ccc6c5)nc4c3n2)cc1] | 73% |

Target compounds were synthesized in the same manner as in Preparation Example 1 except that 1-(9-phenyl-1,10-phenanthrolin-2-yl)ethanone was used instead of 1-(pyridin-2-yl)ethanone, and Intermediate I-1 of the following Table 35 was used instead of 2-chloro-7-phenylquinoline.

TABLE 35

| Compound | | | |
|---|---|---|---|
| No. | Intermediate I-1 | Target Compound | Yield |
| 256 | 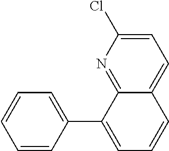 [SMILES: Clc1ccc2cccc(-c3ccccc3)c2n1] | 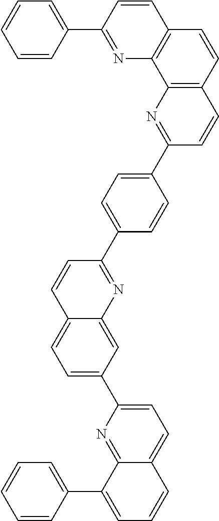 [SMILES: c1ccc(-c2ccc3ccc4ccc(-c5ccc(-c6ccc7ccc(-c8ccc9cccc(-c%10ccccc%10)c9n8)cc7n6)cc5)nc4c3n2)cc1] | 75% |
| 257 | 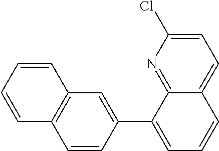 [SMILES: Clc1ccc2cccc(-c3ccc4ccccc4c3)c2n1] | 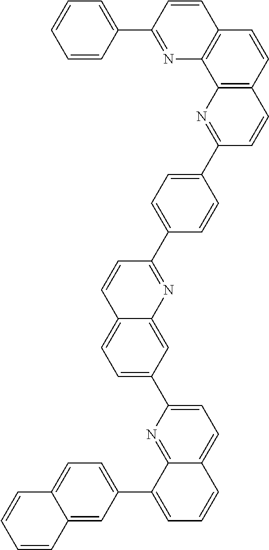 [SMILES: c1ccc(-c2ccc3ccc4ccc(-c5ccc(-c6ccc7ccc(-c8ccc9cccc(-c%10ccc%11ccccc%11c%10)c9n8)cc7n6)cc5)nc4c3n2)cc1] | 71% |
| 258 | 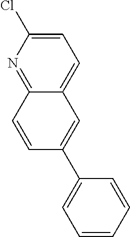 [SMILES: Clc1ccc2cc(-c3ccccc3)ccc2n1] | 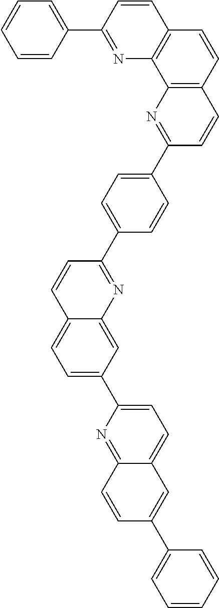 [SMILES: c1ccc(-c2ccc3nc(-c4ccc5ccc(-c6ccc(-c7ccc8ccc9ccc(-c%10ccccc%10)nc9c8n7)cc6)nc5c4)ccc3c2)cc1] | 70% |
| 259 | 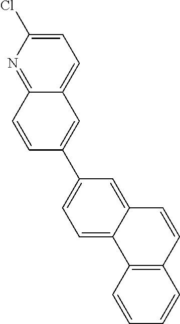 [SMILES: Clc1ccc2cc(-c3ccc4c(ccc5ccccc54)c3)ccc2n1] | 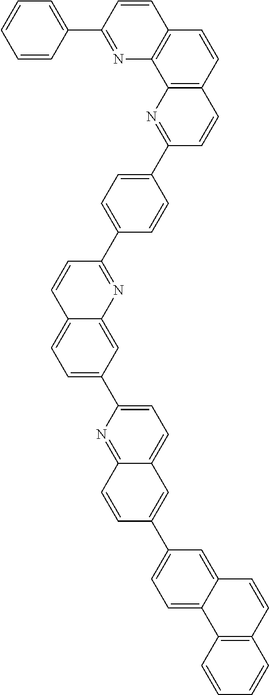 [SMILES: c1ccc(-c2ccc3ccc4ccc(-c5ccc(-c6ccc7ccc(-c8ccc9cc(-c%10ccc%11c(ccc%12ccccc%12%11)c%10)ccc9n8)cc7n6)cc5)nc4c3n2)cc1] | 65% |
| 260 | 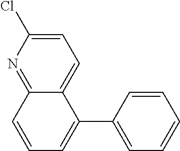 [SMILES: Clc1ccc2c(-c3ccccc3)cccc2n1] | 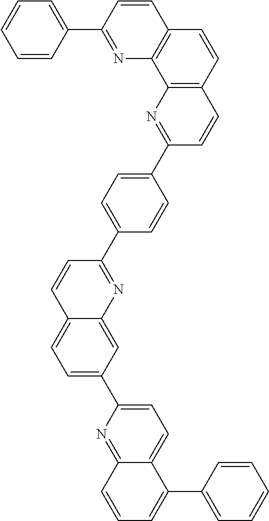 [SMILES: c1ccc(-c2ccc3ccc4ccc(-c5ccc(-c6ccc7ccc(-c8ccc9c(-c%10ccccc%10)cccc9n8)cc7n6)cc5)nc4c3n2)cc1] | 75% |
| 261 | 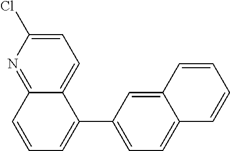 [SMILES: Clc1ccc2c(-c3ccc4ccccc4c3)cccc2n1] | 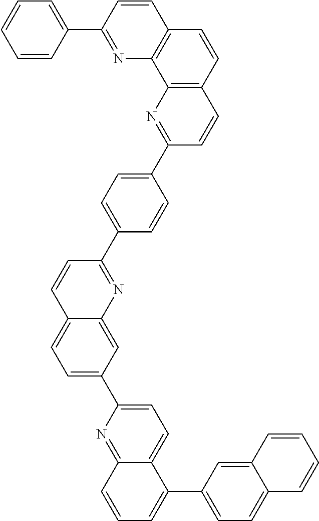 [SMILES: c1ccc(-c2ccc3ccc4ccc(-c5ccc(-c6ccc7ccc(-c8ccc9c(-c%10ccc%11ccccc%11c%10)cccc9n8)cc7n6)cc5)nc4c3n2)cc1] | 73% |

Synthesis identification results for the compounds prepared using the above-described methods are shown in the following Tables 36 and 37.

TABLE 36

| NO | ¹H NMR (CDCl₃, 300 Mz) |
|---|---|
| 1 | δ = 9.30(1H, d), 8.78(1H, d), 8.53~8.54(2H, m), 8.27~8.31(3H, m), |
| | 8.03~8.12(4H, m), 7.70(1H, m), 7.35~7.51(6H, m), 7.14(1H, t) |
| 3 | δ = 9.30(1H, d), 8.78(1H, s), 8.53~8.54(2H, m), 8.27~8.31(3H, m), |
| | 7.92~8.15(7H, m), 7.70~7.73(2H, m), 7.58~7.59(3H, m), |
| | 7.35(1H, d), 7.14(1H, m) |
| 8 | δ = 9.30(1H, d), 8.78(1H, d), 8.53~8.54(2H, m), 8.23~8.31(6H, m), |
| | 8.03~8.15(4H, m), 7.70~7.79(5H, m), 7.35~7.57(19H, m), |
| | 7.14(1H, t) |
| 12 | δ = 9.30(1H, d), 8.78(1H, d), 8.53~8.54(2H, m), 8.27~8.31(7H, m), |
| | 8.03~8.15(4H, m), 7.85(2H, d), 7.70(1H, m), 7.35~7.51(9H, m), |
| | 7.14(1H, t) |
| 15 | δ = 9.30(1H, d), 8.78(1H, s), 8.53~8.55(3H, m), 8.23~8.31(8H, m), |
| | 8.03~8.15(5H, m), 7.94(1H, d), 7.63~7.85(8H, m), |
| | 7.25~7.51(8H, m), 7.14(1H, t) |
| 17 | δ = 9.30(1H, d), 8.78(1H, s), 8.53~8.54(2H, m), 8.27~8.31(7H, m), |
| | 8.03~8.15(4H, m), 7.85(2H, d), 7.70(1H, m), 7.35~7.51(7H, m), |
| | 7.25(2H, d), 7.14(1H, t) |
| 19 | δ = 9.30(1H, d), 8.78(1H, s), 8.53~8.57(3H, m), |
| | 7.98~8.31(10H, m), 7.48~7.78(7H, m), 7.35(1H, d), 7.14(1H, t) |
| 22 | δ = 9.30(1H, d), 8.78(2H, s), 8.53~8.54(3H, m), 8.29~8.31(4H, s), |
| | 8.06~8.15(6H, m), 7.81(1H, d), 7.70(1H, t), 7.47~7.54(3H, m), |
| | 7.35(3H, d), 7.14(1H, t) |
| 27 | δ = 9.30(1H, d), 8.78~8.81(3H, m), 8.53~8.54(2H, m), |
| | 8.27~8.31(3H, m), 8.03~8.15(7H, m), 7.81~7.88(3H, m), |
| | 7.70(1H, m), 7.47~7.54(3H, m), 7.35(3H, d), 77.14(1H, t) |
| 30 | δ = 9.30(1H, d), 8.78(1H, s), 8.52~8.55(5H, m), 8.27~8.31(5H, m), |
| | 8.03~8.15(8H, m), 7.81(1H, d), 7.70(1H, t), 7.47~7.55(5H, m), |
| | 7.35(3H, d), 7.14(1H, t) |
| 33 | δ = 9.75(1H, s), 8.93(1H, d), 8.76~8.78(2H, m), 8.53~8.54(2H, m), |
| | 8.42~8.44(2H, m), 8.27(1H, s), 8.03~8.15(6H, m), |
| | 7.55~7.61(4H, m), 7.35~7.41(2H, m) |
| 37 | δ = 9.75(1H, s), 8.93(1H, d), 8.76~8.81(4H, m), 8.54(1H, d), |
| | 8.44(1H, d), 8.27~8.30(3H, m), 8.03~8.15(7H, m), |
| | 7.81~7.88(3H, m), 7.35~7.60(8H, m) |
| 40 | δ = 9.75(1H, s), 8.93(1H, d), 8.76~8.78(2H, d), 8.44~8.55(5H, m), |
| | 8.27~8.30(3H, m), 8.03~8.15(8H, m), 7.81(1H, d), |
| | 7.35~7.60(10H, m) |
| 42 | δ = 8.78(3H, d), 8.44~8.54(4H, m), 8.27(1H, s), 8.03~8.15(4H, m), |
| | 7.41~7.52(7H, m) |
| 45 | δ = 8.93(1H, d), 8.78(2H, d), 8.44~8.54(4H, m), 7.93(1H, s), |
| | 8.03~8.15(7H, m), 7.82~7.88(3H, m), 7.71(2H, s), 7.35~7.41(2H, d) |
| 48 | δ = 8.78~8.81(5H, m), 8.44~8.54(4H, m), 8.27~8.30(3H, m), |
| | 8.03~8.15(7H, m), 7.81~7.88(3H, m), 7.35~7.54(7H, m) |
| 51 | δ = 8.78(3H, m), 8.44~8.55(7H, m), 8.27~8.30(3H, m), |
| | 8.03~8.15(8H, m), 7.81(1H, d), 7.35~8.55(9H, m) |
| 54 | δ = 8.97(2H, d), 8.78~8.81(3H, m), 8.54(1H, d), 8.44(1H, d), |
| | 8.27~8.30(3H, m), 8.03~8.15(7H, m), 7.81~7.88(3H, m), |
| | 7.35~7.54(8H, m) |
| 59 | δ = 8.97(2H, d), 8.78(1H, s), 8.54(1H, d), 8.44(1H, d), |
| | 8.23~8.30(6H, m), 8.03~8.15(4H, m), 7.79~7.85(4H, m), |
| | 7.35~7.51(9H, m) |
| 64 | δ = 8.78(1H, s), 8.54~8.55(2H, m), 8.37~8.44(3H, m), 8.27(1H, s), |
| | 8.03~8.15(6H, m), 7.79(4H, m), 7.41~7.61(11H, m) |
| 68 | δ = 8.78~8.81(3H, m), 8.54(1H, d), 8.44(1H, m), 8.37(1H, s), |
| | 8.27~8.30(3H, m), 8.03~8.15(7H, m), 7.79~7.88(7H, m), |
| | 7.35~7.54(13H, m) |
| 72 | δ = 9.24(2H, s), 8.78(1H, d), 8.70(2H, d), 8.42~8.54(5H, m), |
| | 8.27(1H, s), 8.03~8.15(4H, m), 7.35~7.57(9H, m) |
| 73 | δ = 9.30(1H, s), 9.05(2H, s), 8.78~8.81(3H, m), 8.54(1H, d), |
| | 8.44(1H, d), 8.27~8.30(3H, m), 8.03~8.15(7H, m), |
| | 7.81~7.88(3H, m), 7.35~7.54(7H, m) |
| 79 | δ = 9.19(1H, s), 8.78(1H, s), 8.54(1H, d), 8.44(1H, d), |
| | 8.27~8.28(3H, m), 7.92~8.15(7H, m), 7.73~7.79(3H, m), |
| | 7.35~7.59(11H, m) |
| 80 | δ = 9.19(1H, s) 8.93(1H, d), 8.78(1H, d), 8.54(1H, d), 8.44(1H, d), |
| | 8.28(2H, m), 8.03~8.15(7H, m), 7.71~7.88(7H, m), |
| | 7.35~7.51(8H, m) |
| 83 | δ = 9.19(1H, s), 8.78~8.81(3H, d), 8.54(1H, d), 8.44(1H, d), |
| | 8.27~8.30(5H, m), 8.03~8.15(7H, m), 7.79~7.88(5H, m), |
| | 7.35~7.54(13H, m) |
| 87 | δ = 9.19(1H, s), 8.85(1H, d), 8.78(1H, s), 8.54(1H, d), |
| | 8.28~8.44(7H, m), 7.92~8.15(9H, m), 7.73~7.79(4H, m), |
| | 7.35~7.58(14H, m) |
| 90 | δ = 8.76~8.79(4H, d), 8.52~8.55(4H, m), 8.44(1H, d), |
| | 8.27~8.30(3H, m), 8.03~8.15(8H, m), 7.81(1H, d), |
| 92 | 7.35~7.55(9H, m) δ = 8.78~8.82(5H, m), 8.54(1H, d), 8.44(1H, d), |
| | 8.27~8.30(3H, m), |
| | 8.03~8.15(7H, m), 7.81~7.88(3H, m), 7.35~7.54(7H, m) |
| 97 | δ = 8.78(1H, s), 8.54(1H, d), 8.44(1H, d), 8.28(4H, d), |
| | 8.03~8.15(4H, m), 7.41~7.54(13H, m) |
| 100 | δ = 8.93(1H, d), 8.78(1H, s), 8.54(1H, d), 8.44(1H, d), |
| | 8.28(4H, d), 8.03~8.15(7H, m), 7.82~7.88(3H, m), 7.71(2H, s), |
| | 7.35~7.51(8H, m) |
| 102 | δ = 8.78~8.81(3H, m), 8.54(1H, d), 8.44(1H, d), 8.27~8.30(7H, m), |
| | 8.03~8.15(7H, m), 7.81~7.88(3H, m), 7.35~7.54(13H, m) |
| 105 | δ = 8.45~8.83(2H, m), 8.52~8.55(4H, m), 8.38~8.44(2H, m), |
| | 8.27~8.28(5H, m), 8.03~8.15(7H, m), 7.81(1H, d), |
| | 7.35~7.58(12H, m) |
| 108 | δ = 8.78~8.88(3H, m), 8.53~8.54(2H, m), 8.38~8.42(2H, m), |
| | 8.27(1H, s), 8.03~8.15(8H, m), 7.55~7.69(5H, m), 7.35(2H, d) |
| 111 | δ = 8.78~8.88(3H, m), 8.54(1H, d), 8.03~8.38(15H, m), |
| | 7.81(1H, d), 7.47~7.60(7H, m), 7.35(4H, d) |
| 114 | δ = 8.78~8.88(4H, m), 8.52~8.55(4H, m), 8.38(2H, m), 8.27(1H, s), |
| | 8.03~8.15(9H, m), 7.81(1H, d), 7.69(1H, m), 7.55~7.58(4H, m), |
| | 7.35(3H, d) |
| 116 | δ = 8.78~8.88(3H, m), 8.54(1H, d), 8.23~8.38(7H, m), |
| | 8.03~8.15(6H, m), 7.79~7.85(4H, m), 7.69(1H, m), |
| | 7.35~7.58(9H, m) |
| 120 | δ = 8.78~8.88(3H, m), 8.54(1H, d), 8.38(1H, d), 8.23~8.28(4H, m), |
| | 8.03~8.12(7H, m), 7.94(1H, d), 7.25~7.79(20H, m) |
| 121 | δ = 8.91(1H, s), 8.78~8.81(3H, m), 8.54(1H, d), 8.45(1H, d), |
| | 8.27~8.30(4H, m), 8.03~8.15(8H, m), 7.81~7.88(3H, m), |
| | 7.47~7.65(6H, m), 7.35(4H, d) |
| 124 | δ = 8.91(1H, s), 8.78(1H, d), 8.54(1H, d), 8.45(1H, d), |
| | 8.27~8.30(4H, m), 8.03~8.15(8H, m), 7.91(4H, d), 7.81(1H, d), |
| | 7.35~7.65(14H, m) |
| 128 | δ = 8.91(1H, s), 8.85(1H, d), 8.78(1H, d), 8.54(1H, d), |
| | 8.27~8.45(6H, m), 7.92~8.15(11H, m), 7.81(1H, m), 7.73(1H, d), |
| | 7.47~7.58(6H, m), 7.35(4H, d) |
| 133 | δ = 8.78~8.83(2H, m), 8.54(2H, d), 7.98~8.38(16H, m), |
| | 7.81(1H, d), 7.47~7.60(6H, m), 7.35(4H, d) |
| 137 | δ = 8.93~8.94(2H, s), 8.78~8.81(3H, m), 8.54(1H, d), 8.44(1H, d), |
| | 8.27~8.30(3H, m), 8.03~8.15(7H, m), 7.76~7.92(5H, m), |
| | 7.35~7.54(9H, m) |
| 145 | δ = 9.08(1H, s), 8.73~8.78(2H, d), 8.54(1H, d), 8.44(1H, d), |
| | 8.27(1H, s), 7.98~8.15(6H, m), 7.78(1H, m), 7.35~7.60(8H, m) |
| 147 | δ = 9.08(1H, s), 8.93(2H, d), 8.73~8.78(2H, m), 8.54(1H, d), |
| | 8.44(1H, d), 8.27(1H, s), 7.78~8.15(14H, m), |
| | 7.60(1H, m), 7.35~7.41(2H, m) |
| 150 | δ = 9.08(1H, s), 8.73~8.81(4H, m), 8.54(1H, d), 8.44(1H, d), |
| | 8.27~8.30(3H, m), 7.98~8.15(9H, m), 7.78~7.88(4H, m), |
| | 7.35~7.54(8H, m) |
| 168 | δ = 8.78(1H, s), 8.54(1H, m), 8.27~8.31(9H, m), 8.03~8.16(6H, m), |
| | 7.81~7.85(3H, m), 7.67(2H, d), 7.51(4H, m), 7.35~7.41(3H, m), |
| | 7.25(2H, d) |
| 170 | δ = 8.78~8.83(2H, m), 8.54~8.55(3H, m), 8.38~8.42(3H, m), |
| | 8.27(1H, s), 8.03~8.15(8H, m), 7.55~7.61(6H, m), 7.35(2H, d) |
| 172 | δ = 8.78(1H, s), 8.54(1H, d), 8.27~8.31(7H, m), 8.03~8.15(6H, m), |
| | 7.81(1H, d), 7.35~7.54(10H, m) |
| 175 | δ = 8.78(2H, s), 8.54(2H, d), 8.29~8.31(8H, m), 8.06~8.15(8H, m), |
| | 7.81(2H, d), 7.47~7.54(6H, m), 7.35(4H, d) |
| 176 | δ = 8.78~8.83(2H, m), 8.68(1H, s), 8.50~8.54(3H, m), |
| | 8.23~8.31(7H, m), 8.03~8.15(5H, m), 7.81(1H, d), |
| | 7.51~7.58(3H, m), 7.35(1H, d), 7.26(2H, d), 7.00(2H, m) |
| 179 | δ = 8.78~8.83(4H, m), 8.54(1H, d), 8.27~8.38(8H, m), |
| | 8.03~8.15(8H, m), 7.81~7.88(4H, m), 7.47~7.54(3H, m), |
| | 7.35(3H, d) |
| 182 | δ = 8.78~8.83(6H, m), 8.54(2H, d), 8.28~8.38(4H, m), |
| | 8.06~8.15(7H, m), 7.81(1H, d), 7.47~7.65(6H, m), |
| | 7.28~7.35(6H, m) |
| 184 | δ = 8.78(2H, s), 8.54(2H, d), 8.06~8.30(16H, m), 7.81(1H, d), |
| | 7.35~7.60(14H, m) |
| 188 | δ = 8.92(1H, d), 8.81~8.83(3H, m), 8.54(1H, d), 8.27~8.44(5H, m), |
| | 7.99~8.15(9H, m), 7.81~7.88(4H, m), 7.35~7.58(8H, m) |
| 192 | δ = 8.78~8.83(3H, m), 8.54(1H, d), 8.38(2H, d), 8.27(1H, s), |
| | 8.03~8.16(6H, m), 7.35~7.58(9H, m) |
| 194 | δ = 8.93(2H, d), 8.78~8.83(3H, m), 8.54(1H, d), 8.38(2H, d), |
| | 8.27(1H, s), 8.03~8.16(8H, m), 7.82~7.93(5H, m), 7.58(2H, m), |
| | 7.35(2H, d) |
| 196 | δ = 8.78~8.83(5H, m), 8.54(1H, d), 8.30~8.38(4H, m), |
| | 8.03~8.16(9H, m), 7.81~7.88(3H, m), 7.47~7.58(5H, m), |
| | 7.35(4H, d) |
| 199 | δ = 8.78~8.83(3H, m), 8.54(1H, d), 8.03~8.38(16H, m), |
| | 8.78~8.83(3H, m), 8.54(1H, d), 8.06~8.38(16H, m), |
| | 7.81(1H, d), 7.47~7.60(7H, m), 7.35(4H, d) |
| 203 | δ = 8.78(1H, s), 8.72(1H, s), 8.50~8.54(2H, m), |
| | 8.03~8.32(15H, m), 7.81(1H, s), 7.47~7.63(7H, m), 7.35(4H, d), |
| | 7.26(1H, d), 7.00(1H, m) |
| 205 | δ = 9.24(1H, s), 8.78(1H, s), 8.70(1H, d), 8.54(1H, m), 8.42(1H, d), |
| | 8.03~8.30(15H, m), 7.81(1H, d), 7.47~7.60(8H, m), 7.35(4H, d) |
| 207 | δ = 8.78~8.83(3H, m), 8.72(1H, d), 8.50~8.54(2H, m), |
| | 8.02~8.38(14H, m), 7.51~7.66(9H, m), 7.35(2H, d), 7.26(1H, d), |
| | 7.00(1H, m) |
| 210 | δ = 8.84(4H, d), 8.78(1H, d), 8.54(1H, d), 8.27~8.30(3H, m), |
| | 8.03~8.15(8H, m), 7.81(1H, d), 7.35~7.54(12H, m) |
| 211 | δ = 8.84~8.78(5H, m), 8.54(1H, d), 8.27~8.30(3H, m), |
| | 8.03~8.15(8H, m), 7.81(1H, d), 7.70(1H, s), 7.35~7.57(15H, m) |
| 214 | δ = 8.84(4H, d), 7.78(1H, d), 8.54(1H, m), 8.42(1H, d), |
| | 8.27~8.30(3H, m), 8.02~8.15(10H, m), 7.81(2H, d), |
| | 7.47~7.55(6H, m), 7.35(4H, d) |
| 219 | δ = 8.78~8.89(5H, m), 8.54(1H, d), 8.38(1H, m), 8.27(1H, s), |
| | 8.03~8.15(6H, m), 7.81(1H, d), 7.28~7.58(11H, m) |
| 230 | δ = 8.78~8.83(5H, m), 8.54~8.55(2H, m), 8.38~8.42(3H, m), |
| | 8.27(1H, s), 8.03~8.15(7H, m), 7.55~7.65(6H, m), |
| | 7.28~7.35(4H, m) |
| 234 | δ = 8.78(1H, s), 8.54~8.55(5H, m), 8.27~8.30(3H, m), |
| | 8.03~8.15(8H, m), 7.81(1H, d), 7.35~7.55(14H, m) |
| 238 | δ = 8.78(1H, s), 8.54~8.55(6H, m), 8.42(1H, m), 8.27~8.30(3H, m), |
| | 8.03~8.15(10H, m), 7.81(1H, d), 7.47~7.61(8H, m), 7.35(4H, d) |
| 241 | δ = 8.85(2H, d), 8.78(1H, s), 8.54(1H, d), 8.27~8.38(5H, m), |
| | 8.03~8.15(10H, m), 7.81(1H, d), 7.35~7.54(12H, m) |
| 247 | δ = 8.78(1H, s), 8.54(1H, d), 8.27~8.30(3H, m), 8.03~8.15(8H, m), |
| | 7.91(4H, m), 7.81(1H, d), 7.35~7.54(16H, m) |
| 250 | δ = 9.30(1H, d), 8.78(1H, s), 8.53~8.54(2H, m), |
| | 8.03~8.31(14H, m), 7.81(1H, d), 7.70(1H, m), 7.47~7.60(4H, m), |
| | 7.35(3H, d), 7.14(1H, m) |
| 251 | δ = 9.19(1H, s), 8.78(1H, s), 8.54(1H, d), 8.44(1H, m), |
| | 8.03~8.30(14H, m), 7.79~7.81(3H, m), 7.35~7.60(15H, m) |
| 253 | δ = 8.78(1H, s), 8.72(1H, s), 8.54(1H, d), 8.30~8.32(4H, m), |
| | 7.98~8.15(8H, m), 7.78~7.81(2H, m), 7.47~7.63(5H, m), |
| | 7.35(4H, d) |
| 254 | δ = 8.78~8.84(5H, m), 8.54(1H, d), 8.30(2H, d), 7.98~8.15(8H, m), |
| | 7.78(1H, m), 7.47~7.60(4H, m), 7.35(4H, d) |
| 255 | δ = 8.85(2H, d), 8.78(1H, s), 8.54(1H, d), 8.30~8.38(4H, d), |
| | 7.95~8.10(10H, m), 7.78~7.81(2H, m), 7.47~7.60(4H, m), |
| | 7.35(4H, d) |

TABLE 37

| Compound | FD-MS | Compound | FD-MS |
|---|---|---|---|
| 1 | m/z = 409.48 (C29H19N3 = 409.16) | 3 | m/z = 459.54 (C33H21N3 = 459.17) |
| 8 | m/z = 639.75 (C45H29N5 = 639.24) | 12 | m/z = 640.73 (C44H28N6 = 640.24) |
| 15 | m/z = 804.94 (C57H36N6 = 804.30) | 17 | m/z = 640.73 (C44H28N6 = 640.24) |
| 19 | m/z = 536.62 (C38H24N4 = 536.20) | 22 | m/z = 587.67 (C41H25N5 = 587.21) |
| 27 | m/z = 663.77 (C47H29N5 = 663.24) | 30 | m/z = 713.83 (C51H31N5 = 713.26) |
| 33 | m/z = 459.54 (C33H21N3 = 459.17) | 37 | m/z = 663.77 (C47H29N5 = 663.24) |
| 40 | m/z = 713.83 (C51H31N5 = 713.26) | 42 | m/z = 409.48 (C29H19N3 = 409.16) |
| 45 | m/z = 509.60 (C37H23N3 = 509.19) | 48 | m/z = 663.77 (C47H29N5 = 663.24) |
| 51 | m/z = 713.83 (C51H31N5 = 713.26) | 54 | m/z = 664.75 (C46H28N6 = 664.24) |
| 59 | m/z = 640.73 (C44H28N6 = 640.24) | 64 | m/z = 612.72 (C44H28N4 = 612.23) |
| 68 | m/z = 816.95 (C58H36N6 = 816.30) | 72 | m/z = 564.64 (C38H24N6 = 564.21) |
| 73 | m/z = 664.75 (C46H28N6 = 664.24) | 79 | m/z = 612.72 (C44H28N4 = 612.23) |
| 80 | m/z = 662.78 (C48H30N4 = 662.25) | 83 | m/z = 816.95 (C58H36N6 = 816.30) |
| 87 | m/z = 867.01 (C62H38N6 = 866.32) | 90 | m/z = 714.81 (C50H30N6 = 714.25) |
| 92 | m/z = 665.74 (C45H27N7 = 665.23) | 97 | m/z = 563.65 (C39H25N5 = 563.21) |
| 100 | m/z = 663.77 (C47H29N5 = 663.24) | 102 | m/z = 817.93 (C57H35N7 = 817.30) |
| 105 | m/z = 791.90 (C55H33N7 = 791.28) | 108 | m/z = 509.60 (C37H23N3 = 509.19) |
| 111 | m/z = 713.83 (C51H31N5 = 713.26) | 114 | m/z = 687.79 (C49H29N5 = 687.24) |
| 116 | m/z = 689.80 (C49H31N5 = 689.26) | 120 | m/z = 854.99 (C61H38N6 = 854.32) |
| 121 | m/z = 713.83 (C51H31N5 = 713.26) | 124 | m/z = 813.94 (C59H35N5 = 813.29) |
| 128 | m/z = 763.88 (C55H33N5 = 763.27) | 133 | m/z = 713.83 (C51H31N5 = 713.26) |
| 137 | m/z = 713.83 (C51H31N5 = 713.26) | 140 | m/z = 763.88 (C55H33N5 = 763.27) |
| 145 | m/z = 459.54 (C33H21N3 = 459.17) | 147 | m/z = 559.66 (C41H25N3 = 559.20) |
| 150 | m/z = 713.83 (C51H31N5 = 713.26) | 168 | m/z = 740.85 (C52H32N6 = 740.27) |
| 170 | m/z = 559.66 (C41H25N3 = 559.20) | 172 | m/z = 586.68 (C42H26N4 = 586.22) |
| 175 | m/z = 764.87 (C54H32N6 = 764.27) | 176 | m/z = 664.75 (C46H28N6 = 664.24) |
| 179 | m/z = 764.87 (C54H32N6 = 764.27) | 182 | m/z = 764.87 (C54H32N6 = 764.27) |
| 184 | m/z = 817.93 (C57H35N7 = 817.30) | 188 | m/z = 764.87 (C54H32N6 = 764.27) |
| 192 | m/z = 510.59 (C36H22N4 = 510.18) | 194 | m/z = 610.70 (C44H26N4 = 610.22) |
| 196 | m/z = 764.87 (C54H32N6 = 764.27) | 199 | m/z = 764.87 (C54H32N6 = 764.27) |
| 203 | m/z = 739.86 (C53H33N5 = 739.27) | 205 | m/z = 739.86 (C53H33N5 = 739.27) |
| 207 | m/z = 662.78 (C48H30N4 = 662.25) | 210 | m/z = 662.78 (C48H30N4 = 662.25) |
| 211 | m/z = 738.87 (C54H34N4 = 738.28) | 214 | m/z = 712.84 (C52H32N4 = 712.26) |
| 219 | m/z = 586.68 (C42H26N4 = 586.22) | 230 | m/z = 636.74 (C46H28N4 = 636.23) |
| 234 | m/z = 712.84 (C52H32N4 = 712.26) | 238 | m/z = 762.90 (C56H34N4 = 762.28) |
| 241 | m/z = 712.84 (C52H32N4 = 712.26) | 247 | m/z = 762.90 (C56H34N4 = 762.28) |
| 250 | m/z = 663.77 (C47H29N5 = 663.24) | 251 | m/z = 816.95 (C58H36N6 = 816.30) |
| 253 | m/z = 586.68 (C42H26N4 = 586.22) | 254 | m/z = 586.68 (C42H26N4 = 586.22) |
| 255 | m/z = 636.74 (C46H28N4 = 636.23) | | |

### <Experimental Example 1> Organic Light Emitting Device

1) Manufacture of Organic Light Emitting Device

### Examples 1 to 76 and Comparative Examples 1 to 5

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), organic materials were formed in a 2-stack white organic light emitting device (WOLED) structure. As for the first stack, TAPC was thermal vacuum deposited first to a thickness of 300 Å to form a hole transfer layer. After forming the hole transfer layer, a light emitting layer was thermal vacuum deposited thereon as follows. As the light emitting layer, TCz1, a host, was 8% doped with FIrpic, a blue phosphorescent dopant, and deposited to 300 Å. After forming an electron transfer layer to 400 Å using TmPyPB, a compound described in the following Table 38 was 20% doped with Cs₂CO₃ to form as a charge generation layer to 100 Å.

As for the second stack, MoO₃ was thermal vacuum deposited first to a thickness of 50 Å to form a hole injection layer. A hole transfer layer, a common layer, was formed to 100 Å by 20% doping MoO₃ to TAPC and then depositing TAPC to 300 Å. A light emitting layer was formed thereon by 8% doping Ir(ppy)₃, a green phosphorescent dopant, to TCz1, a host, and depositing the result to 300 Å, and then an electron transfer layer was formed to 600 Å using TmPyPB. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

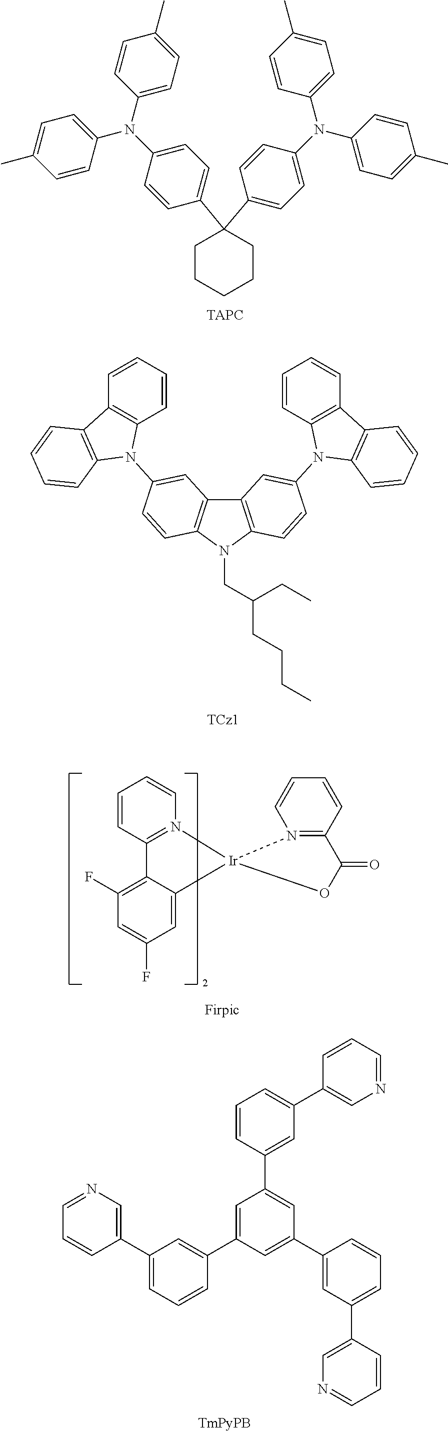

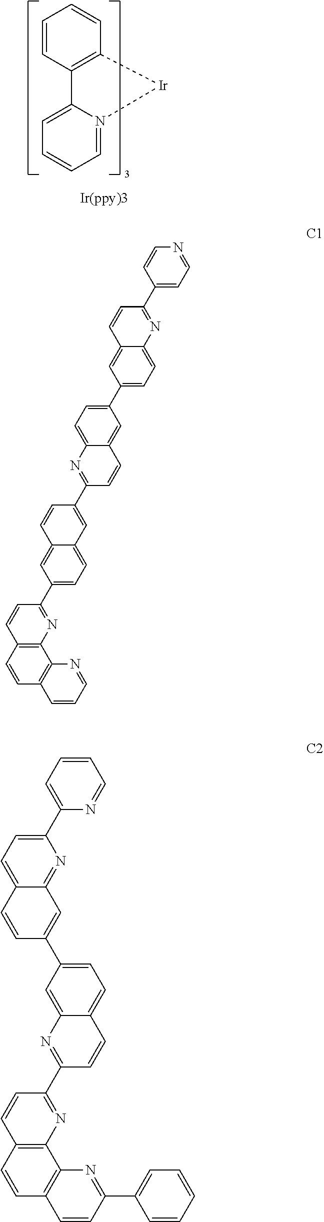

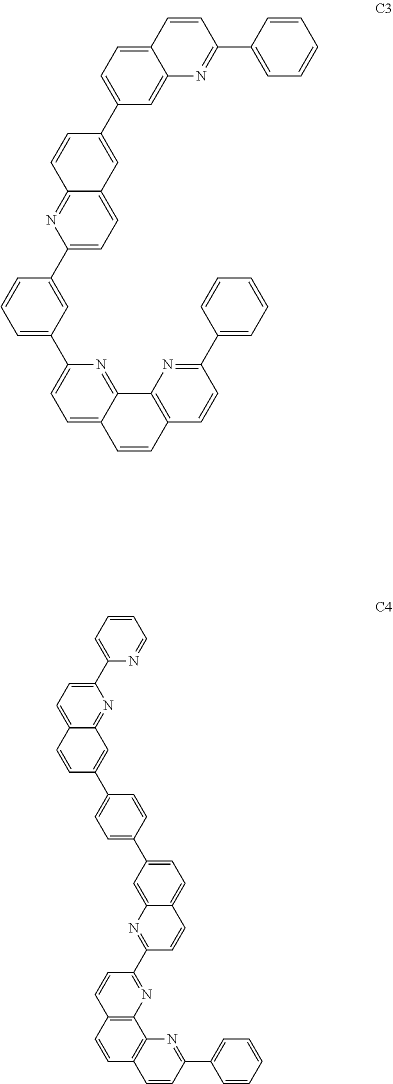 [SMILES: c1ccc(-c2ccc3ccc(-c4ccc5nc(-c6cccc(-c7ccc8ccc9ccc(-c%10ccccc%10)nc9c8n7)c6)ccc5c4)cc3n2)cc1 ; c1ccc(-c2ccc3ccc4ccc(-c5ccc6ccc(-c7ccc(-c8ccc9ccc(-c%10ccccn%10)nc9c8)cc7)cc6n5)nc4c3n2)cc1]

2) Driving Voltage and Light Emission Efficiency of Organic Electroluminescent Device

For each of the organic electroluminescent devices manufactured as above, electroluminescent (EL) properties were measured using M7000 manufactured by McScience Inc., and with the measurement results, a lifetime T₉₅ was measured when standard luminance was 3500 cd/m² through a lifetime measurement system (M6000) manufactured by McScience Inc. Results of measuring driving voltage, light emission efficiency, external quantum efficiency, color coordinate (CIE) and lifetime of the white organic electroluminescent devices manufactured according to the present disclosure are as shown in Table 38.

TABLE 38

| | | Driv- | Light | External | | |
|---|---|---|---|---|---|---|
| | | ing | Emission | Quantum | | |
| | | Volt- | Effi- | Effi- | | Life- |
| | Com- | age | ciency | ciency | | time |
| | pound | (V) | (cd/A) | (%) | CIE (x, y) | (T₉₅) |
| Example 1 | 1 | 7.51 | 66.42 | 32.15 | 0.207, 0.416 | 34 |
| Example 2 | 3 | 7.72 | 62.87 | 25.17 | 0.211, 0.424 | 36 |
| Example 3 | 8 | 7.17 | 65.16 | 35.12 | 0.231, 0.482 | 40 |
| Example 4 | 12 | 7.33 | 61.92 | 31.28 | 0.226, 0.434 | 39 |
| Example 5 | 15 | 7.66 | 64.76 | 26.95 | 0.207, 0.421 | 35 |
| Example 6 | 17 | 7.88 | 65.44 | 32.02 | 0.209, 0.421 | 37 |
| Example 7 | 19 | 7.65 | 68.13 | 34.24 | 0.231, 0.463 | 37 |
| Example 8 | 22 | 7.78 | 67.15 | 30.06 | 0.208, 0.421 | 36 |
| Example 9 | 27 | 7.54 | 66.73 | 31.23 | 0.208, 0.421 | 40 |
| Example 10 | 30 | 7.54 | 68.26 | 32.24 | 0.210, 0.419 | 45 |
| Example 11 | 33 | 8.24 | 58.88 | 24.65 | 0.211, 0.391 | 42 |
| Example 12 | 37 | 7.44 | 63.18 | 27.06 | 0.211, 0.426 | 38 |
| Example 13 | 40 | 7.37 | 65.81 | 34.82 | 0.207, 0.421 | 41 |
| Example 14 | 42 | 7.56 | 62.24 | 26.04 | 0.211, 0.422 | 39 |
| Example 15 | 45 | 7.61 | 66.06 | 34.46 | 0.233, 0.478 | 37 |
| Example 16 | 48 | 7.30 | 60.47 | 31.52 | 0.207, 0.419 | 41 |
| Example 17 | 51 | 7.59 | 68.67 | 22.66 | 0.216, 0.464 | 40 |
| Example 18 | 54 | 7.28 | 66.51 | 32.51 | 0.211, 0.422 | 43 |
| Example 19 | 59 | 8.13 | 60.44 | 28.63 | 0.216, 0.484 | 38 |
| Example 20 | 64 | 7.97 | 58.25 | 22.20 | 0.201, 0.483 | 34 |
| Example 21 | 68 | 7.43 | 68.58 | 32.46 | 0.208, 0.417 | 38 |
| Example 22 | 72 | 7.47 | 65.28 | 33.90 | 0.211, 0.422 | 39 |
| Example 23 | 73 | 7.55 | 67.60 | 29.65 | 0.207, 0.416 | 40 |
| Example 24 | 79 | 7.44 | 63.18 | 27.06 | 0.211, 0.426 | 41 |
| Example 25 | 80 | 7.37 | 65.81 | 34.82 | 0.207, 0.421 | 37 |
| Example 26 | 83 | 7.71 | 67.23 | 32.19 | 0.212, 0.426 | 33 |
| Example 27 | 87 | 7.54 | 66.73 | 31.23 | 0.208, 0.421 | 40 |
| Example 28 | 90 | 7.54 | 68.26 | 32.24 | 0.210, 0.419 | 39 |
| Example 29 | 92 | 7.43 | 66.38 | 29.26 | 0.211, 0.421 | 41 |
| Example 30 | 97 | 8.11 | 62.83 | 25.82 | 0.209, 0.416 | 35 |
| Example 31 | 100 | 7.52 | 63.86 | 32.92 | 0.220, 0.480 | 41 |
| Example 32 | 102 | 7.39 | 65.27 | 35.23 | 0.234, 0.483 | 39 |
| Example 33 | 105 | 7.56 | 66.35 | 31.83 | 0.206, 0.415 | 40 |
| Example 34 | 108 | 7.51 | 66.42 | 32.15 | 0.207, 0.416 | 43 |
| Example 35 | 111 | 7.72 | 62.87 | 25.17 | 0.211, 0.424 | 41 |
| Example 36 | 114 | 7.42 | 68.81 | 32.14 | 0.207, 0.422 | 43 |
| Example 37 | 116 | 7.37 | 65.98 | 34.82 | 0.208, 0.421 | 39 |
| Example 38 | 120 | 7.45 | 62.25 | 26.14 | 0.213, 0.422 | 41 |
| Example 39 | 121 | 7.48 | 71.18 | 30.11 | 0.211, 0.421 | 42 |
| Example 40 | 124 | 7.32 | 61.37 | 31.58 | 0.207, 0.418 | 44 |
| Example 41 | 128 | 7.58 | 68.66 | 25.65 | 0.215, 0.463 | 40 |
| Example 42 | 133 | 7.48 | 65.28 | 33.81 | 0.210, 0.421 | 38 |
| Example 43 | 137 | 7.55 | 67.64 | 29.64 | 0.208, 0.419 | 35 |
| Example 44 | 145 | 7.78 | 64.77 | 25.95 | 0.208, 0.420 | 22 |
| Example 45 | 147 | 7.48 | 66.73 | 33.32 | 0.207, 0.420 | 40 |
| Example 46 | 150 | 7.44 | 68.26 | 32.24 | 0.208, 0.419 | 39 |
| Example 47 | 168 | 7.45 | 62.25 | 26.14 | 0.213, 0.422 | 42 |
| Example 48 | 170 | 7.63 | 66.26 | 34.35 | 0.233, 0.477 | 39 |
| Example 49 | 172 | 7.57 | 66.77 | 31.22 | 0.208, 0.421 | 37 |
| Example 50 | 175 | 7.34 | 68.16 | 32.35 | 0.206, 0.419 | 43 |
| Example 51 | 176 | 8.18 | 64.85 | 31.90 | 0.207, 0.421 | 38 |
| Example 52 | 179 | 7.59 | 67.20 | 32.83 | 0.209, 0.418 | 42 |
| Example 53 | 182 | 7.44 | 68.81 | 33.24 | 0.208, 0.421 | 35 |
| Example 54 | 184 | 7.37 | 65.85 | 34.82 | 0.209, 0.422 | 37 |
| Example 55 | 188 | 7.48 | 71.18 | 32.21 | 0.212, 0.421 | 45 |
| Example 56 | 192 | 7.31 | 71.18 | 30.21 | 0.211, 0.421 | 40 |
| Example 57 | 194 | 7.44 | 66.45 | 29.36 | 0.211, 0.421 | 40 |
| Example 58 | 196 | 8.16 | 63.83 | 25.73 | 0.209, 0.416 | 36 |
| Example 59 | 199 | 8.19 | 64.88 | 31.90 | 0.207, 0.421 | 34 |
| Example 60 | 203 | 7.68 | 67.21 | 32.83 | 0.208, 0.419 | 47 |
| Example 61 | 205 | 7.77 | 67.15 | 31.06 | 0.208, 0.421 | 39 |
| Example 62 | 207 | 7.48 | 71.18 | 32.21 | 0.212, 0.421 | 41 |
| Example 63 | 210 | 7.42 | 67.34 | 31.02 | 0.205, 0.421 | 34 |
| Example 64 | 211 | 7.31 | 66.31 | 33.45 | 0.229, 0.481 | 45 |
| Example 65 | 214 | 7.52 | 63.86 | 32.92 | 0.220, 0.480 | 39 |
| Example 66 | 219 | 7.39 | 65.27 | 35.23 | 0.234, 0.483 | 40 |
| Example 67 | 230 | 7.48 | 66.73 | 33.32 | 0.207, 0.420 | 33 |
| Example 68 | 234 | 7.44 | 68.26 | 32.24 | 0.208, 0.419 | 43 |
| Example 69 | 238 | 7.44 | 66.85 | 29.46 | 0.212, 0.420 | 39 |
| Example 70 | 241 | 7.68 | 64.77 | 26.85 | 0.208, 0.422 | 40 |
| Example 71 | 247 | 7.79 | 65.34 | 32.52 | 0.210, 0.421 | 36 |
| Example 72 | 250 | 7.37 | 65.85 | 34.82 | 0.209, 0.422 | 45 |
| Example 73 | 251 | 7.48 | 71.18 | 32.21 | 0.212, 0.421 | 44 |
| Example 74 | 253 | 7.42 | 67.34 | 31.02 | 0.205, 0.421 | 40 |
| Example 75 | 254 | 8.18 | 64.85 | 31.90 | 0.207, 0.421 | 40 |
| Example 76 | 255 | 7.59 | 67.20 | 32.83 | 0.209, 0.418 | 33 |
| Comparative | TmPyP | 8.68 | 53.95 | 20.73 | 0.213, 0.443 | 20 |
| Example 1 | B | | | | | |
| Comparative | C1 | 7.56 | 62.05 | 26.04 | 0.215, 0.422 | 22 |
| Example 2 | | | | | | |
| Comparative | C2 | 7.57 | 61.95 | 26.24 | 0.214, 0.423 | 22 |
| Example 3 | | | | | | |
| Comparative | C3 | 7.55 | 62.11 | 25.98 | 0.215, 0.422 | 20 |
| Example 4 | | | | | | |
| Comparative | C4 | 7.57 | 61.93 | 26.25 | 0.214, 0.423 | 21 |
| Example 5 | | | | | | |

As seen from the results of Table 38, the organic electroluminescent devices using the charge generation layer material of the white organic electroluminescent device of the present disclosure had a lower driving voltage and significantly improved light emission efficiency compared to Comparative Examples 1 to 5.

### <Experimental Example 2> Organic Light Emitting Device

1) Manufacture of Organic Light Emitting Device

### Examples 77 to 152 and Comparative Examples 6 to 10

A transparent ITO electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, an ITO substrate was installed in a substrate folder of a vacuum deposition apparatus, and the following 4,4′,4″-tris(N,N-(2-naphthyl)-phenylamino)triphenylamine (2-TNATA) was introduced to a cell in the vacuum deposition apparatus.

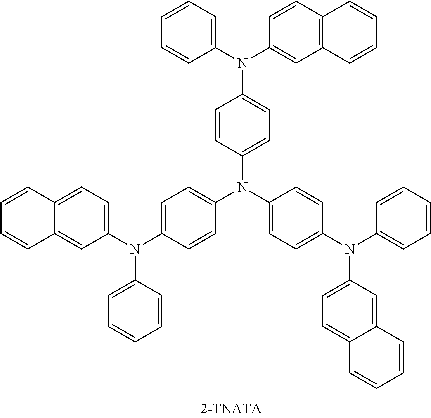 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate.

To another cell of the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

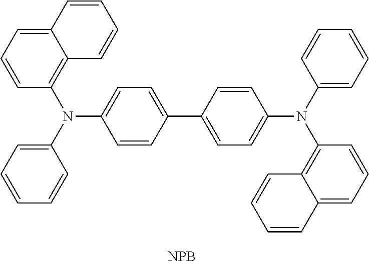 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

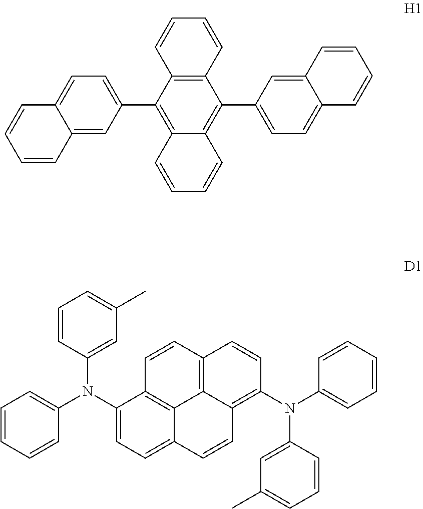

After forming an electron transfer layer to 300 Å using TmPyPB, a compound described in the following Table 39 was 20% doped with Cs₂CO₃ to form as a charge generation layer to 100 Å.

As an electron injection layer, lithium fluoride (LiF) was deposited to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁸ torr to 10⁻⁶ torr by each material to be used in the OLED manufacture.

Results of measuring driving voltage, light emission efficiency, external quantum efficiency, color coordinate (CIE) and lifetime of the blue organic light emitting devices manufactured according to the present disclosure are as shown in Table 39.

TABLE 39

| | | Driv- | Light | External | | |
|---|---|---|---|---|---|---|
| | | ing | Emission | Quantum | | |
| | | Volt- | Effi- | Effi- | | Life- |
| | Com- | age | ciency | ciency | | time |
| | pound | (V) | (cd/A) | (%) | CIE (x, y) | (T₉₅) |
| Example 77 | 1 | 7.53 | 65.83 | 31.45 | 0.228, 0.481 | 41 |
| Example 78 | 3 | 7.30 | 67.57 | 32.33 | 0.211, 0.423 | 34 |
| Example 79 | 8 | 7.41 | 60.33 | 29.52 | 0.216, 0.484 | 45 |
| Example 80 | 12 | 7.32 | 61.92 | 32.29 | 0.226, 0.434 | 39 |
| Example 81 | 15 | 7.46 | 62.38 | 29.16 | 0.211, 0.424 | 40 |
| Example 82 | 17 | 7.44 | 68.88 | 31.23 | 0.209, 0.423 | 33 |
| Example 83 | 19 | 7.54 | 69.25 | 33.16 | 0.233, 0.463 | 43 |
| Example 84 | 22 | 7.55 | 67.14 | 31.06 | 0.208, 0.420 | 39 |
| Example 85 | 27 | 7.49 | 71.21 | 30.16 | 0.211, 0.420 | 40 |
| Example 86 | 30 | 7.39 | 68.38 | 33.22 | 0.207, 0.422 | 36 |
| Example 87 | 33 | 7.64 | 59.75 | 29.75 | 0.210, 0.391 | 45 |
| Example 88 | 37 | 7.45 | 62.39 | 29.36 | 0.211, 0.425 | 44 |
| Example 89 | 40 | 7.40 | 66.73 | 33.92 | 0.208, 0.421 | 42 |
| Example 90 | 42 | 7.54 | 62.25 | 27.25 | 0.214, 0.422 | 39 |
| Example 91 | 45 | 7.54 | 65.16 | 33.55 | 0.234, 0.478 | 37 |
| Example 92 | 48 | 7.34 | 64.37 | 32.81 | 0.207, 0.419 | 43 |
| Example 93 | 51 | 7.48 | 67.69 | 29.43 | 0.217, 0.464 | 38 |
| Example 94 | 54 | 7.30 | 67.57 | 32.33 | 0.211, 0.423 | 42 |
| Example 95 | 59 | 7.41 | 60.33 | 29.52 | 0.216, 0.484 | 35 |
| Example 96 | 64 | 7.44 | 59.91 | 28.21 | 0.202, 0.483 | 37 |
| Example 97 | 68 | 7.43 | 64.57 | 32.87 | 0.208, 0.416 | 45 |
| Example 98 | 72 | 7.47 | 65.18 | 31.91 | 0.211, 0.422 | 40 |
| Example 99 | 73 | 7.49 | 67.53 | 29.77 | 0.208, 0.416 | 40 |
| Example 100 | 79 | 7.58 | 65.77 | 28.87 | 0.207, 0.422 | 36 |
| Example 101 | 80 | 7.48 | 66.26 | 31.83 | 0.209, 0.421 | 34 |
| Example 102 | 83 | 7.51 | 67.33 | 32.29 | 0.212, 0.427 | 40 |
| Example 103 | 87 | 7.47 | 68.72 | 30.31 | 0.209, 0.421 | 45 |
| Example 104 | 90 | 7.44 | 68.56 | 33.27 | 0.209, 0.419 | 42 |
| Example 105 | 92 | 7.40 | 66.25 | 29.56 | 0.212, 0.421 | 38 |
| Example 106 | 97 | 7.35 | 62.84 | 28.71 | 0.208, 0.416 | 41 |
| Example 107 | 100 | 7.34 | 64.99 | 31.90 | 0.207, 0.420 | 39 |
| Example 108 | 102 | 7.51 | 67.35 | 31.64 | 0.208, 0.418 | 37 |
| Example 109 | 105 | 7.46 | 66.13 | 31.88 | 0.206, 0.414 | 41 |
| Example 110 | 108 | 7.51 | 67.42 | 31.94 | 0.206, 0.416 | 40 |
| Example 111 | 111 | 7.72 | 63.67 | 25.12 | 0.211, 0.423 | 43 |
| Example 112 | 114 | 7.32 | 67.83 | 33.24 | 0.208, 0.422 | 38 |
| Example 113 | 116 | 7.45 | 69.13 | 35.84 | 0.234, 0.462 | 34 |
| Example 114 | 120 | 7.51 | 67.84 | 31.06 | 0.209, 0.421 | 38 |
| Example 115 | 121 | 7.39 | 70.19 | 31.57 | 0.211, 0.420 | 39 |
| Example 116 | 124 | 7.37 | 68.57 | 33.68 | 0.208, 0.418 | 40 |
| Example 117 | 128 | 7.48 | 67.68 | 29.79 | 0.216, 0.463 | 41 |
| Example 118 | 133 | 7.49 | 66.61 | 32.91 | 0.210, 0.421 | 37 |
| Example 119 | 137 | 7.59 | 67.42 | 29.68 | 0.207, 0.419 | 33 |
| Example 120 | 145 | 7.58 | 63.36 | 28.91 | 0.208, 0.421 | 42 |
| Example 121 | 147 | 7.38 | 66.37 | 32.88 | 0.207, 0.420 | 38 |
| Example 122 | 150 | 7.33 | 65.94 | 33.72 | 0.208, 0.422 | 41 |
| Example 123 | 168 | 7.45 | 62.25 | 28.67 | 0.214, 0.422 | 39 |
| Example 124 | 170 | 7.52 | 66.66 | 34.45 | 0.233, 0.478 | 37 |
| Example 125 | 172 | 7.57 | 67.75 | 32.61 | 0.209, 0.421 | 41 |
| Example 126 | 175 | 7.34 | 68.37 | 31.25 | 0.207, 0.419 | 40 |
| Example 127 | 176 | 7.36 | 67.91 | 32.88 | 0.212, 0.421 | 43 |
| Example 128 | 179 | 7.49 | 64.85 | 28.33 | 0.208, 0.416 | 38 |
| Example 129 | 182 | 7.43 | 68.76 | 30.24 | 0.209, 0.421 | 34 |
| Example 130 | 184 | 7.31 | 68.53 | 33.67 | 0.233, 0.463 | 38 |
| Example 131 | 188 | 7.63 | 67.35 | 30.27 | 0.208, 0.422 | 39 |
| Example 132 | 192 | 7.37 | 70.18 | 31.01 | 0.211, 0.420 | 40 |
| Example 133 | 194 | 7.43 | 67.55 | 29.80 | 0.212, 0.421 | 41 |
| Example 134 | 196 | 7.49 | 65.16 | 27.73 | 0.208, 0.416 | 37 |
| Example 135 | 199 | 8.00 | 64.78 | 31.81 | 0.208, 0.421 | 33 |
| Example 136 | 203 | 7.69 | 69.20 | 33.46 | 0.208, 0.418 | 40 |
| Example 137 | 205 | 7.78 | 68.35 | 30.88 | 0.207, 0.421 | 42 |
| Example 138 | 207 | 7.48 | 71.09 | 30.71 | 0.212, 0.420 | 35 |
| Example 139 | 210 | 7.33 | 67.44 | 31.08 | 0.206, 0.421 | 37 |
| Example 140 | 211 | 7.39 | 65.81 | 34.91 | 0.229, 0.482 | 45 |
| Example 141 | 214 | 7.51 | 64.86 | 31.89 | 0.221, 0.480 | 40 |
| Example 142 | 219 | 7.39 | 64.16 | 30.23 | 0.234, 0.484 | 40 |
| Example 143 | 230 | 7.37 | 66.73 | 31.82 | 0.208, 0.420 | 36 |
| Example 144 | 234 | 7.54 | 69.27 | 32.15 | 0.208, 0.418 | 34 |
| Example 145 | 238 | 7.40 | 67.35 | 29.94 | 0.213, 0.420 | 33 |
| Example 146 | 241 | 7.61 | 64.76 | 28.86 | 0.208, 0.421 | 42 |
| Example 147 | 247 | 7.39 | 65.44 | 33.42 | 0.210, 0.420 | 38 |
| Example 148 | 250 | 7.47 | 66.98 | 30.62 | 0.208, 0.422 | 41 |
| Example 149 | 251 | 8.68 | 53.95 | 20.73 | 0.213, 0.443 | 39 |
| Example 150 | 253 | 7.56 | 62.05 | 26.04 | 0.215, 0.422 | 37 |
| Example 151 | 254 | 8.00 | 64.78 | 31.81 | 0.208, 0.421 | 41 |
| Example 152 | 255 | 7.69 | 69.20 | 33.46 | 0.208, 0.418 | 41 |
| Comparative | TmPyP | 8.68 | 53.95 | 20.73 | 0.213, 0.443 | 20 |
| Example 6 | B | | | | | |
| Comparative | C1 | 7.56 | 62.05 | 26.04 | 0.215, 0.422 | 22 |
| Example 7 | | | | | | |
| Comparative | C2 | 7.57 | 61.95 | 26.24 | 0.214, 0.423 | 22 |
| Example 8 | | | | | | |
| Comparative | C3 | 7.55 | 62.11 | 25.98 | 0.215, 0.422 | 20 |
| Example 9 | | | | | | |
| Comparative | C4 | 7.57 | 61.93 | 26.25 | 0.214, 0.423 | 21 |
| Example 10 | | | | | | |

As seen from the results of Table 39, the organic electroluminescent devices using the charge generation layer material of the blue organic electroluminescent device of the present disclosure had a lower driving voltage and significantly improved light emission efficiency compared to Comparative Examples 6 to 10.

### <Experimental Example 3> Organic Light Emitting Device

1) Manufacture of Organic Light Emitting Device

### Comparative Example 11

A transparent ITO electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, an ITO substrate was installed in a substrate folder of a vacuum deposition apparatus, and the following 4,4′,4″-tris(N,N-(2-naphthyl)-phenylamino)triphenylamine (2-TNATA) was introduced to a cell in the vacuum deposition apparatus.

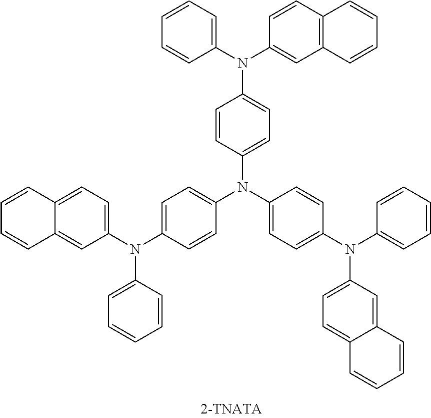 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate.

To another cell of the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

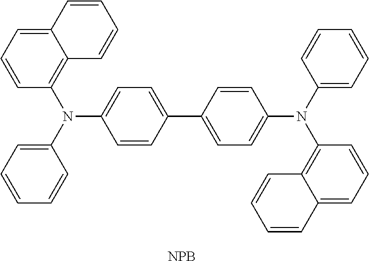 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

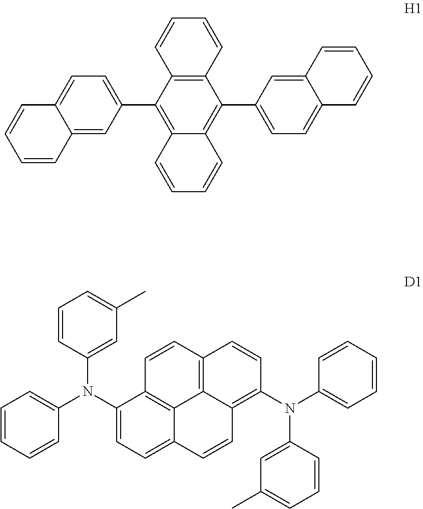

After forming an electron transfer layer to 300 Å using TmPyPB, a compound of the following Structural Formula C5 was 20% doped with Cs₂CO₃ to form as a charge generation layer to 100 Å.

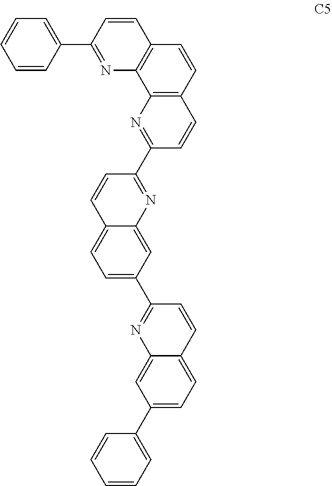 [SMILES: c1ccc(-c2ccc3ccc(-c4ccc5ccc(-c6ccc7ccc8ccc(-c9ccccc9)nc8c7n6)nc5c4)nc3c2)cc1]

As an electron injection layer, lithium fluoride (LiF) was deposited on the charge generation layer to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁸ torr to 10⁻⁶ torr by each material to be used in the OLED manufacture.

### Examples 153 to 228 and Comparative Examples 12 to 15

Organic light emitting devices were manufactured in the same manner as in Comparative Example 11 except that, after forming an electron transfer layer to 250 Å using TmPyPB, a hole blocking layer having a thickness of 50 Å was formed on the electron transfer layer using a compound presented in the following Table 40.

Results of measuring driving voltage, light emission efficiency, external quantum efficiency, color coordinate (CIE) and lifetime of the blue organic light emitting devices manufactured according to the present disclosure are as shown in Table 40.

TABLE 40

| | | Driv- | Light | External | | |
|---|---|---|---|---|---|---|
| | | ing | Emission | Quantum | | |
| | | Volt- | Effi- | Effi- | | Life- |
| | Com- | age | ciency | ciency | | time |
| | pound | (V) | (cd/A) | (%) | CIE (x, y) | (T₉₅) |
| Example 153 | 1 | 7.44 | 64.22 | 31.56 | 0.228, 0.481 | 40 |
| Example 154 | 3 | 7.38 | 64.78 | 32.83 | 0.220, 0.481 | 43 |
| Example 155 | 8 | 7.36 | 63.86 | 34.49 | 0.232, 0.482 | 38 |
| Example 156 | 12 | 7.41 | 62.92 | 32.18 | 0.226, 0.434 | 34 |
| Example 157 | 15 | 7.39 | 62.57 | 29.99 | 0.211, 0.424 | 38 |
| Example 158 | 17 | 7.43 | 67.97 | 33.28 | 0.209, 0.423 | 39 |
| Example 159 | 19 | 7.45 | 68.03 | 34.59 | 0.233, 0.463 | 40 |
| Example 160 | 22 | 7.50 | 67.14 | 32.86 | 0.208, 0.420 | 41 |
| Example 161 | 27 | 7.39 | 70.87 | 30.92 | 0.211, 0.420 | 37 |
| Example 162 | 30 | 7.47 | 69.35 | 33.48 | 0.207, 0.422 | 33 |
| Example 163 | 33 | 7.46 | 61.85 | 29.49 | 0.210, 0.391 | 42 |
| Example 164 | 37 | 7.33 | 62.99 | 31.26 | 0.211, 0.425 | 38 |
| Example 165 | 40 | 7.28 | 66.80 | 34.62 | 0.208, 0.421 | 41 |
| Example 166 | 42 | 7.51 | 62.75 | 30.04 | 0.214, 0.422 | 39 |
| Example 167 | 45 | 7.55 | 64.17 | 34.56 | 0.234, 0.478 | 37 |
| Example 168 | 48 | 7.34 | 63.48 | 33.91 | 0.207, 0.419 | 41 |
| Example 169 | 51 | 7.39 | 68.49 | 29.65 | 0.217, 0.464 | 40 |
| Example 170 | 54 | 7.33 | 68.49 | 32.41 | 0.211, 0.423 | 43 |
| Example 171 | 59 | 7.38 | 63.64 | 31.95 | 0.216, 0.484 | 38 |
| Example 172 | 64 | 7.43 | 58.95 | 29.05 | 0.202, 0.483 | 34 |
| Example 173 | 68 | 7.39 | 67.72 | 32.36 | 0.208, 0.416 | 38 |
| Example 174 | 72 | 7.41 | 65.18 | 34.09 | 0.211, 0.422 | 39 |
| Example 175 | 73 | 7.44 | 67.19 | 31.78 | 0.208, 0.416 | 40 |
| Example 176 | 79 | 7.51 | 64.77 | 33.01 | 0.207, 0.422 | 41 |
| Example 177 | 80 | 7.46 | 66.39 | 32.69 | 0.209, 0.421 | 37 |
| Example 178 | 83 | 7.42 | 66.58 | 33.17 | 0.212, 0.427 | 33 |
| Example 179 | 87 | 7.47 | 65.82 | 30.56 | 0.209, 0.421 | 40 |
| Example 180 | 90 | 7.51 | 66.61 | 33.84 | 0.209, 0.419 | 42 |
| Example 181 | 92 | 7.38 | 66.59 | 29.16 | 0.212, 0.421 | 35 |
| Example 182 | 97 | 7.35 | 62.73 | 29.62 | 0.208, 0.416 | 37 |
| Example 183 | 100 | 7.41 | 63.94 | 31.25 | 0.207, 0.420 | 45 |
| Example 184 | 102 | 7.51 | 66.23 | 32.78 | 0.208, 0.418 | 40 |
| Example 185 | 105 | 7.50 | 66.67 | 31.15 | 0.206, 0.414 | 36 |
| Example 186 | 108 | 7.49 | 67.42 | 32.59 | 0.206, 0.416 | 34 |
| Example 187 | 111 | 7.71 | 63.46 | 30.67 | 0.211, 0.423 | 33 |
| Example 188 | 114 | 7.45 | 67.71 | 33.59 | 0.208, 0.422 | 42 |
| Example 189 | 116 | 7.45 | 69.85 | 35.12 | 0.234, 0.462 | 38 |
| Example 190 | 120 | 7.51 | 66.34 | 33.19 | 0.209, 0.421 | 41 |
| Example 191 | 121 | 7.48 | 70.04 | 31.91 | 0.211, 0.420 | 39 |
| Example 192 | 124 | 7.38 | 63.59 | 32.69 | 0.208, 0.418 | 37 |
| Example 193 | 128 | 7.37 | 66.87 | 29.94 | 0.216, 0.463 | 40 |
| Example 194 | 133 | 7.42 | 68.28 | 31.68 | 0.210, 0.421 | 36 |
| Example 195 | 137 | 7.50 | 67.18 | 29.51 | 0.207, 0.419 | 45 |
| Example 196 | 145 | 7.68 | 65.45 | 29.95 | 0.208, 0.421 | 44 |
| Example 197 | 147 | 7.41 | 65.57 | 31.48 | 0.207, 0.420 | 42 |
| Example 198 | 150 | 7.40 | 65.79 | 34.63 | 0.208, 0.422 | 39 |
| Example 199 | 168 | 7.46 | 61.58 | 28.32 | 0.214, 0.422 | 37 |
| Example 200 | 170 | 7.53 | 66.62 | 33.41 | 0.233, 0.478 | 43 |
| Example 201 | 172 | 7.40 | 66.64 | 31.38 | 0.209, 0.421 | 38 |
| Example 202 | 175 | 7.35 | 67.66 | 32.31 | 0.207, 0.419 | 42 |
| Example 203 | 176 | 7.36 | 63.27 | 32.07 | 0.212, 0.421 | 35 |
| Example 204 | 179 | 7.45 | 64.74 | 29.88 | 0.208, 0.416 | 37 |
| Example 205 | 182 | 7.38 | 67.15 | 30.67 | 0.209, 0.421 | 45 |
| Example 206 | 184 | 7.51 | 68.66 | 33.96 | 0.233, 0.463 | 40 |
| Example 207 | 188 | 7.61 | 67.17 | 32.22 | 0.208, 0.422 | 40 |
| Example 208 | 192 | 7.36 | 69.09 | 31.51 | 0.211, 0.420 | 36 |
| Example 209 | 194 | 7.43 | 68.45 | 29.19 | 0.212, 0.421 | 34 |
| Example 210 | 196 | 8.15 | 64.67 | 26.71 | 0.208, 0.416 | 40 |
| Example 211 | 199 | 8.07 | 64.78 | 30.83 | 0.208, 0.421 | 45 |
| Example 212 | 203 | 7.68 | 66.15 | 31.96 | 0.208, 0.418 | 37 |
| Example 213 | 205 | 7.60 | 66.35 | 34.96 | 0.207, 0.421 | 43 |
| Example 214 | 207 | 7.45 | 72.08 | 31.77 | 0.212, 0.420 | 38 |
| Example 215 | 210 | 7.36 | 66.37 | 33.50 | 0.206, 0.421 | 42 |
| Example 216 | 211 | 7.32 | 66.81 | 31.45 | 0.229, 0.482 | 35 |
| Example 217 | 214 | 7.50 | 64.79 | 32.87 | 0.221, 0.480 | 37 |
| Example 218 | 219 | 7.41 | 65.06 | 31.25 | 0.234, 0.484 | 45 |
| Example 219 | 230 | 7.46 | 66.85 | 31.73 | 0.208, 0.420 | 40 |
| Example 220 | 234 | 7.47 | 68.77 | 31.24 | 0.208, 0.418 | 40 |
| Example 221 | 238 | 7.44 | 67.51 | 30.56 | 0.213, 0.420 | 36 |
| Example 222 | 241 | 7.51 | 65.77 | 29.85 | 0.208, 0.421 | 34 |
| Example 223 | 247 | 7.62 | 65.39 | 30.53 | 0.210, 0.420 | 40 |
| Example 224 | 250 | 7.31 | 65.77 | 32.65 | 0.208, 0.422 | 45 |
| Example 225 | 251 | 8.68 | 53.95 | 20.73 | 0.213, 0.443 | 38 |
| Example 226 | 253 | 7.56 | 62.05 | 26.04 | 0.215, 0.422 | 42 |
| Example 227 | 254 | 7.60 | 66.35 | 34.96 | 0.207, 0.421 | 35 |
| Example 228 | 255 | 7.45 | 72.08 | 31.77 | 0.212, 0.420 | 37 |
| Comparative | TmPyP | 8.68 | 53.95 | 20.73 | 0.213, 0.443 | 20 |
| Example 11 | B | | | | | |
| Comparative | C1 | 7.56 | 62.05 | 26.04 | 0.215, 0.422 | 22 |
| Example 12 | | | | | | |
| Comparative | C2 | 7.57 | 61.95 | 26.24 | 0.214, 0.423 | 22 |
| Example 13 | | | | | | |
| Comparative | C3 | 7.55 | 62.11 | 25.98 | 0.215, 0.422 | 20 |
| Example 14 | | | | | | |
| Comparative | C4 | 7.57 | 61.93 | 26.25 | 0.214, 0.423 | 21 |
| Example 15 | | | | | | |

As seen from the results of Table 40, the organic light emitting devices using the hole blocking layer material of the blue organic light emitting device of the present disclosure had a lower driving voltage and significantly improved light emission efficiency and lifetime compared to Comparative Examples 11 to 15.

1. A heterocyclic compound represented by the following Chemical Formula 1: 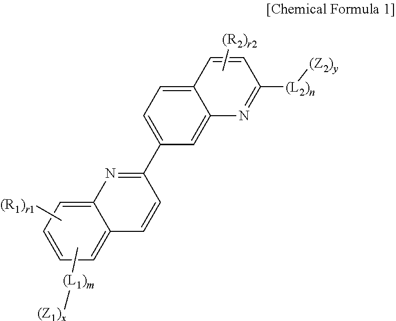
wherein, in Chemical Formula 1,
L₁ and L₂ are the same as or different from each other, and each independently a direct bond; a substituted or unsubstituted C6 to C60 arylene group; or a substituted or unsubstituted C2 to C60 heteroarylene group,
Z₁ and Z₂ are the same as or different from each other, and each independently hydrogen; deuterium; a substituted or unsubstituted C6 to C60 aryl group; or a substituted or unsubstituted C2 to C60 heteroaryl group,
R₁ and R₂ are the same as or different from each other, and each independently hydrogen; deuterium; a halogen group; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,
r1 is an integer of 1 to 3,
r2 is 1 or 2,
m, n, x and y are each an integer of 1 to 5,
when r2 is 2, R₂s are the same as or different from each other, and
when r1, m, n, x and y are each 2 or greater, substituents in the parentheses are the same as or different from each other.

2. The heterocyclic compound of claim 1, wherein Chemical Formula 1 is represented by any one of the following Chemical Formulae 2 to 5: 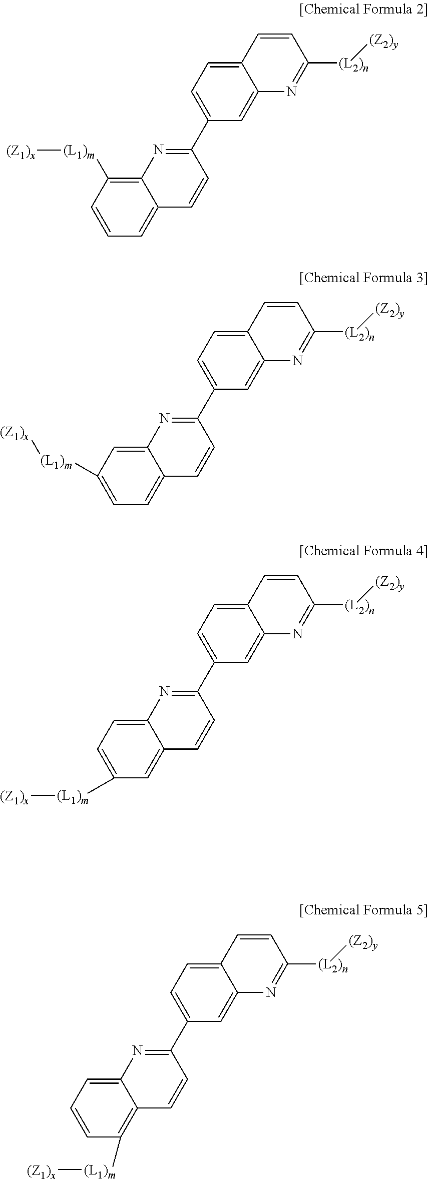
in Chemical Formulae 2 to 5,
each substituent has the same definition as in Chemical Formula 1.

3. The heterocyclic compound of claim 1, wherein L₂ is a direct bond; or a substituted or unsubstituted C6 to C30 arylene group.

4. The heterocyclic compound of claim 1, wherein Z₂ is a substituted or unsubstituted pyridine group; a substituted or unsubstituted pyrimidine group; a substituted or unsubstituted pyrazine group; a substituted or unsubstituted triazine group; a substituted or unsubstituted quinoline group; a substituted or unsubstituted quinazoline group; a substituted or unsubstituted benzoquinoline group; or a substituted or unsubstituted phenanthroline group.

5. The heterocyclic compound of claim 1, wherein the “substituted or unsubstituted” means being substituted with one or more substituents selected from the group consisting of a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group, a C2 to C60 monocyclic or polycyclic heteroaryl group; —SiRR′R″; —P(═O)RR′; and an amine group, or being substituted with a substituent linking two or more substituents selected from among the substituents illustrated above, or being unsubstituted, and R, R′ and R″ are the same as or different from each other, and each independently hydrogen; deuterium; a cyano group; a C1 to C60 alkyl group; a C3 to C60 cycloalkyl group; a C6 to C60 aryl group; or a C2 to C60 heteroaryl group.

6. The heterocyclic compound of claim 1, wherein Chemical Formula 1 is represented by any one of the following compounds:

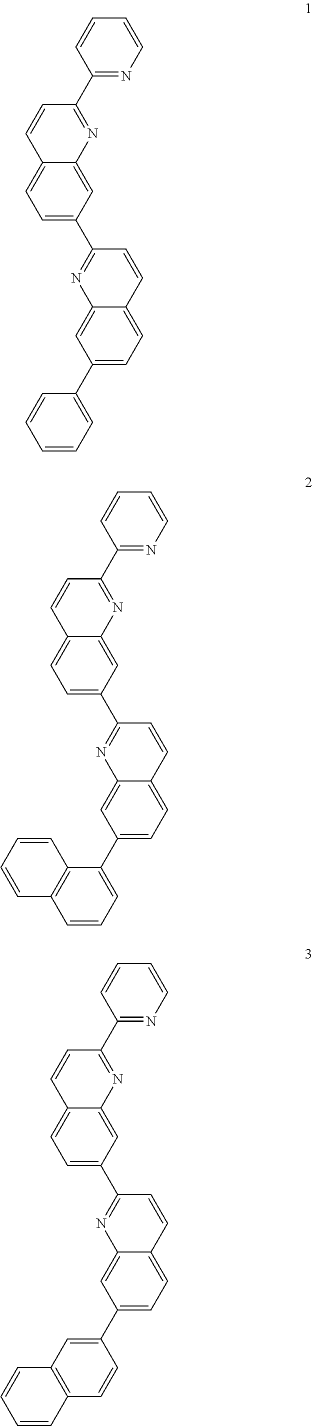

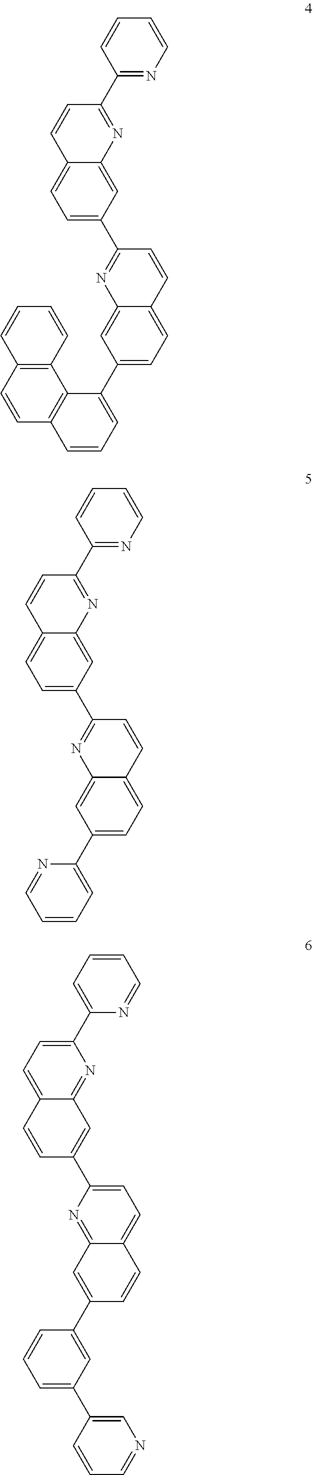

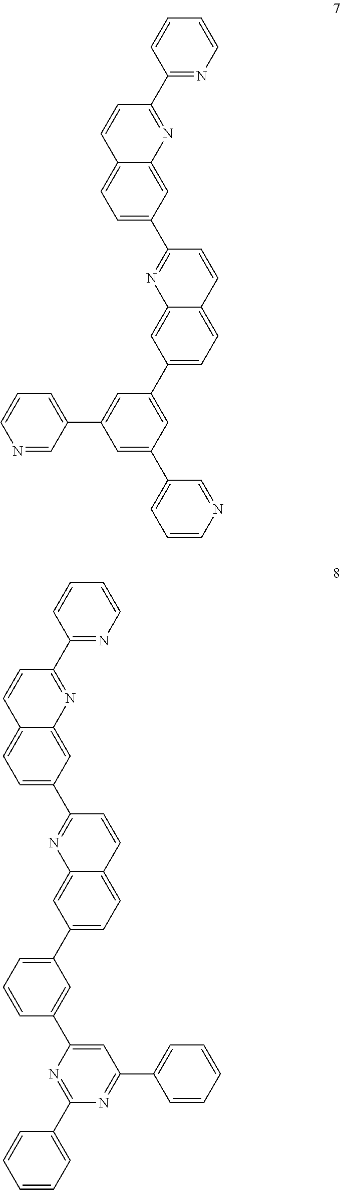 [SMILES: c1ccc(-c2ccc3ccc(-c4ccc5ccc(-c6cc(-c7cccnc7)cc(-c7cccnc7)c6)cc5n4)cc3n2)nc1 ; c1ccc(-c2cc(-c3cccc(-c4ccc5ccc(-c6ccc7ccc(-c8ccccn8)nc7c6)nc5c4)c3)nc(-c3ccccc3)n2)cc1]

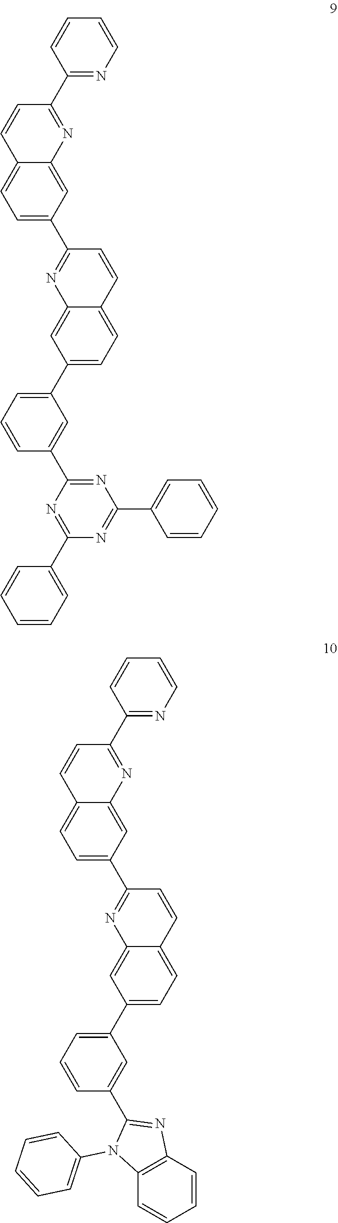 [SMILES: c1ccc(-c2nc(-c3ccccc3)nc(-c3cccc(-c4ccc5ccc(-c6ccc7ccc(-c8ccccn8)nc7c6)nc5c4)c3)n2)cc1 ; c1ccc(-n2c(-c3cccc(-c4ccc5ccc(-c6ccc7ccc(-c8ccccn8)nc7c6)nc5c4)c3)nc3ccccc32)cc1]

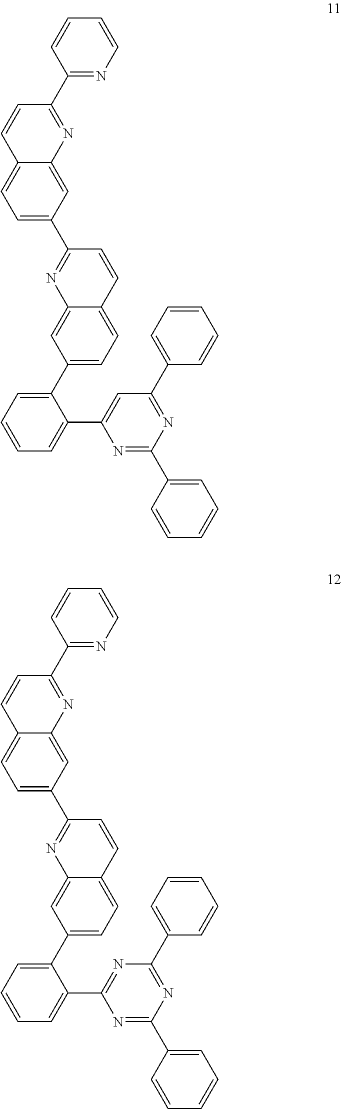 [SMILES: c1ccc(-c2cc(-c3ccccc3-c3ccc4ccc(-c5ccc6ccc(-c7ccccn7)nc6c5)nc4c3)nc(-c3ccccc3)n2)cc1 ; c1ccc(-c2nc(-c3ccccc3)nc(-c3ccccc3-c3ccc4ccc(-c5ccc6ccc(-c7ccccn7)nc6c5)nc4c3)n2)cc1]

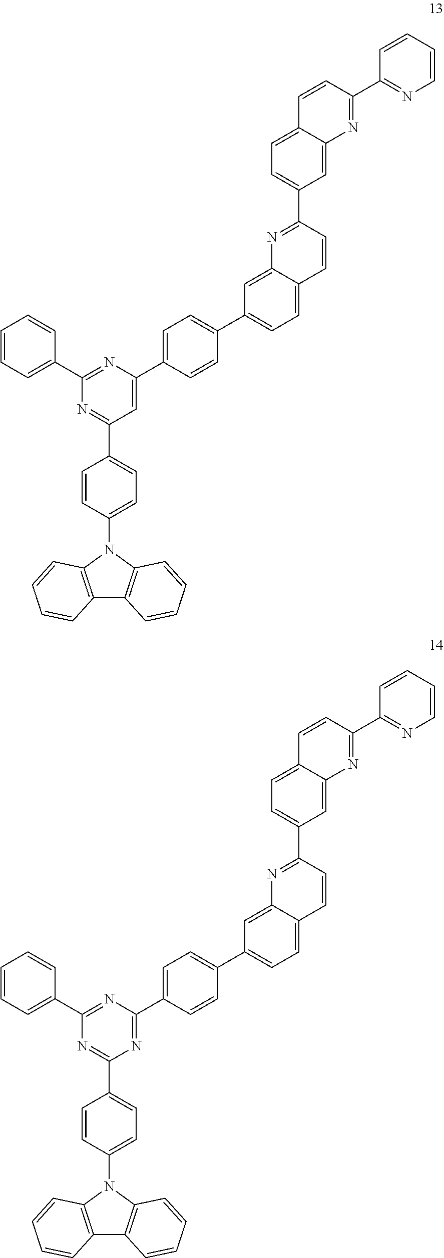 [SMILES: c1ccc(-c2nc(-c3ccc(-c4ccc5ccc(-c6ccc7ccc(-c8ccccn8)nc7c6)nc5c4)cc3)cc(-c3ccc(-n4c5ccccc5c5ccccc54)cc3)n2)cc1 ; c1ccc(-c2nc(-c3ccc(-c4ccc5ccc(-c6ccc7ccc(-c8ccccn8)nc7c6)nc5c4)cc3)nc(-c3ccc(-n4c5ccccc5c5ccccc54)cc3)n2)cc1]

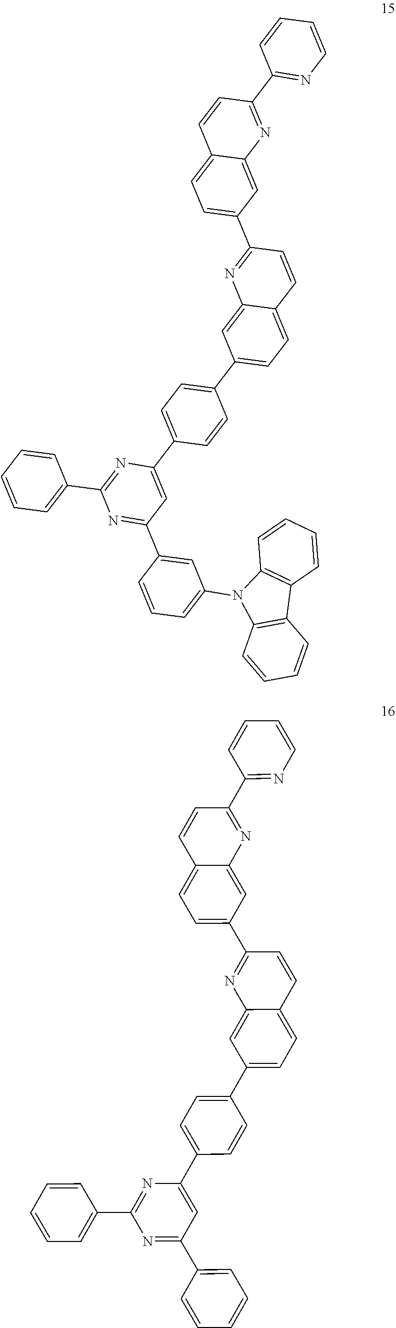

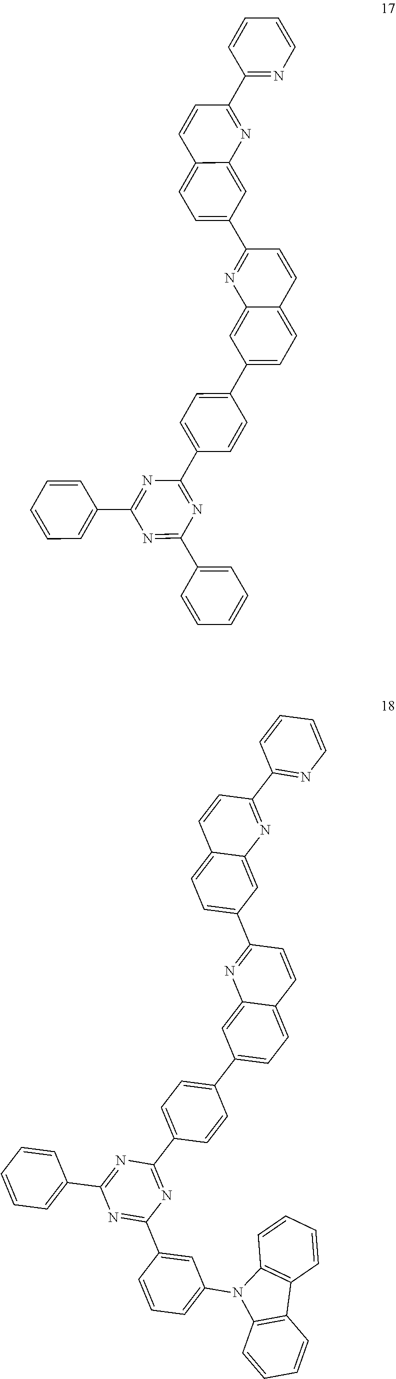 [SMILES: c1ccc(-c2nc(-c3ccccc3)nc(-c3ccc(-c4ccc5ccc(-c6ccc7ccc(-c8ccccn8)nc7c6)nc5c4)cc3)n2)cc1 ; c1ccc(-c2nc(-c3ccc(-c4ccc5ccc(-c6ccc7ccc(-c8ccccn8)nc7c6)nc5c4)cc3)nc(-c3cccc(-n4c5ccccc5c5ccccc54)c3)n2)cc1]

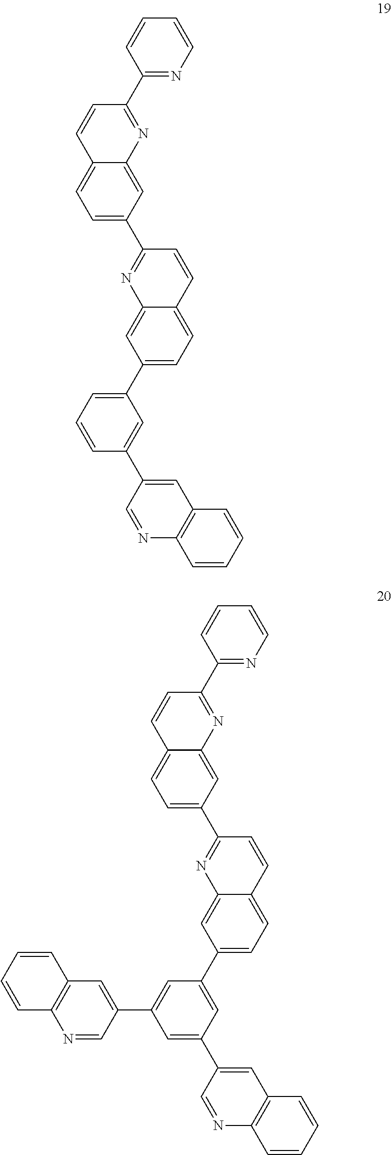

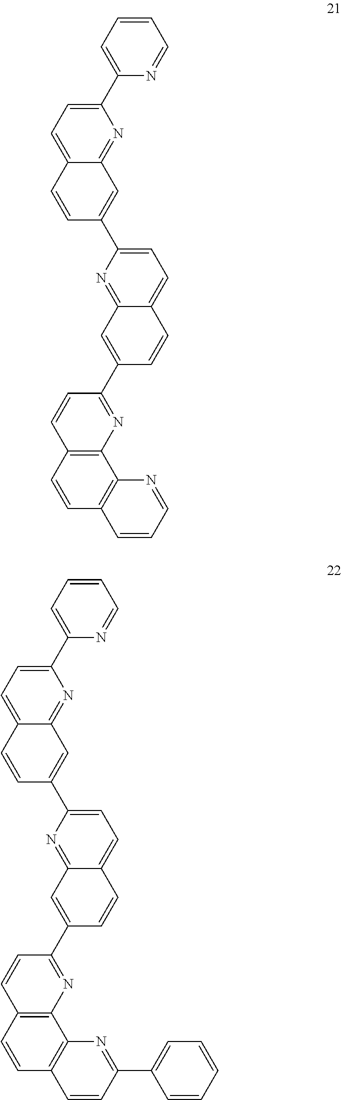

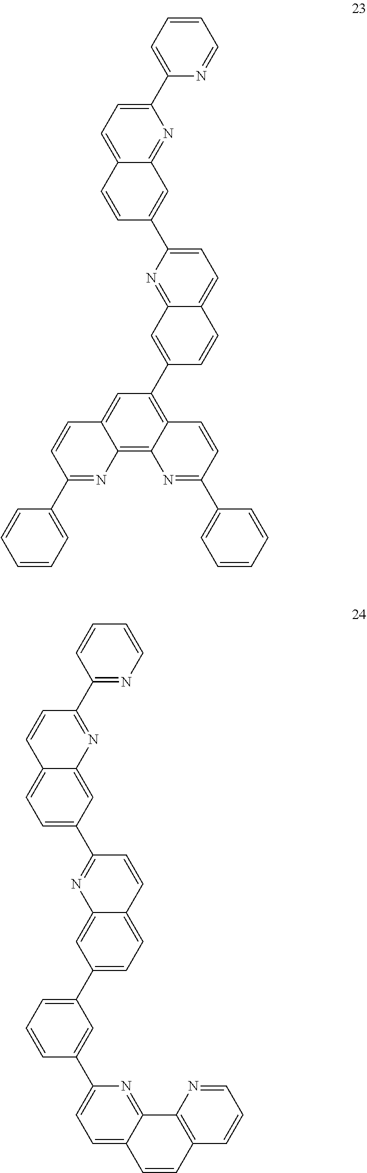 [SMILES: c1ccc(-c2ccc3cc(-c4ccc5ccc(-c6ccc7ccc(-c8ccccn8)nc7c6)nc5c4)c4ccc(-c5ccccc5)nc4c3n2)cc1 ; c1ccc(-c2ccc3ccc(-c4ccc5ccc(-c6cccc(-c7ccc8ccc9cccnc9c8n7)c6)cc5n4)cc3n2)nc1]

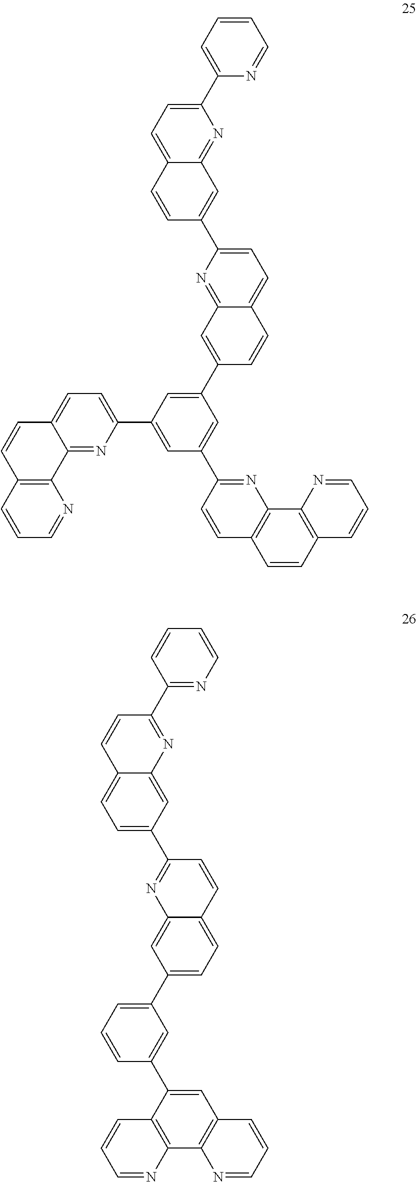 [SMILES: c1ccc(-c2ccc3ccc(-c4ccc5ccc(-c6cc(-c7ccc8ccc9cccnc9c8n7)cc(-c7ccc8ccc9cccnc9c8n7)c6)cc5n4)cc3n2)nc1 ; c1ccc(-c2ccc3ccc(-c4ccc5ccc(-c6cccc(-c7cc8cccnc8c8ncccc78)c6)cc5n4)cc3n2)nc1]

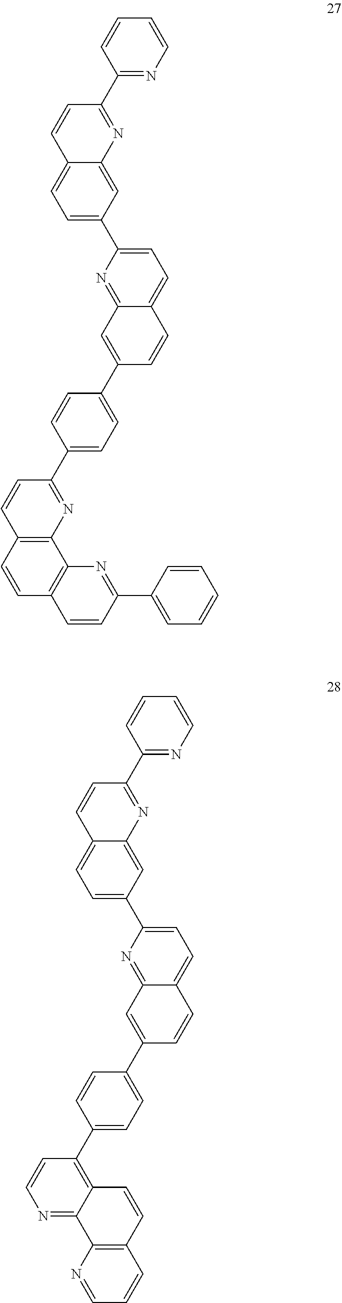 [SMILES: c1ccc(-c2ccc3ccc4ccc(-c5ccc(-c6ccc7ccc(-c8ccc9ccc(-c%10ccccn%10)nc9c8)nc7c6)cc5)nc4c3n2)cc1 ; c1ccc(-c2ccc3ccc(-c4ccc5ccc(-c6ccc(-c7ccnc8c7ccc7cccnc78)cc6)cc5n4)cc3n2)nc1]

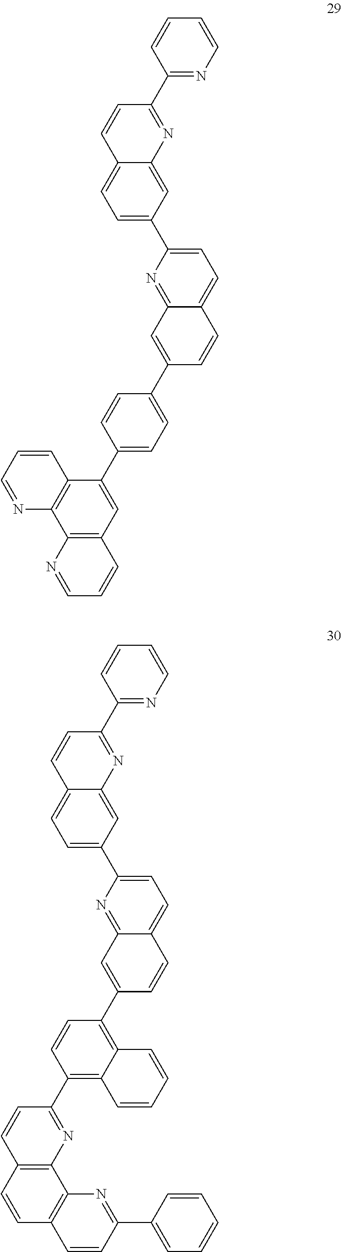 [SMILES: c1ccc(-c2ccc3ccc(-c4ccc5ccc(-c6ccc(-c7cc8cccnc8c8ncccc78)cc6)cc5n4)cc3n2)nc1 ; c1ccc(-c2ccc3ccc4ccc(-c5ccc(-c6ccc7ccc(-c8ccc9ccc(-c%10ccccn%10)nc9c8)nc7c6)c6ccccc56)nc4c3n2)cc1]

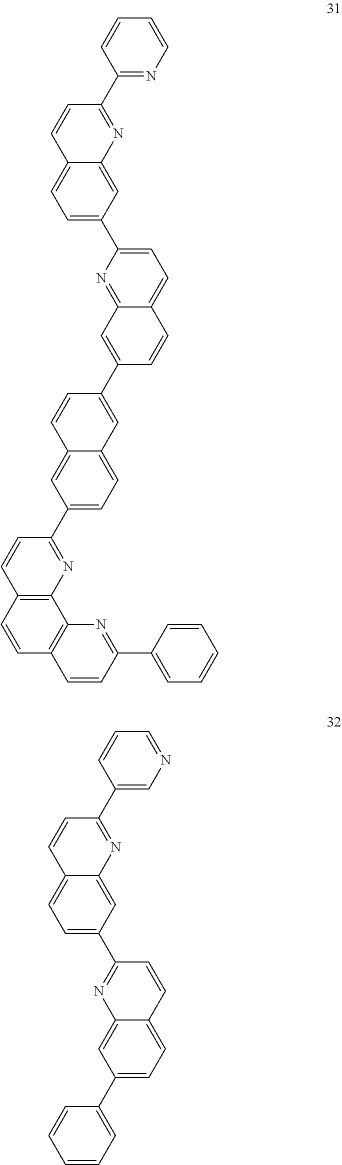 [SMILES: c1ccc(-c2ccc3ccc4ccc(-c5ccc6cc(-c7ccc8ccc(-c9ccc%10ccc(-c%11ccccn%11)nc%10c9)nc8c7)ccc6c5)nc4c3n2)cc1 ; c1ccc(-c2ccc3ccc(-c4ccc5ccc(-c6cccnc6)nc5c4)nc3c2)cc1]

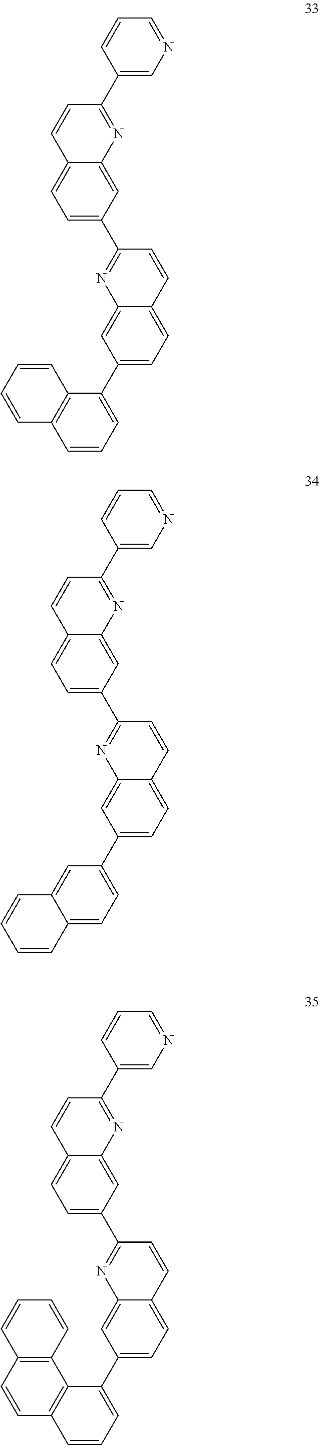

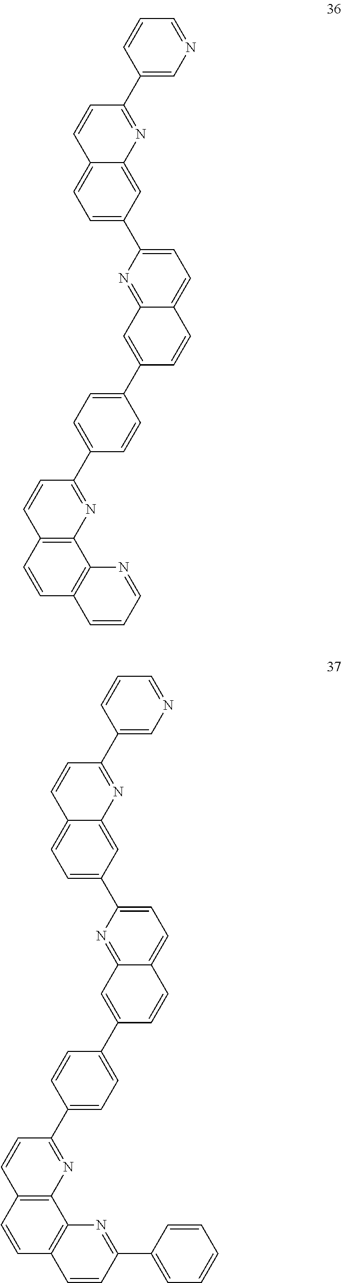 [SMILES: c1cncc(-c2ccc3ccc(-c4ccc5ccc(-c6ccc(-c7ccc8ccc9cccnc9c8n7)cc6)cc5n4)cc3n2)c1 ; c1ccc(-c2ccc3ccc4ccc(-c5ccc(-c6ccc7ccc(-c8ccc9ccc(-c%10cccnc%10)nc9c8)nc7c6)cc5)nc4c3n2)cc1]

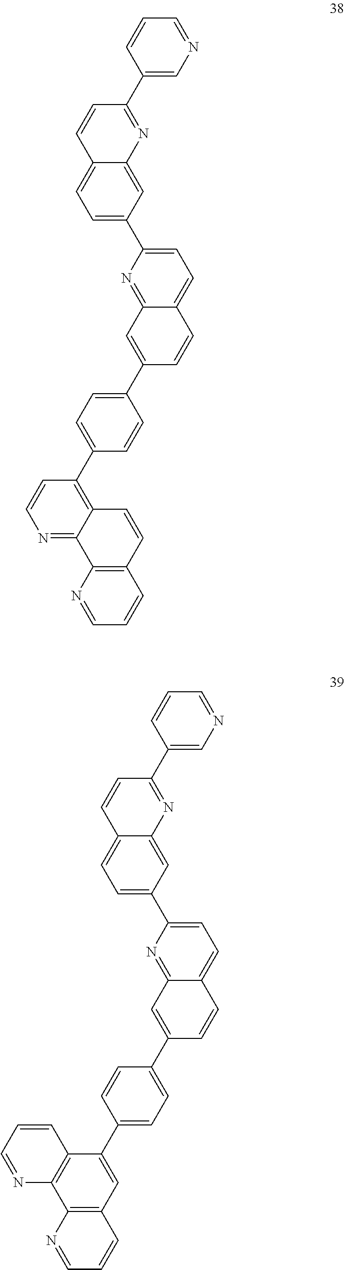 [SMILES: c1cncc(-c2ccc3ccc(-c4ccc5ccc(-c6ccc(-c7ccnc8c7ccc7cccnc78)cc6)cc5n4)cc3n2)c1 ; c1cncc(-c2ccc3ccc(-c4ccc5ccc(-c6ccc(-c7cc8cccnc8c8ncccc78)cc6)cc5n4)cc3n2)c1]

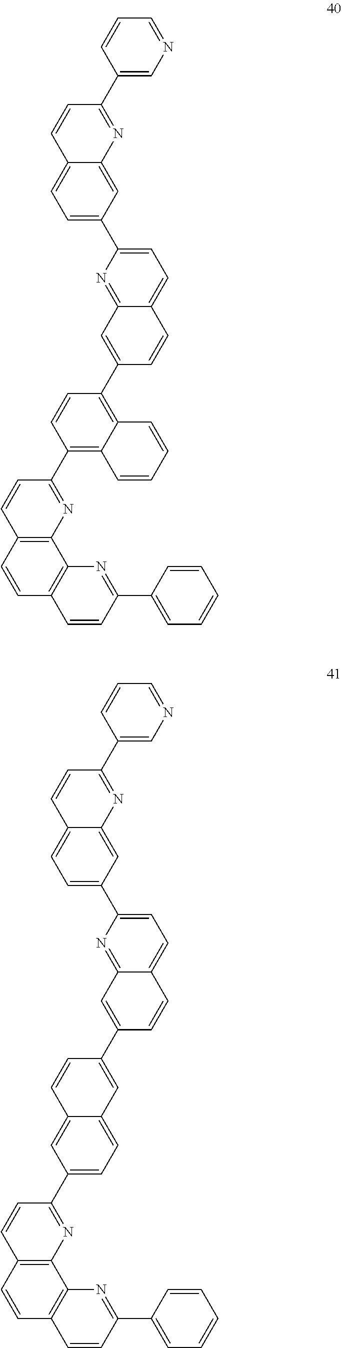 [SMILES: c1ccc(-c2ccc3ccc4ccc(-c5ccc(-c6ccc7ccc(-c8ccc9ccc(-c%10cccnc%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11cccnc%11)nc%10c9)nc8c7)ccc6c5)nc4c3n2)cc1]

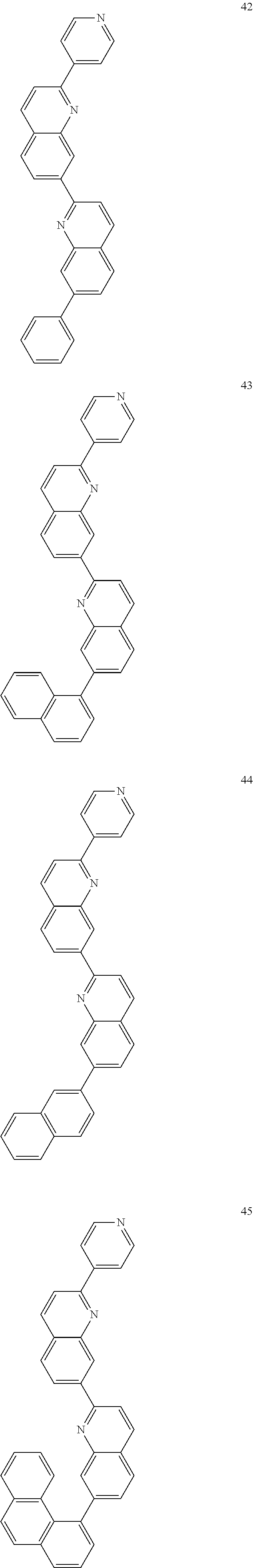 [SMILES: c1ccc(-c2ccc3ccc(-c4ccc5ccc(-c6ccncc6)nc5c4)nc3c2)cc1 ; c1ccc2c(-c3ccc4ccc(-c5ccc6ccc(-c7ccncc7)nc6c5)nc4c3)cccc2c1 ; c1ccc2cc(-c3ccc4ccc(-c5ccc6ccc(-c7ccncc7)nc6c5)nc4c3)ccc2c1 ; c1ccc2c(c1)ccc1cccc(-c3ccc4ccc(-c5ccc6ccc(-c7ccncc7)nc6c5)nc4c3)c12]

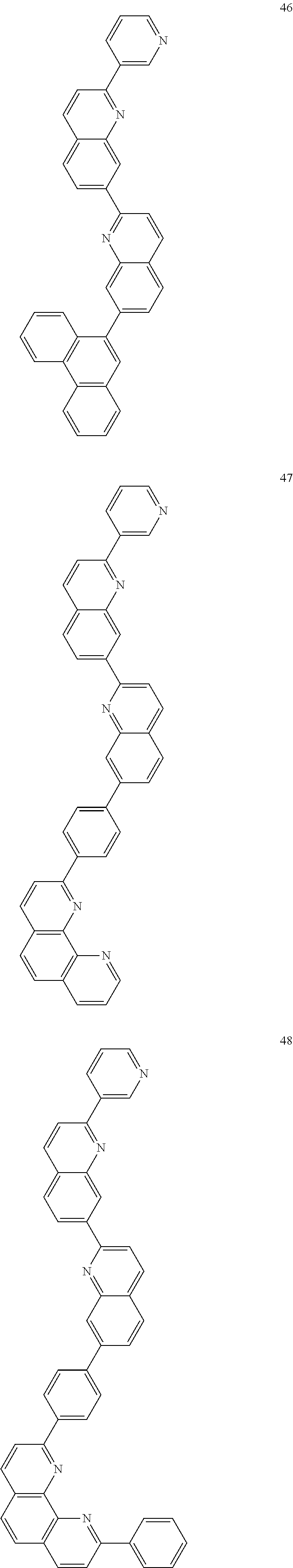 [SMILES: c1cncc(-c2ccc3ccc(-c4ccc5ccc(-c6cc7ccccc7c7ccccc67)cc5n4)cc3n2)c1 ; c1cncc(-c2ccc3ccc(-c4ccc5ccc(-c6ccc(-c7ccc8ccc9cccnc9c8n7)cc6)cc5n4)cc3n2)c1 ; c1ccc(-c2ccc3ccc4ccc(-c5ccc(-c6ccc7ccc(-c8ccc9ccc(-c%10cccnc%10)nc9c8)nc7c6)cc5)nc4c3n2)cc1]

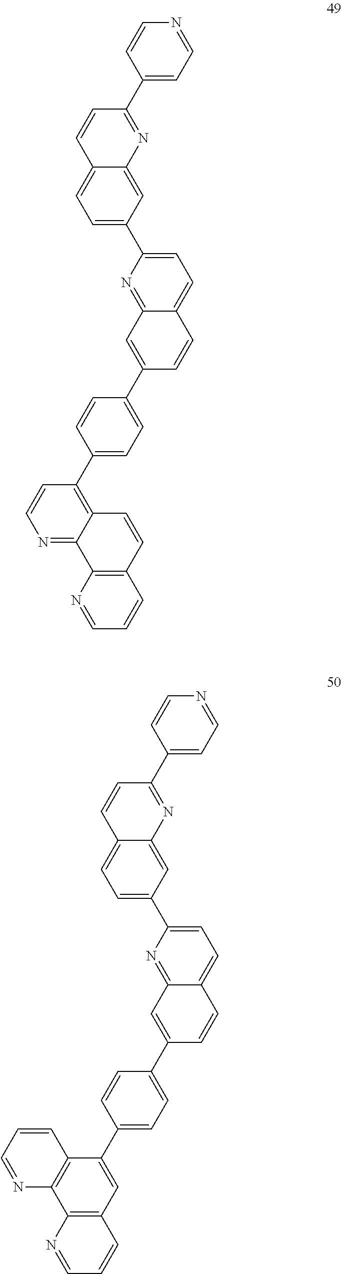 [SMILES: c1cnc2c(c1)ccc1c(-c3ccc(-c4ccc5ccc(-c6ccc7ccc(-c8ccncc8)nc7c6)nc5c4)cc3)ccnc12 ; c1cnc2c(c1)cc(-c1ccc(-c3ccc4ccc(-c5ccc6ccc(-c7ccncc7)nc6c5)nc4c3)cc1)c1cccnc12]

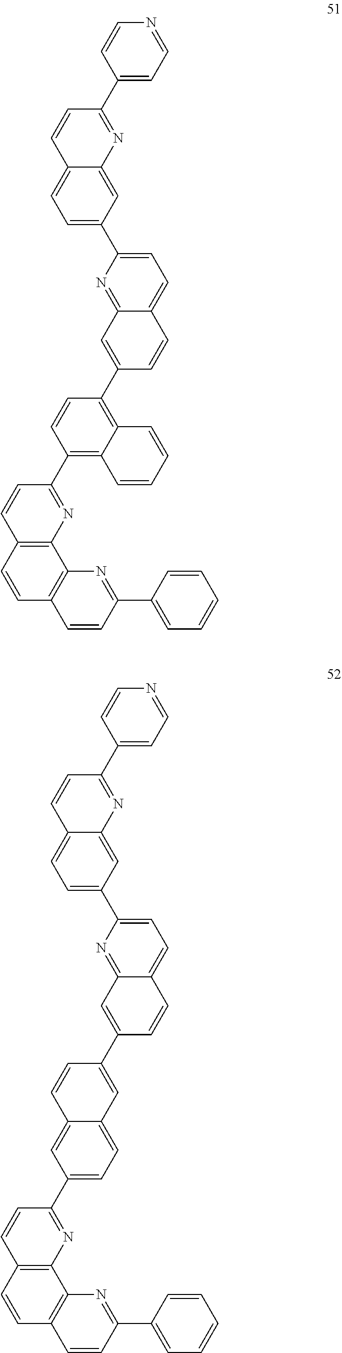 [SMILES: c1ccc(-c2ccc3ccc4ccc(-c5ccc(-c6ccc7ccc(-c8ccc9ccc(-c%10ccncc%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11ccncc%11)nc%10c9)nc8c7)ccc6c5)nc4c3n2)cc1]

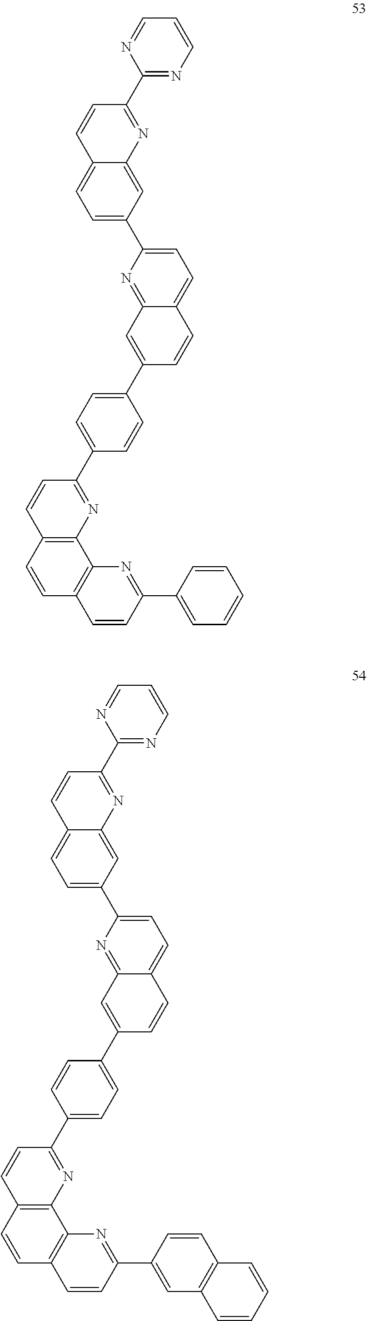 [SMILES: c1ccc(-c2ccc3ccc4ccc(-c5ccc(-c6ccc7ccc(-c8ccc9ccc(-c%10ncccn%10)nc9c8)nc7c6)cc5)nc4c3n2)cc1 ; c1cnc(-c2ccc3ccc(-c4ccc5ccc(-c6ccc(-c7ccc8ccc9ccc(-c%10ccc%11ccccc%11c%10)nc9c8n7)cc6)cc5n4)cc3n2)nc1]

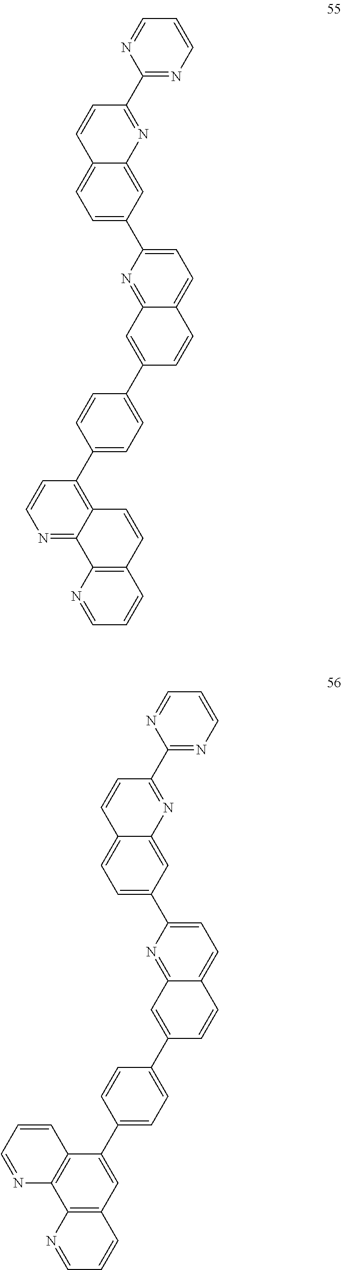 [SMILES: c1cnc(-c2ccc3ccc(-c4ccc5ccc(-c6ccc(-c7ccnc8c7ccc7cccnc78)cc6)cc5n4)cc3n2)nc1 ; c1cnc(-c2ccc3ccc(-c4ccc5ccc(-c6ccc(-c7cc8cccnc8c8ncccc78)cc6)cc5n4)cc3n2)nc1]

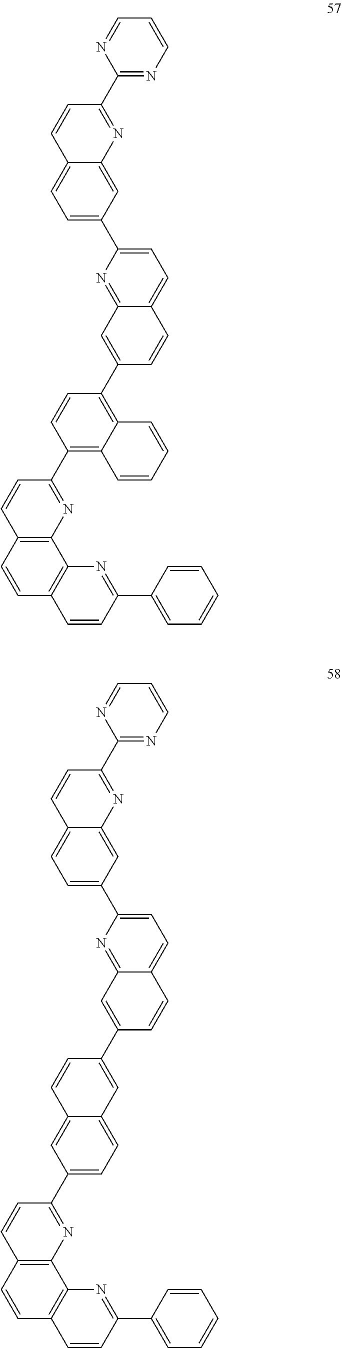 [SMILES: c1ccc(-c2ccc3ccc4ccc(-c5ccc(-c6ccc7ccc(-c8ccc9ccc(-c%10ncccn%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11ncccn%11)nc%10c9)nc8c7)ccc6c5)nc4c3n2)cc1]

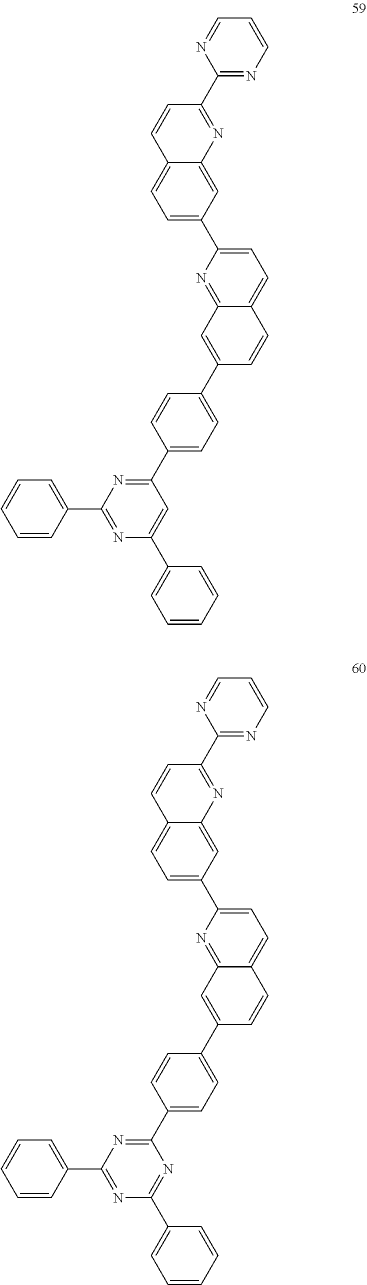 [SMILES: c1ccc(-c2cc(-c3ccc(-c4ccc5ccc(-c6ccc7ccc(-c8ncccn8)nc7c6)nc5c4)cc3)nc(-c3ccccc3)n2)cc1 ; c1ccc(-c2nc(-c3ccccc3)nc(-c3ccc(-c4ccc5ccc(-c6ccc7ccc(-c8ncccn8)nc7c6)nc5c4)cc3)n2)cc1]

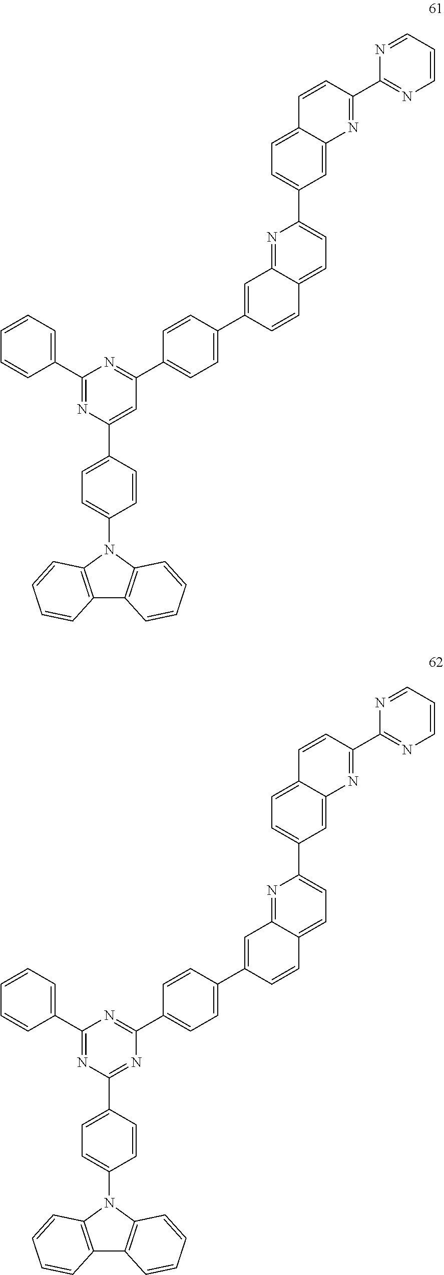 [SMILES: c1ccc(-c2nc(-c3ccc(-c4ccc5ccc(-c6ccc7ccc(-c8ncccn8)nc7c6)nc5c4)cc3)cc(-c3ccc(-n4c5ccccc5c5ccccc54)cc3)n2)cc1 ; c1ccc(-c2nc(-c3ccc(-c4ccc5ccc(-c6ccc7ccc(-c8ncccn8)nc7c6)nc5c4)cc3)nc(-c3ccc(-n4c5ccccc5c5ccccc54)cc3)n2)cc1]

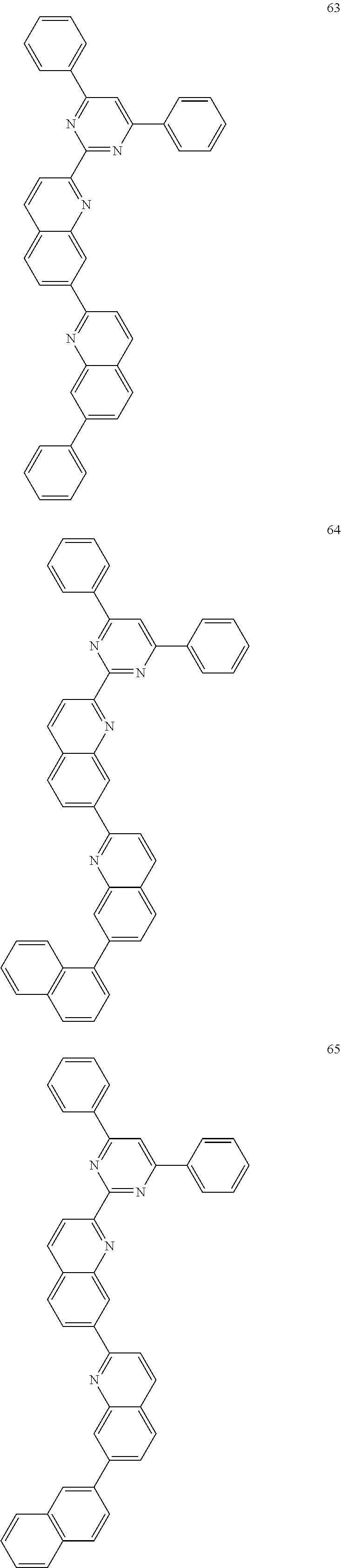

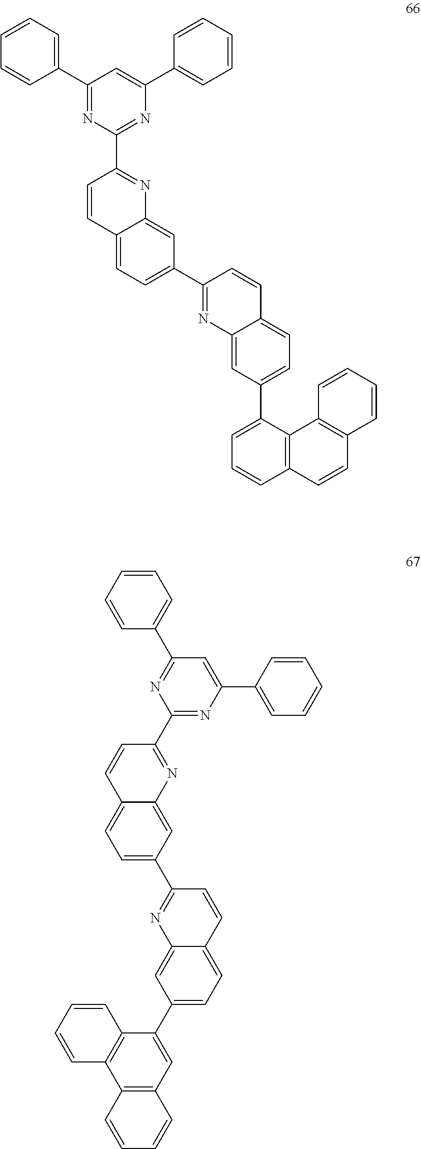 [SMILES: c1ccc(-c2cc(-c3ccccc3)nc(-c3ccc4ccc(-c5ccc6ccc(-c7cccc8ccc9ccccc9c78)cc6n5)cc4n3)n2)cc1 ; c1ccc(-c2cc(-c3ccccc3)nc(-c3ccc4ccc(-c5ccc6ccc(-c7cc8ccccc8c8ccccc78)cc6n5)cc4n3)n2)cc1]

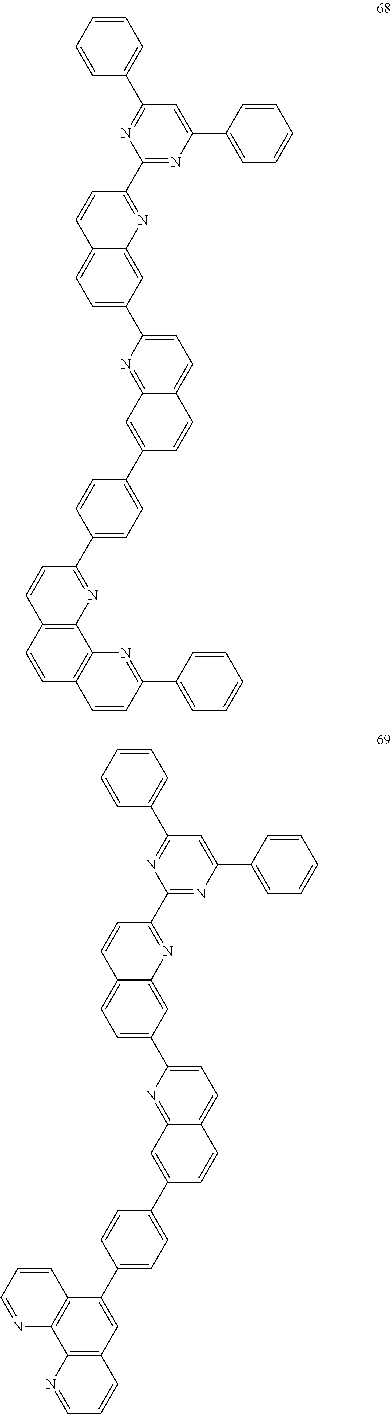

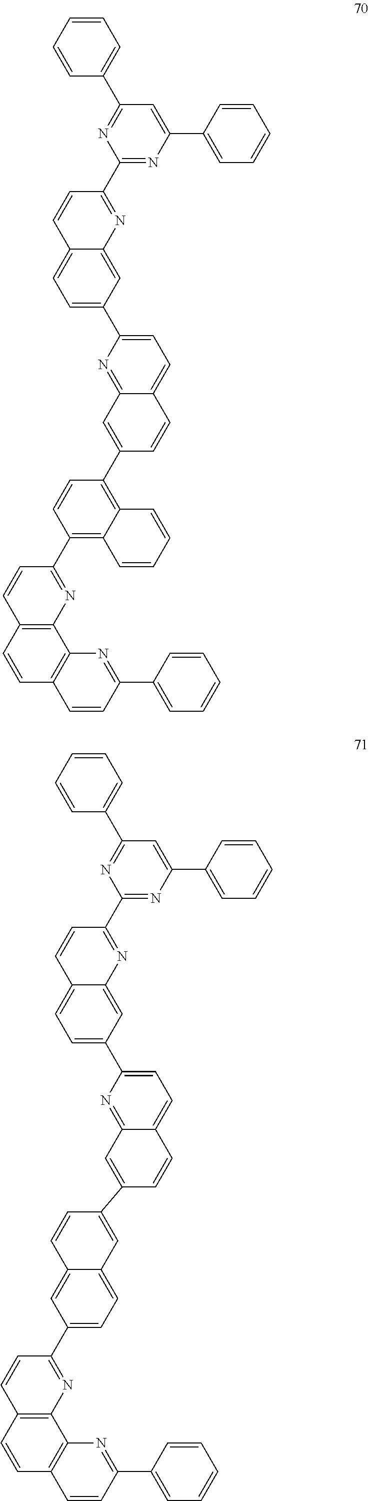

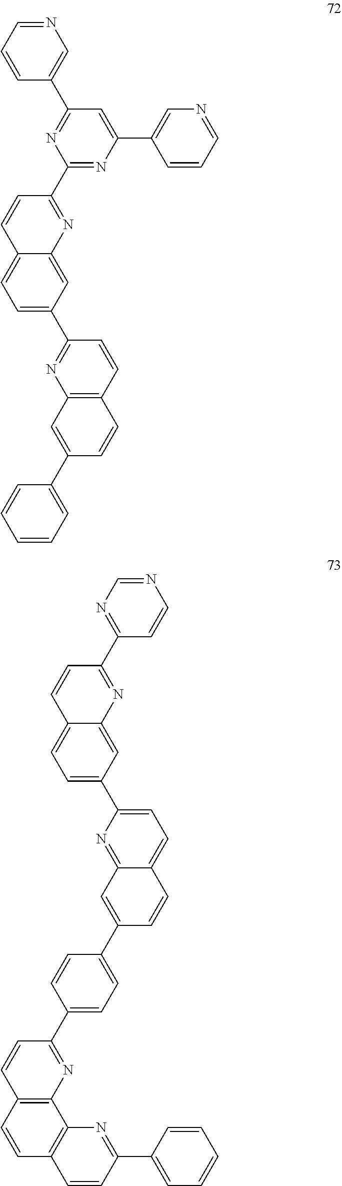 [SMILES: c1ccc(-c2ccc3ccc(-c4ccc5ccc(-c6nc(-c7cccnc7)cc(-c7cccnc7)n6)nc5c4)nc3c2)cc1 ; c1ccc(-c2ccc3ccc4ccc(-c5ccc(-c6ccc7ccc(-c8ccc9ccc(-c%10ccncn%10)nc9c8)nc7c6)cc5)nc4c3n2)cc1]

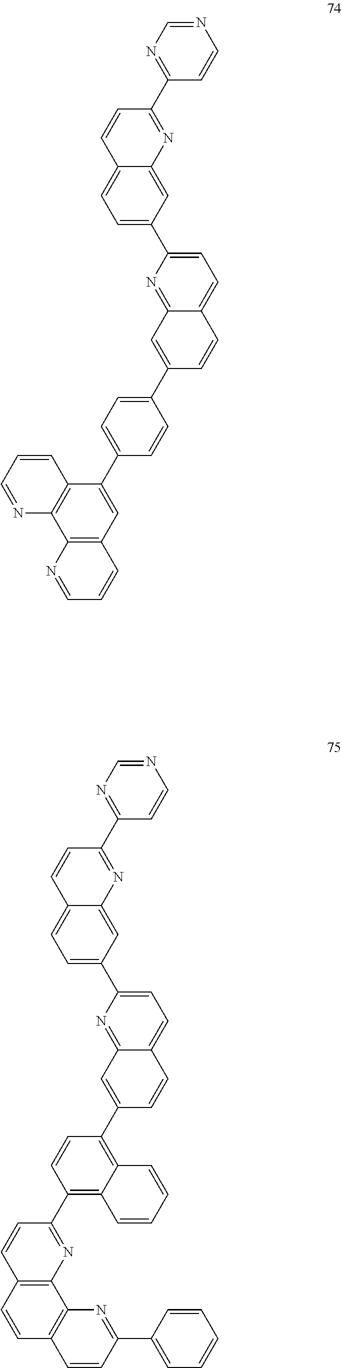 [SMILES: c1cnc2c(c1)cc(-c1ccc(-c3ccc4ccc(-c5ccc6ccc(-c7ccncn7)nc6c5)nc4c3)cc1)c1cccnc12 ; c1ccc(-c2ccc3ccc4ccc(-c5ccc(-c6ccc7ccc(-c8ccc9ccc(-c%10ccncn%10)nc9c8)nc7c6)c6ccccc56)nc4c3n2)cc1]

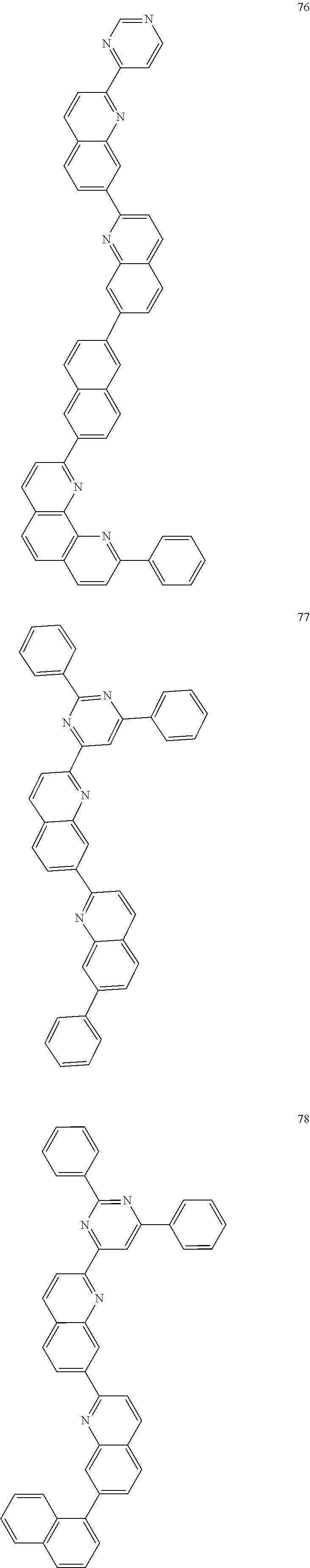 [SMILES: c1ccc(-c2ccc3ccc4ccc(-c5ccc6cc(-c7ccc8ccc(-c9ccc%10ccc(-c%11ccncn%11)nc%10c9)nc8c7)ccc6c5)nc4c3n2)cc1 ; c1ccc(-c2ccc3ccc(-c4ccc5ccc(-c6cc(-c7ccccc7)nc(-c7ccccc7)n6)nc5c4)nc3c2)cc1 ; c1ccc(-c2cc(-c3ccc4ccc(-c5ccc6ccc(-c7cccc8ccccc78)cc6n5)cc4n3)nc(-c3ccccc3)n2)cc1]

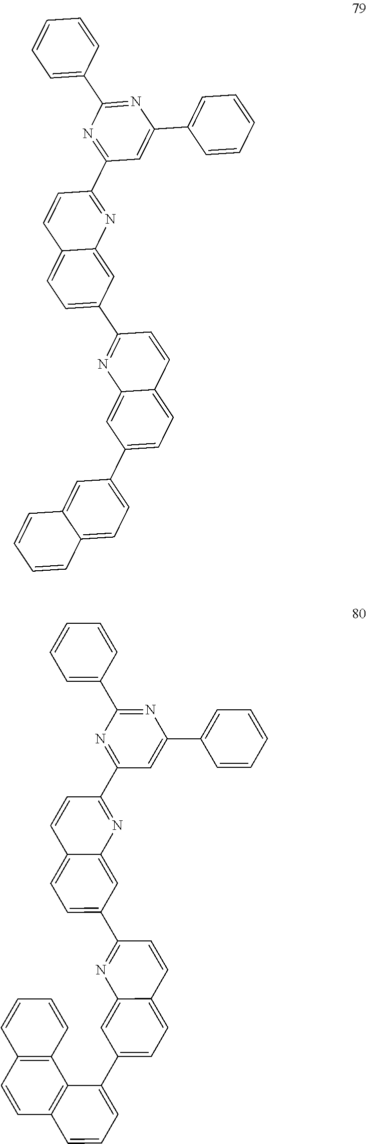 [SMILES: c1ccc(-c2cc(-c3ccc4ccc(-c5ccc6ccc(-c7ccc8ccccc8c7)cc6n5)cc4n3)nc(-c3ccccc3)n2)cc1 ; c1ccc(-c2cc(-c3ccc4ccc(-c5ccc6ccc(-c7cccc8ccc9ccccc9c78)cc6n5)cc4n3)nc(-c3ccccc3)n2)cc1]

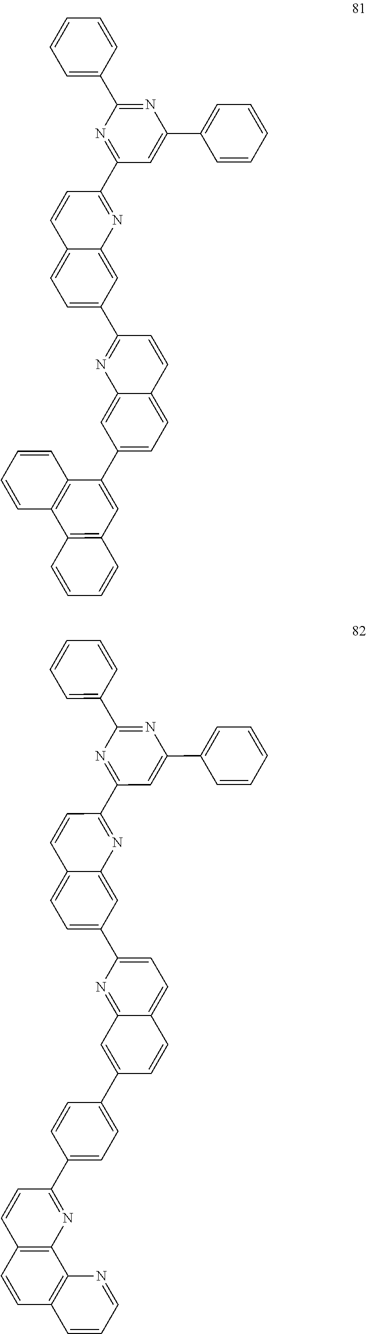

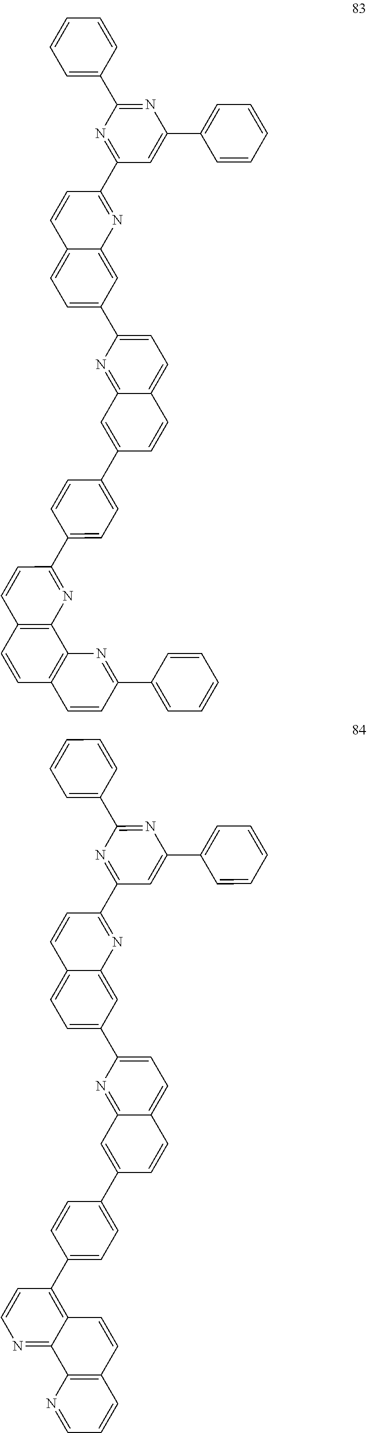

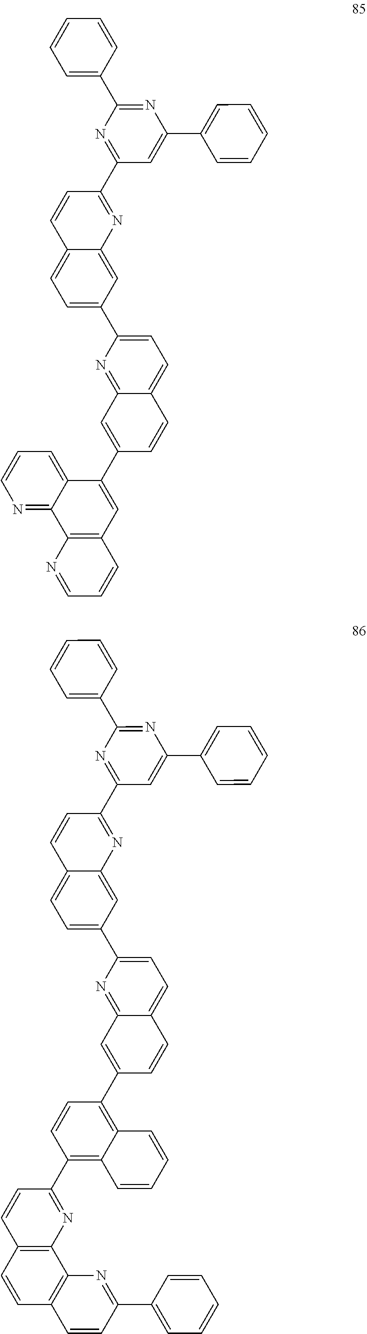

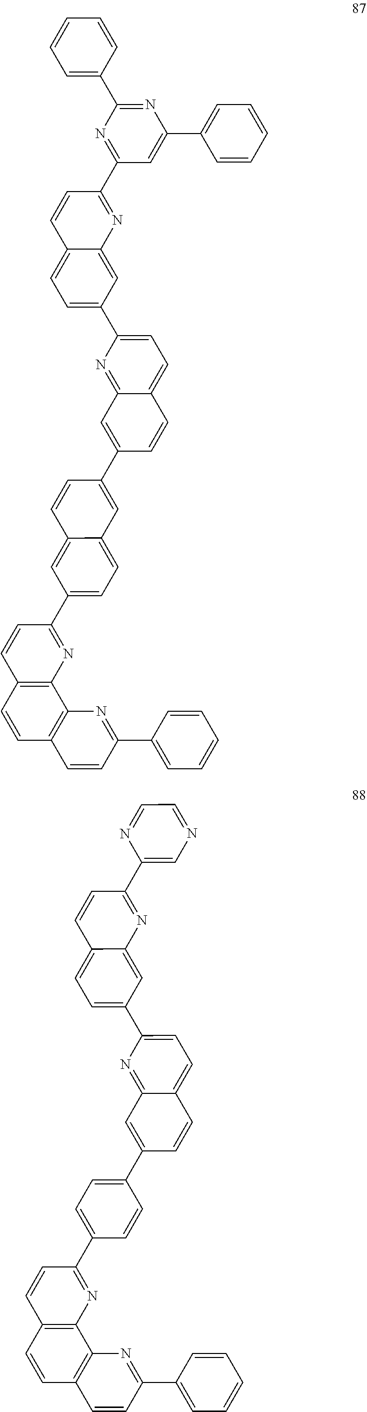

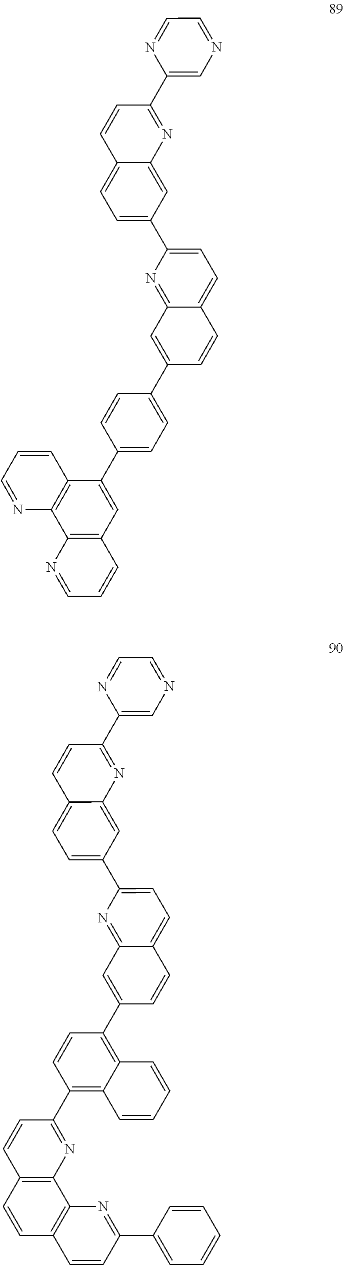 [SMILES: c1cnc2c(c1)cc(-c1ccc(-c3ccc4ccc(-c5ccc6ccc(-c7cnccn7)nc6c5)nc4c3)cc1)c1cccnc12 ; c1ccc(-c2ccc3ccc4ccc(-c5ccc(-c6ccc7ccc(-c8ccc9ccc(-c%10cnccn%10)nc9c8)nc7c6)c6ccccc56)nc4c3n2)cc1]

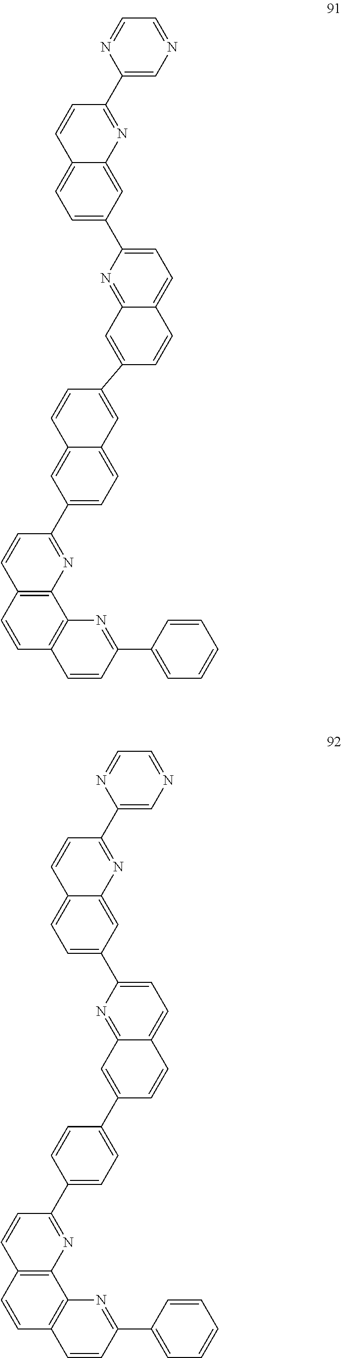 [SMILES: c1ccc(-c2ccc3ccc4ccc(-c5ccc6cc(-c7ccc8ccc(-c9ccc%10ccc(-c%11cnccn%11)nc%10c9)nc8c7)ccc6c5)nc4c3n2)cc1 ; c1ccc(-c2ccc3ccc4ccc(-c5ccc(-c6ccc7ccc(-c8ccc9ccc(-c%10cnccn%10)nc9c8)nc7c6)cc5)nc4c3n2)cc1]

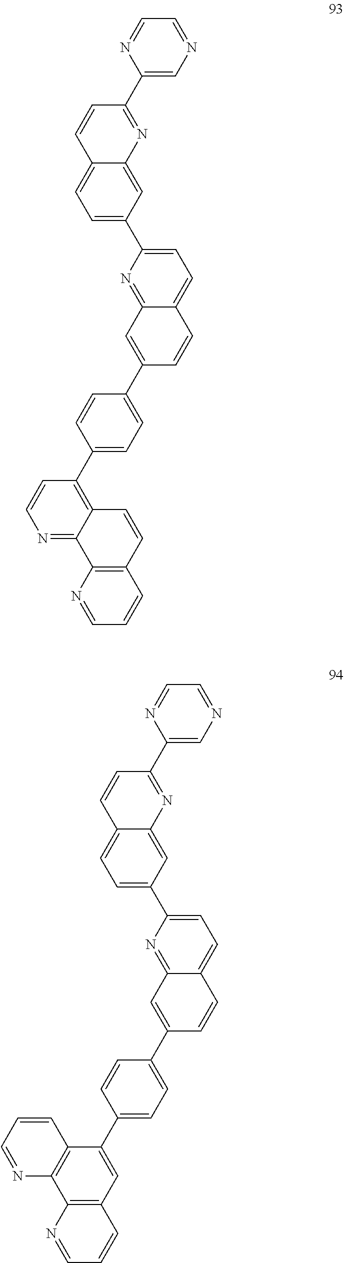 [SMILES: c1cnc2c(c1)ccc1c(-c3ccc(-c4ccc5ccc(-c6ccc7ccc(-c8cnccn8)nc7c6)nc5c4)cc3)ccnc12 ; c1cnc2c(c1)cc(-c1ccc(-c3ccc4ccc(-c5ccc6ccc(-c7cnccn7)nc6c5)nc4c3)cc1)c1cccnc12]

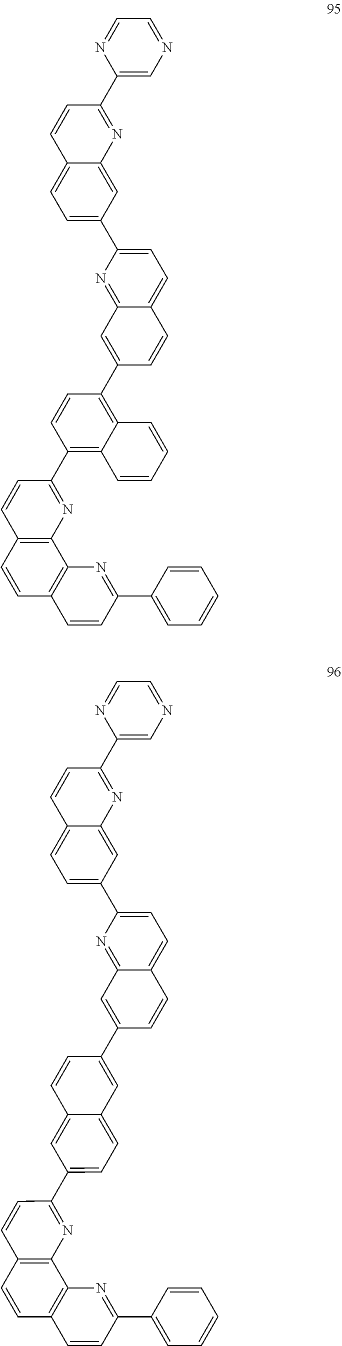 [SMILES: c1ccc(-c2ccc3ccc4ccc(-c5ccc(-c6ccc7ccc(-c8ccc9ccc(-c%10cnccn%10)nc9c8)nc7c6)c6ccccc56)nc4c3n2)cc1 ; c1ccc(-c2ccc3ccc4ccc(-c5ccc6cc(-c7ccc8ccc(-c9ccc%10ccc(-c%11cnccn%11)nc%10c9)nc8c7)ccc6c5)nc4c3n2)cc1]

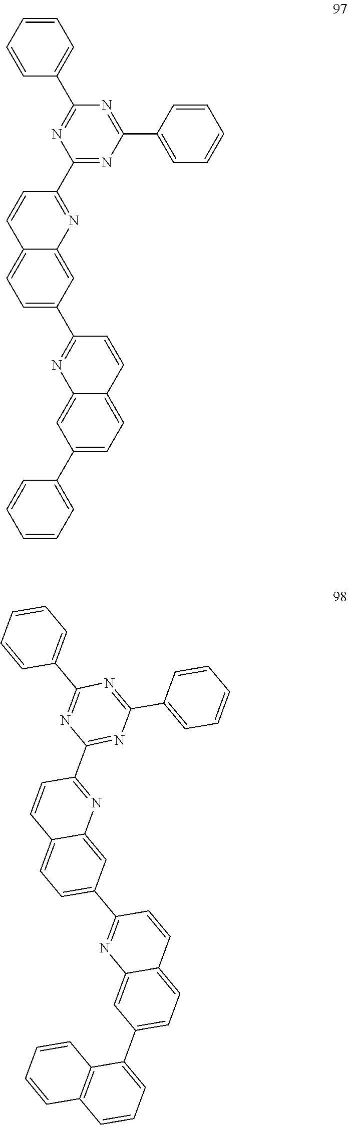 [SMILES: c1ccc(-c2ccc3ccc(-c4ccc5ccc(-c6nc(-c7ccccc7)nc(-c7ccccc7)n6)nc5c4)nc3c2)cc1 ; c1ccc(-c2nc(-c3ccccc3)nc(-c3ccc4ccc(-c5ccc6ccc(-c7cccc8ccccc78)cc6n5)cc4n3)n2)cc1]

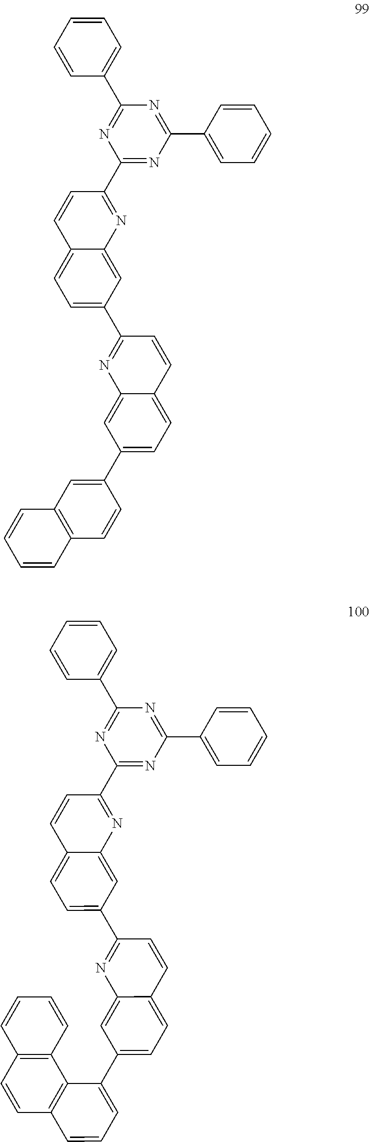 [SMILES: c1ccc(-c2nc(-c3ccccc3)nc(-c3ccc4ccc(-c5ccc6ccc(-c7ccc8ccccc8c7)cc6n5)cc4n3)n2)cc1 ; c1ccc(-c2nc(-c3ccccc3)nc(-c3ccc4ccc(-c5ccc6ccc(-c7cccc8ccc9ccccc9c78)cc6n5)cc4n3)n2)cc1]

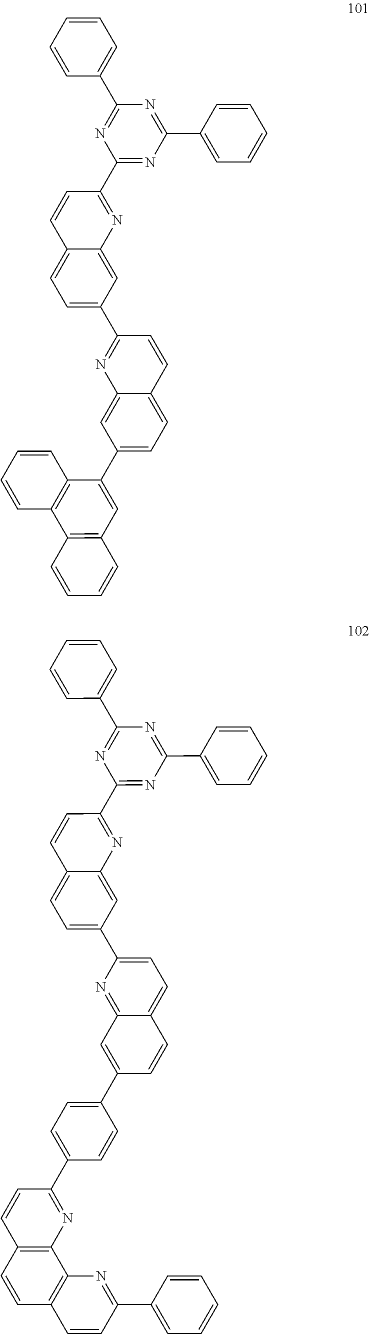

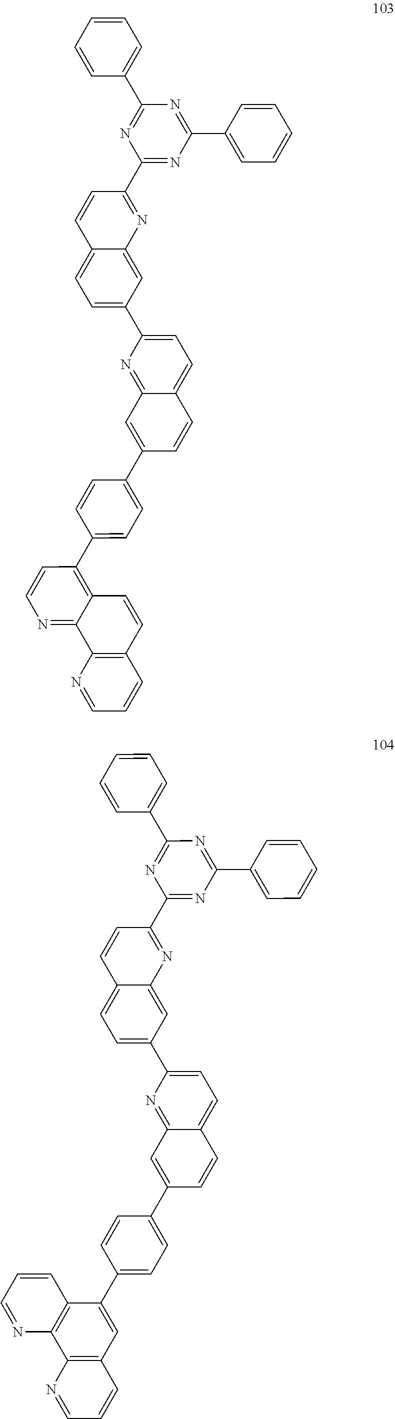

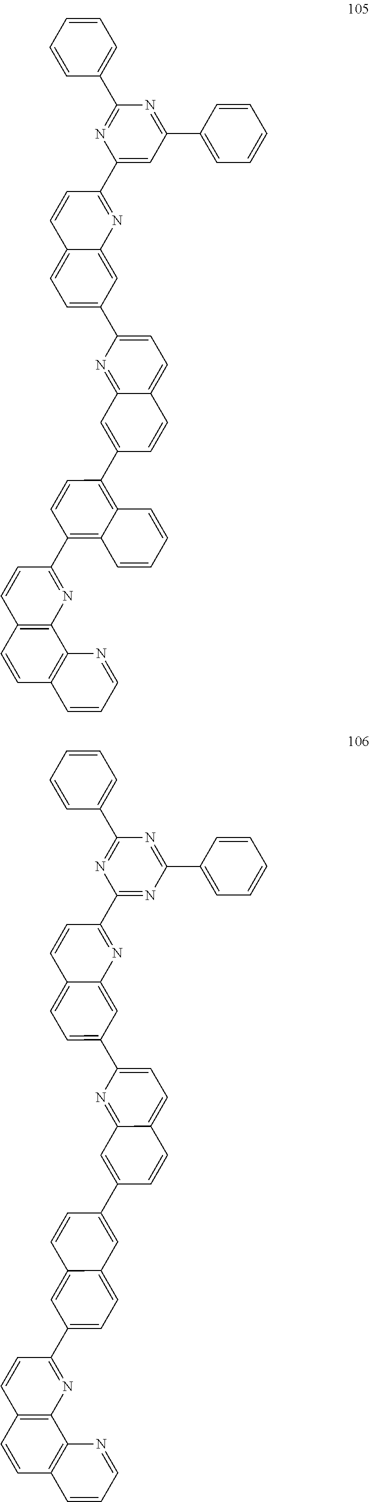

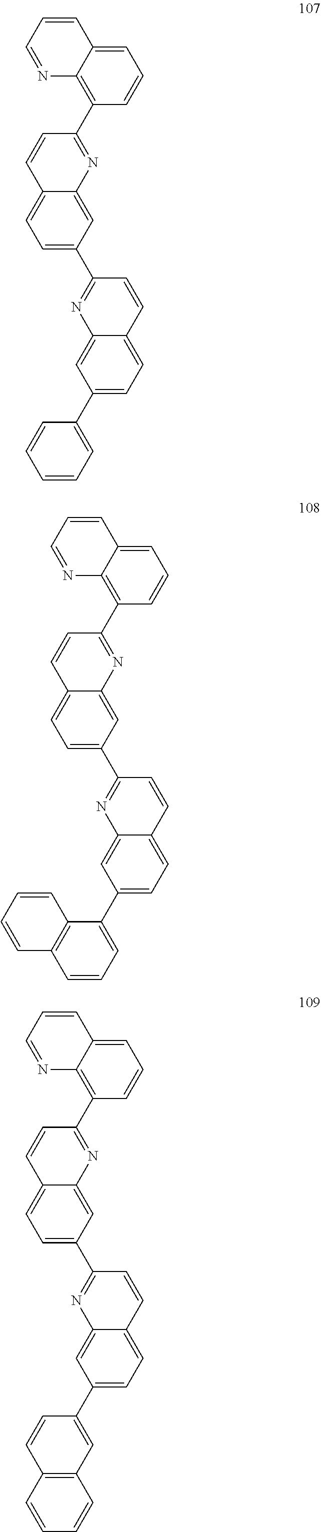

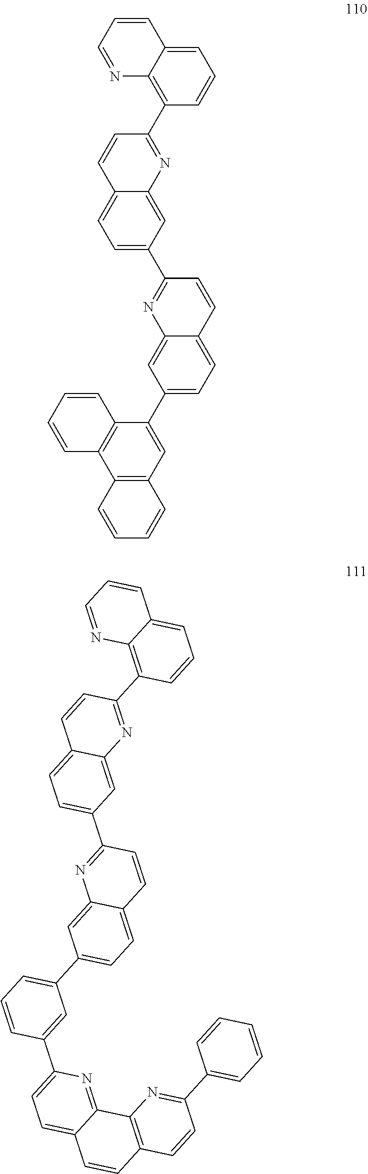

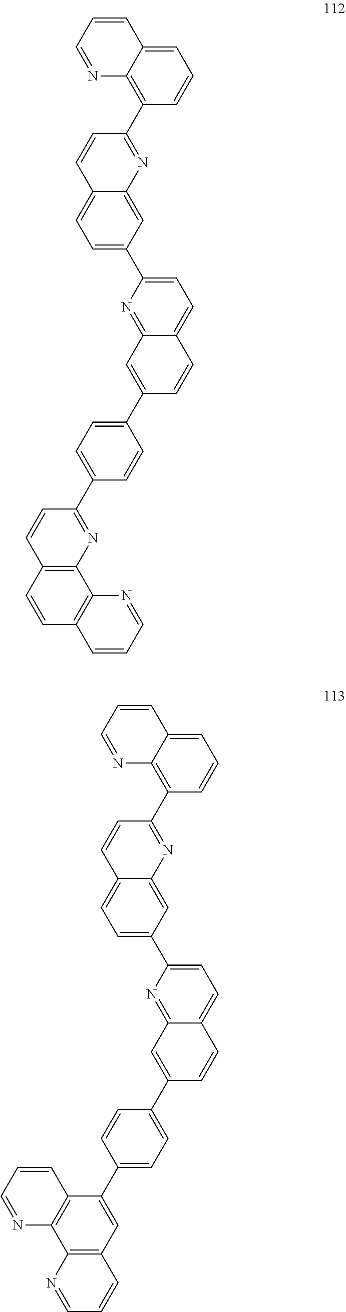 [SMILES: c1cnc2c(-c3ccc4ccc(-c5ccc6ccc(-c7ccc(-c8ccc9ccc%10cccnc%10c9n8)cc7)cc6n5)cc4n3)cccc2c1 ; c1cnc2c(-c3ccc4ccc(-c5ccc6ccc(-c7ccc(-c8cc9cccnc9c9ncccc89)cc7)cc6n5)cc4n3)cccc2c1]

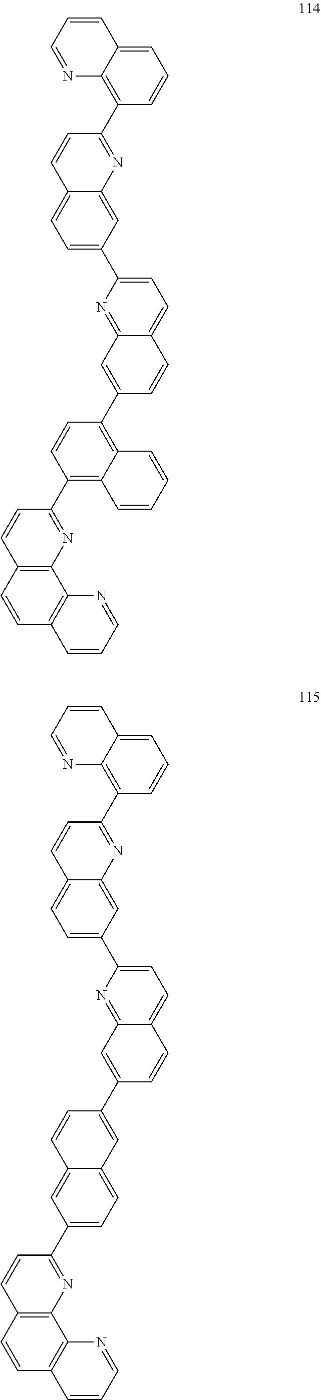 [SMILES: c1cnc2c(-c3ccc4ccc(-c5ccc6ccc(-c7ccc(-c8ccc9ccc%10cccnc%10c9n8)c8ccccc78)cc6n5)cc4n3)cccc2c1 ; c1cnc2c(-c3ccc4ccc(-c5ccc6ccc(-c7ccc8cc(-c9ccc%10ccc%11cccnc%11c%10n9)ccc8c7)cc6n5)cc4n3)cccc2c1]

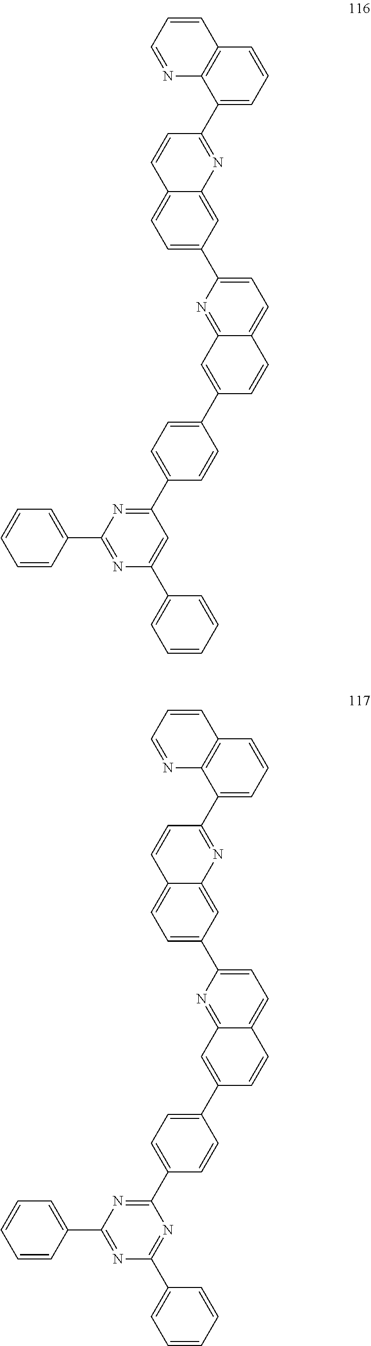 [SMILES: c1ccc(-c2cc(-c3ccc(-c4ccc5ccc(-c6ccc7ccc(-c8cccc9cccnc89)nc7c6)nc5c4)cc3)nc(-c3ccccc3)n2)cc1 ; c1ccc(-c2nc(-c3ccccc3)nc(-c3ccc(-c4ccc5ccc(-c6ccc7ccc(-c8cccc9cccnc89)nc7c6)nc5c4)cc3)n2)cc1]

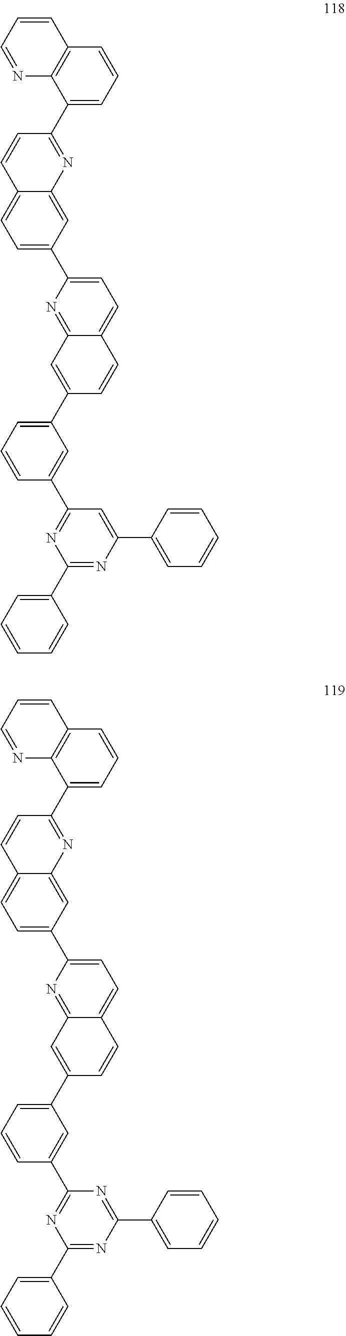 [SMILES: c1ccc(-c2cc(-c3cccc(-c4ccc5ccc(-c6ccc7ccc(-c8cccc9cccnc89)nc7c6)nc5c4)c3)nc(-c3ccccc3)n2)cc1 ; c1ccc(-c2nc(-c3ccccc3)nc(-c3cccc(-c4ccc5ccc(-c6ccc7ccc(-c8cccc9cccnc89)nc7c6)nc5c4)c3)n2)cc1]

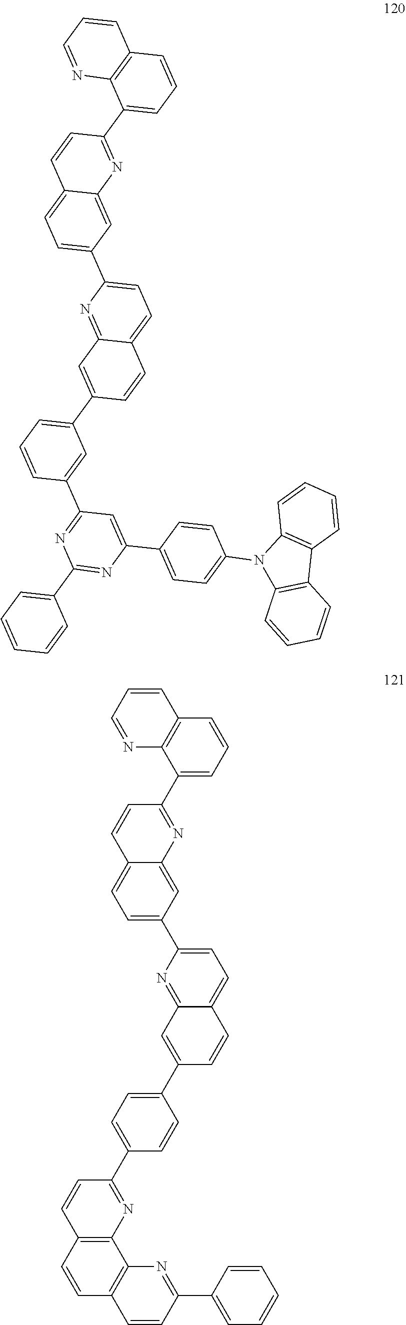 [SMILES: c1ccc(-c2nc(-c3ccc(-n4c5ccccc5c5ccccc54)cc3)cc(-c3cccc(-c4ccc5ccc(-c6ccc7ccc(-c8cccc9cccnc89)nc7c6)nc5c4)c3)n2)cc1 ; c1ccc(-c2ccc3ccc4ccc(-c5ccc(-c6ccc7ccc(-c8ccc9ccc(-c%10cccc%11cccnc%10%11)nc9c8)nc7c6)cc5)nc4c3n2)cc1]

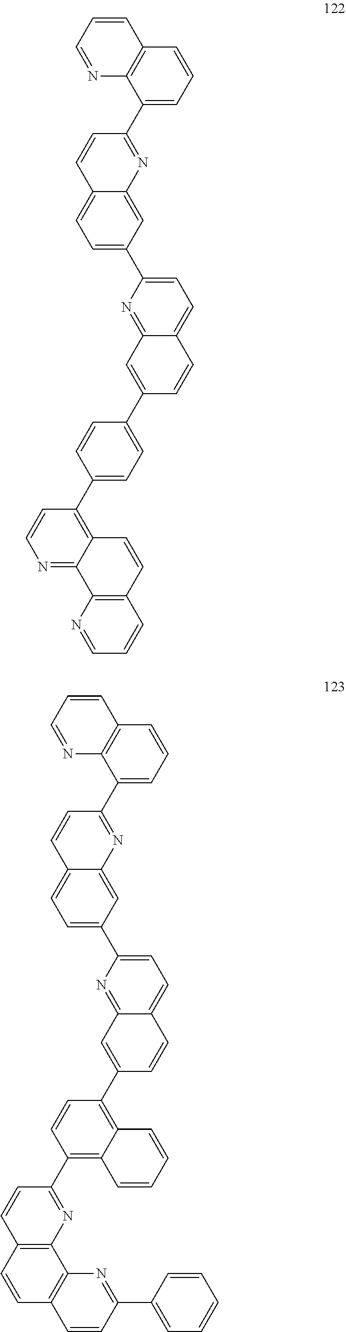

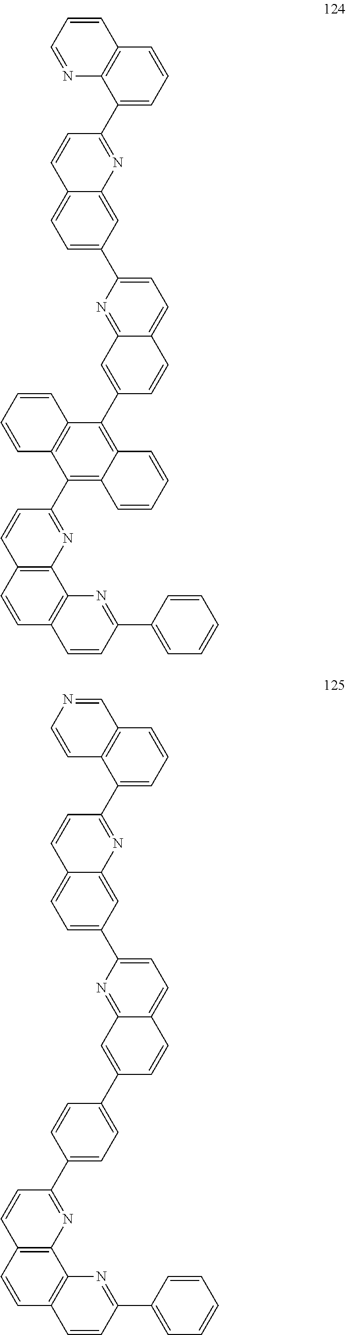

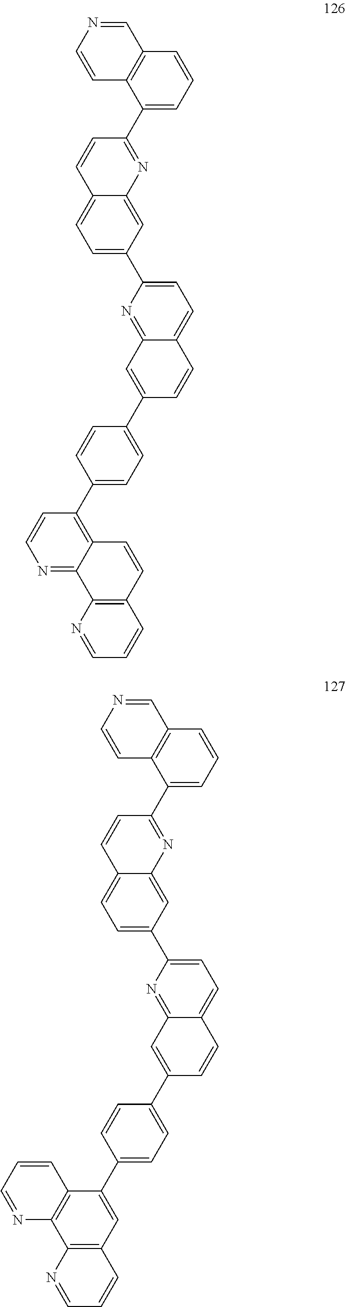

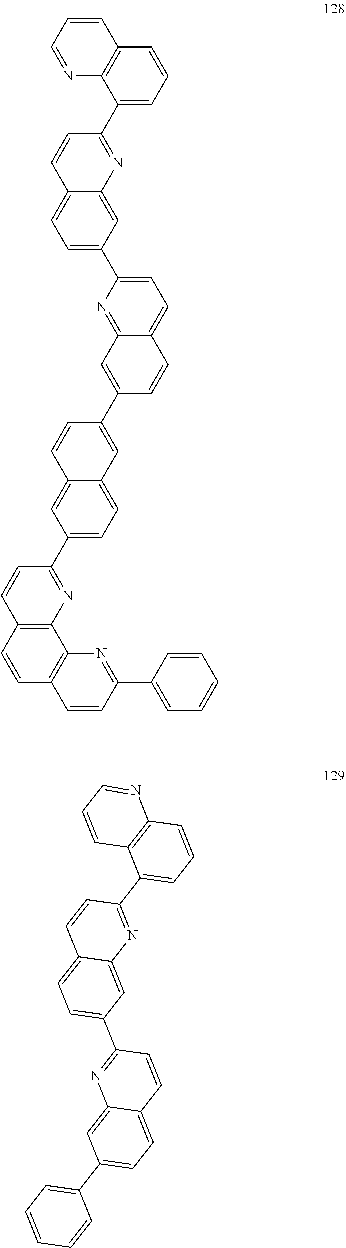 [SMILES: c1ccc(-c2ccc3ccc4ccc(-c5ccc6cc(-c7ccc8ccc(-c9ccc%10ccc(-c%11cccc%12cccnc%11%12)nc%10c9)nc8c7)ccc6c5)nc4c3n2)cc1 ; c1ccc(-c2ccc3ccc(-c4ccc5ccc(-c6cccc7ncccc67)nc5c4)nc3c2)cc1]

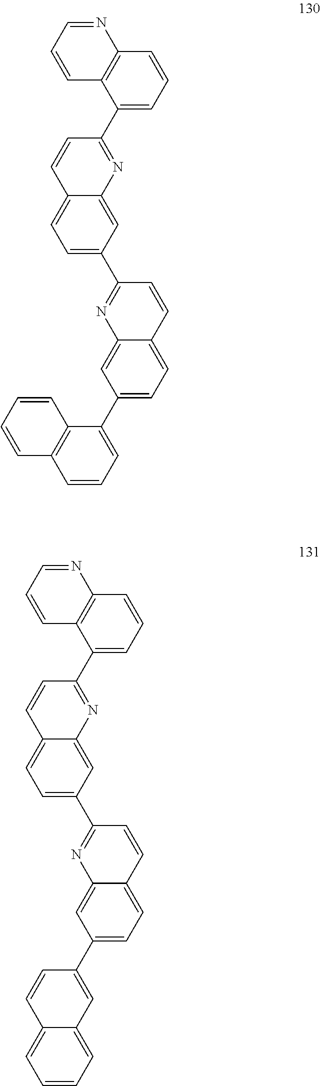 [SMILES: c1ccc2c(-c3ccc4ccc(-c5ccc6ccc(-c7cccc8ncccc78)nc6c5)nc4c3)cccc2c1 ; c1ccc2cc(-c3ccc4ccc(-c5ccc6ccc(-c7cccc8ncccc78)nc6c5)nc4c3)ccc2c1]

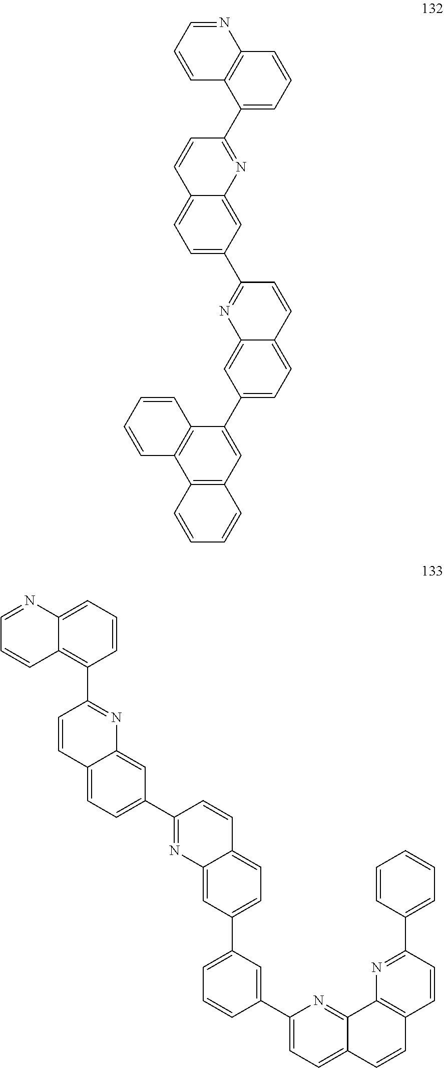 [SMILES: c1ccc2c(c1)cc(-c1ccc3ccc(-c4ccc5ccc(-c6cccc7ncccc67)nc5c4)nc3c1)c1ccccc12 ; c1ccc(-c2ccc3ccc4ccc(-c5cccc(-c6ccc7ccc(-c8ccc9ccc(-c%10cccc%11ncccc%10%11)nc9c8)nc7c6)c5)nc4c3n2)cc1]

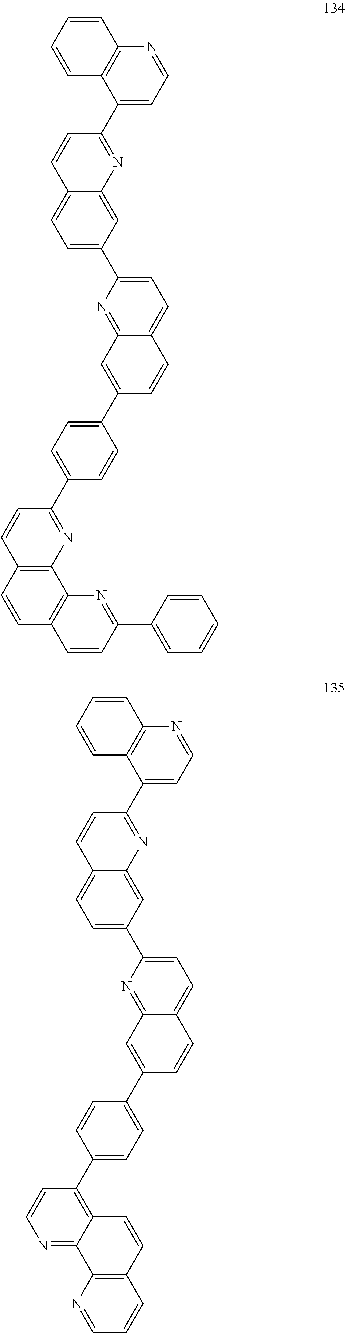

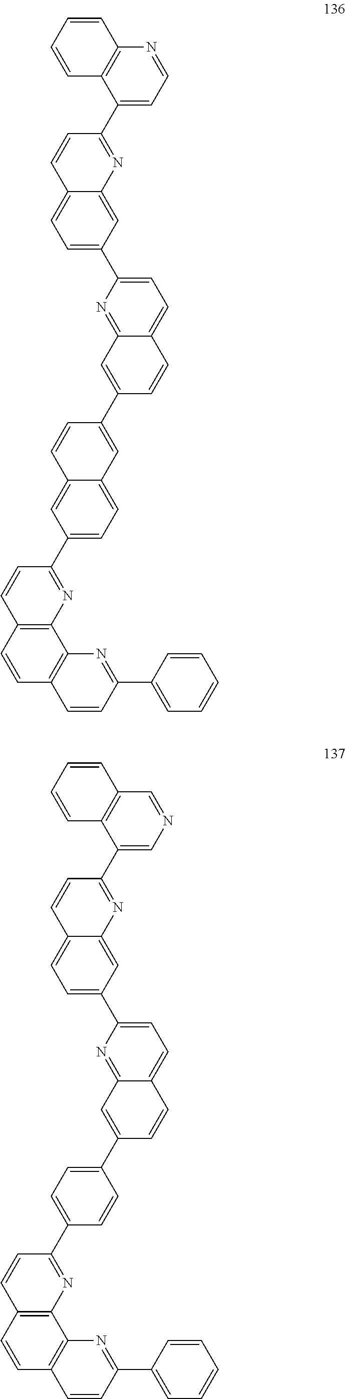 [SMILES: c1ccc(-c2ccc3ccc4ccc(-c5ccc6cc(-c7ccc8ccc(-c9ccc%10ccc(-c%11ccnc%12ccccc%11%12)nc%10c9)nc8c7)ccc6c5)nc4c3n2)cc1 ; c1ccc(-c2ccc3ccc4ccc(-c5ccc(-c6ccc7ccc(-c8ccc9ccc(-c%10cncc%11ccccc%10%11)nc9c8)nc7c6)cc5)nc4c3n2)cc1]

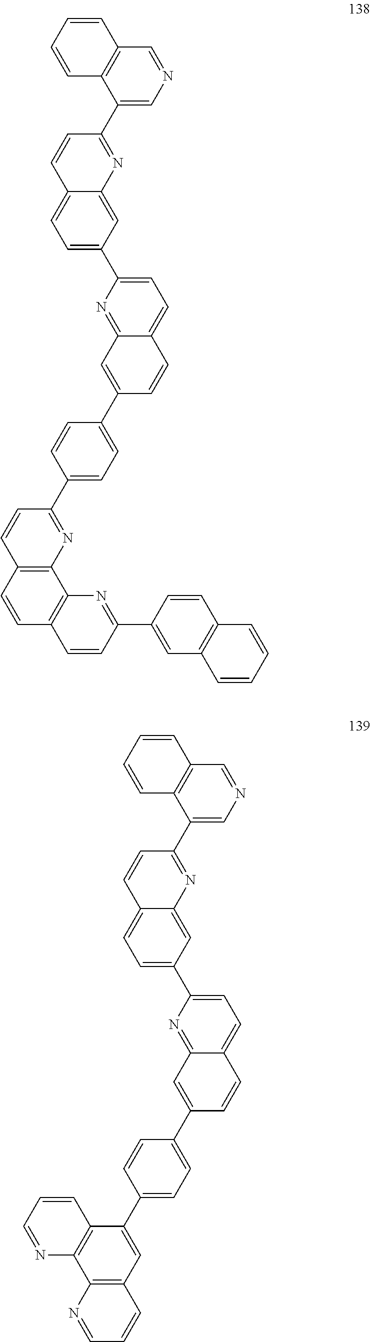 [SMILES: c1ccc2cc(-c3ccc4ccc5ccc(-c6ccc(-c7ccc8ccc(-c9ccc%10ccc(-c%11cncc%12ccccc%11%12)nc%10c9)nc8c7)cc6)nc5c4n3)ccc2c1 ; c1ccc2c(-c3ccc4ccc(-c5ccc6ccc(-c7ccc(-c8cc9cccnc9c9ncccc89)cc7)cc6n5)cc4n3)cncc2c1]

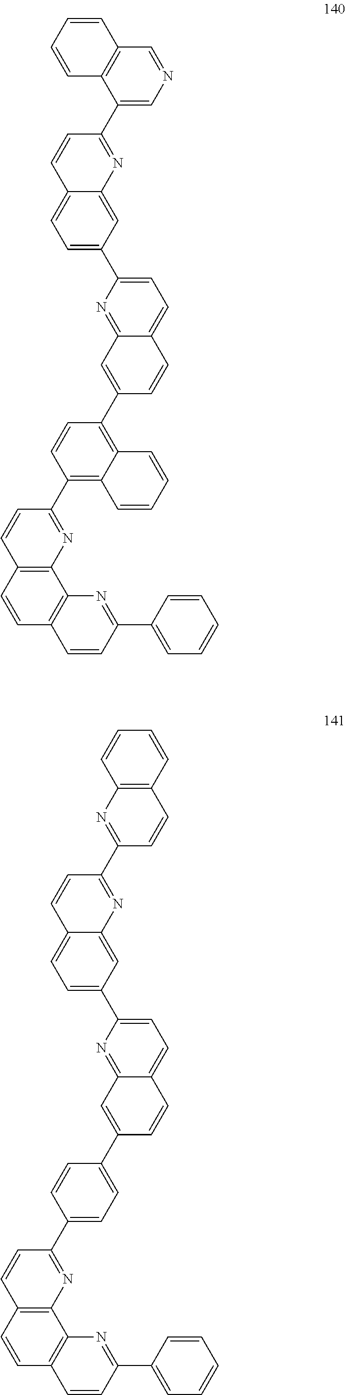 [SMILES: c1ccc(-c2ccc3ccc4ccc(-c5ccc(-c6ccc7ccc(-c8ccc9ccc(-c%10cncc%11ccccc%10%11)nc9c8)nc7c6)c6ccccc56)nc4c3n2)cc1 ; c1ccc(-c2ccc3ccc4ccc(-c5ccc(-c6ccc7ccc(-c8ccc9ccc(-c%10ccc%11ccccc%11n%10)nc9c8)nc7c6)cc5)nc4c3n2)cc1]

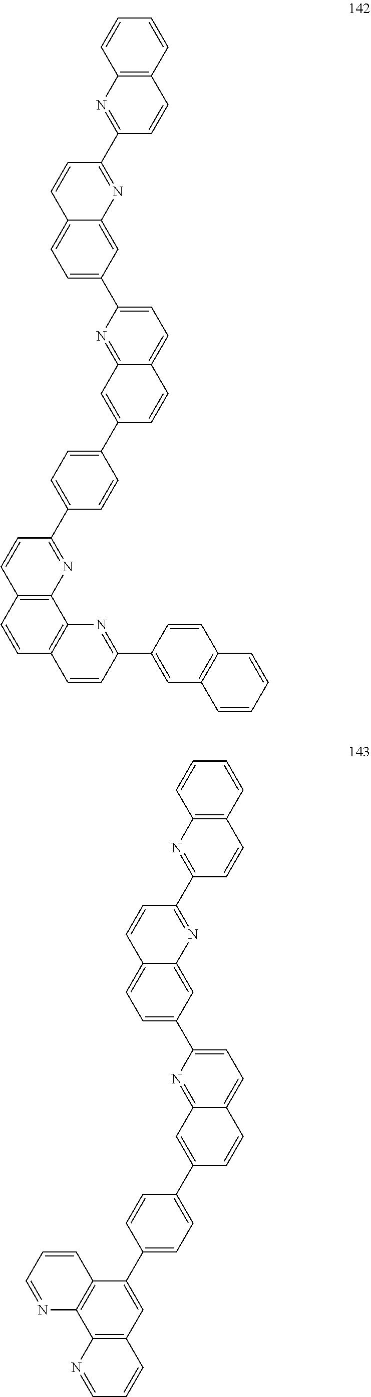 [SMILES: c1ccc2cc(-c3ccc4ccc5ccc(-c6ccc(-c7ccc8ccc(-c9ccc%10ccc(-c%11ccc%12ccccc%12n%11)nc%10c9)nc8c7)cc6)nc5c4n3)ccc2c1 ; c1ccc2nc(-c3ccc4ccc(-c5ccc6ccc(-c7ccc(-c8cc9cccnc9c9ncccc89)cc7)cc6n5)cc4n3)ccc2c1]

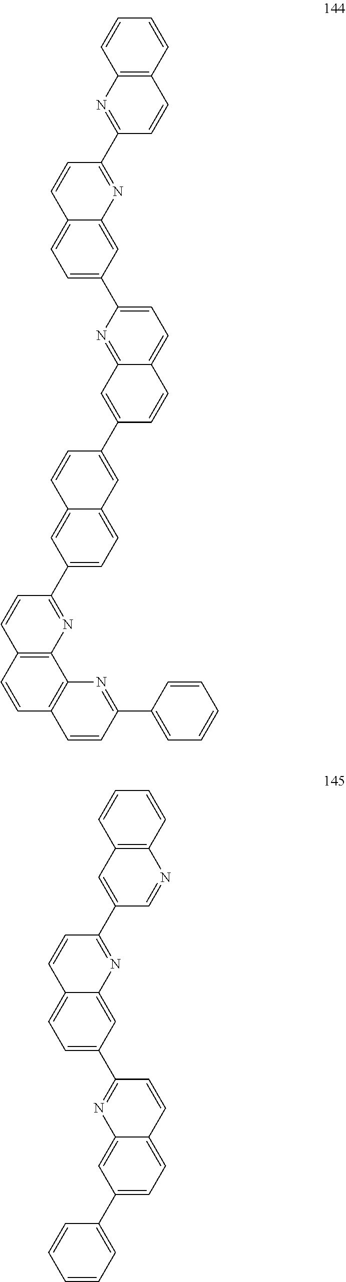 [SMILES: c1ccc(-c2ccc3ccc4ccc(-c5ccc6cc(-c7ccc8ccc(-c9ccc%10ccc(-c%11ccc%12ccccc%12n%11)nc%10c9)nc8c7)ccc6c5)nc4c3n2)cc1 ; c1ccc(-c2ccc3ccc(-c4ccc5ccc(-c6cnc7ccccc7c6)nc5c4)nc3c2)cc1]

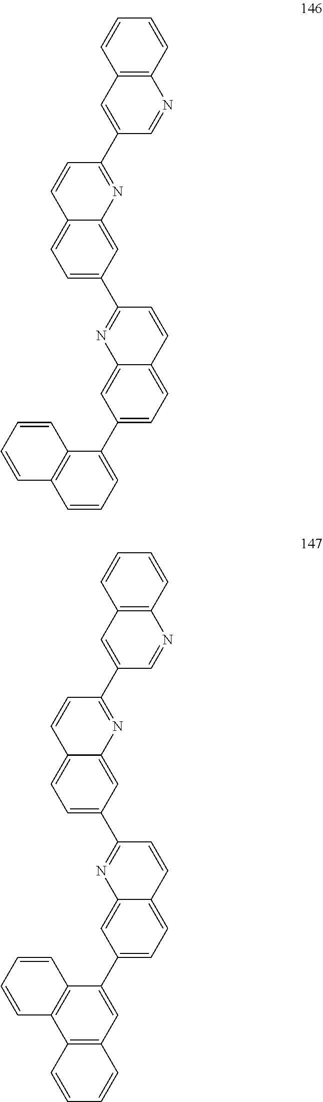 [SMILES: c1ccc2ncc(-c3ccc4ccc(-c5ccc6ccc(-c7cccc8ccccc78)cc6n5)cc4n3)cc2c1 ; c1ccc2ncc(-c3ccc4ccc(-c5ccc6ccc(-c7cc8ccccc8c8ccccc78)cc6n5)cc4n3)cc2c1]

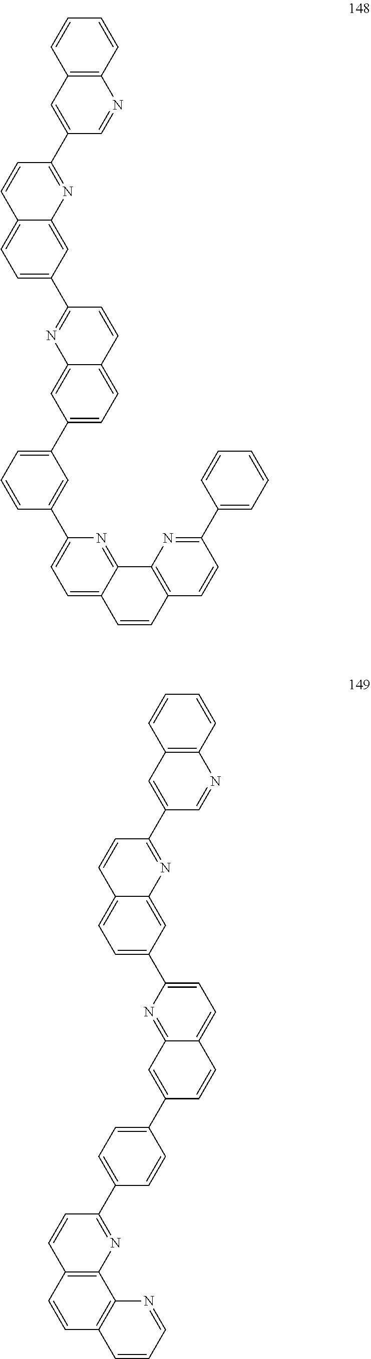 [SMILES: c1ccc(-c2ccc3ccc4ccc(-c5cccc(-c6ccc7ccc(-c8ccc9ccc(-c%10cnc%11ccccc%11c%10)nc9c8)nc7c6)c5)nc4c3n2)cc1 ; c1ccc2ncc(-c3ccc4ccc(-c5ccc6ccc(-c7ccc(-c8ccc9ccc%10cccnc%10c9n8)cc7)cc6n5)cc4n3)cc2c1]

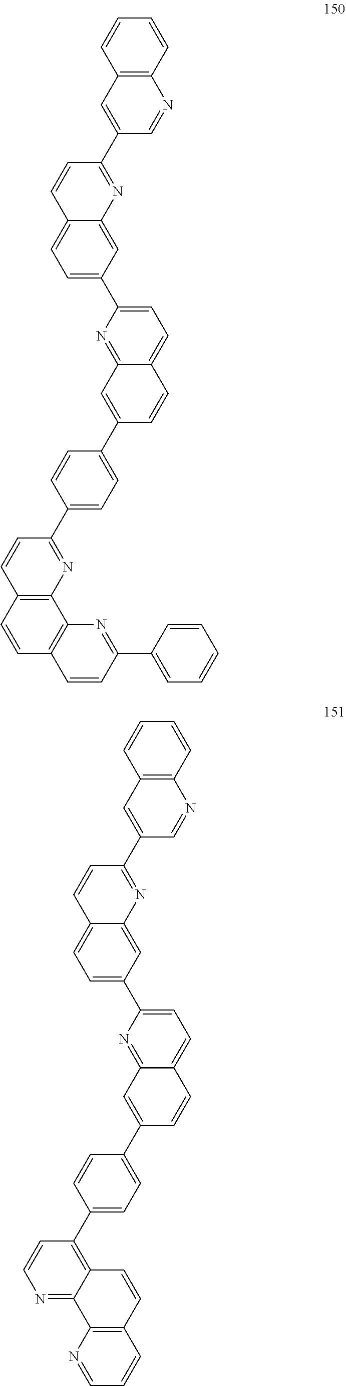

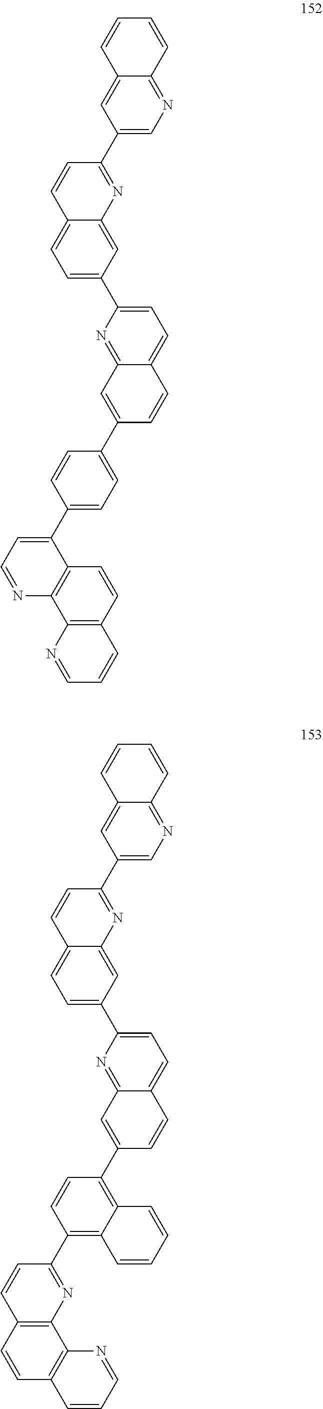 [SMILES: c1ccc2ncc(-c3ccc4ccc(-c5ccc6ccc(-c7ccc(-c8ccnc9c8ccc8cccnc89)cc7)cc6n5)cc4n3)cc2c1 ; c1ccc2ncc(-c3ccc4ccc(-c5ccc6ccc(-c7ccc(-c8ccc9ccc%10cccnc%10c9n8)c8ccccc78)cc6n5)cc4n3)cc2c1]

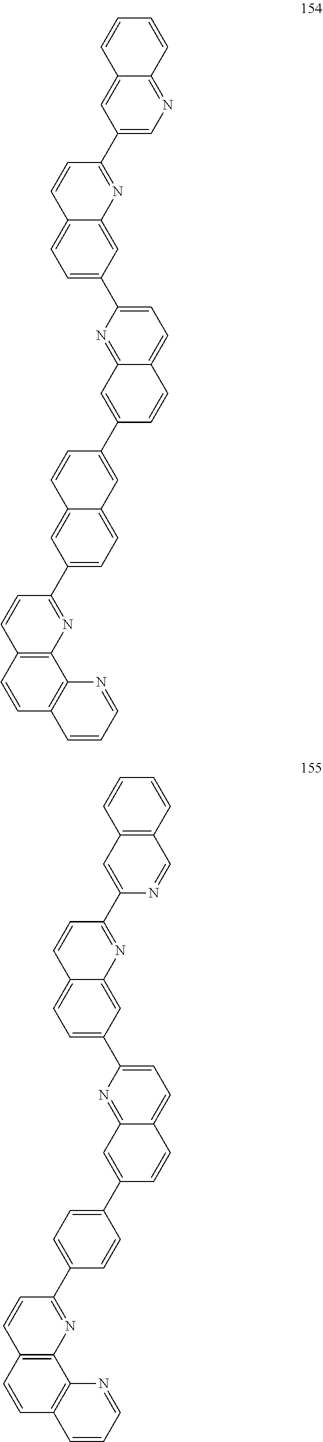

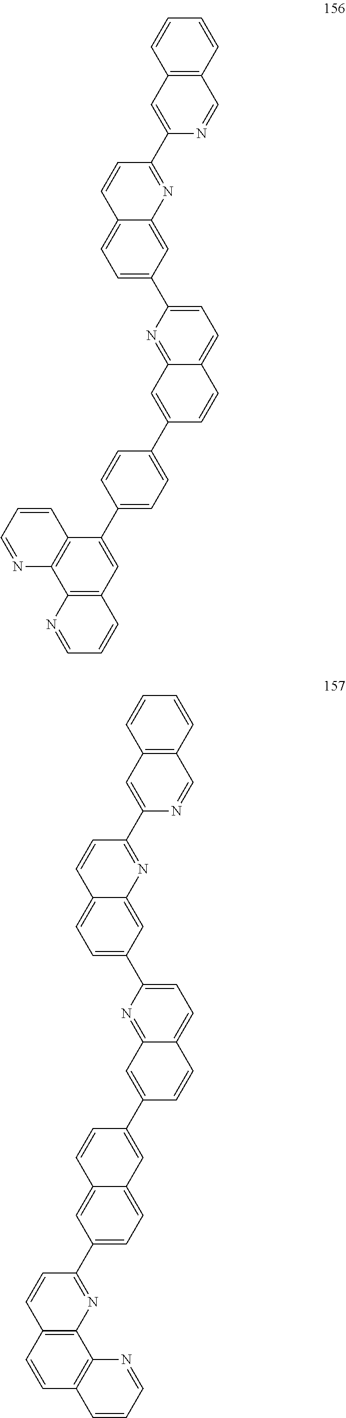 [SMILES: c1ccc2cc(-c3ccc4ccc(-c5ccc6ccc(-c7ccc(-c8cc9cccnc9c9ncccc89)cc7)cc6n5)cc4n3)ncc2c1 ; c1ccc2cc(-c3ccc4ccc(-c5ccc6ccc(-c7ccc8cc(-c9ccc%10ccc%11cccnc%11c%10n9)ccc8c7)cc6n5)cc4n3)ncc2c1]

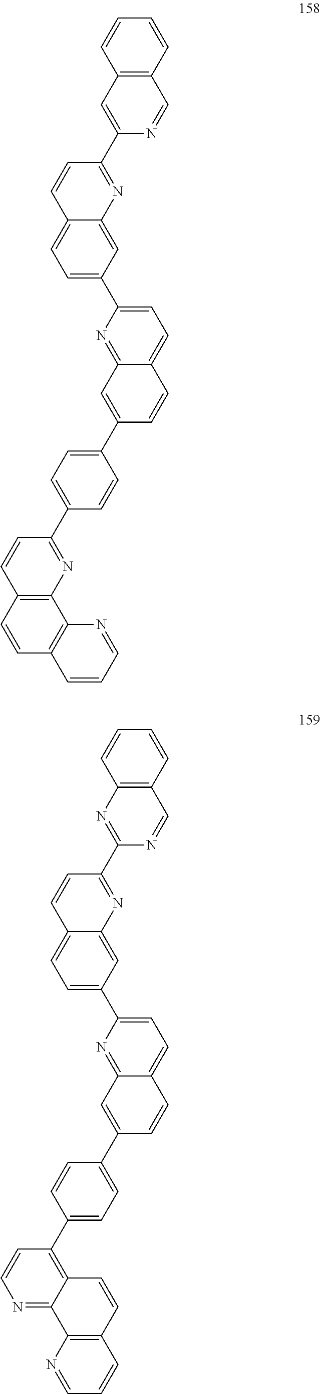 [SMILES: c1ccc2cc(-c3ccc4ccc(-c5ccc6ccc(-c7ccc(-c8ccc9ccc%10cccnc%10c9n8)cc7)cc6n5)cc4n3)ncc2c1 ; c1ccc2nc(-c3ccc4ccc(-c5ccc6ccc(-c7ccc(-c8ccnc9c8ccc8cccnc89)cc7)cc6n5)cc4n3)ncc2c1]

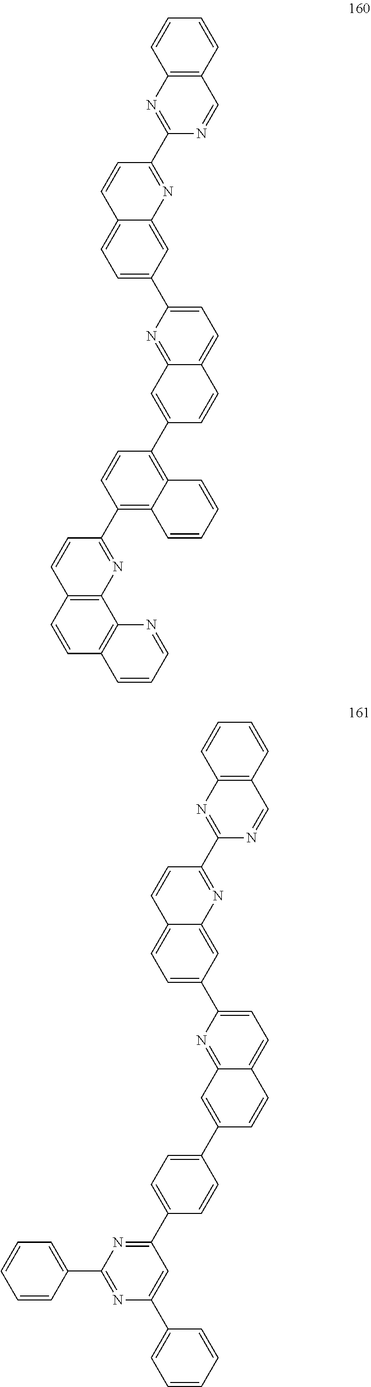 [SMILES: c1ccc2nc(-c3ccc4ccc(-c5ccc6ccc(-c7ccc(-c8ccc9ccc%10cccnc%10c9n8)c8ccccc78)cc6n5)cc4n3)ncc2c1 ; c1ccc(-c2cc(-c3ccc(-c4ccc5ccc(-c6ccc7ccc(-c8ncc9ccccc9n8)nc7c6)nc5c4)cc3)nc(-c3ccccc3)n2)cc1]

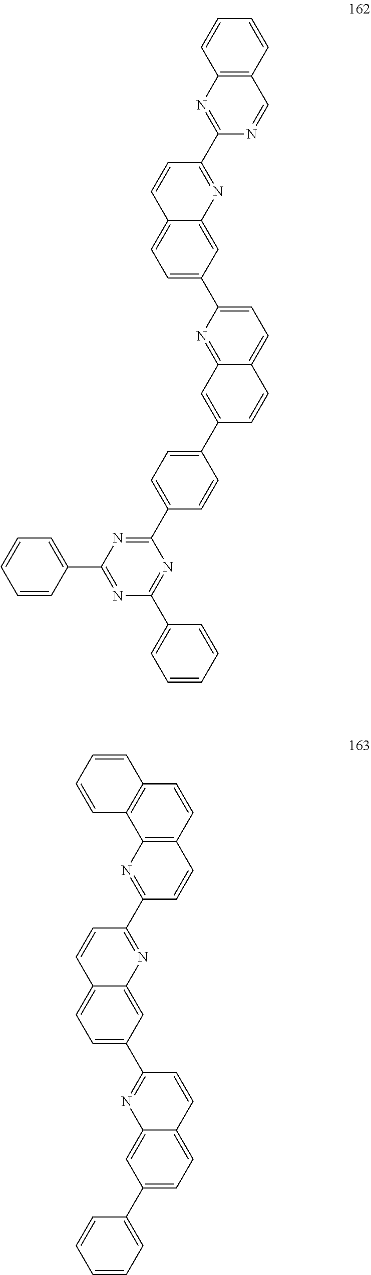 [SMILES: c1ccc(-c2nc(-c3ccccc3)nc(-c3ccc(-c4ccc5ccc(-c6ccc7ccc(-c8ncc9ccccc9n8)nc7c6)nc5c4)cc3)n2)cc1 ; c1ccc(-c2ccc3ccc(-c4ccc5ccc(-c6ccc7ccc8ccccc8c7n6)nc5c4)nc3c2)cc1]

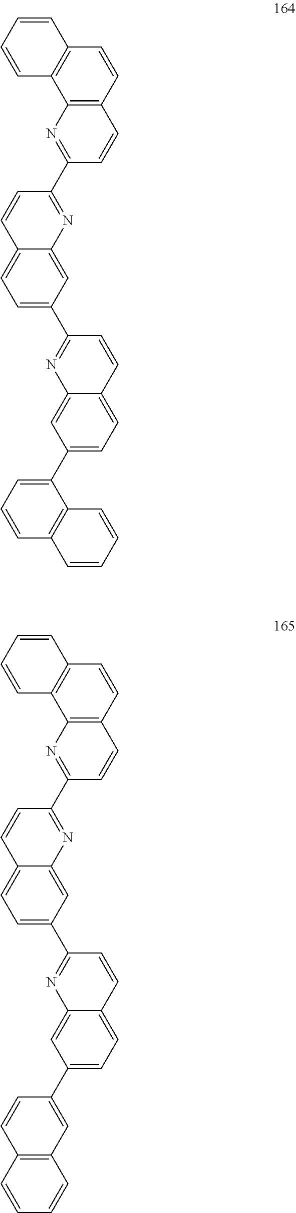 [SMILES: c1ccc2c(-c3ccc4ccc(-c5ccc6ccc(-c7ccc8ccc9ccccc9c8n7)nc6c5)nc4c3)cccc2c1 ; c1ccc2cc(-c3ccc4ccc(-c5ccc6ccc(-c7ccc8ccc9ccccc9c8n7)nc6c5)nc4c3)ccc2c1]

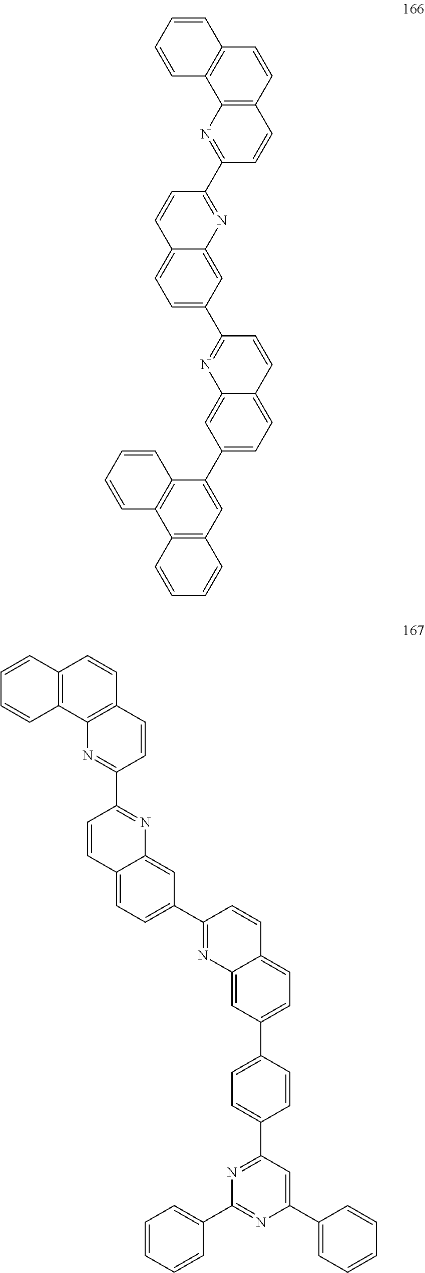 [SMILES: c1ccc2c(c1)cc(-c1ccc3ccc(-c4ccc5ccc(-c6ccc7ccc8ccccc8c7n6)nc5c4)nc3c1)c1ccccc12 ; c1ccc(-c2cc(-c3ccc(-c4ccc5ccc(-c6ccc7ccc(-c8ccc9ccc%10ccccc%10c9n8)nc7c6)nc5c4)cc3)nc(-c3ccccc3)n2)cc1]

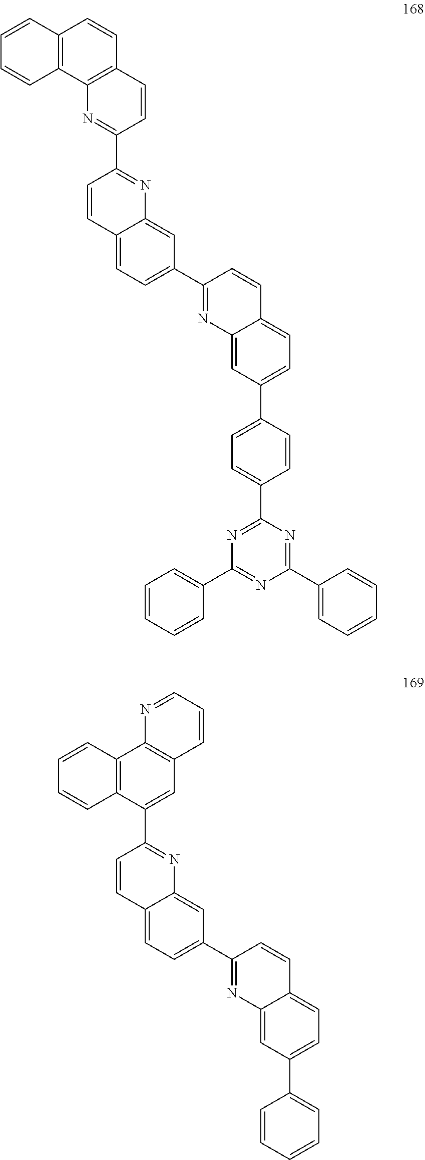 [SMILES: c1ccc(-c2nc(-c3ccccc3)nc(-c3ccc(-c4ccc5ccc(-c6ccc7ccc(-c8ccc9ccc%10ccccc%10c9n8)nc7c6)nc5c4)cc3)n2)cc1 ; c1ccc(-c2ccc3ccc(-c4ccc5ccc(-c6cc7cccnc7c7ccccc67)nc5c4)nc3c2)cc1]

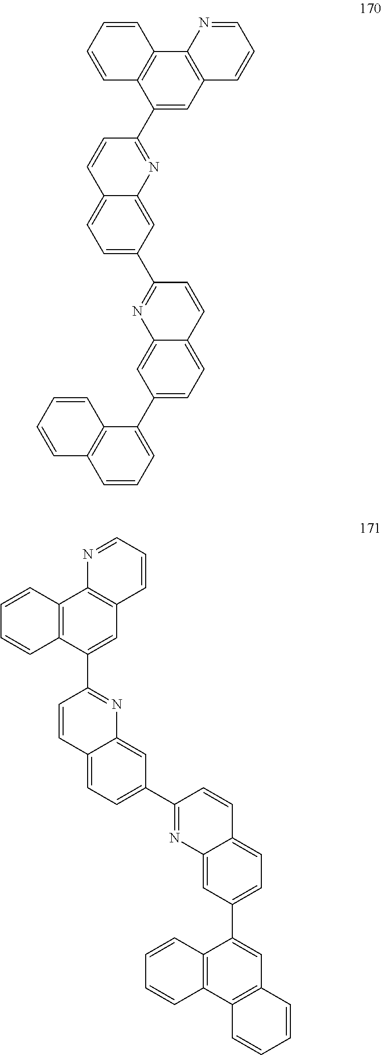 [SMILES: c1ccc2c(-c3ccc4ccc(-c5ccc6ccc(-c7cc8cccnc8c8ccccc78)nc6c5)nc4c3)cccc2c1 ; c1ccc2c(c1)cc(-c1ccc3ccc(-c4ccc5ccc(-c6cc7cccnc7c7ccccc67)nc5c4)nc3c1)c1ccccc12]

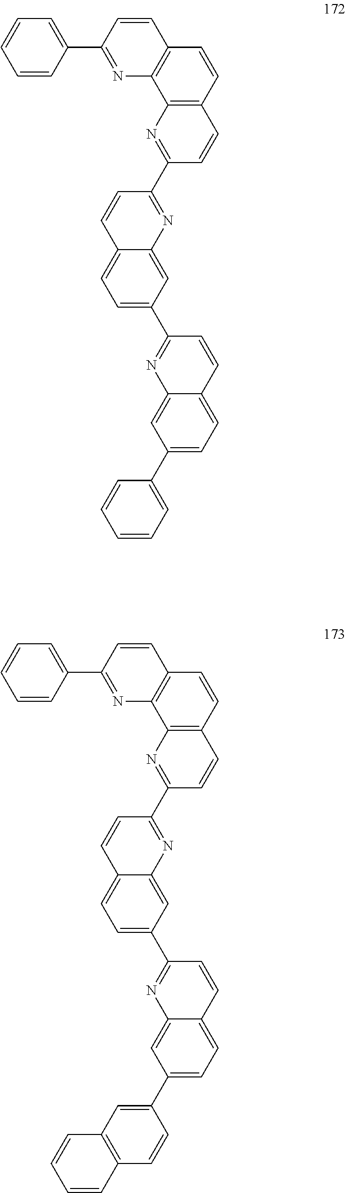 [SMILES: c1ccc(-c2ccc3ccc(-c4ccc5ccc(-c6ccc7ccc8ccc(-c9ccccc9)nc8c7n6)nc5c4)nc3c2)cc1 ; c1ccc(-c2ccc3ccc4ccc(-c5ccc6ccc(-c7ccc8ccc(-c9ccc%10ccccc%10c9)cc8n7)cc6n5)nc4c3n2)cc1]

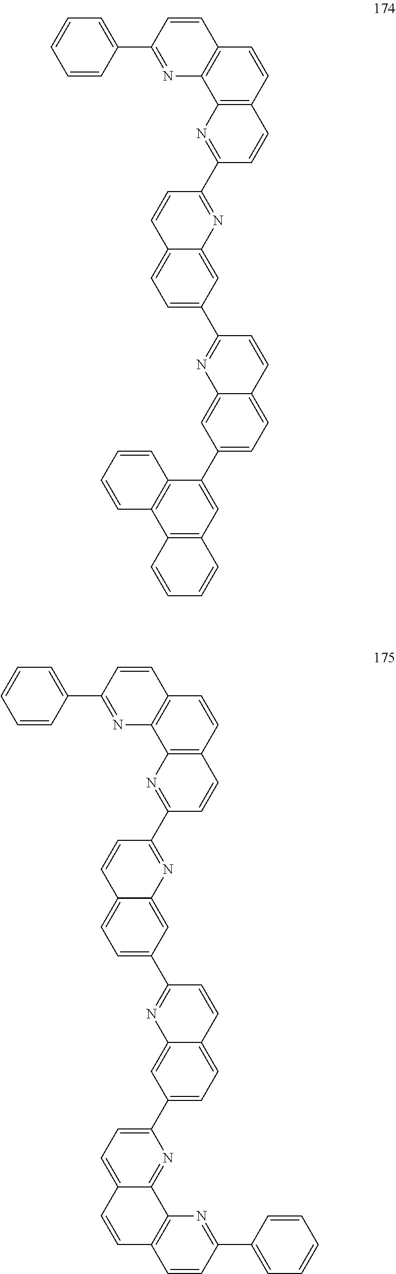 [SMILES: c1ccc(-c2ccc3ccc4ccc(-c5ccc6ccc(-c7ccc8ccc(-c9cc%10ccccc%10c%10ccccc9%10)cc8n7)cc6n5)nc4c3n2)cc1 ; c1ccc(-c2ccc3ccc4ccc(-c5ccc6ccc(-c7ccc8ccc(-c9ccc%10ccc%11ccc(-c%12ccccc%12)nc%11c%10n9)nc8c7)nc6c5)nc4c3n2)cc1]

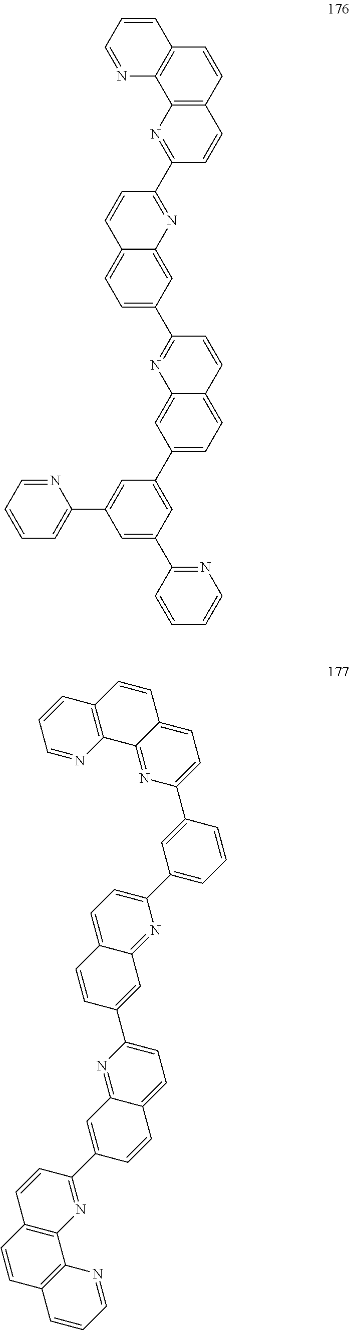 [SMILES: c1ccc(-c2cc(-c3ccc4ccc(-c5ccc6ccc(-c7ccc8ccc9cccnc9c8n7)nc6c5)nc4c3)cc(-c3ccccn3)c2)nc1 ; c1cc(-c2ccc3ccc(-c4ccc5ccc(-c6ccc7ccc8cccnc8c7n6)cc5n4)cc3n2)cc(-c2ccc3ccc4cccnc4c3n2)c1]

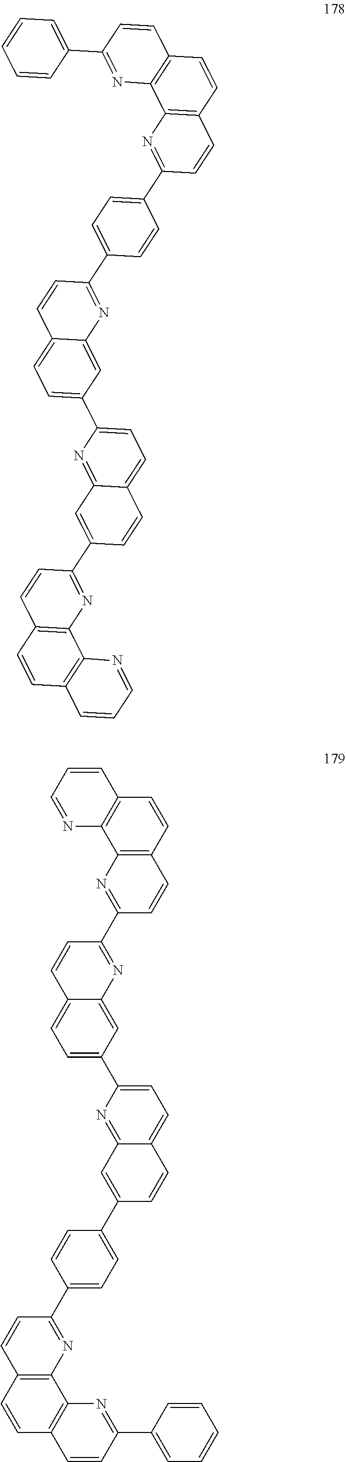 [SMILES: c1ccc(-c2ccc3ccc4ccc(-c5ccc(-c6ccc7ccc(-c8ccc9ccc(-c%10ccc%11ccc%12cccnc%12c%11n%10)cc9n8)cc7n6)cc5)nc4c3n2)cc1 ; c1ccc(-c2ccc3ccc4ccc(-c5ccc(-c6ccc7ccc(-c8ccc9ccc(-c%10ccc%11ccc%12cccnc%12c%11n%10)nc9c8)nc7c6)cc5)nc4c3n2)cc1]

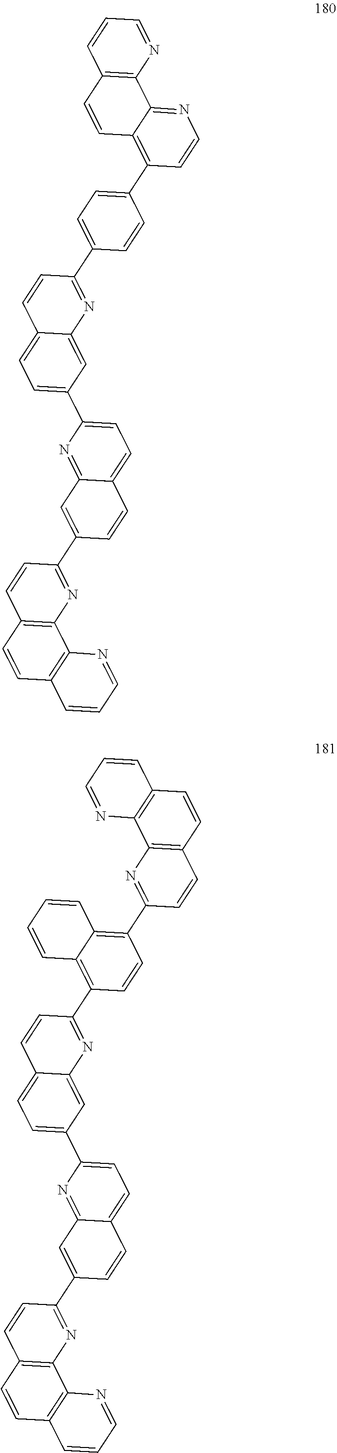

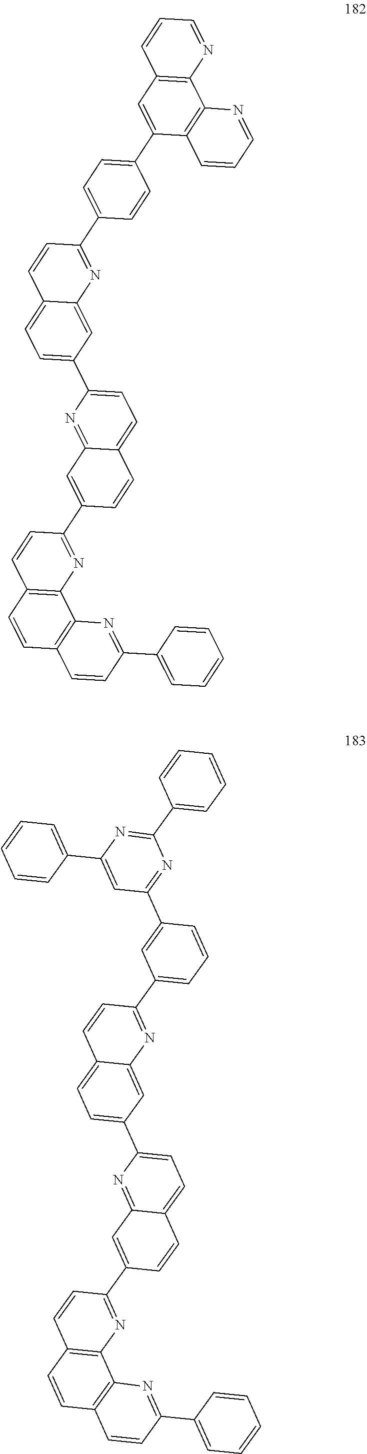 [SMILES: c1ccc(-c2ccc3ccc4ccc(-c5ccc6ccc(-c7ccc8ccc(-c9ccc(-c%10cc%11cccnc%11c%11ncccc%10%11)cc9)nc8c7)nc6c5)nc4c3n2)cc1 ; c1ccc(-c2cc(-c3cccc(-c4ccc5ccc(-c6ccc7ccc(-c8ccc9ccc%10ccc(-c%11ccccc%11)nc%10c9n8)cc7n6)cc5n4)c3)nc(-c3ccccc3)n2)cc1]

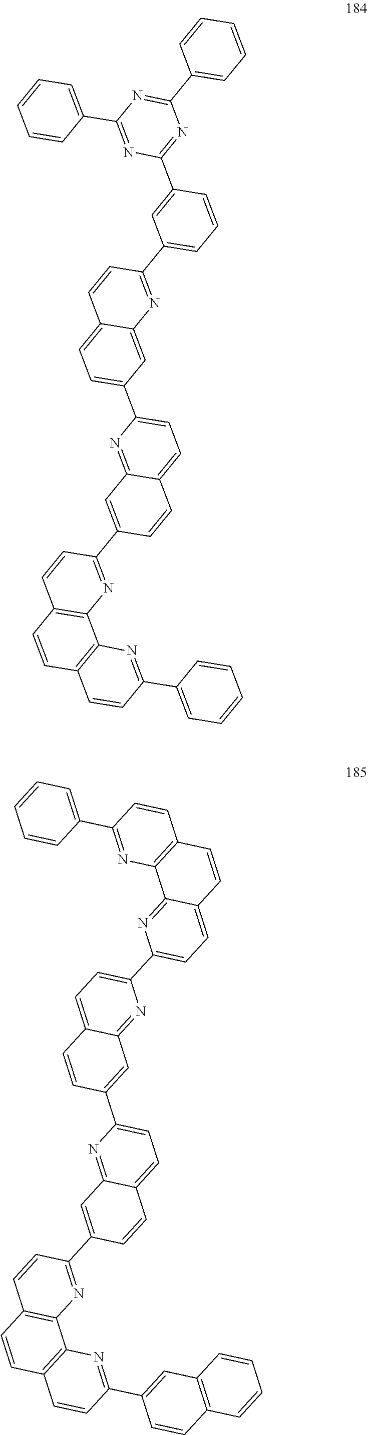 [SMILES: c1ccc(-c2ccc3ccc4ccc(-c5ccc6ccc(-c7ccc8ccc(-c9cccc(-c%10nc(-c%11ccccc%11)nc(-c%11ccccc%11)n%10)c9)nc8c7)nc6c5)nc4c3n2)cc1 ; c1ccc(-c2ccc3ccc4ccc(-c5ccc6ccc(-c7ccc8ccc(-c9ccc%10ccc%11ccc(-c%12ccc%13ccccc%13c%12)nc%11c%10n9)cc8n7)cc6n5)nc4c3n2)cc1]

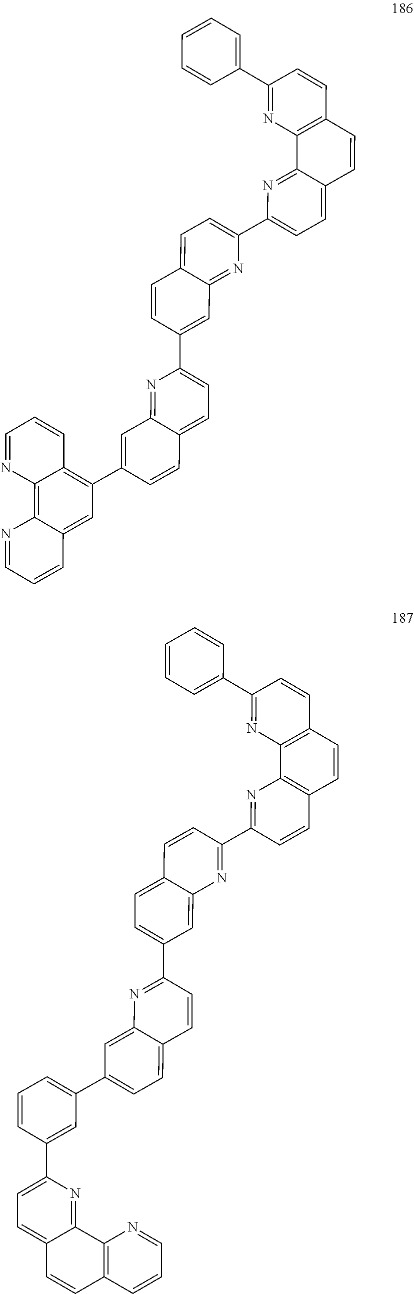 [SMILES: c1ccc(-c2ccc3ccc4ccc(-c5ccc6ccc(-c7ccc8ccc(-c9cc%10cccnc%10c%10ncccc9%10)cc8n7)cc6n5)nc4c3n2)cc1 ; c1ccc(-c2ccc3ccc4ccc(-c5ccc6ccc(-c7ccc8ccc(-c9cccc(-c%10ccc%11ccc%12cccnc%12c%11n%10)c9)cc8n7)cc6n5)nc4c3n2)cc1]

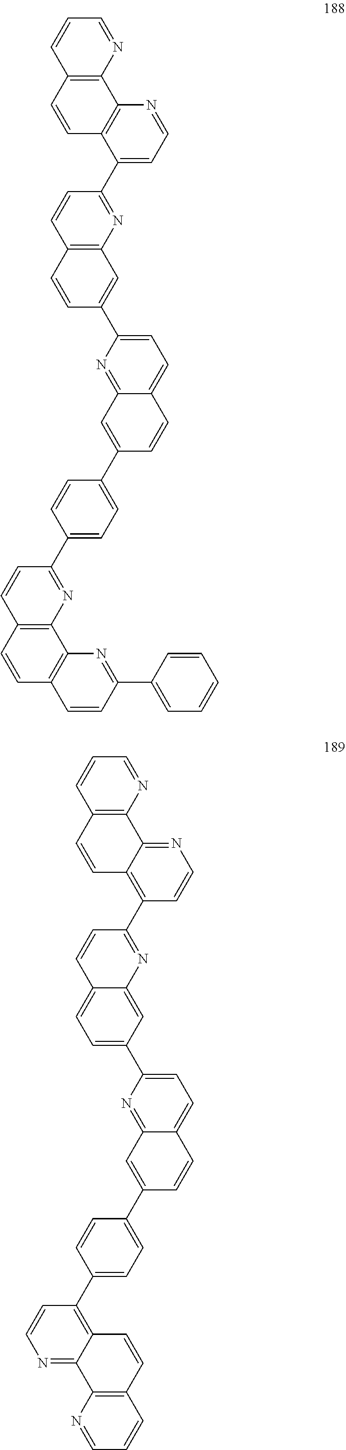

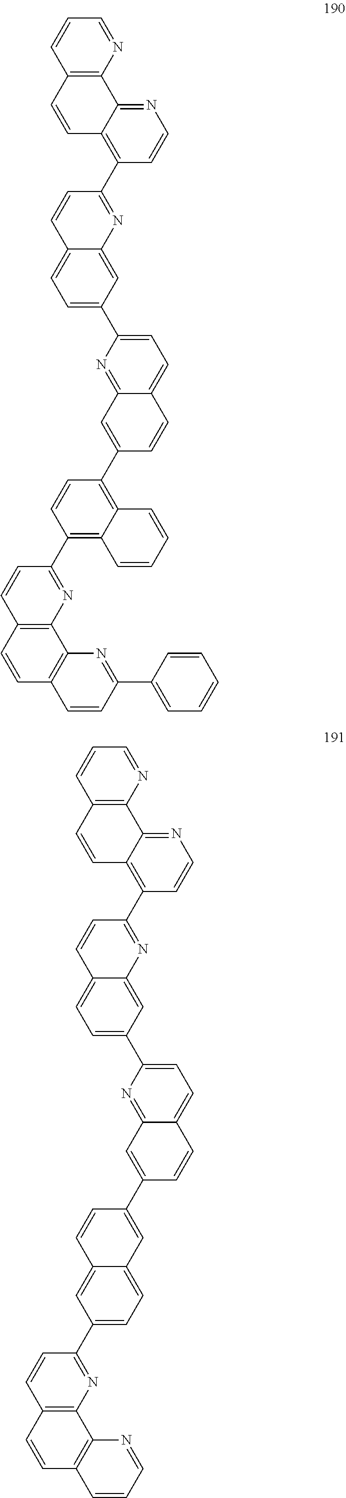

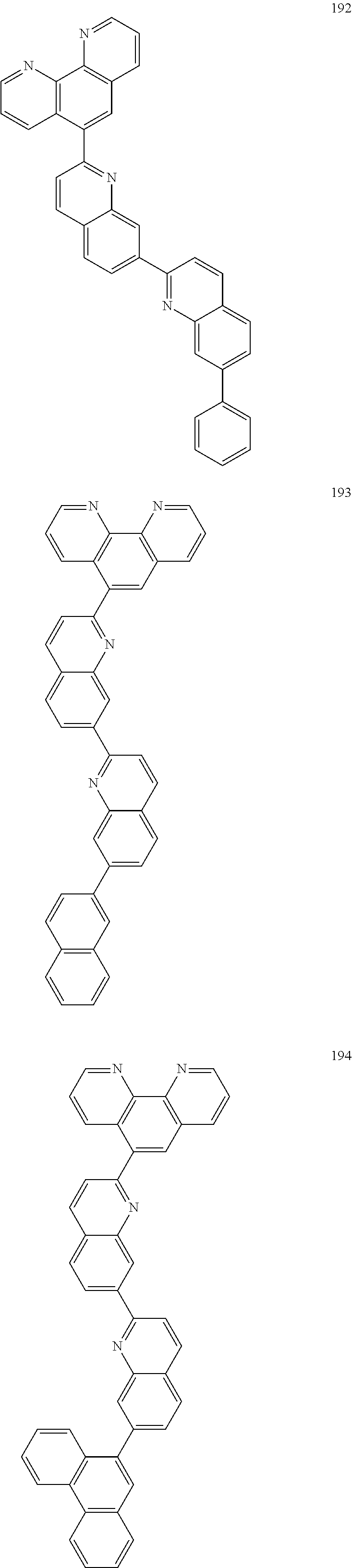 [SMILES: c1ccc(-c2ccc3ccc(-c4ccc5ccc(-c6cc7cccnc7c7ncccc67)nc5c4)nc3c2)cc1 ; c1ccc2cc(-c3ccc4ccc(-c5ccc6ccc(-c7cc8cccnc8c8ncccc78)nc6c5)nc4c3)ccc2c1 ; c1ccc2c(c1)cc(-c1ccc3ccc(-c4ccc5ccc(-c6cc7cccnc7c7ncccc67)nc5c4)nc3c1)c1ccccc12]

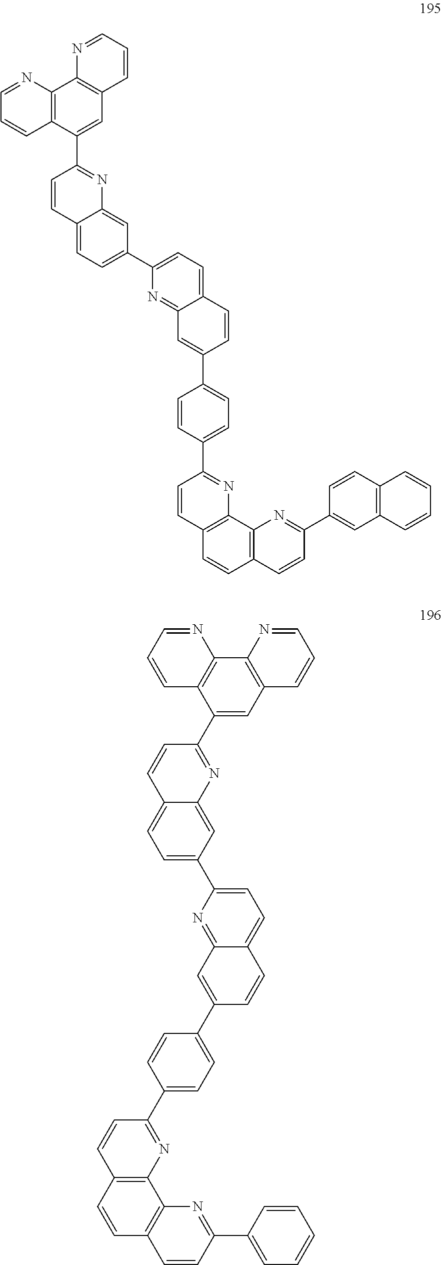 [SMILES: c1ccc2cc(-c3ccc4ccc5ccc(-c6ccc(-c7ccc8ccc(-c9ccc%10ccc(-c%11cc%12cccnc%12c%12ncccc%11%12)nc%10c9)nc8c7)cc6)nc5c4n3)ccc2c1 ; c1ccc(-c2ccc3ccc4ccc(-c5ccc(-c6ccc7ccc(-c8ccc9ccc(-c%10cc%11cccnc%11c%11ncccc%10%11)nc9c8)nc7c6)cc5)nc4c3n2)cc1]

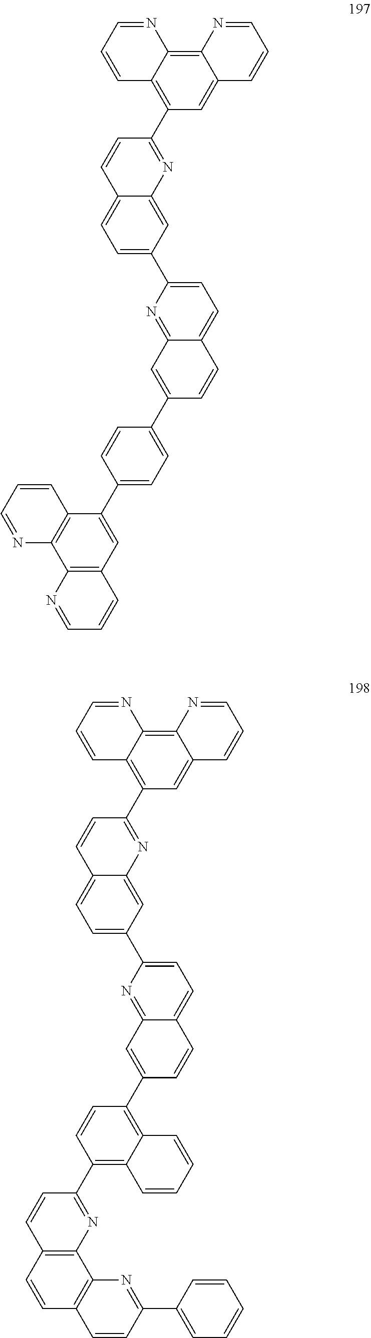 [SMILES: c1cnc2c(c1)cc(-c1ccc(-c3ccc4ccc(-c5ccc6ccc(-c7cc8cccnc8c8ncccc78)nc6c5)nc4c3)cc1)c1cccnc12 ; c1ccc(-c2ccc3ccc4ccc(-c5ccc(-c6ccc7ccc(-c8ccc9ccc(-c%10cc%11cccnc%11c%11ncccc%10%11)nc9c8)nc7c6)c6ccccc56)nc4c3n2)cc1]

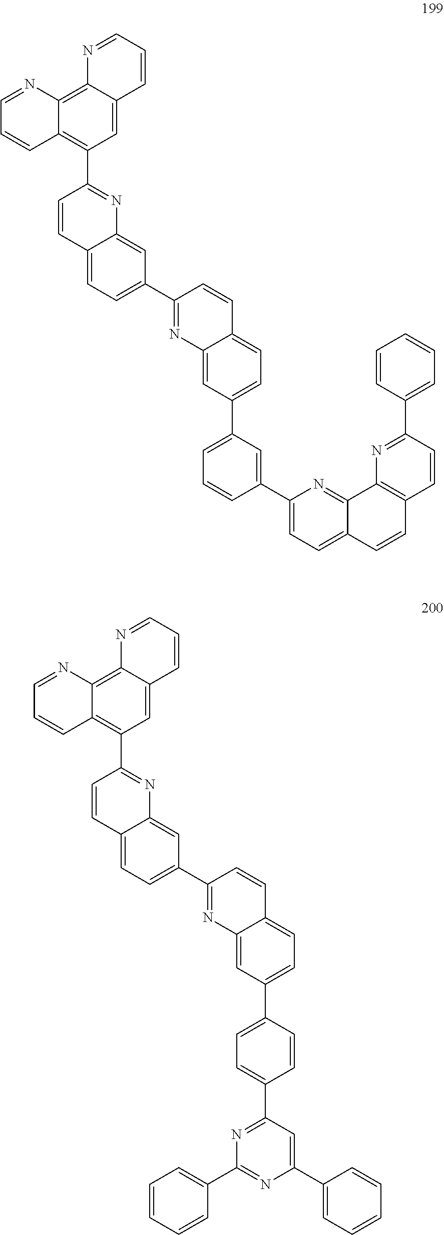 [SMILES: c1ccc(-c2ccc3ccc4ccc(-c5cccc(-c6ccc7ccc(-c8ccc9ccc(-c%10cc%11cccnc%11c%11ncccc%10%11)nc9c8)nc7c6)c5)nc4c3n2)cc1 ; c1ccc(-c2cc(-c3ccc(-c4ccc5ccc(-c6ccc7ccc(-c8cc9cccnc9c9ncccc89)nc7c6)nc5c4)cc3)nc(-c3ccccc3)n2)cc1]

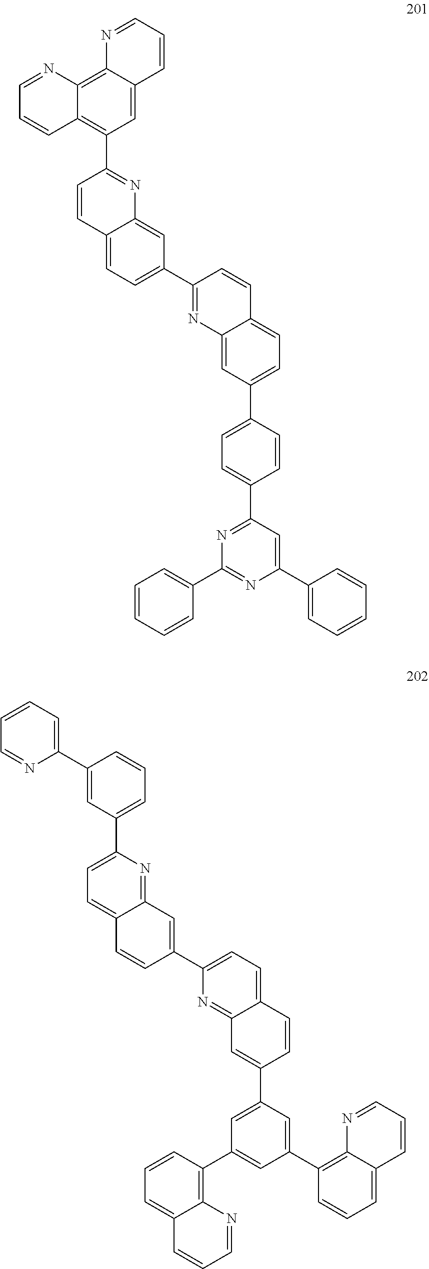 [SMILES: c1ccc(-c2cc(-c3ccc(-c4ccc5ccc(-c6ccc7ccc(-c8cc9cccnc9c9ncccc89)nc7c6)nc5c4)cc3)nc(-c3ccccc3)n2)cc1 ; c1ccc(-c2cccc(-c3ccc4ccc(-c5ccc6ccc(-c7cc(-c8cccc9cccnc89)cc(-c8cccc9cccnc89)c7)cc6n5)cc4n3)c2)nc1]

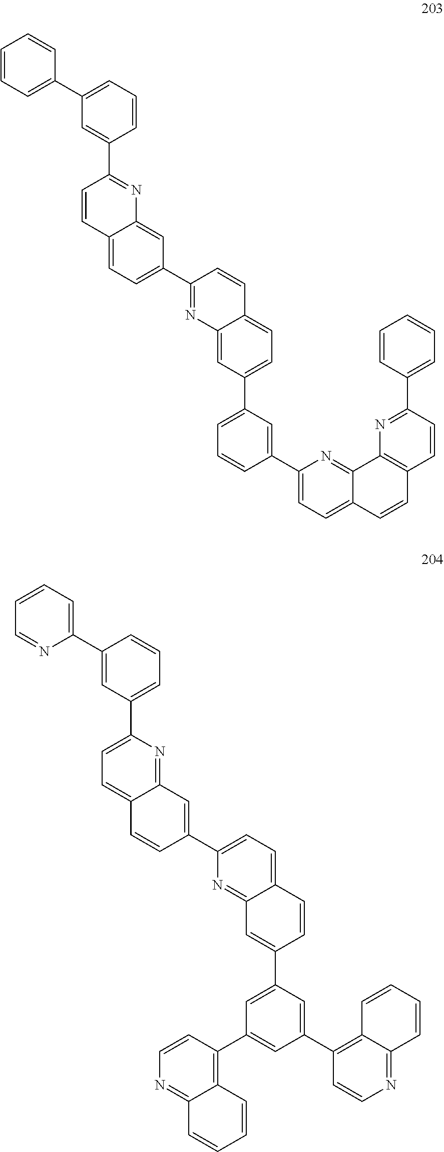 [SMILES: c1ccc(-c2cccc(-c3ccc4ccc(-c5ccc6ccc(-c7cccc(-c8ccc9ccc%10ccc(-c%11ccccc%11)nc%10c9n8)c7)cc6n5)cc4n3)c2)cc1 ; c1ccc(-c2cccc(-c3ccc4ccc(-c5ccc6ccc(-c7cc(-c8ccnc9ccccc89)cc(-c8ccnc9ccccc89)c7)cc6n5)cc4n3)c2)nc1]

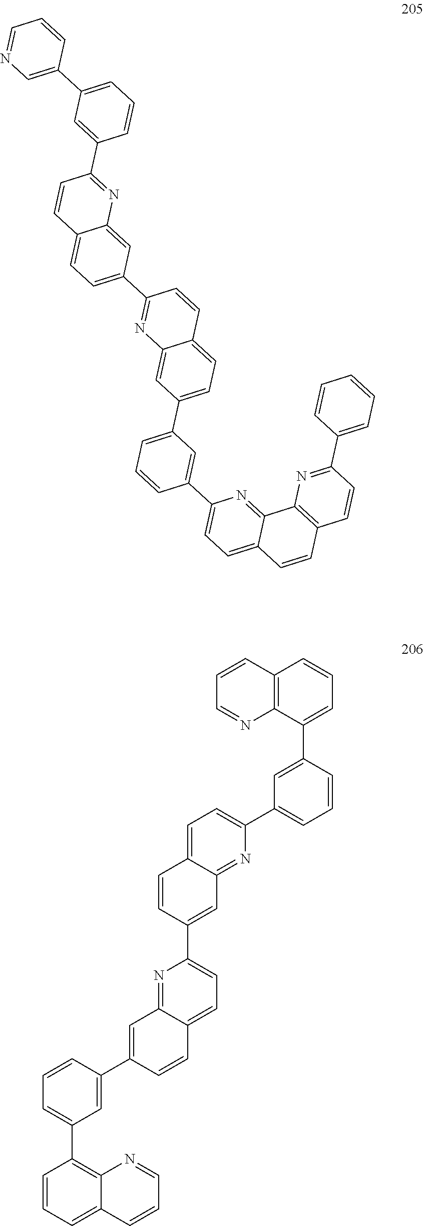 [SMILES: c1ccc(-c2ccc3ccc4ccc(-c5cccc(-c6ccc7ccc(-c8ccc9ccc(-c%10cccc(-c%11cccnc%11)c%10)nc9c8)nc7c6)c5)nc4c3n2)cc1 ; c1cc(-c2ccc3ccc(-c4ccc5ccc(-c6cccc(-c7cccc8cccnc78)c6)nc5c4)nc3c2)cc(-c2cccc3cccnc23)c1]

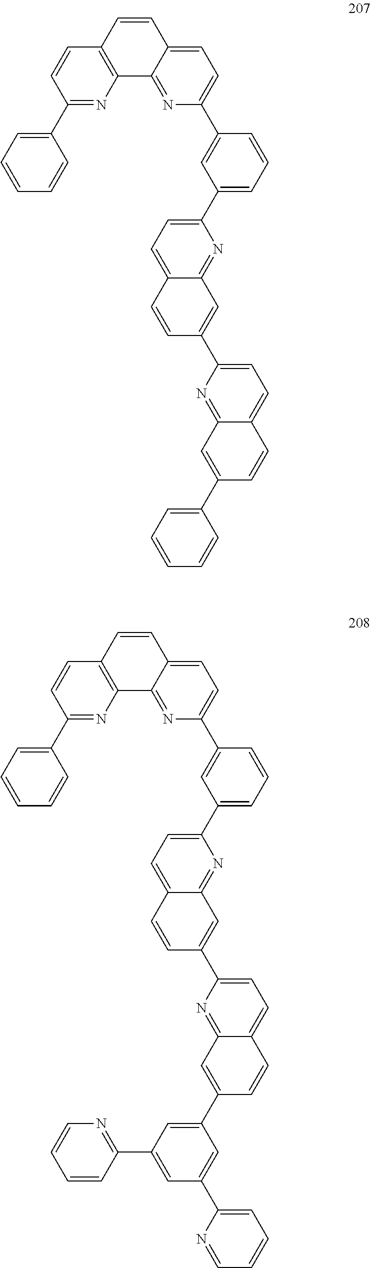 [SMILES: c1ccc(-c2ccc3ccc(-c4ccc5ccc(-c6cccc(-c7ccc8ccc9ccc(-c%10ccccc%10)nc9c8n7)c6)nc5c4)nc3c2)cc1 ; c1ccc(-c2ccc3ccc4ccc(-c5cccc(-c6ccc7ccc(-c8ccc9ccc(-c%10cc(-c%11ccccn%11)cc(-c%11ccccn%11)c%10)cc9n8)cc7n6)c5)nc4c3n2)cc1]

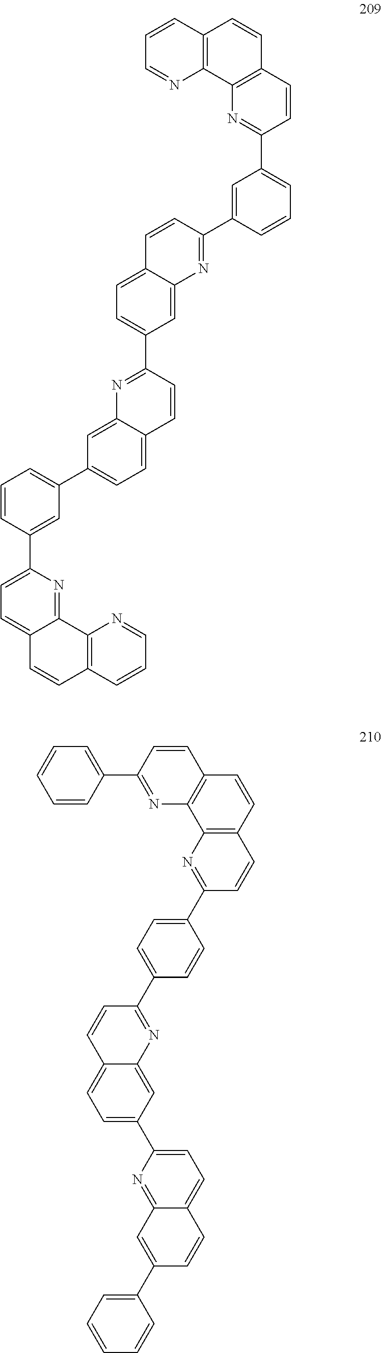 [SMILES: c1cc(-c2ccc3ccc(-c4ccc5ccc(-c6cccc(-c7ccc8ccc9cccnc9c8n7)c6)nc5c4)nc3c2)cc(-c2ccc3ccc4cccnc4c3n2)c1 ; c1ccc(-c2ccc3ccc(-c4ccc5ccc(-c6ccc(-c7ccc8ccc9ccc(-c%10ccccc%10)nc9c8n7)cc6)nc5c4)nc3c2)cc1]

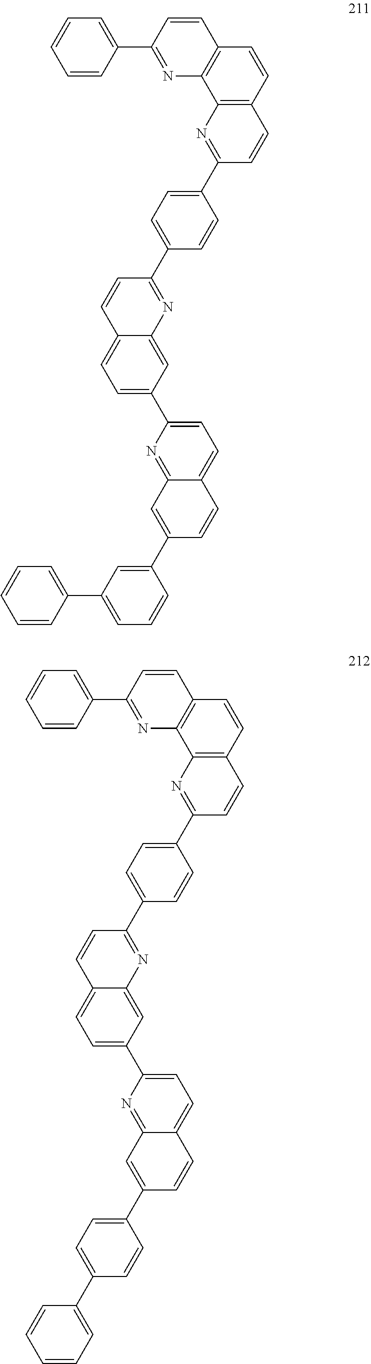 [SMILES: c1ccc(-c2cccc(-c3ccc4ccc(-c5ccc6ccc(-c7ccc(-c8ccc9ccc%10ccc(-c%11ccccc%11)nc%10c9n8)cc7)nc6c5)nc4c3)c2)cc1 ; c1ccc(-c2ccc(-c3ccc4ccc(-c5ccc6ccc(-c7ccc(-c8ccc9ccc%10ccc(-c%11ccccc%11)nc%10c9n8)cc7)nc6c5)nc4c3)cc2)cc1]

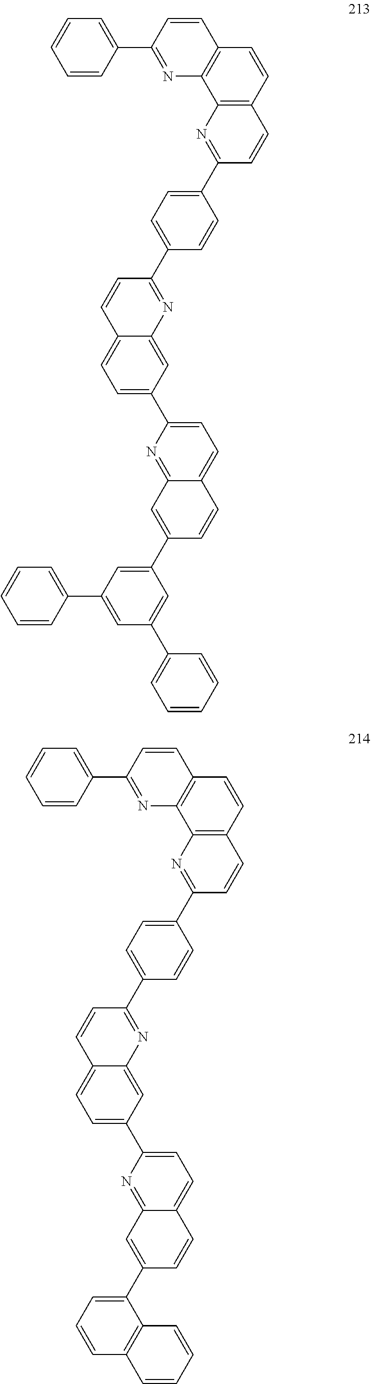

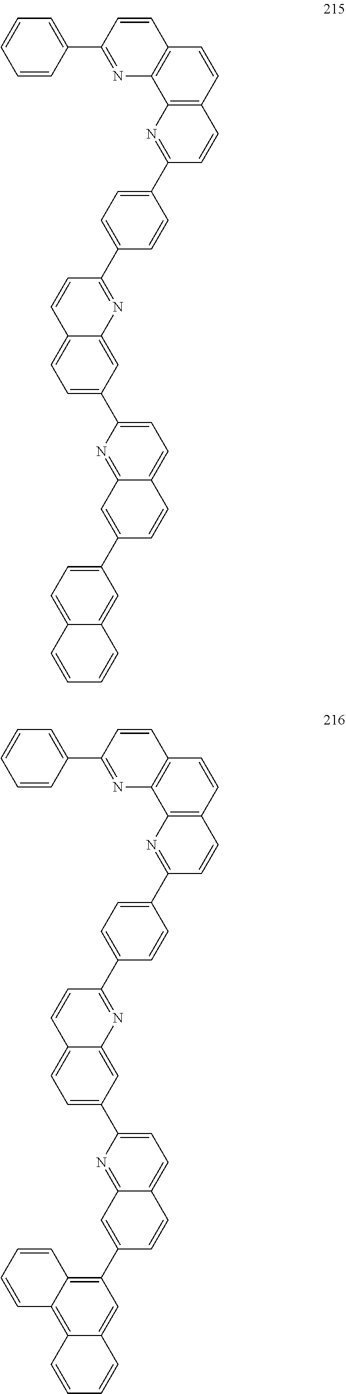 [SMILES: c1ccc(-c2ccc3ccc4ccc(-c5ccc(-c6ccc7ccc(-c8ccc9ccc(-c%10ccc%11ccccc%11c%10)cc9n8)cc7n6)cc5)nc4c3n2)cc1 ; c1ccc(-c2ccc3ccc4ccc(-c5ccc(-c6ccc7ccc(-c8ccc9ccc(-c%10cc%11ccccc%11c%11ccccc%10%11)cc9n8)cc7n6)cc5)nc4c3n2)cc1]

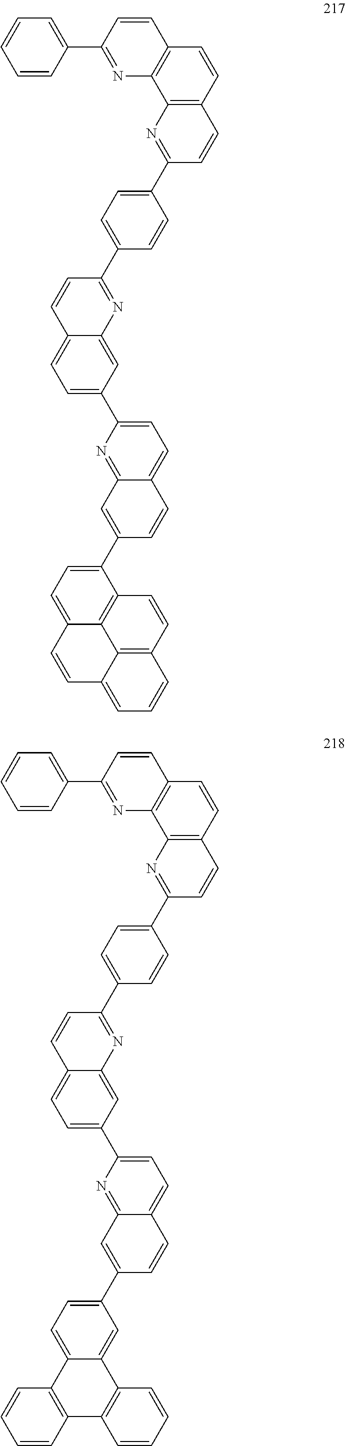

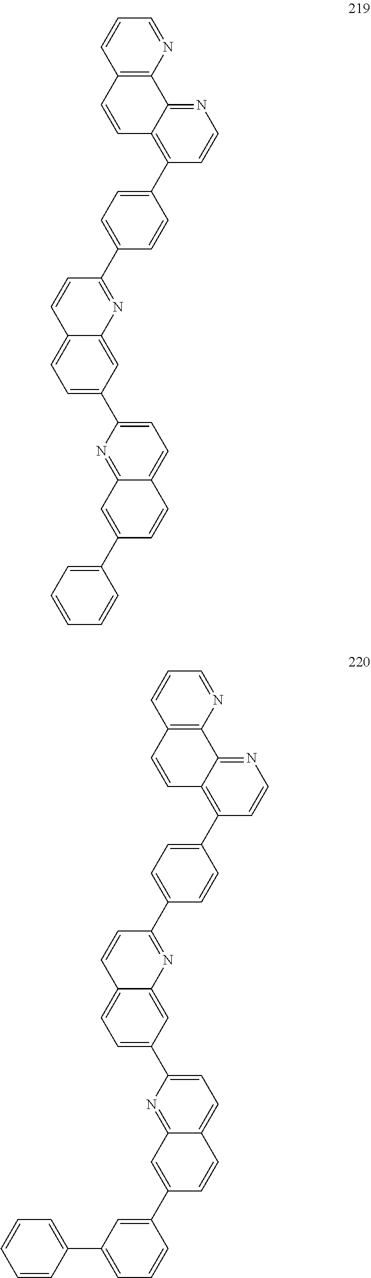 [SMILES: c1ccc(-c2ccc3ccc(-c4ccc5ccc(-c6ccc(-c7ccnc8c7ccc7cccnc78)cc6)nc5c4)nc3c2)cc1 ; c1ccc(-c2cccc(-c3ccc4ccc(-c5ccc6ccc(-c7ccc(-c8ccnc9c8ccc8cccnc89)cc7)nc6c5)nc4c3)c2)cc1]

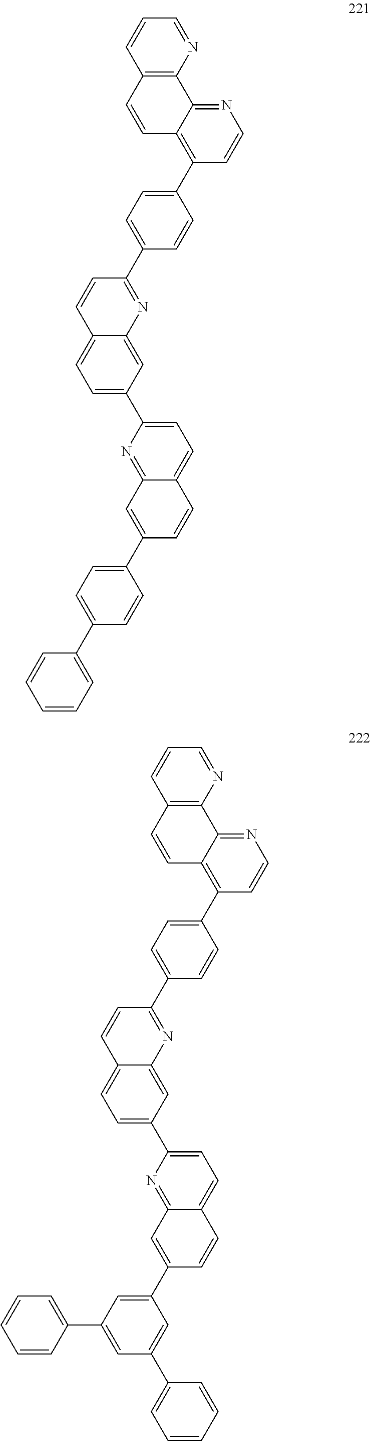 [SMILES: c1ccc(-c2ccc(-c3ccc4ccc(-c5ccc6ccc(-c7ccc(-c8ccnc9c8ccc8cccnc89)cc7)nc6c5)nc4c3)cc2)cc1 ; c1ccc(-c2cc(-c3ccccc3)cc(-c3ccc4ccc(-c5ccc6ccc(-c7ccc(-c8ccnc9c8ccc8cccnc89)cc7)nc6c5)nc4c3)c2)cc1]

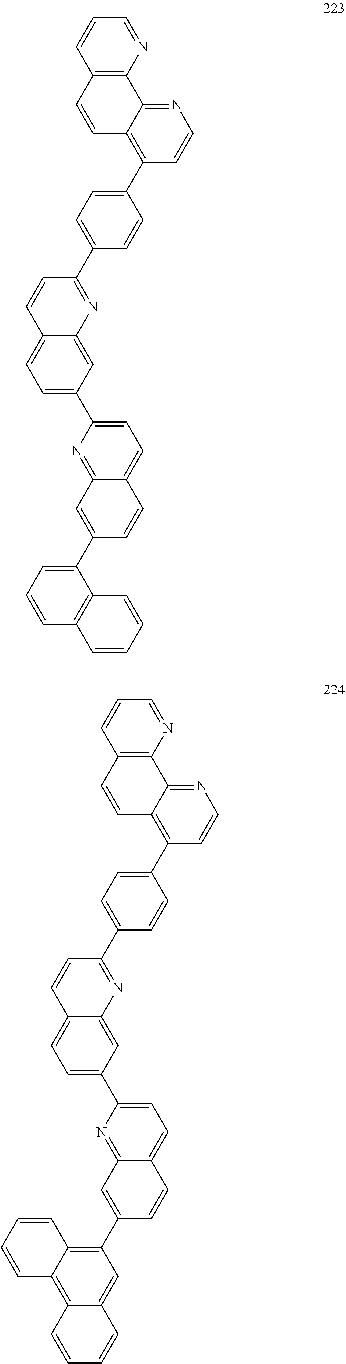 [SMILES: c1ccc2c(-c3ccc4ccc(-c5ccc6ccc(-c7ccc(-c8ccnc9c8ccc8cccnc89)cc7)nc6c5)nc4c3)cccc2c1 ; c1ccc2c(c1)cc(-c1ccc3ccc(-c4ccc5ccc(-c6ccc(-c7ccnc8c7ccc7cccnc78)cc6)nc5c4)nc3c1)c1ccccc12]

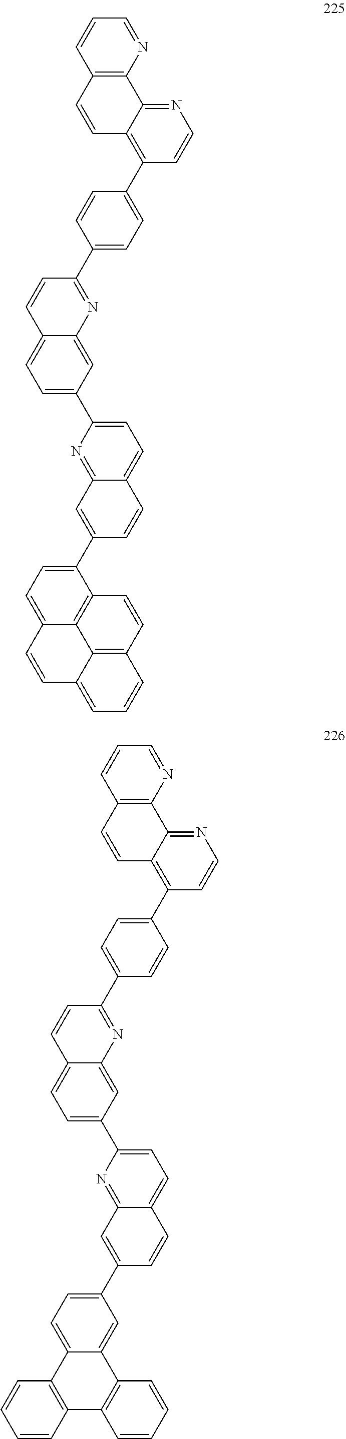

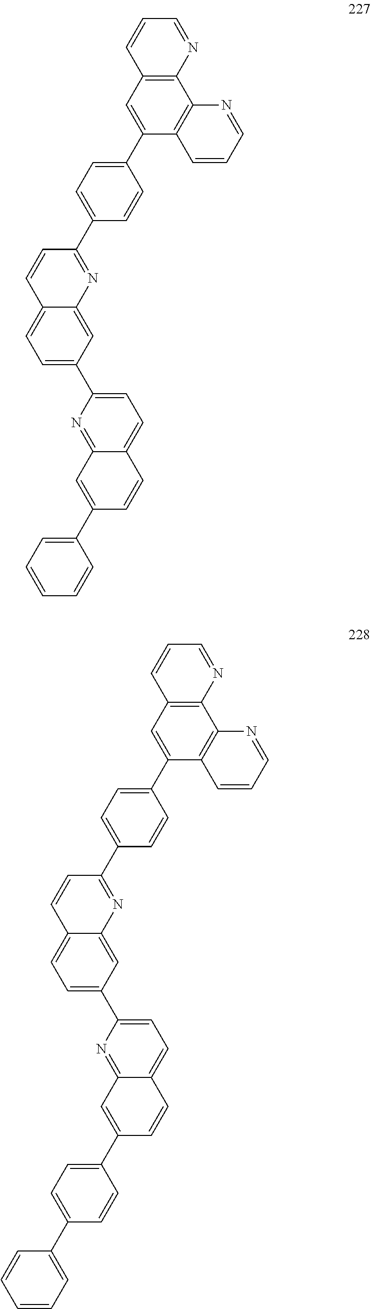 [SMILES: c1ccc(-c2ccc3ccc(-c4ccc5ccc(-c6ccc(-c7cc8cccnc8c8ncccc78)cc6)nc5c4)nc3c2)cc1 ; c1ccc(-c2ccc(-c3ccc4ccc(-c5ccc6ccc(-c7ccc(-c8cc9cccnc9c9ncccc89)cc7)nc6c5)nc4c3)cc2)cc1]

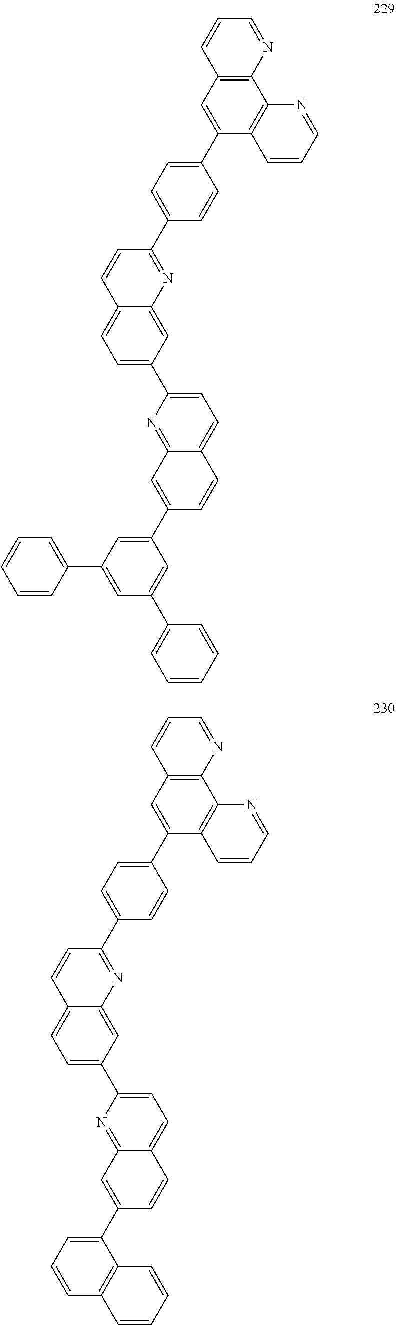

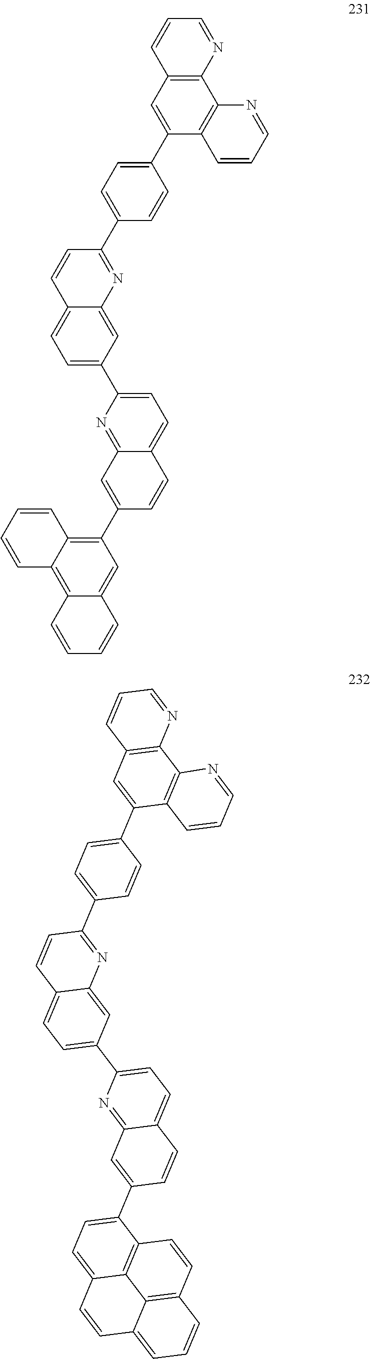

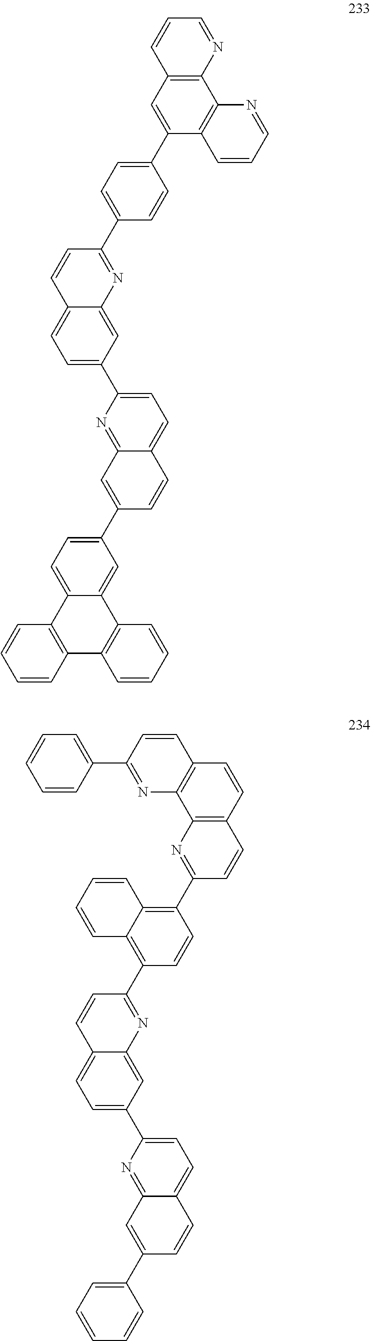 [SMILES: c1cnc2c(c1)cc(-c1ccc(-c3ccc4ccc(-c5ccc6ccc(-c7ccc8c9ccccc9c9ccccc9c8c7)cc6n5)cc4n3)cc1)c1cccnc12 ; c1ccc(-c2ccc3ccc(-c4ccc5ccc(-c6ccc(-c7ccc8ccc9ccc(-c%10ccccc%10)nc9c8n7)c7ccccc67)nc5c4)nc3c2)cc1]

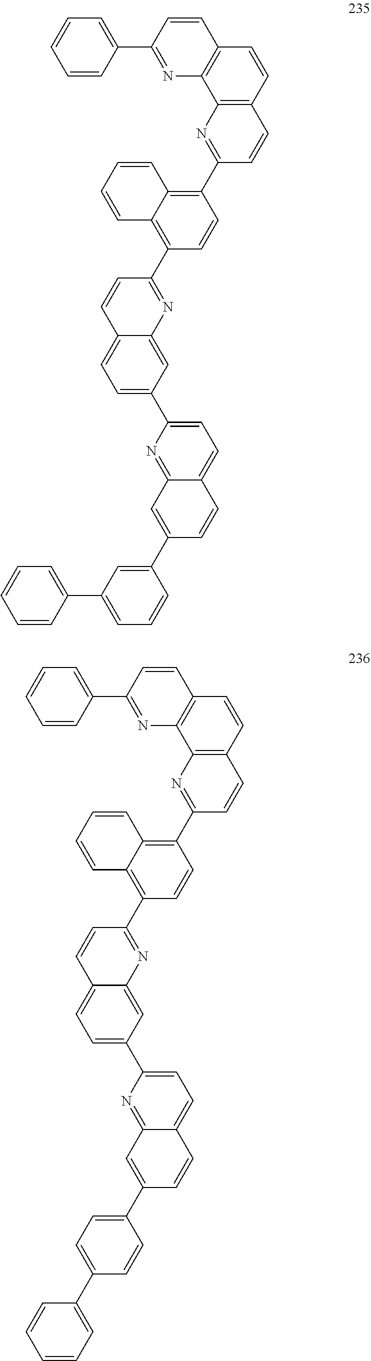

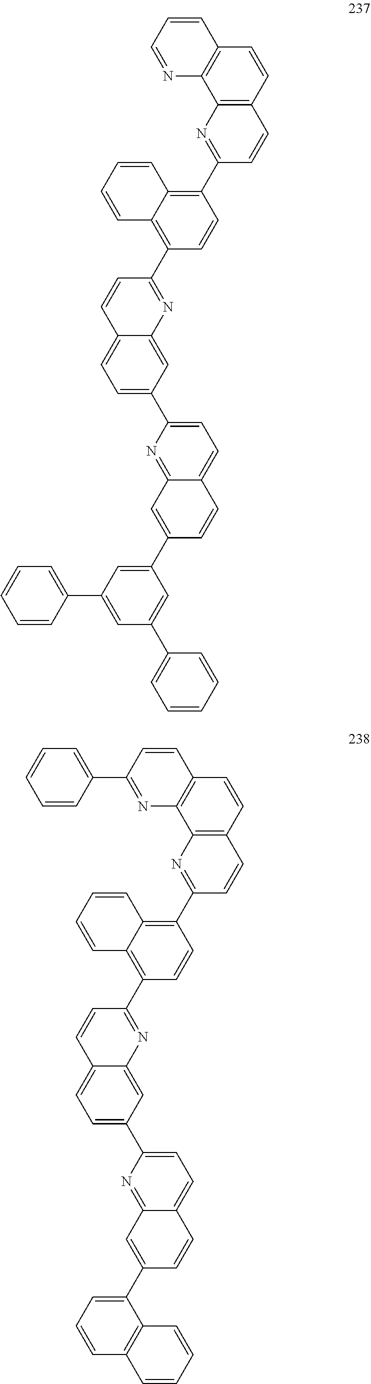

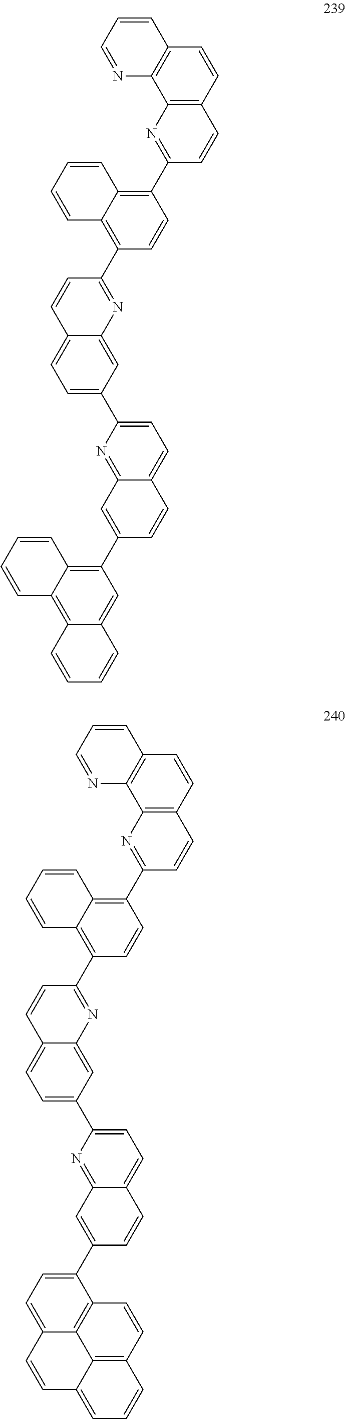

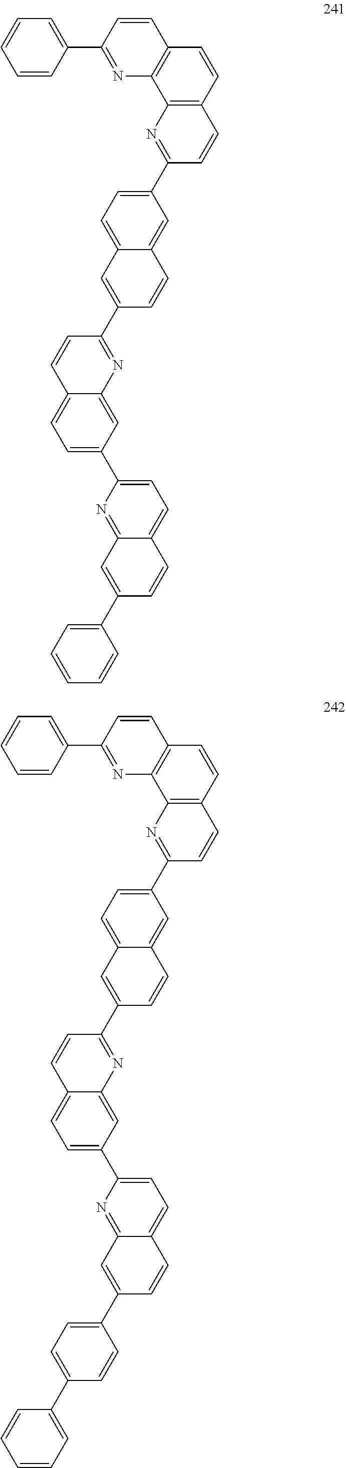

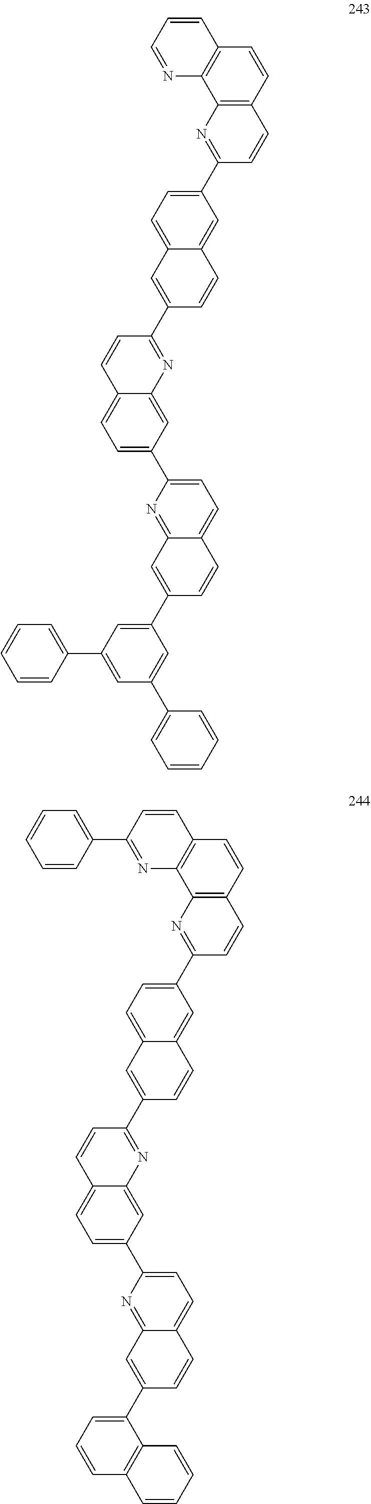

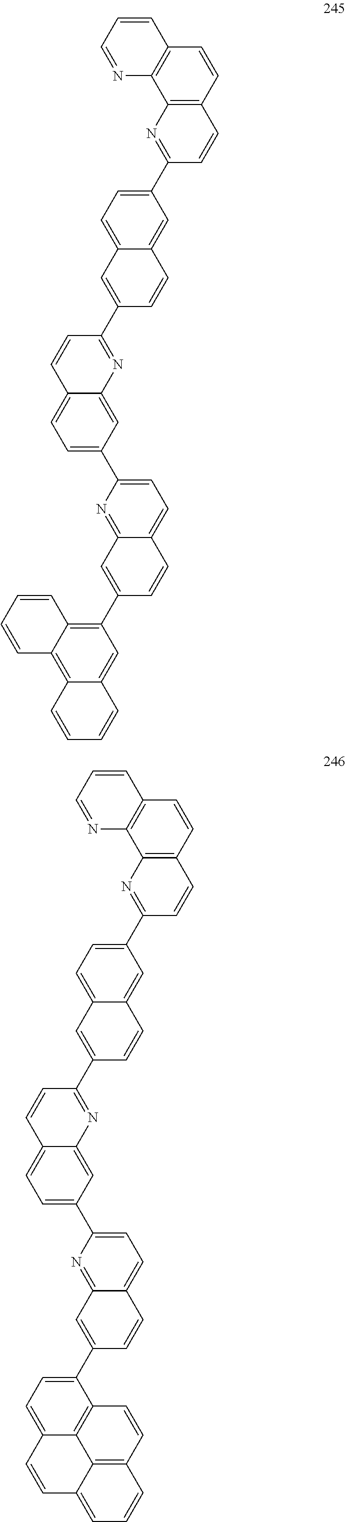

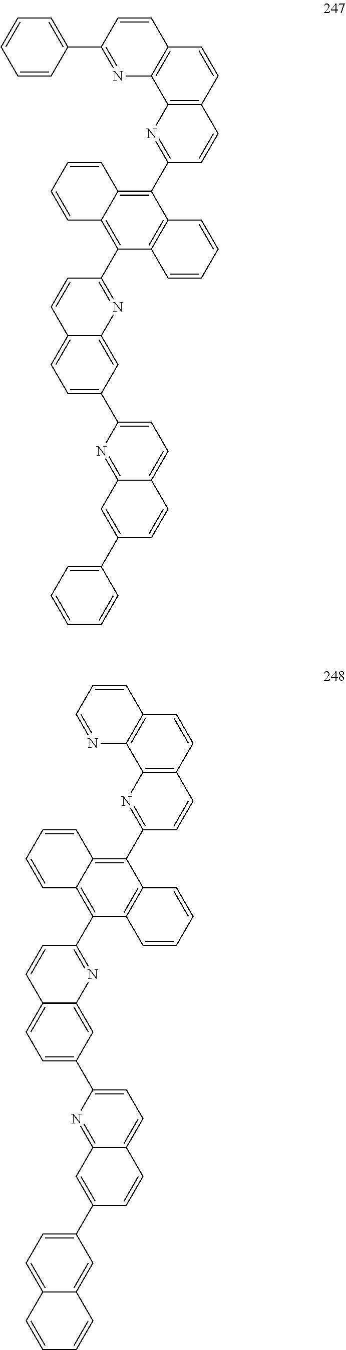 [SMILES: c1ccc(-c2ccc3ccc(-c4ccc5ccc(-c6c7ccccc7c(-c7ccc8ccc9ccc(-c%10ccccc%10)nc9c8n7)c7ccccc67)nc5c4)nc3c2)cc1 ; c1ccc2cc(-c3ccc4ccc(-c5ccc6ccc(-c7c8ccccc8c(-c8ccc9ccc%10cccnc%10c9n8)c8ccccc78)nc6c5)nc4c3)ccc2c1]

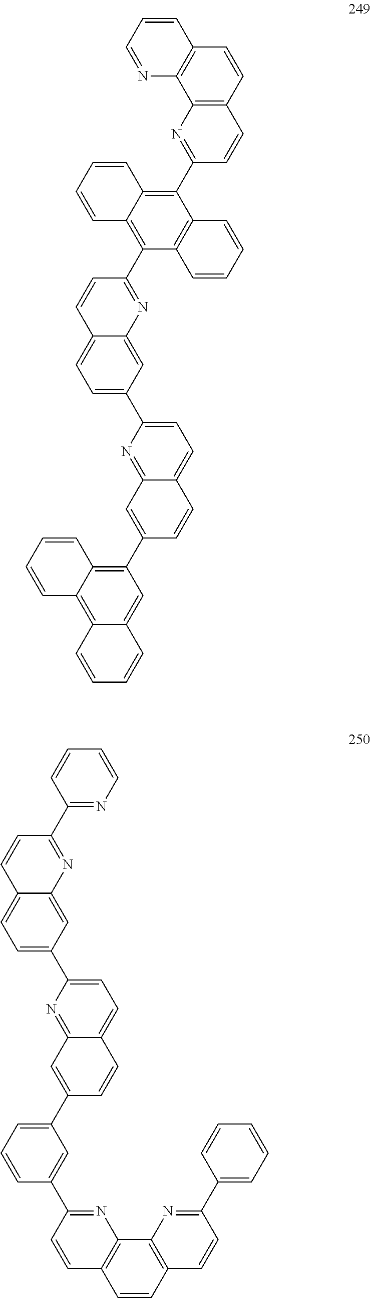 [SMILES: c1ccc2c(c1)cc(-c1ccc3ccc(-c4ccc5ccc(-c6c7ccccc7c(-c7ccc8ccc9cccnc9c8n7)c7ccccc67)nc5c4)nc3c1)c1ccccc12 ; c1ccc(-c2ccc3ccc4ccc(-c5cccc(-c6ccc7ccc(-c8ccc9ccc(-c%10ccccn%10)nc9c8)nc7c6)c5)nc4c3n2)cc1]

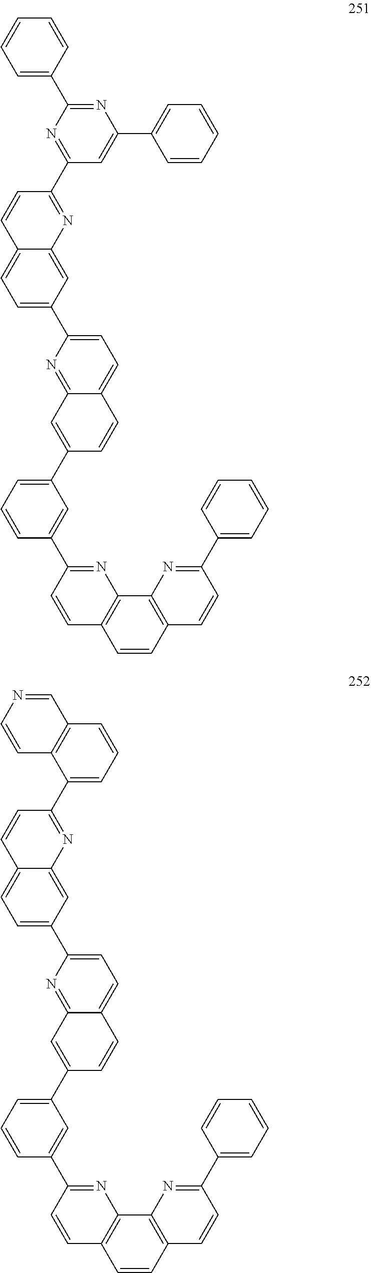 [SMILES: c1ccc(-c2cc(-c3ccc4ccc(-c5ccc6ccc(-c7cccc(-c8ccc9ccc%10ccc(-c%11ccccc%11)nc%10c9n8)c7)cc6n5)cc4n3)nc(-c3ccccc3)n2)cc1 ; c1ccc(-c2ccc3ccc4ccc(-c5cccc(-c6ccc7ccc(-c8ccc9ccc(-c%10cccc%11cnccc%10%11)nc9c8)nc7c6)c5)nc4c3n2)cc1]

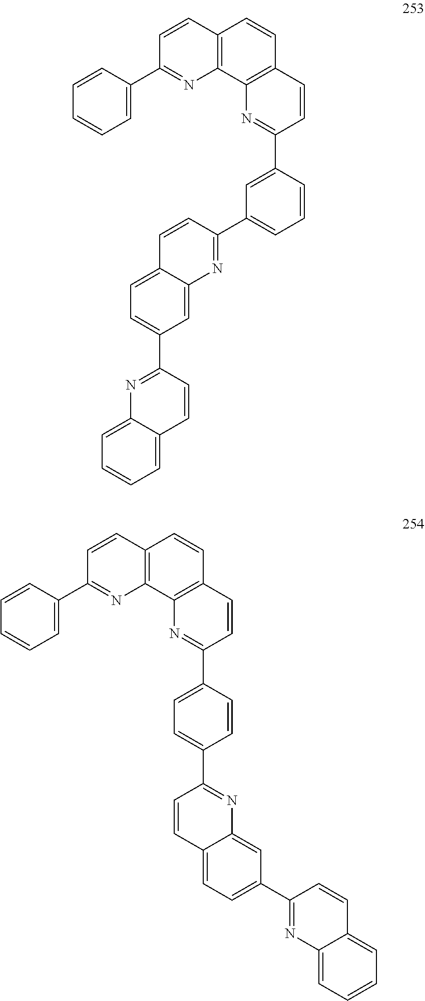 [SMILES: c1ccc(-c2ccc3ccc4ccc(-c5cccc(-c6ccc7ccc(-c8ccc9ccccc9n8)cc7n6)c5)nc4c3n2)cc1 ; c1ccc(-c2ccc3ccc4ccc(-c5ccc(-c6ccc7ccc(-c8ccc9ccccc9n8)cc7n6)cc5)nc4c3n2)cc1]

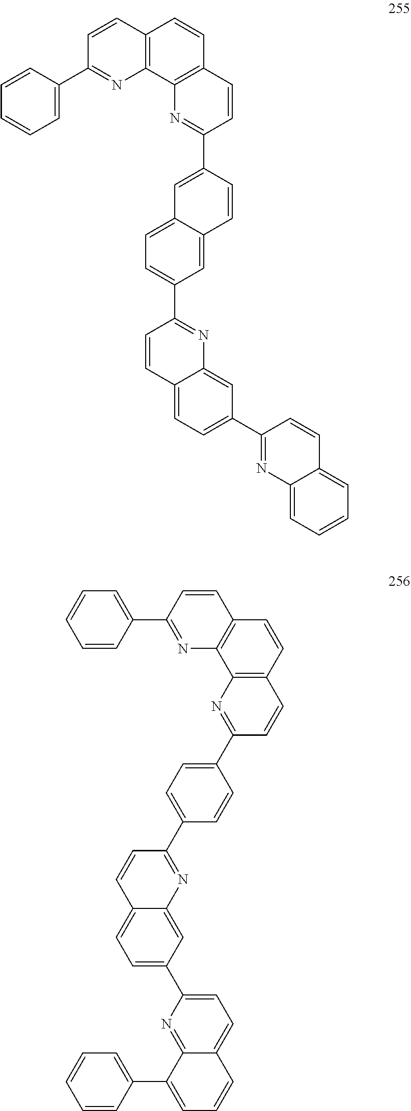 [SMILES: c1ccc(-c2ccc3ccc4ccc(-c5ccc6cc(-c7ccc8ccc(-c9ccc%10ccccc%10n9)cc8n7)ccc6c5)nc4c3n2)cc1 ; c1ccc(-c2ccc3ccc4ccc(-c5ccc(-c6ccc7ccc(-c8ccc9cccc(-c%10ccccc%10)c9n8)cc7n6)cc5)nc4c3n2)cc1]

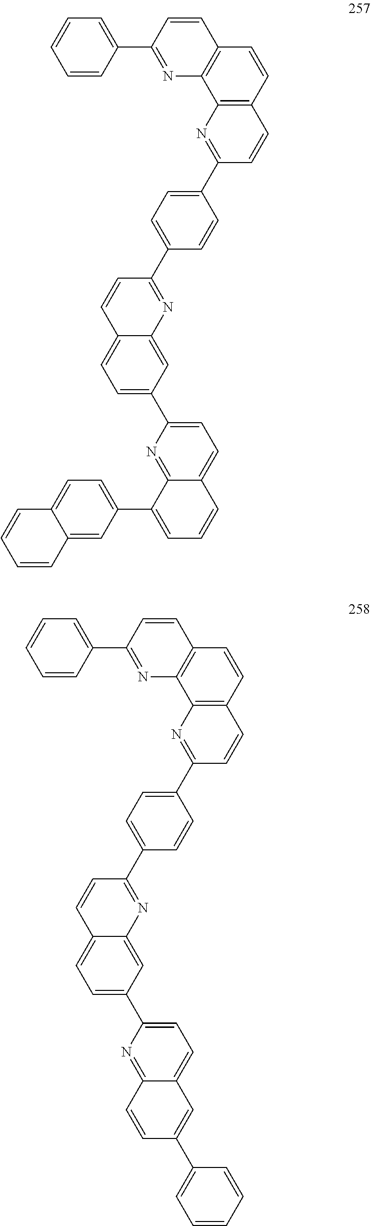 [SMILES: c1ccc(-c2ccc3ccc4ccc(-c5ccc(-c6ccc7ccc(-c8ccc9cccc(-c%10ccc%11ccccc%11c%10)c9n8)cc7n6)cc5)nc4c3n2)cc1 ; c1ccc(-c2ccc3nc(-c4ccc5ccc(-c6ccc(-c7ccc8ccc9ccc(-c%10ccccc%10)nc9c8n7)cc6)nc5c4)ccc3c2)cc1]

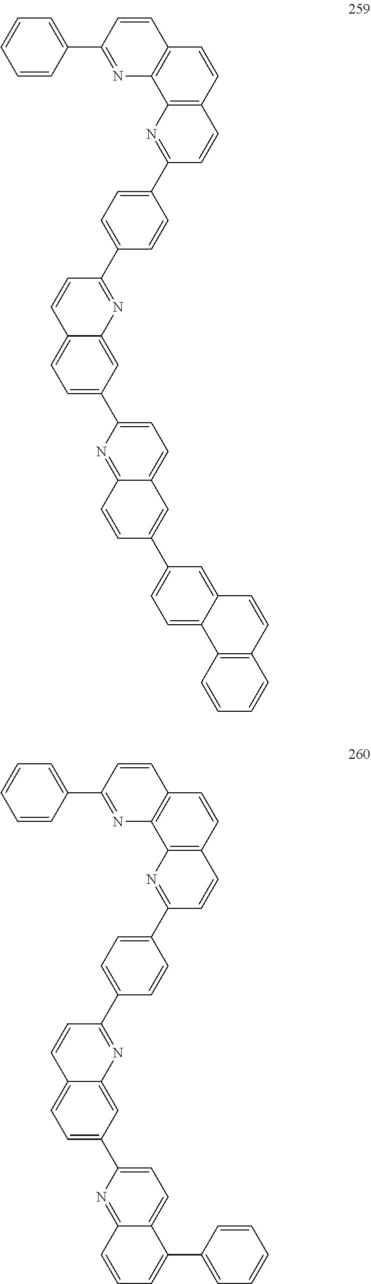 [SMILES: c1ccc(-c2ccc3ccc4ccc(-c5ccc(-c6ccc7ccc(-c8ccc9cc(-c%10ccc%11c(ccc%12ccccc%12%11)c%10)ccc9n8)cc7n6)cc5)nc4c3n2)cc1 ; c1ccc(-c2ccc3ccc4ccc(-c5ccc(-c6ccc7ccc(-c8ccc9c(-c%10ccccc%10)cccc9n8)cc7n6)cc5)nc4c3n2)cc1]

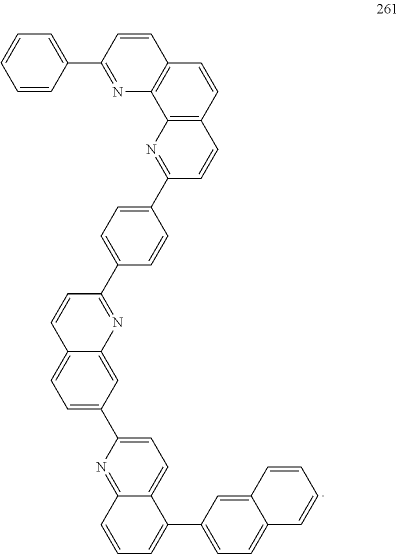 [SMILES: c1ccc(-c2ccc3ccc4ccc(-c5ccc(-c6ccc7ccc(-c8ccc9c(-c%10ccc%11ccccc%11c%10)cccc9n8)cc7n6)cc5)nc4c3n2)cc1]

7. An organic light emitting device comprising:
a first electrode;
a second electrode; and
an organic material layer provided between the first electrode and the second electrode,
wherein the organic material layer includes the heterocyclic compound of claim 1.

8. The organic light emitting device of claim 7, wherein the organic material layer includes a charge generation layer, and the charge generation layer includes the heterocyclic compound.

9. The organic light emitting device of claim 7, wherein the organic material layer includes a hole blocking layer, and the hole blocking layer includes the heterocyclic compound.

10. The organic light emitting device of claim 7, further comprising one, two or more layers selected from the group consisting of a light emitting layer, a hole injection layer, a hole transfer layer, an electron injection layer, an electron transfer layer, an electron blocking layer and a hole blocking layer.

11. The organic light emitting device of claim 7 comprising:
the first electrode;
a first stack provided on the first electrode and including a first light emitting layer;
a charge generation layer provided on the first stack;
a second stack provided on the charge generation layer and including a second light emitting layer; and
the second electrode provided on the second stack.

12. The organic light emitting device of claim 11, wherein the charge generation layer includes the heterocyclic compound.

13. The organic light emitting device of claim 11, wherein the charge generation layer is an N-type charge generation layer, and the N-type charge generation layer includes the heterocyclic compound.
